(19) United States
(12) Patent Application Publication
(10) Pub. No.: US 2024/0002397 A1
(43) Pub. Date: Jan. 4, 2024

(54) 3-AMINO PYRROLIDINE AND PIPERIDINE MACROCYCLIC OREXIN RECEPTOR AGONISTS

(71) Applicant: Merck Sharp & Dohme LLC, Rahway, NJ (US)

(72) Inventors: Stephane L. Bogen, Somerset, NJ (US); Ping Chen, Edison, NJ (US); Dane James Clausen, Rahway, NJ (US); Jian Liu, Edison, NJ (US); Michael T. Rudd, Collegeville, PA (US); Li Xiao, Cranbury, NJ (US); Dexi Yang, Livingston, NJ (US); Shishi Lin, Somerset, NJ (US)

(73) Assignee: Merck Sharp & Dohme LLC, Rahway, NJ (US)

(21) Appl. No.: 18/252,217

(22) PCT Filed: Nov. 18, 2021
(86) PCT No.: PCT/US2021/059862

§ 371 Date: May 9, 2023

Related U.S. Application Data
(60) Provisional application No. 63/279,900, filed on Nov. 16, 2021.
(60) Provisional application No. 63/117,031, filed on Nov. 23, 2020.

(51) Int. Cl. C07D 498/04; C07D 498/18

(52) U.S. Cl. CPC C07D 498/04; C07D 498/18

(57) ABSTRACT

The present invention is directed to 3-amino pyrrolidine and piperidine macrocyclic compounds which are agonists of orexin receptors. The present invention is also directed to uses of the compounds described herein in the potential treatment or prevention of neurological and psychiatric disorders and diseases in which orexin receptors are involved. The present invention is also directed to compositions comprising these compounds. The present invention is also directed to uses of these compositions in the potential prevention or treatment of such diseases in which orexin receptors are involved.

### BACKGROUND OF THE INVENTION

The orexins (hypocretins) comprise two neuropeptides produced in the hypothalamus: orexin A (OX-A) (a 33 amino acid peptide) and the orexin B (OX-B) (a 28 amino acid peptide) (Sakurai T. et al., Cell, 1998, 92, 573-585). Orexins regulate states of sleep and wakefulness opening potentially novel therapeutic approaches for narcolepsy, idiopathic hypersomnia, excessive daytime sleepiness, shift work disorder, obstructive sleep apnea and insomnia (Chemelli R. M. et al., Cell, 1999, 98, 437-451). Orexins are found to stimulate food consumption in rats suggesting a physiological role for these peptides as mediators in the central feedback mechanism that regulates feeding behavior (Sakurai T. et al., Cell, 1998, 92, 573-585). Orexins have also been indicated as playing a role in arousal, emotion, energy homeostasis, reward, learning and memory (Peyron, et al., Journal Neurosci., 1998, 18(23):9996-100150, Harris, et al., Trends Neurosci., 2006, 29 (10), 571-577). Two orexin receptors have been cloned and characterized in mammals. They belong to the super family of G-protein coupled receptors (Sakurai T. et al., Cell, 1998, 92, 573-585): the orexin-1 receptor (OX or OX1R) is partially selective for OX-A and the orexin-2 receptor (OX2 or OX2R) is capable of binding OX-A as well as OX-B with similar affinity. The physiological actions in which orexins are presumed to participate are thought to be expressed via one or both of OX1 receptor and OX2 receptor as the two subtypes of orexin receptors.

### SUMMARY OF THE INVENTION

The present invention is directed to 3-amino pyrrolidine and piperidine macrocyclic compounds which are agonists of orexin receptors. The present invention is also directed to uses of the compounds described herein in the potential treatment or prevention of neurological and psychiatric disorders and diseases in which orexin receptors are involved. The present invention is also directed to compositions comprising these compounds. The present invention is also directed to uses of these compositions in the potential prevention or treatment of such diseases in which orexin receptors are involved.

### DETAILED DESCRIPTION OF THE INVENTION

The present invention is directed to compounds of the formula I:

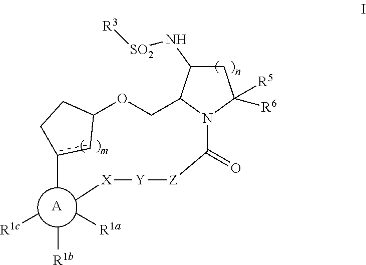

wherein:
[symbol] represents a line that may be absent or present as a double bond;
m is 1 or 2;
n is 1 or 2;
A is a phenyl or pyridyl ring;
X is —O— or —NR—, or X may be a direct bond to Y;
Y is C1-6alkyl or C2-6alkenyl;
Z is —O— or —NR—, or Z may be a direct bond to Y;
R is independently selected from H or C1-6alkyl;
R1a, R1b and R1c as present are independently selected from:
   (1) hydrogen,
   (2) halogen,
   (3) hydroxyl,
   (4) C₁₋₆alkyl, which is unsubstituted or substituted with one to three substituents selected from: hydroxy, fluoro and phenyl,
   (5) —O—C₁₋₆alkyl, which is unsubstituted or substituted with one to three substituents selected from: fluoro and phenyl,
   (6) C₃₋₆cycloalkyl,
   (7) C₂₋₆alkynyl,
   (8) —NH₂,
   (9) —NH(C₁₋₆alkyl),
   (10) —N(C₁₋₆alkyl)₂,
   (11) —(CO)—O—C₁₋₆alkyl,
   (12) keto,
   (13) -phenyl,
   (14) -pyridyl, and
   (15) —CN;
R³ is selected from:
   (1) —C₁₋₆alkyl, where the alkyl is unsubstituted or substituted with one to three fluoro,
   (2) —C₃₋₆cycloalkyl,
   (3) —NH₂,
   (4) —NH(C₁₋₆alkyl),
   (5) —N(C₁₋₆alkyl)(C₁₋₆alkyl), and
   (6) -phenyl;
R⁵ and R⁶ are independently selected from:
   (1) hydrogen, and
   (2) —C₁₋₆alkyl, where the alkyl is unsubstituted or substituted with OR, NR₂, —C(O)NR₂, or one to three fluoro, and
   (3) —C₃₋₆cycloalkyl;
or a pharmaceutically acceptable salt thereof.

Another embodiment of the instant invention is directed to compounds of the formula I′:

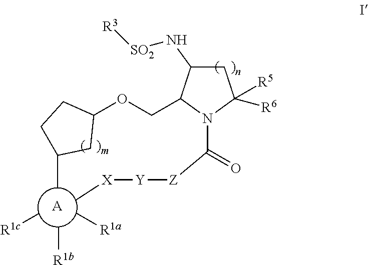

wherein:
m is 1 or 2;
n is 1 or 2;
A is a phenyl or pyridyl ring;
X is —O— or —NR—, or X may be a direct bond to Y;
Y is C₁₋₆alkyl or C₂₋₆alkenyl;
Z is —O— or —NH—, or Z may be a direct bond to Y;
R is independently selected from H or C₁₋₆alkyl;
R^{1a}, R^{1b} and R^{1c} as present are independently selected from:
   (1) hydrogen,
   (2) halogen,
   (3) hydroxyl,
   (4) C₁₋₆alkyl, which is unsubstituted or substituted with one to three substituents selected from: hydroxy, fluoro and phenyl,
   (5) —O—C₁₋₆alkyl, which is unsubstituted or substituted with one to three substituents selected from: fluoro and phenyl,
   (6) C₃₋₆cycloalkyl,
   (7) C₂₋₆alkynyl,
   (8) —NH₂,
   (9) —NH(C₁₋₆alkyl),
   (10) —N(C₁₋₆alkyl)₂,
   (11) —(CO)—O—C₁₋₆alkyl,
   (12) keto,
   (13) -phenyl,
   (14) -pyridyl, and
   (15) —CN;
R³ is selected from:
   (1) —C₁₋₆alkyl, where the alkyl is unsubstituted or substituted with one to three fluoro,
   (2) —C₃₋₆cycloalkyl,
   (3) —NH₂,
   (4) —NH(C₁₋₆alkyl),
   (5) —N(C₁₋₆alkyl)(C₁₋₆alkyl), and
   (6) -phenyl;
R⁵ and R⁶ are independently selected from:
   (1) hydrogen, and
   (2) C₁₋₆alkyl, where the alkyl is unsubstituted or substituted with one to three substituents selected from hydroxyl, —O—C₁₋₆alkyl, —NR₂, —C(O)NR₂. or one to three fluoro, and
   (3) —C₃₋₆cycloalkyl;
or a pharmaceutically acceptable salt thereof.

An embodiment of the present invention includes compounds of the formula Ia:

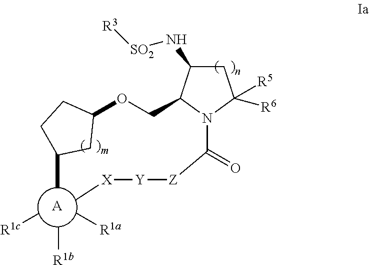

wherein m, n, A, X, Y, Z, R, RIa, R1b, R1c, R³, R⁵ and R⁶ are defined hereinabove; or a pharmaceutically acceptable salt thereof.

An embodiment of the present invention includes compounds of the formula Ib:

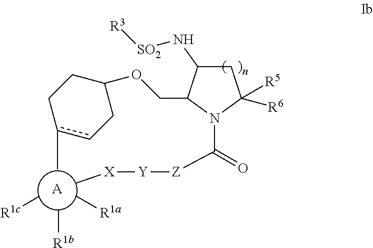

wherein the dashed line ( [symbol] ), n, A, X, Y, Z, R, R^{1a}, R^{1b}, R^{1c}, R³, R⁵ and R⁶ are defined herein; or a pharmaceutically acceptable salt thereof.

An embodiment of the present invention includes compounds of the formula Ib′:

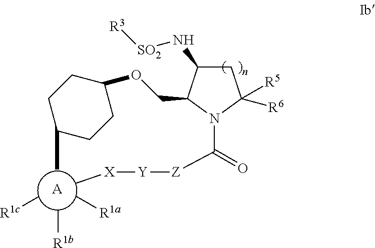

wherein n, A, X, Y, Z, R, R^{1a}, R^{1b}, R^{1c}, R³, R⁵ and R⁶ are defined herein; or a pharmaceutically acceptable salt thereof.

An embodiment of the present invention includes compounds of the formula Ib″:

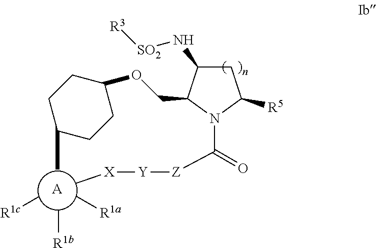

wherein n, A, X, Y, Z, R, R^{1a}, R^{1b}, R^{1c}, R³ and R⁵ are defined herein; or a pharmaceutically acceptable salt thereof.

An embodiment of the present invention includes compounds of the formula Ic:

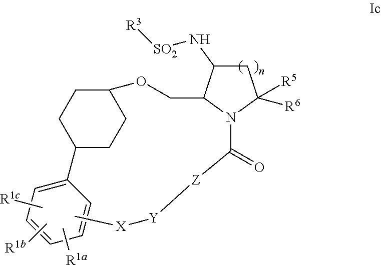

wherein n, X, Y, Z, R, R^{1a}, R^{1b}, R^{1c}, R³, R⁵ and R⁶ are defined herein; or a pharmaceutically acceptable salt thereof.

An embodiment of the present invention includes compounds of the formula Ic′:

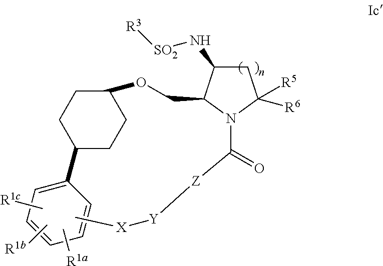

wherein n, X, Y, Z, R, R^{1a}, R^{1b}, R^{1c}, R³, R⁵ and R⁶ are defined herein; or a pharmaceutically acceptable salt thereof.

An embodiment of the present invention includes compounds of the formula Ic″:

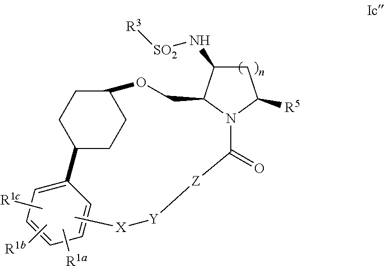

wherein n, X, Y, Z, R, R^{1a}, R^{1b}, R^{1c}, R³, and R⁵ are defined herein; or a pharmaceutically acceptable salt thereof.

An embodiment of the present invention includes compounds of the formula Id:

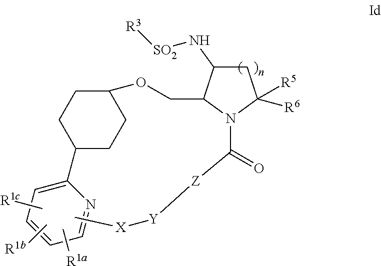

wherein n, X, Y, Z, R, R^{1a}, R^{1b}, R^{1c}, R³, R⁵ and R⁶ are defined herein; or a pharmaceutically acceptable salt thereof.

An embodiment of the present invention includes compounds wherein m is 1 (to form a cyclopentyl ring). An embodiment of the present invention includes compounds wherein m is 2 (to form a cyclohexyl ring).

An embodiment of the present invention includes compounds wherein n is 1 (to form a pyrrolidine ring). An embodiment of the present invention includes compounds wherein n is 2 (to form a piperidine ring). An embodiment of the present invention includes compounds wherein m is 2 (to form a cyclohexyl ring) and n is 1 (to form a pyrrolidine ring).

An embodiment of the present invention includes compounds wherein A is phenyl. An embodiment of the present invention includes compounds wherein A is a pyridyl. An embodiment of the present invention includes compounds wherein A is 1,2-phenyl, 1,3-phenyl or 2,6-pyridyl. An embodiment of the present invention includes compounds wherein A is 1,3-phenyl. An embodiment of the present invention includes compounds wherein A is 2,6-pyridyl.

An embodiment of the present invention includes compounds wherein X is —O—. An embodiment of the present invention includes compounds wherein X is —NR—. An embodiment of the present invention includes compounds wherein X is —NH— or —N(CH₃)—. An embodiment of the present invention includes compounds wherein X is a direct bond to Y.

An embodiment of the present invention includes compounds wherein Y is selected from:
(1) —C₂₋₅alkyl, and
(2) —C₂₋₄alkenyl.

An embodiment of the present invention includes compounds wherein Y is selected from:
(1) —CH₂CH₂—,
(2) —CH₂CH₂CH₂—,
(3) —CH₂CH₂CH₂CH₂—,
(4) —CH₂CH₂CH₂CH₂CH₂—,
(5) —CH═CH—,
(6) —CH═CHCH₂—,
(7) —CH₂CH═CH—,
(8) —CH═CHCH₂CH₂—,
(9) —CH₂CH═CHCH₂—, and
(10) —CH₂CH₂CH═CH—.

An embodiment of the present invention includes compounds wherein Y is selected from:
(1) —CH₂CH₂—,
(2) —CH₂CH₂CH₂—, and
(3) —CH₂CH₂CH₂CH₂—.

An embodiment of the present invention includes compounds wherein Z is —O—. An embodiment of the present invention includes compounds wherein Z is —NR—. An embodiment of the present invention includes compounds wherein Z is a direct bond to Y.

An embodiment of the present invention includes compounds wherein R^{1a}, R^{1b} and R^{1c} as are present are independently selected from:
(1) hydrogen,
(2) fluoro,
(3) chloro,
(4) hydroxyl,
(5) C₁₋₃alkyl, which is unsubstituted or substituted with one to three substituents selected from: hydroxy or fluoro,
(6) —O—C₁₋₃alkyl, which is unsubstituted or substituted with one or more fluoro,
(7) C₃₋₆cycloalkyl,
(8) —NH₂,
(9) —NH(C₁₋₃alkyl),
(10) —N(C₁₋₃alkyl)₂,
(11) keto, and
(12) -phenyl.

An embodiment of the present invention includes compounds wherein R^{1a}, R^{1b} and R^{1c} as are present are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCF₃,
(12) —OCHF₂,
(13) —OCH₂CH₂F,
(14) —N(CH₃)₂,
(15) cyclopropyl, and
(16) phenyl.

An embodiment of the present invention includes compounds wherein R^{1c} is hydrogen and R^{1a} and R^{1b}, as are present, are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl,
(15) phenyl, and
(16) —OCF₃.

An embodiment of the present invention includes compounds wherein R^{1c} and R^{1b}, as are present, are hydrogen and R^{1a} is selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl,
(15) phenyl,
(16) —OCF₃.

An embodiment of the present invention includes compounds wherein R^{1c} and R^{1b}, as are present, are hydrogen and R^{1a} is selected from:
(1) hydrogen,
(2) fluoro,
(3) —CH₃,
(4) —CHF₂,
(5) —CF₃,
(6) —OCH₃,
(7) —OCHF₂, and
(8) —OCF₃.

An embodiment of the present invention includes compounds wherein R³ is selected from:
(1) methyl,
(2) —CF₃,
(3) —CH₂F,
(4) ethyl,
(5) cyclopropyl,
(6) —CH(CH₃)₂,
(7) —NH(CH₃),
(8) —N(CH₃)₂, and
(9) -phenyl.

An embodiment of the present invention includes compounds wherein R³ is selected from:
(1) —NH(CH₃), and
(2) —N(CH₃)₂.

An embodiment of the present invention includes compounds wherein R³ is selected from:
(1) methyl,
(2) —CH₂CH₃,
(2) —CF₃,
(3) —CH₂F,
(4) —CHF₂,
(5) cyclopropyl, and
(6) —CH(CH₃)₂.

An embodiment of the present invention includes compounds wherein R³ is selected from:
(1) methyl,
(2) —CF₃,
(3) —CH₂F, and
(4) —CHF₂.

An embodiment of the present invention includes compounds wherein R⁵ and R⁶ are independently selected from:
(1) hydrogen,
(2) methyl,
(3) ethyl,
(4) —CHF₂,
(5) —CF₃,
(6) —CH₂OH,
(7) —CH₂OCH₃, and
(8) cyclopropyl.

An embodiment of the present invention includes compounds wherein R⁶ is hydrogen. An embodiment of the present invention includes compounds wherein R⁵ is methyl or —CH₂OCH₃, and R⁶ is hydrogen. An embodiment of the present invention includes compounds wherein R⁵ is methyl and R⁶ is hydrogen.

Certain embodiments of the present invention include a compound which is selected from the group consisting of the subject compounds of the Examples herein or a pharmaceutically acceptable salt thereof.

Certain embodiments of the present invention include a compound which is selected from:
N′-((2¹R,2⁴R,5²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)-N,N-dimethyl-sulfamide;
N-((2¹R,2⁴R,5²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)methanesulfonamide;
N-((2¹R,2⁴R,5²R,5³S,5⁵S,E)-1³-fluoro-5⁵-(methoxymethyl)-6-oxo-3,7-dioxa-5(2,1)-pyrrolidina-1(1,2)-benzena-2(1,4)-cyclohexanacyclononaphan-8-en-5³-yl)methanesulfonamide;
N′-((2¹R,2⁴R,5²R,5³S,5⁵R)-5⁵-methyl-6-oxo-3,7-dioxa-5(2,1)-pyrrolidina-1(1,2)-benzena-2(1,4)- cyclohexanacyclononaphane-5³-yl)-N,N-dimethyl-sulfamide;
N-((2¹R,2⁴R,5²R,5³S,5⁵R)-5⁵-methyl-6-oxo-3,7-dioxa-5(2,1)-pyrrolidina-1(1,2)-benzena-2(1,4)- cyclohexanacyclononaphane-5³-yl)methanesulfonamide;
N-((2¹R,2⁴R,5²R,5³S,5⁵S)-1³-fluoro-5⁵-(methoxymethyl)-6-oxo-3,7-dioxa-5(2,1)-pyrrolidina-1(1,2)-benzena-2(1,4)-cyclohexanacyclononaphane-5³-yl)methanesulfonamide;
N′-((2¹S,2⁴S,5²R,5³S,5⁵R)-6-oxo-3,11-dioxa-5(2,1)-pyrrolidina-1(1,3)-benzena-2(1,4) cyclohexanacycloundecaphane-5³-yl)-N,N-dimethyl-sulfamide;
N′-((2¹S,2⁴S,5²R,5³S,5⁵R)-5⁵-methyl-6-oxo-3,12-dioxa-5(2,1)-pyrrolidina-1(1,3)-benzena-2(1,4)- cyclohexanacyclododecaphane-5³-yl)-N,N-dimethyl-sulfamide;
N′-((2¹S,2⁴S,5²R,5³S, 5⁵R)-5⁵-methyl-6-oxo-3,11-dioxa-5(2,1)-pyrrolidina-1(1,3)-benzena-2(1,4)-cyclohexanacycloundecaphan-7-en-5³-yl)-N,N-dimethyl-sulfamide;
N′-((2¹S,2⁴S,5²R,5³S, 5⁵R)-5⁵-methyl-6-oxo-3,11-dioxa-5(2,1)-pyrrolidina-1(1,3)-benzena-2(1,4)-cyclohexanacycloundecaphane-5³-yl)-N,N-dimethyl-sulfamide;
N′-((2¹S,2⁴S,5²R,5³S,5⁵R)-5⁵-methyl-6-oxo-3,7,12-trioxa-5(2,1)-pyrrolidina-1(1,3)-benzena-2(1,4)-cyclohexanacyclododecaphane-5³-yl)-N,N-dimethyl-sulfamide;
N′-((2¹S,2⁴S,5²R,5³S,5⁵R)-5⁵-methyl-6-oxo-3-oxa-5(2,1)-pyrrolidina-1(1,3)-benzena-2(1,4)cyclohexanacycloundecaphane-5³-yl)-N,N-didimethyl-sulfamide;
N′-((2¹S,2⁴S,5²R,5³S,5⁵R)-)-5⁵-methyl-6-oxo-3,7,11-trioxa-5(2,1)-pyrrolidina-1(1,3)-benzena-2(1,4)-cyclohexanacycloundecaphane-5³-yl)-N,N-dimethyl-sulfamide;
N′-((2¹S,2⁴S,5²R,5³S,5⁵R)-5⁵-methyl-6-oxo-3,10-dioxa-5(2,1)-pyrrolidina-1(1,3)-benzena-2(1,4)- cyclohexanacyclodecaphane-5³-yl)-N,N-methyl-sulfamide;
N′-((2¹S,2⁴S,5²R,5³S,5⁵R)-5⁵-methyl-6-oxo-3,7,10-trioxa-5(2,1)-pyrrolidina-1(1,3)-benzena-2(1,4)-cyclohexanacyclodecaphane-5³-yl)-N,N-dimethyl-sulfamide;
N′-((2¹S,2⁴S,5²R,5³S)-6-oxo-3,7,11-trioxa-5(2,1)-pyrrolidina-1(1,3)-benzena-2(1,4)-cyclohexanacycloundecaphane-5³-yl)-N,N-dimethyl-sulfamide;
N-((2¹S,2⁴S,5²R,5³S,5⁵R)-5⁵-methyl-6-oxo-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,5³S,5⁵R)-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-3,11-dioxa-5(2,1)-pyrrolidina-1(1,3)-benzena-2(1,4)- cyclohexanacycloundecaphane-5³-yl)methanesulfonamide;
N-((2¹S,2⁴S,5²R,5³S,5⁵R)-5⁵-methyl-6-oxo-3,10-dioxa-5(2,1)-pyrrolidina-1(1,3)-benzena-2(1,4)- cyclohexanacyclodecaphane-5³-yl)methanesulfonamide;
N′-((2¹S,2⁴S,5²R,5³S)-6-oxo-3,7-dioxa-5(2,1)-pyrrolidina-1(1,3)-benzena-2(1,4)-cyclohexanacycloundecaphane-5³-yl)-N,N-dimethyl-sulfamide;
N′-((2¹S,2⁴S,5²R,5³S,5⁵R)-5⁵-methyl-6-oxo-3,7-dioxa-5(2,1)-pyrrolidina-1(1,3)-benzena-2(1,4)- cyclohexanacycloundecaphane-5³-yl)-N,N-dimethysulfamide;
N′-((2¹S,2⁴S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide;
N-((2¹S,2⁴S,5²R,5³S)-6-oxo-3,1′-dioxa-5(2,1)-piperidina-1(1,2)-benzena-2(1,4)-cyclohexanacycloundecaphane-5³-yl)methanesulfonamide;
N′-((2¹S,2⁴S,5²R,5³S)-6-oxo-3,7,11-trioxa-5(2,1)-piperidina-1(1,3)-benzena-2(1,4)-cyclohexanacycloundecaphane-5³-yl)-N,N-dimethyl-sulfamide;
N′-((2¹S,2⁴S,5²R,5³S)-6-oxo-3,7,12-trioxa-5(2,1)-piperidina-1(1,3)-benzena-2(1,4)-cyclohexanacyclododecaphane-5³-yl)-N,N-dimethy-sulfamide;
N′-((2¹R,2⁴R,5²S,5³R)-6-oxo-3,7,12-trioxa-5(2,1)-piperidina-1(1,3)-benzena-2(1,4)-cyclohexanacyclododecaphane-5³-yl)-N,N-dimethyl-sulfamide;
N-((2¹S,2⁴S,5²R,5³S,E)-6-oxo-3,11-dioxa-5(2,1)-piperidina-1(1,3)-benzena-2(1,4)-cyclohexanacycloundecaphan-7-en-5³-yl)methanesulfonamide;
N-((2¹S,2⁴S,5²R,5³S)-6-oxo-3,11-dioxa-5(2,1)-piperidina-1(1,3)-benzena-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide; and
N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide;
N-(2⁴S,5²R,5³S,5⁵R)-1⁵fluoro-5⁵-methyl-6-oxo-3,7,11-trioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,5³S,5⁵R)-1⁵-fluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,11-dioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,5³S,5⁵R)-1⁵-fluoro-5⁵-methyl-6-oxo-3,11-dioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1⁴-cyano-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1²-fluoro-5⁵-methyl-6-oxo-3,7,11-trioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,5³S,5⁵R)-1²-fluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1²,1⁵-difluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-1⁵-(trifluoromethyl)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-1⁶-(trifluoromethoxy)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-14-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁴-(trifluoromethyl)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-16-(trifluoromethoxy)-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,5³S,5⁵R)-5⁵-methyl-6-oxo-1⁴-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁶-fluoro-5⁵-methyl-6-oxo-3,7,11-trioxa-1(3,5)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁵-fluoro-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1³-(trifluoromethoxy)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1³-(trifluoromethoxy)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁶-isopropoxy-5⁵-methyl-6-oxo-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,5³S,5⁵R)-1⁶-isopropoxy-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-1⁵-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1⁵-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴,5⁵-dimethyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴,5⁵-dimethyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1³-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁵-(trifluoromethoxy)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1⁵-(trifluoromethoxy)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-fluoro-1³-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-ethoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-ethoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1³,5⁵-dimethyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1³,5⁵-dimethyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1³,1⁵-difluoro-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1³,1⁵-difluoro-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵S)-5⁵-(methoxymethyl)-6-oxo-1⁴-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵S)-5⁵-(methoxymethyl)-6-oxo-1⁴-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵S)-14-(difluoromethyl)-5⁵-(methoxymethyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-(difluoromethyl)-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵S)-5⁵-(methoxymethyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-methoxy-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵,1¹-dimethyl-6-oxo-1⁴-(trifluoromethyl)-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵,1¹-dimethyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7-dioxa-10-aza-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁵-fluoro-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵,1¹-dimethyl-6-oxo-1³-(trifluoromethyl)-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵,1¹-dimethyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹R,2⁴R,5²R,5³S,5⁵S)-5⁵-(hydroxymethyl)-6-oxo-1³-(tifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵S)-1⁴-(difluoromethyl)-5⁵-(hydroxymethyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹R,2⁴R,5²R,5³S,5⁵S)-5⁵-((dimethylamino)methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl) methanesulfonamide;
2-((2¹R,2⁴R,5²R,5³S,5⁵S)-5³-(methylsulfonamido)-6-oxo-1³-(trifluoromethyl)-3,7,10-trioxa-1(2,6)-pyridina-5(2,1)-pyrrolidina-2(1,4)-cyclohexanacyclodecaphane-5⁵-yl)acetamide;
N-((2¹S,2⁴S,5²R,5³S,5⁵S)-1⁴-(difluoromethyl)-5⁵-((dimethylamino)methyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵S)-1⁴-(difluoromethyl)-5⁵-((methylamino)methyl)-6-oxo-3,7,11-trioxa-1(2,6)-pyridina-5(2,1)-pyrrolidina-2(1,4)-cyclohexanacycloundecaphane-5³-yl)methanesulfonamide;
2-((2¹S,2⁴S,5²R,5³S,5⁵S)-1⁴-(difluoromethyl)-5³-(methylsulfonamido)-6-oxo-3,7,11-trioxa-1(2,6)-pyridina-5(2,1)-pyrrolidina-2(1,4)-cyclohexanacycloundecaphane-5⁵-yl)acetamide;
N-((2¹R,2⁴R,5²R,5³S,5⁵S)-5⁵-((dimethylamino)methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
1-fluoro-N-((2¹R,2⁴R,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
1,1-difluoro-N-((2¹R,2⁴R,5²R,5³S,5⁵R)-14-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹R,2⁴R,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)ethanesulfonamide;
1-fluoro-N-((2¹R,2⁴R,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
1,1-difluoro-N-((2¹R,2⁴R,5²R,5³S,5⁵R)-14-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁴-(trifluoromethyl)-3,7-dioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7-dioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵-methyl-6-oxo-3,7-dioxa-1(2,6)-pyridina-5(2,1)-pyrrolidina-2(1,4)-cyclohexanacycloundecaphane-5³-yl)methanesulfonamide;
   or a pharmaceutically acceptable salt thereof.

Certain embodiments of the present invention include a compound which is selected from Example Numbers: 51, 56, 58, 67, 68, 70, 71, 89 and 105 or a pharmaceutically acceptable salt thereof.

Alternate embodiments of the present invention may also exclude any of the compounds which are recited in the list above.

It is understood that reference to “Formula I” also encompasses compounds of Formula I′, Formula Ia, Formula Ib, Formula Ib′, Formula Ib″, Formula Ic, Formula Ic′, Formula Ic″, and Formula Id, unless indicated otherwise.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Likewise, the present invention includes tautomeric forms of the compounds disclosed herein. Formula I shows the structure of the class of compounds without specific stereochemistry. At least some of the chemical names of compounds of the invention as set forth in this application may have been generated on an automated basis by use of commercially available chemical naming software programs, and have not been independently verified.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

As appreciated by those of skill in the art, halogen or halo as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature. Such compounds are identical to those disclosed herein, but for the fact that one or more atoms are replaced by an atom having an atomic mass or mass number different from the atomic mass or mass number usually found in nature. Examples of isotopes suitable for inclusion in the compounds of the invention include isotopes of hydrogen such as ²H and ³H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ¹²³I and ¹²⁵I, and chlorine such as ³⁶Cl. Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as IC, ¹⁸F, ¹⁵O and ¹³N, can be useful in Positron Emission Topography (PET) studies for examining substrate receptor occupancy. An embodiment of the present invention includes compounds that are substituted with a positron emitting isotope. An embodiment of the present invention includes compounds that are substituted with a IC isotope. An embodiment of the present invention includes compounds that are substituted with an ¹⁸F isotope. In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the schemes and examples herein using appropriate isotopically-enriched reagents and/or intermediates.

Those skilled in the art will recognize those instances in which the compounds of the invention may form salts. In such instances, another embodiment provides pharmaceutically acceptable salts of the compounds of the invention. Thus, reference to a compound of the invention herein is understood to include reference to salts thereof, unless otherwise indicated. The term “pharmaceutically acceptable salts” refers to salts prepared from pharmaceutically acceptable non-toxic bases or acids including inorganic or organic bases and inorganic or organic acids. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the present invention. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates or solvates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments include the citric, hydrobromic, hydrochloric, maleic, phosphoric, sulfuric, fumaric, and tartaric acids. It will be understood that, as used herein, references to the compounds of Formula I are meant to also include the pharmaceutically acceptable salts. Salts of the compounds of the invention may be formed by methods known to those of ordinary skill in the art, for example, by reacting a compound of the invention with an amount of acid or base, such as an equivalent amount, in a medium such as one in which the salt precipitates or in an aqueous medium followed by lyophilization.

Exemplifying the invention is the use of the compounds disclosed in the Examples and herein. Specific compounds within the present invention include a compound which is selected from the compounds disclosed in the following Examples and pharmaceutically acceptable salts thereof and individual enantiomers or diastereomers thereof.

The present invention is also directed to the use of the compounds disclosed herein as agonists of orexin receptor activity. The subject compounds and pharmaceutically acceptable salts thereof are useful in a method of agonizing orexin receptor activity in a subject such as a mammal comprising the administration of an amount of the compound. In addition to primates, especially humans, a variety of other mammals may be administered with a compound of the present invention. The present invention is directed to a compound of the present invention or a pharmaceutically acceptable salt thereof that could be useful in therapy. The present invention may further be directed to a use of a compound of the present invention or a pharmaceutically acceptable salt thereof for the manufacture of a medicament for agonizing orexin receptor activity or treating the disorders and diseases noted herein in humans and animals.

A subject administered with a compound of the present invention, or a pharmaceutically acceptable salt thereof, is generally a mammal, such as a human being, male or female. The amount of compound administered to the subject is an amount sufficient to agonize the orexin receptor in the subject. In an embodiment, the amount of compound can be an “effective amount”, wherein the subject compound is administered in an amount that will elicit the biological or medical response of a tissue, system, animal or human that is being sought by the researcher, veterinarian, medical doctor or other clinician. An effective amount does not necessarily include considerations of toxicity and safety related to the administration of the compound. It is recognized that one skilled in the art may affect neurological and psychiatric disorders associated with orexin receptor activation by treating a subject presently afflicted with the disorders, or by prophylactically treating a subject likely to be afflicted with the disorders, with an effective amount of a compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a subject that is predisposed to such disease or disorder. The terms “administration of” and or “administering a” compound should be understood to mean providing a compound of the invention or a prodrug of a compound of the invention to the subject.

The term “composition” as used herein is intended to encompass a product comprising the specified ingredients in the specified amounts, as well as any product which results, directly or indirectly, from combination of the specified ingredients in the specified amounts. Such term is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The utility of the compounds in accordance with the present invention as orexin receptor OX1R and/or OX2R agonists may be readily determined without undue experimentation by methodology well known in the art. Both the OX1R and/or OX2R G-coupled protein receptors (GPCRs) couple through the Gaq signaling pathway, which ultimately promotes calcium mobilization via inositol triphosphate (IP3) production. The half-life of IP-3 is relatively short, being rapidly metabolized to inositol monophosphate (IP-1), which can be readily detected using a commercially available assay kit (IP-One; Cisbio; cat #621PAPEC) coupled with a cell line expressing the target receptor(s) of interest. The utility of the compounds in accordance with the present invention as orexin receptor OX1R and/or OX2R agonists may be determined utilizing this assay.

In atypical experiment, the OX1 and OX2 receptor agonist activity is determined in accordance with the following general experimental method. Chinese hamster ovary (CHO) cells expressing human OX1R and/or the human OX2R were grown in Iscove's modified DMEM containing glutaMAX™, 1% G418, 100 U/mL penicillin, 100 μg/mL streptomycin and 10% heat-inactivated qualified fetal bovine serum (FBS). The OX2R cells were seeded at 10,000 cells/well/50 μL and the OX1R cells were seeded at 20,000 cells/well/50 μL into 384-well white tissue culture plates (Greiner; cat #781080). All cell/media reagents were from GIBCO-Invitrogen Corp. The seeded cell plate(s) were incubated at 37° C. with 5% CO₂ and 85% humidity for 20-24 hours. On the day of the assay, assay-ready compound plates were prepared using an acoustic liquid handler (ECHO; Labcyte), which dispensed sufficient volume of test compound stock (10 mM in DMSO) or 100% DMSO to prepare 10 point, ½-log dilutions in a final volume of 202.5 nL/well in all test wells of a 384-well diamond plate (Labcyte). Following completion of assay-ready plates, importantly, the next three steps were performed with minimal delay: 1) 20 μl of 1× stimulation buffer was added to the compound plate using a Multidrop Combi (small cassette, Thermo Fisher Scientific cat #24073290); 2) culture medium was removed from the cell plate using the Bluewasher plate washer (gentle spin; BlueCatBio); 3) 14 μ1 of compound/stimulation buffer mixture was added to the cell plate using a Bravo liquid handler (Agilent) prior to incubating cell plates at 37° C. with 5% CO₂ and 85% humidity for 1 or 2 hours (OX1R and OX2R, respectively). During this incubation, IP-one detection reagents were prepared (38:1:1 lysis buffer:D2:AB-cryptate reagents). Six μL of mixed detection reagents were added to the cell plate using a Multidrop Combi (small cassette, Thermo Fisher Scientific cat #24073290) and incubated 60 minutes at room temperature in the dark. Fluorescence signal was detected using an Envision plate reader (Perkin Elmer) [LANCE/DELFIA Dual Enh (Em: APC 665; Ex: Cy5 620)].

For each compound, data were fit to a four parameter logistic fit (ActivityBase software) and the EC₅₀ was reported as the inflection point of the resulting curve. Percent effect for each test compound was determined as the percentage of sample raw value/mean max effect, where the mean max effect was derived from the mean raw value of 32 control wells per assay plate (using Orexin A (cat #003-30) at 1 μM for human OX1R and a reference compound at 1 uM with 100% activity previously established by comparison to Orexin A for human OX2R). The intrinsic orexin receptor agonist activity of a compound which may be used in the present invention may be determined by these assays.

All of the final compounds of the following examples had activity in agonizing the human orexin-2 receptor in the aforementioned IPOne assay with an EC₅₀ of about 0.01 nM to 5000 nM. Additional data is provided in the following Examples. Such a result is indicative of the intrinsic activity of the compounds in use as agonists of orexin-1 receptor and/or the orexin-2 receptor. In general, one of ordinary skill in the art would appreciate that a substance is considered to effectively agonize the orexin receptor if it has an EC₅₀ in the IPOne assay of less than about 50 μM, or more specifically less than about 1000 nM.

The orexin receptors have been implicated in a wide range of biological functions. This has suggested a potential role for these receptors in a variety of disease processes in humans or other species. The compounds of the present invention could therefore potentially have utility in treating, preventing, ameliorating, controlling or reducing the risk of a variety of disorders associated with orexin receptors, including one or more of the following conditions or diseases: narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, wakefulness, nocturnal myoclonus, disturbances of consciousness, such as coma, REM sleep interruptions, jet-lag, excessive daytime sleepiness, shift workers' sleep disturbances, dyssomnias, sleep disorders, sleep disturbances, hypersomnia associated with depression, emotional/mood disorders, Alzheimer's disease or cognitive impairment, Parkinson's disease, Guillain-Barre syndrome, Kleine Levin syndrome, and sleep disorders which accompany aging; Alzheimer's sundowning; conditions associated with circadian rhythmicity as well as mental and physical disorders associated with travel across time zones and with rotating shift-work schedules; fibromyalgia; cardiac failure; diseases related to bone loss; sepsis; syndromes which are manifested by non-restorative sleep and muscle pain or sleep apnea which is associated with respiratory disturbances during sleep; conditions which result from a diminished quality of sleep; and other diseases related to general orexin system dysfunction.

Thus, in certain embodiments the present invention may provide methods for: treating or controlling narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, disturbances of consciousness, REM sleep interruptions, jet-lag, shift workers' sleep disturbances, dyssomnias, night terror, insomnias associated with depression, emotional/mood disorders, Alzheimer's disease or cognitive impairment; treating or controlling sleep disturbances associated with diseases such as neurological disorders including neuropathic pain and restless leg syndrome; treating or controlling addiction disorders; treating or controlling psychoactive substance use and abuse; enhancing cognition; increasing memory retention; treating or controlling obesity; treating or controlling diabetes and appetite, taste, eating, or drinking disorders; treating or controlling insulin resistance syndrome; treating or controlling hypothalamic diseases; treating or controlling depression; treating, controlling, ameliorating or reducing the risk of epilepsy, including absence epilepsy; treating or controlling pain, including neuropathic pain; treating or controlling Parkinson's disease; treating or controlling Guillain-Barre syndrome; treating or controlling Klein Levin syndrome; treating or controlling psychosis; treating or controlling dysthymic, mood, psychotic and anxiety disorders; treating side effects or complications due to anesthesia; reversal of anesthesia; reversal of anesthesia following surgery; treating or controlling depression, including major depression and major depression disorder; treating or controlling bipolar disorder; or treating, controlling, ameliorating or reducing the risk of schizophrenia, in a mammalian subject which comprises administering to the subject a compound of the present invention.

The compounds of the present invention may also potentially have utility in treating, preventing, ameliorating, controlling or reducing the risk of a variety of other disorders associated with orexin receptors, including one or more of the following conditions or diseases including enhancing sleep quality, improving sleep quality, increasing sleep efficiency, augmenting sleep maintenance; increasing the value which is calculated from the time that a subject sleeps divided by the time that a subject is attempting to sleep; improving sleep initiation; decreasing sleep latency or onset (the time it takes to fall asleep); decreasing difficulties in falling asleep; increasing sleep continuity; decreasing the number of awakenings during sleep; decreasing intermittent wakings during sleep; decreasing nocturnal arousals; decreasing the time spent awake following the initial onset of sleep; increasing the total amount of sleep; reducing the fragmentation of sleep; altering the timing, frequency or duration of REM sleep bouts; altering the timing, frequency or duration of slow wave (i.e. stages 3 or 4) sleep bouts; increasing the amount and percentage of stage 2 sleep; promoting slow wave sleep; enhancing EEG-delta activity during sleep; decreasing nocturnal arousals, especially early morning awakenings; increasing daytime alertness; reducing daytime drowsiness; treating or reducing excessive daytime sleepiness; increasing satisfaction with the intensity of sleep; increasing sleep maintenance; idiopathic insomnia; sleep problems; insomnia; night terror, insomnias associated with depression, emotional/mood disorders, Alzheimer's disease or cognitive impairment, as well as sleep walking and enuresis, and sleep disorders which accompany aging; Alzheimer's sundowning; conditions associated with circadian rhythmicity as well as mental and physical disorders associated with travel across time zones and with rotating shift-work schedules, conditions due to drugs which cause reductions in REM sleep as a side effect; fibromyalgia; syndromes which are manifested by non-restorative sleep and muscle pain or sleep apnea which is associated with respiratory disturbances during sleep; conditions which result from a diminished quality of sleep; increasing learning; augmenting memory; increasing retention of memory; eating disorders associated with excessive food intake and complications associated therewith, compulsive eating disorders, obesity (due to any cause, whether genetic or environmental), obesity-related disorders overeating, anorexia, bulimia, cachexia, dysregulated appetite control, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, lung disease, abnormal heart rhythms and arrythmias, myocardial infarction, congestive heart failure, coronary heart disease, acute and congestive heart failure; hypotension; hypertension; urinary retention; osteoporosis; angina pectoris; myocardinal infarction; ischemic or haemorrhagic stroke; subarachnoid haemorrhage; ulcers; allergies; benign prostatic hypertrophy; chronic renal failure; renal disease; impaired glucose tolerance; sudden death, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia, metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, intestinal motility dyskinesias, obesity-related gastro-esophageal reflux, hypothalmic diseases, hypophysis diseases, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, increased anesthetic risk, reducing the risk of secondary outcomes of obesity, such as reducing the risk of left ventricular hypertrophy; diseases or disorders where abnormal oscillatory activity occurs in the brain, including depression, migraine, neuropathic pain, Parkinson's disease, psychosis and schizophrenia, as well as diseases or disorders where there is abnormal coupling of activity, particularly through the thalamus; enhancing cognitive function, including cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders; treating or controlling Guillain-Barre syndrome; treating or controlling Klein Levin syndrome; treating or controlling psychosis; treating or controlling dysthymic, mood, psychotic and anxiety disorders; treating complications due to anesthesia; enhancing memory; increasing memory retention; increasing immune response; increasing immune function; hot flashes; night sweats; extending life span; schizophrenia; muscle-related disorders that are controlled by the excitation/relaxation rhythms imposed by the neural system such as cardiac rhythm and other disorders of the cardiovascular system; conditions related to proliferation of cells such as vasodilation or vasorestriction and blood pressure; cancer; cardiac arrhythmia; hypertension; congestive heart failure; conditions of the genital/urinary system; disorders of sexual function and fertility; adequacy of renal function; responsivity to anesthetics; mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder, mood disorders due to a general medical condition, and substance-induced mood disorders; affective neurosis; depressive neurosis; anxiety neurosis; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; acute neurological and psychiatric disorders such as cerebral deficits subsequent to cardiac bypass surgery and grafting, stroke, ischemic stroke, cerebral ischemia, spinal cord trauma, head trauma, perinatal hypoxia, cardiac arrest, hypoglycemic neuronal damage; Huntington's Chorea; Huntington's disease and Tourette syndrome; Cushing's syndrome/disease; basophile adenoma; prolactinoma; hyperprolactinemia; hypophysis tumor/adenoma; hypothalamic diseases; inflammatory bowel disease; gastric diskinesia; gastric ulcers; Froehlich's syndrome; adrenohypophysis disease; hypophysis disease; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; amyotrophic lateral sclerosis; multiple sclerosis; ocular damage; retinopathy; cognitive disorders; idiopathic and drug-induced Parkinson's disease; muscular spasms and disorders associated with muscular spasticity including tremors, epilepsy, convulsions, seizure disorders, absence seisures, complex partial and generalized seizures; Lennox-Gastaut syndrome; cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; dissociative disorders including multiple personality syndromes and psychogenic amnesias; substance-related disorders, substance use, substance abuse, substance seeking, substance reinstatement, all types of psychological and physical addictions and addictive behaviors, reward-related behaviors (including substance-induced delirium, persisting dementia, persisting amnestic disorder, psychotic disorder or anxiety disorder; tolerance, addictive feeding, addictive feeding behaviors, binge/purge feeding behaviors, dependence, withdrawal or relapse from substances including alcohol, amphetamines, cannabis, cocaine, hallucinogens, inhalants, morphine, nicotine, opioids, phencyclidine, sedatives, hypnotics or anxiolytics); appetite, taste, eating or drinking disorders; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), chronic fatigue syndrome, fatigue, including Parkinson's fatigue, multiple sclerosis fatigue, fatigue caused by a sleep disorder or a circadian rhythm disorder, medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias [including tremor (such as rest tremor, essential tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), restless leg syndrome and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia); neurodegenerative disorders including nosological entities such as disinhibition-dementia-parkinsonism-amyotrophy complex; pallido-ponto-nigral degeneration; epilepsy; seizure disorders; attention deficit/hyperactivity disorder (ADHD); conduct disorder; migraine (including migraine headache); headache; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; emesis, nausea, vomiting; gastric dyskinesia; gastric ulcers; Kallman's syndrome (anosmia); asthma; cancer; conditions associated with visceral pain such as irritable bowel syndrome, and angina; eating disorders; urinary incontinence; substance tolerance, substance withdrawal (including, substances such as opiates, nicotine, tobacco products, alcohol, benzodiazepines, cocaine, sedatives, hypnotics, etc.); psychosis; schizophrenia; anxiety (including generalized anxiety disorder, panic disorder, and obsessive compulsive disorder); mood disorders (including depression, mania, bipolar disorders); trigeminal neuralgia; hearing loss; tinnitus; neuronal damage including ocular damage; retinopathy; macular degeneration of the eye; emesis; brain edema; pain, including acute and chronic pain states, severe pain, intractable pain, inflammatory pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain, post-traumatic pain, trigeminal neuralgia, migraine and migraine headache and other diseases related to general orexin system dysfunction.

The subject compounds could further be of potential use in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to subjects (animals and human) in need of such treatment in dosages that will provide optimal pharmaceutical efficacy. The selected dosage depends upon the desired therapeutic effect, on the route of administration, and on the duration of the treatment. The dose will vary from subject to subject depending upon the nature and severity of disease, the subject's weight, special diets then being followed by a subject, concurrent medication, and other factors which those skilled in the art will recognize.

Generally, dosage levels of between 0.0001 to 100 mg/kg. of body weight daily are administered to the subject, e.g., humans, adolescent humans and elderly humans, to obtain effective agonism of orexin receptors. The dosage range will generally be about 0.5 mg to 10.0 g. per subject per day which may be administered in single or multiple doses. In one embodiment, the dosage range will be about 0.5 mg to 500 mg per subject per day; in another embodiment about 0.5 mg to 200 mg per subject per day; and in yet another embodiment about 5 mg to 50 mg per subject per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 25 mg, 30 mg, 50 mg, 80 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the subject to be treated. The compounds may be administered on a regimen of 1 to 4 times per day, such as once or twice per day. The compounds may be administered once or multiple times during the day. The compounds may be administered upon awakening or otherwise in the morning, or during waking hours. For example, the compounds may be administered about 1 hour after awakening, about 30 minutes after awakening or immediately after awakening.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention is contemplated. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used. In such combinations the compound of the present invention and other active agents may be administered separately or in conjunction. In addition, the administration of one element may be prior to, concurrent to, or subsequent to the administration of other agent(s).

The compounds of the present invention may be administered in combination with compounds which are known in the art to be useful for treating or controlling narcolepsy, including e.g., methylphenidate, amphetamine, pemoline, phenelzine, protriptyline, gamma-hydroxybutyric acid, sodium oxybate, or other oxybate salts, modafinil, armodafinil, caffeine, and salts thereof, and combinations thereof, and the like,

The compounds of the present invention may be administered in combination with compounds which are known in the art to be useful for preventing and treating sleep disorders and sleep disturbances, including e.g., sedatives, hypnotics, anxiolytics, antipsychotics, antianxiety agents, antihistamines, benzodiazepines, barbiturates, cyclopyrrolones, GABA agonists, 5HT-2 antagonists including 5HT-2A antagonists and 5HT-2A/2C antagonists, histamine antagonists including histamine H3 antagonists, histamine H3 inverse agonists, imidazopyridines, minor tranquilizers, melatonin agonists and antagonists, melatonergic agents, orexin antagonists, other orexin agonists, prokineticin agonists and antagonists, pyrazolopyrimidines, T-type calcium channel antagonists, triazolopyridines, and the like, such as: adinazolam, allobarbital, alonimid, alprazolam, amitriptyline, amobarbital, amoxapine, armodafinil, APD-125, bentazepam, benzoctamine, brotizolam, bupropion, busprione, butabarbital, butalbital, capromorelin, capuride, carbocloral, chloral betaine, chloral hydrate, chlordiazepoxide, clomipramine, clonazepam, cloperidone, clorazepate, clorethate, clozapine, conazepam, cyprazepam, desipramine, dexclamol, diazepam, dichloralphenazone, divalproex, diphenhydramine, doxepin, EMD-281014, eplivanserin, estazolam, eszopiclone, ethchlorynol, etomidate, fenobam, flunitrazepam, flurazepam, fluvoxamine, fluoxetine, fosazepam, gaboxadol, glutethimide, halazepam, hydroxyzine, ibutamoren, imipramine, indiplon, lithium, lorazepam, lormetazepam, LY-156735, maprotiline, MDL-100907, mecloqualone, melatonin, mephobarbital, meprobamate, methaqualone, methyprylon, midaflur, midazolam, modafinil, nefazodone, NGD-2-73, nisobamate, nitrazepam, nortriptyline, ornortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, TAK-375, temazepam, thioridazine, tiagabine, tracazolate, tranylcypromaine, trazodone, triazolam, trepipam, tricetamide, triclofos, trifluoperazine, trimetozine, trimipramine, uldazepam, venlafaxine, zaleplon, zolazepam, zopiclone, zolpidem, and salts thereof, and combinations thereof, and the like, or the compound of the present invention may be administered in conjunction with the use of physical methods such as with light therapy or electrical stimulation.

In another embodiment, the subject compound may be employed in combination with other compounds which are known in the art, either administered separately or in the same pharmaceutical compositions, including, but are not limited to: insulin sensitizers including (i) PPARy antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like); (iii) biguanides such as metformin and phenformin; (b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂); (c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide; (d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like; (e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activater receptor a agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics; (f) PPARu agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARU agonists as described in WO 97/36579; (g) PPAR6 agonists, such as those disclosed in WO97/28149; (h) PPAR a/6 agonists, such as muraglitazar, and the compounds disclosed in U.S. Pat. No. 6,414,002; (i) anti-obesity agents, such as (1) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CBI receptor antagonists or inverse agonists, such as rimonabant, taranabant, AMT-251, and SR-14778 and SR 141716A (Sanofi Synthelabo), SLV-319 (Solvay), BAY 65-2520 (Bayer) and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; (4) anti-obesity serotonergic agents, such as fenfluramine, dexfenfluramine, phentermine, and sibutramine; (5) 03-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027; (11) melanin-concentrating hormone 2 receptor (MCH2R) agonist/antagonists; (12) orexin receptor antagonists, such as SB-334867-A, and those disclosed in patent publications herein; (13) serotonin reuptake inhibitors such as fluoxetine, paroxetine, and sertraline; (14) melanocortin agonists, such as Melanotan II; (15) Mc4r (melanocortin 4 receptor) agonists, such as CHIR86036 (Chiron), ME-10142, and ME-10145 (Melacure), CHIR86036 (Chiron); PT-141, and PT-14 (Palatin); (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR14613, and those discribed in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and O-[3-(1H-imidazol-4-yl)propanol]-carbamates; (25) O-hydroxy steroid dehydrogenase-1 inhibitors (0-HSD-1); (26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron); (35) monoamine reuptake inhibitors, such as sibutramine; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid; (37) thyroid hormone β agonists, such as KB-2611 (KaroBioBMS); (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444, sitagliptin; and the compounds disclosed in U.S. Pat. No. 6,699,871, WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); (50) Topiramate (Topimax®); (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)); (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP), and other Y4 agonists such as 1229U91; (54) cyclooxygenase-2 inhibitors such as etoricoxib, celecoxib, valdecoxib, parecoxib, lumiracoxib, BMS347070, tiracoxib or JTE522, ABT963, CS502 and GW406381; (55) Neuropeptide Y1 (NPY1) antagonists such as BIBP3226, J-115814, BIBO 3304, LY-357897, CP-671906, GI-264879A; (56) Opioid antagonists such as nalmefene (Revex®), 3-methoxynaltrexone, naloxone, naltrexone; (57) 110 HSD-1 (11-beta hydroxy steroid dehydrogenase type 1) inhibitors such as BVT 3498, BVT 2733, and those disclosed in WO 01/90091, WO 01/90090, WO 01/90092, U.S. Pat. No. 6,730,690 and US 2004-0133011; (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; and (88) zonisamide., (89) neuromedin U and analogs or derivatives thereof, (90) oxyntomodulin and analogs or derivatives thereof, and (91) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HTiA agonists or antagonists, especially 5-HTiA partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; citalopram, duloxetine, fluoxetine, fluvoxamine, paroxetine and sertraline; isocarboxazid, phenelzine, tranylcypromine and selegiline; moclobemide: venlafaxine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with anti-Alzheimer's agents; beta-secretase inhibitors, such as verubecestat; gamma-secretase inhibitors; growth hormone secretagogues; recombinant growth hormone; HMG-CoA reductase inhibitors; NSAID's including ibuprofen; vitamin E; anti-amyloid antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H3 antagonists; AMPA agonists; PDE IV inhibitors; GABAA inverse agonists; or neuronal nicotinic agonists.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with acetophenazine, alentemol, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, risperidone, sulpiride, tetrabenazine, trihexyphenidyl, thioridazine, thiothixene or trifluoperazine.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone.

In another embodiment, the subject compound may be employed in combination with a nicotine agonist or a nicotine receptor partial agonist such as varenicline, opioid antagonists (e.g., naltrexone (including naltrexone depot), antabuse, and nalmefene), dopaminergic agents (e.g., apomorphine), ADD/ADHD agents (e.g., methylphenidate hydrochloride (e.g., Ritalin® and Concerta®), atomoxetine (e.g., Strattera®), a monoamine oxidase inhibitor (MAOI), amphetamines (e.g., Adderall®)) and anti-obesity agents, such as apo-B/MTP inhibitors, 11Beta-hydroxy steroid dehydrogenase-1 (11 Beta-HSD type 1) inhibitors, peptide YY3-36 or analogs thereof, MCR-4 agonists, CCK-A agonists, monoamine reuptake inhibitors, sympathomimetic agents, 03 adrenergic receptor agonists, dopamine receptor agonists, melanocyte-stimulating hormone receptor analogs, 5-HT2c receptor agonists, melanin concentrating hormone receptor antagonists, leptin, leptin analogs, leptin receptor agonists, galanin receptor antagonists, lipase inhibitors, bombesin receptor agonists, neuropeptide-Y receptor antagonists (e.g., NPY Y5 receptor antagonists), thyromimetic agents, dehydroepiandrosterone or analogs thereof, glucocorticoid receptor antagonists, orexin receptor antagonists, such as suvorexant, other orexin agonists, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists, and pharmaceutically acceptable salts thereof.

In another embodiment, the subject compound may be employed in combination with an agent such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptable salts thereof.

In another embodiment, the subject compound may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention may be effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives. Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

Several methods for preparing the compounds of this invention are illustrated in the following Schemes and Examples. Starting materials are made according to procedures known in the art or as illustrated herein. The following abbreviations are used herein: Me: methyl; Et: ethyl; t-Bu: tert-butyl; Ar: aryl; Ph: phenyl; BINAP: 2,2′-bis(diphenylphosphino)-1,1′-binaphthyl; Bn: benzyl; Ac: acetyl; Boc: tert-butyloxy carbonyl; BSA: bovine serum albumin; CbzCl: benzylchloroformate; CDI: carbonyl diimidazole; DCM (CH₂Cl2): dichloromethane; DCE: dichloroethane; DEAD: diethylazodicarboxylate; DIPEA: N,N-diisopropylethylamine; DMF: N,N-dimethylformamide; DMSO: dimethylsulfoxide; EDC: N-(3-Dimethylaminopropyl)-N′-ethylcarbodiimide; Et3N: triethylamine; EtOAc: ethyl acetate; EtOH: ethanol; HCl: hydrogen chloride; HOAt: 1-hydroxy-7-aza-benzotriazole; HOBT: hydroxybenzotriazole hydrate; HPLC: high performance liquid chromatography; Hunig's base: N,N-diisopropylethylamine; MeOH: methanol; MgSO4: magnesium sulfate; Ms: methanesulfonyl; MTBE: methyl tert-butyl ether; NaHCO₃: sodium bicarbonate; NaOH: sodium hydroxide; NMM: N-methylmorpholine; PtO2: platinum oxide; PyClu: 1-(chloro-1-pyrrolidinylmethylene)-pyrrolidinium hexafluorophosphate; rt: room temperature; SOCl2: thionyl chloride; T3P: 2,4,6-tripropyl-1,3,5,2,4,6-trioxatriphosphorinane-2,4,6-trioxide; THF: tetrahydrofuran; TFA: trifluoracetic acid; X-Phos: 2-(dicyclohexyl-phosphino)-2′,4′,6′-triisopropylbiphenyl.

The compounds of the present invention can be prepared in a variety of fashions. In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Intermediate A

### Benzyl (2R,3S,5R)-3-((N,N-dimethylsulfamoyl)(4-methoxybenzyl)amino)-5-methyl-2-(((triethylsilyl)oxy)methyl)pyrrolidine-1-carboxylate

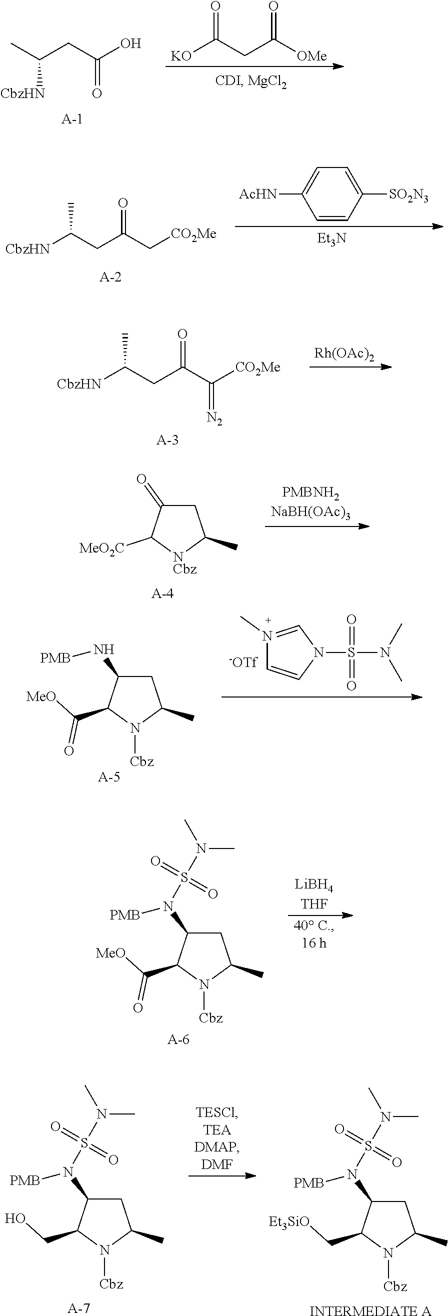

### Step 1: methyl (R)-5-(((benzyloxy)carbonyl)amino)-3-oxohexanoate (A-2)

To a solution of (R)-3-(((benzyloxy)carbonyl)amino)butanoic acid (A-1) (6.25 g, 26.3 mmol) in anhydrous THF (100 ml) under N₂ was added di(1H-imidazol-1-yl)methanone (6.41 g, 39.5 mmol). After stirring at rt for 1 h, pre-mixed MgCl₂ (4.64 ml, 52.7 mmol) and potassium 3-methoxy-3-oxopropanoate (8.23 g, 52.7 mmol) was added. The resulting mixture was stirred at rt for additional 18 h under N₂. The solvent was evaporated and the residue was dissolved in ethyl acetate (100 mL) and washed with brine (20 mL). The organic layer was dried over MgSO₄, filtered and concentrated in vacuo. The residue was purified by column chromatography (silica gel, 0-100% EtOAc in hexanes) to afford the title compound. LC-MS 294 (M+1).

### Step 2: methyl (R)-5-(((benzyloxy)carbonyl)amino)-2-diazo-3-oxohexanoate (A-3)

To a solution of methyl (R)-5-(((benzyloxy)carbonyl)amino)-3-oxohexanoate (A-2) (6.2 g, 21.14 mmol) in CH₂Cl₂ (200 ml) was added Et₃N (6.42 g, 63.4 mmol) and 4-acetamidobenzene-sulfonyl azide (5.08 g, 21.14 mmol) at rt under N₂. The reaction mixture was stirred for 12 h. LC-MS shown reaction completed. The crude was diluted with 200 ml of DCM, then was washed with 50 ml of H₂O. The organic phase was collected and dried over MgSO₄, concentrated and chromatographed over silica gel (0-100% Ethyl acetate in hexanes) to give the title compound. LC-MS 320 (M+1).

### Step 3: 1-benzyl 2-methyl (5R)-5-methyl-3-oxopyrrolidine-1,2-dicarboxylate (A-4)

To a solution of methyl (R)-5-(((benzyloxy)carbonyl)amino)-2-diazo-3-oxohexanoate (A-3) (2.0 g, 6.26 mmol) in toluene (50 ml) was added diacetoxyrhodium (0.138 g, 0.313 mmol) under N₂ at rt. The reaction mixture was degassed for 10 min, then was stirred at 80° C. for 2 h. LC-MS shown reaction completed. The reaction mixture was concentrated and chromatographed over silica gel (0-100% EtOAc in hexanes) to give the title compound. LC-MS 292.28 (M+1).

### Step 4: methyl (R)-5-(((benzyloxy)carbonyl)amino)-3-oxohexanoate (A-5)

To a solution of 1-benzyl 2-methyl (5R)-5-methyl-3-oxopyrrolidine-1,2-dicarboxylate (A-4) (5000 mg, 17.16 mmol) in DCM (100 mL) was added 4-methoxybenzylamine (2.467 mL, 18.88 mmol) and catalytic amount of acetic acid (0.049 mL, 0.858 mmol). The mixture was stirred at rt for 30 mins, then sodium triacetoxyborohydride (4.37 g, 20.6 mmol) was added to the mixture. The reaction was stirred at rt overnight. The reaction was quenched with sat. aq. NaHCO₃(50 mL), extracted with DCM (3×50 mL). The combined organic phases were dried over MgSO₄, filtered and concentrated. The residue was purified by column chromatography on silica gel (EtOAc in Hexane 0-100%) to afford the title compound. LC-MS 413 (M+1).

### Step 5: 1-benzyl 2-methyl (2R,3S,5R)-3-((N,N-dimethylsulfamoyl)(4-methoxybenzyl)amino)-5-methylpyrrolidine-1,2-dicarboxylate (A-6)

Into a 2000-mL 4-necked round-bottom flask, was placed DCM (450 ml), 1-benzyl 2-methyl (5R)-3-((4-methoxybenzyl)amino)-5-methylpyrrolidine-1,2-dicarboxylate (A-5) (150 g, 1 eq) and 1-(N,N-dimethylsulfamoyl)-3-methyl-1H-imidazol-3-ium trifluoromethanesulfonate (370 g, 3 equiv), The resulting solution was stirred for 3 d at 80° C. in an oil bath. The reaction mixture was cooled to room temperature and concentrated. The residue was applied onto a silica gel column with ethyl acetate/petroleum ether (1/5-1/4) to give the desired product.

### Step 6: benzyl (2R,3S,5R)-3-((N,N-dimethylsulfamoyl)(4-methoxybenzyl)amino)-2-(hydroxymethyl)-5-methylpyrrolidine-1-carboxylate (A-7)

Into a 2000-mL 3-necked round-bottom flask purged and maintained with an inert atmosphere of nitrogen, was placed a solution of 1-benzyl 2-methyl (2R,3S,5R)-3-((N,N-dimethylsulfamoyl)(4-methoxybenzyl)amino)-5-methylpyrrolidine-1,2-dicarboxylate (A-6) (40 g, 80 mmol) in THF (400 ml). This was followed by the addition of LiBH4 (7 g, 315 mmol) with stirring at 0° C. The resulting solution was stirred at 40° C. for 16 h. The reaction was then quenched by the addition of water/ice. The resulting solution was extracted with 3×500 ml of EA and the organic layers combined and dried over Na₂SO₄ and concentrated to give the desired product. (ESI, m/z): (M+Na)+: 514

### Step 7: benzyl (2R,3S,5R)-3-((N,N-dimethylsulfamoyl)(4-methoxybenzyl)amino)-5-methyl-2-(((triethylsilyl)oxy)methyl)pyrrolidine-1-carboxylate (Intermediate A)

Into a 1000-mL 3-necked round-bottom flask purged and maintained with an inert atmosphere of nitrogen, was placed a solution of benzyl (2R,3S,5R)-3-((N,N-dimethyl-sulfamoyl)(4-methoxybenzyl)amino)-2-(hydroxymethyl)-5-methylpyrrolidine-1-carboxylate (A-7) (34 g, 69 mmol) in DMF (340 ml) was added TEA (8.36 g, 83 mmol) at r.t under N2. Then add DMAP (1.68 g, 14 mmol) to the system. This was followed by the addition of TESCl (12.5 g, 83 mmol) dropwise with stirring at 0° C. The resulting solution was stirred at 25° C. for 3 h. The reaction was then quenched by the addition of water/ice. The resulting solution was extracted with 2×300 mL of EA. The organic layer was washed with 200 mL of brine and the organic layers combined and dried over Na₂SO₄ and concentrated. The residue was applied onto a silica gel column with petroleum ether/ethyl acetate (15/1) to give the desired product. (ESI, m/z): (M+Na)⁺ 606.

### Intermediate B

### Benzyl (2R,3S,5R)-3-(N-(4-methoxybenzyl)methylsulfonamido)-5-methyl-2-(((triethylsilyl)oxy)methyl)pyrrolidine-1-carboxylate

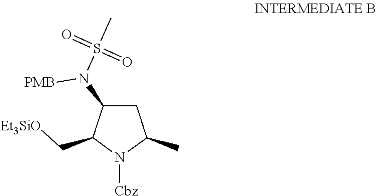

Benzyl (2R,3S,5R)-3-(N-(4-methoxybenzyl)methylsulfonamido)-5-methyl-2-(((triethylsilyl)oxy)methyl)pyrrolidine-1-carboxylate (INTERMEDIATE B) was prepared according to the same procedure provided in INTERMEDIATE A by substituting the appropriate reagent with methylsulfonyl chloride.

### Intermediate C

### Benzyl (2R,3S,5S)-5-(methoxymethyl)-2-(((triethylsilyl)oxy)methyl)-3-(2,2,2-trifluoro-N-(4-methoxybenzyl)acetamido)pyrrolidine-1-carboxylate

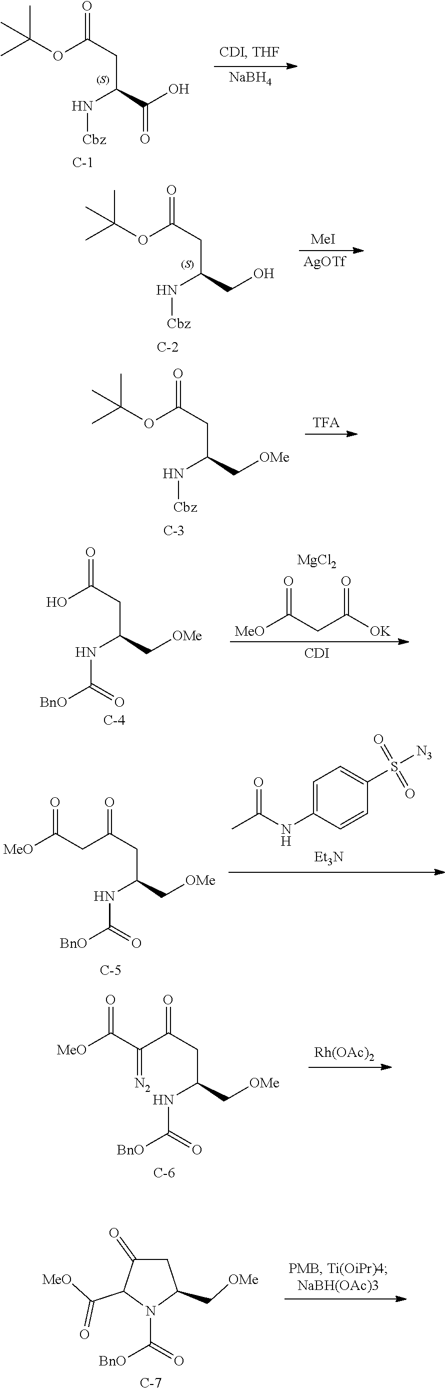

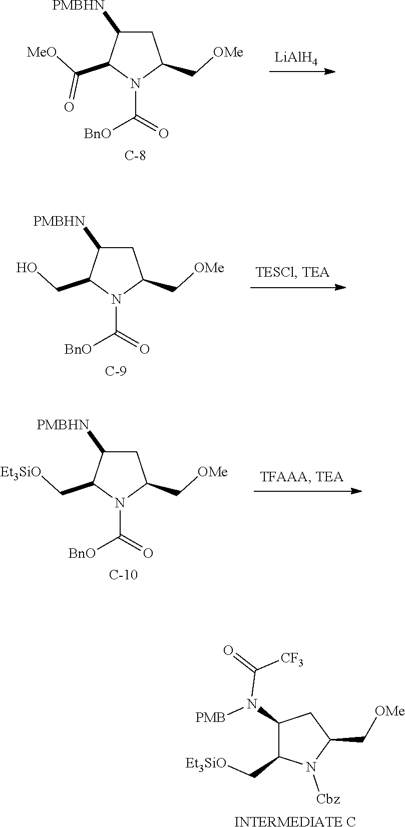

### Step 1: tert-butyl (3S)-3-[[(benzyloxy)carbonyl]amino]-4-hydroxybutanoate (C-2)

Into a 20-L 4-necked round-bottom flask purged and maintained with an inert atmosphere of nitrogen, was placed a solution of (2S)-2-[[(benzyloxy)carbonyl]amino]-4-(tert-butoxy)-4-oxobutanoic acid monohydrate (C-1) (800.00 g, 2.34 mol, 1.00 equiv) in THF (8 L). To the mixture was added CDI (760.01 g, 4.68 mol, 2.00 equiv). The resulting solution was stirred for 1.5 h at 0 C in an ice/salt bath. After that the mixture was added to a solution of NaBH₄ (177.3 g, 4.68 mol, 2.00 equiv) in H₂O (4 L). The resulting solution was allowed to react, with stirring, for an additional 3 hr at room temperature. The resulting mixture was concentrated. The resulting solution was extracted with 3×4 L of ethyl acetate and the organic layers combined. The resulting mixture was washed with 2×4 L of brine. The mixture was dried over anhydrous sodium sulfate and concentrated to afford the title compound.

### Step 2: tert-butyl (3S)-3-[[(benzyloxy)carbonyl]amino]-4-methoxybutanoate (C-3)

To a mixture of tert-butyl (3S)-3-[[(benzyloxy)carbonyl]amino]-4-hydroxybutanoate (C-2) (650 g, 2.10 mol, 1.00 equiv) in DCM (6.5 L) at 0° C. was added iodomethane (477.16 g, 3.36 mol, 1.60 equiv), 2, 6-di-tert-butyl-4-methylpyridine (862.88 g, 4.20 mol, 2.00 equiv) and silver trifluoromethanesulfonate (863.74 g, 3.36 mol, 1.60 equiv). The mixture was allowed to warm to ambient temperature and stirred overnight. The resulting mixture was filtered through a pad of celite and concentrated. The resulting residue was purified on column with a solvent system of 2% to 75% EtOAc/PE to obtain the title compound.

### Step 3: (3S)-3-[[(benzyloxy)carbonyl]amino]-4-methoxybutanoic acid (C-4)

Into a 5-L 4-necked round-bottom flask purged and maintained with an inert atmosphere of nitrogen, was placed a solution of tert-butyltert-butyl (3S)-3-[[(benzyloxy)carbonyl]amino]-4-methoxybutanoate (C-3) (425 g, 1.314 mol, 1.00 equiv) in DCM (2.20 L). This was followed by the addition of TFA (224.78 g, 1.971 mol, 1.50 equiv) dropwise with stirring at room temperature. The resulting solution was stirred for 4 h at room temperature. The reaction was then quenched by the addition of 2 L of water/ice. The resulting mixture was washed with 3×1.2 L of H₂O. The mixture was dried over anhydrous sodium sulfate and concentrated. The residue was applied onto a silica gel column with ethyl acetate/petroleum ether (0%-25%) to afford the title compound.

### Step 4: ethyl (5S)-5-[[(benzyloxy)carbonyl]amino]-6-methoxy-3-oxohexanoate (C-5)

Into a 10-L 4-necked round-bottom flask purged and maintained with an inert atmosphere of nitrogen, was placed a solution of (3S)-3-[[(benzyloxy)carbonyl]amino]-4-methoxybutanoic acid (C-4) (270 g, 1.010 mol, 1.00 equiv) in THF (2.7 L). This was followed by the addition of CDI (245.7 g, 1.51 mol, 1.50 equiv) dropwise with stirring at room temperature. The resulting solution was stirred for 3 h at room temperature. This was followed by the addition of MgCl2 (192.36 g, 2.020 mol, 2.00 equiv) and 1-ethyl 3-potassium propanedioate (343.87 g, 2.020 mol, 2.00 equiv) dropwise with stirring at room temperature. The resulting solution was allowed to react, with stirring, for an additional 2 days at room temperature. The resulting solution was diluted with 1.4 L of EA. The reaction was then quenched by the addition of 1 L of water/ice. The resulting mixture was washed with 2×2 L of NaHCO₃. The resulting solution was extracted with 2×1 L of ethyl acetate and the organic layers combined. The resulting mixture was washed with 1×2 L of brine. The mixture was dried over anhydrous sodium sulfate and concentrated to afford the title compound.

### Step 5: ethyl (5S)-5-[[(benzyloxy)carbonyl]amino]-2-diazo-6-methoxy-3-oxohexanoate (C-6)

Into a 5 L 4-necked round-bottom flask purged and maintained with an inert atmosphere of nitrogen, was placed a solution of ethyl (5S)-5-[[(benzyloxy)carbonyl]amino]-6-methoxy-3-oxohexanoate (C-5) (220.0 g, crude) in DCM (2.2 L). This was followed by the addition of triethylamine (197.96 g, 1.956 mol, 3.00 equiv) and 4-acetamidobenzenesulfonyl azide (156.66 g, 0.652 mol, 1.00 equiv) in portion wise with stirring at room temperature The resulting solution was stirred overnight at room temperature. The solids were filtered out. The filtrate was washed with 2×660 mL of H2O. The resulting mixture was washed with 1×660 mL of citric acid and 1×1.4 L of brine. The mixture was dried over anhydrous sodium sulfate and concentrated to afford the title compound.

### Step 6: 1-benzyl 2-ethyl (5S)-5-(methoxymethyl)-3-oxopyrrolidine-1,2-dicarboxylate (C-7)

Into a 3 L 3-necked round-bottom flask purged and maintained with an inert atmosphere of nitrogen, was placed a solution of ethyl (5S)-5-[[(benzyloxy)carbonyl]amino]-2-diazo-6-methoxy-3-oxohexanoate (C-6) (116 g, crude) in toluene (1.2 L) and (acetyloxy)rhodio acetate (7.05 g, 31.92 mmol, 0.10 equiv). The resulting solution was stirred for 3 h at 80° C. in an oil bath. The reaction mixture was cooled with a water bath. The solids were filtered out. The filtrate was concentrated. The residue was applied onto a silica gel column with ethyl acetate/petroleum ether (0%-20%) to afford the title compound.

### Step 7: 1-benzyl 2-ethyl (2R,3S,5S)-5-(methoxymethyl)-3-[[(4-methoxyphenyl)methyl]amino]-pyrrolidine-1,2-dicarboxylate (C-8)

Into a 2 L 3-necked round-bottom flask purged and maintained with an inert atmosphere of nitrogen, was placed a solution of 1-benzyl 2-ethyl (5S)-5-(methoxymethyl)-3-oxopyrrolidine-1,2-dicarboxylate (C-7) (95.0 g, 283.28 mmol, 1.00 equiv) in THF (1 L), (4-methoxyphenyl)methanamine (46.63 g, 339.94 mmol, 1.20 equiv). This was followed by the addition of Ti(Oi-Pr)₄ (80.51 g, 283.27 mmol, 1.00 equiv) dropwise with stirring at 0° C. The resulting solution was stirred overnight at room temperature. To this was added STAB (420.27 g, 1982.96 mmol, 7.00 equiv) in several batches. The resulting solution was allowed to react, with stirring, for an additional 2 days at room temperature. The resulting solution was diluted with 500 mL of EA. The reaction was then quenched by the addition of 1 L of NaHCO₃(aq.). The solids were filtered out. The resulting solution was extracted with 2×500 mL of ethyl acetate and the organic layers combined. The resulting mixture was washed with 2×1 L of brine. The mixture was dried over anhydrous sodium sulfate and concentrated. The residue was applied onto a silica gel column with ethyl acetate/petroleum ether (0%-45%) to give the title compound.

### Step 8: benzyl (2R,3S,5S)-2-(hydroxymethyl)-5-(methoxymethyl)-3-[[(4-methoxyphenyl)-methyl]amino]pyrrolidine-1-carboxylate (C-9)

Into a 2 L 3-necked round-bottom flask purged and maintained with an inert atmosphere of nitrogen, was placed a solution of 1-benzyl 2-ethyl (2R,3S,5S)-5-(methoxymethyl)-3-[[(4-methoxyphenyl)methyl]amino]pyrrolidine-1,2-dicarboxylate (C-8) (106 g, 232.18 mmol, 1.00 equiv) in THF (0.6 L). This was followed by the addition of LiBH4 (15.17 g, 696.38 mmol, 3.00 equiv) dropwise with stirring at room temperature. The resulting solution was stirred overnight at room temperature. The reaction was then quenched by the addition of 300 mL of EA. The resulting solution was diluted with 600 mL of H2O/ice. The resulting solution was extracted with 2×300 mL of ethyl acetate and the organic layers combined. The resulting mixture was washed with 2×600 mL of brine. The mixture was dried over anhydrous sodium sulfate and concentrated. The crude product was purified by Prep-HPLC with MeCN/H2O 35%-57% to obtain the title compound.

### Step 9: benzyl (2R,3S,5S)-5-(methoxymethyl)-3-[[(4-methoxyphenyl)methyl]amino]-2-[[(triethylsilyl)oxy]methyl]pyrrolidine-1-carboxylate (C-10)

Into a 1 L 3-necked round-bottom flask purged and maintained with an inert atmosphere of nitrogen, was placed a solution of benzyl (2R,3S,5S)-2-(hydroxymethyl)-5-(methoxymethyl)-3-[[(4-methoxyphenyl)methyl]amino]pyrrolidine-1-carboxylate (C-9) (54.00 g, 130.28 mmol, 1.00 equiv) in DCM (540 mL), TEA (17.14 g,169.36 mmol, 1.30 equiv), DMAP (1.59 g, 13.03 mmol, 0.10 equiv). This was followed by the addition of chlorotriethylsilane (21.6 g, 143.31 mmol, 1.10 equiv) dropwise with stirring at 0 C. The resulting solution was stirred for 2 h at room temperature. The reaction was then quenched by the addition of 540 mL of water/ice. The resulting solution was extracted with 2×260 mL of DCM and the organic layers combined. The resulting mixture was washed with 2×540 mL of brine. The mixture was dried over anhydrous sodium sulfate and concentrated to afford the title compound.

### Step 10: benzyl (2R,3S,5S)-5-(methoxymethyl)-2-[[(triethylsilyl)oxy]methyl]-3-[2,2,2-trifluoro-N-[(4-methoxyphenyl)methyl]acetamido]pyrrolidine-1-carboxylate (Intermediate C)

Into a 2 L 3-necked round-bottom flask purged and maintained with an inert atmosphere of nitrogen, was placed a solution of benzyl (2R,3S,5S)-5-(methoxymethyl)-3-[[(4-methoxyphenyl)methyl]amino]-2-[[(triethylsilyl)oxy]methyl]pyrrolidine-1-carboxylate (C-10) (67.50 g, 127.66 mmol, 1.00 equiv) in DCM (680 mL), TEA (25.84 g, 255.36 mmol, 2.00 equiv). This was followed by the addition of TFAA (32.17 g, 153.17 mmol, 1.20 equiv) dropwise with stirring at 0° C. The resulting solution was stirred for 2 h at room temperature. The resulting mixture was concentrated. The crude product was purified by Prep-HPLC with MeCN/H2O=87%-100% to obtain the title compound. (ES, m/z): 625 [M+1]+. 1H-NMR: (300 MHz, CDCl3, ppm): δ 7.36 (s, 5H), 7.03 (d, J=8.5 Hz, 2H), 6.90 (d, J=8.7 Hz, 2H), 5.26-5.00 (m, 2H), 4.94-4.80 (m, 2H), 4.67-4.37 (m, 2H), 3.98-3.71 (m, 6H), 3.60-3.44 (m, 1H), 3.40-3.15 (m, 4H), 2.42-2.07 (m, 1H), 1.85 (m, J=12.9, 7.1 Hz, 1H), 0.96 (t, J=8.0 Hz, 9H), 0.71-0.52 (m, 6H).

### Intermediate D

### Benzyl (2R,3S,5S)-2-(((tert-butyldimethylsilyl)oxy)methyl)-3-(N-(4-methoxybenzyl)methylsulfonamido)-5-(methoxymethyl)pyrrolidine-1-carboxylate

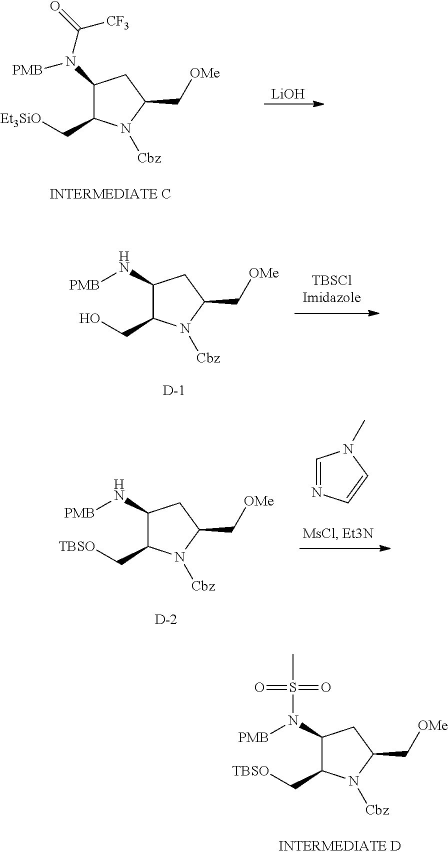

### Step 1: Benzyl (2R,3S,5S)-2-(hydroxymethyl)-3-((4-methoxybenzyl)amino)-5-(methoxymethyl)pyrrolidine-1-carboxylate (D-1)

To a mixture of benzyl (2R,3S,5S)-5-(methoxymethyl)-2-(((triethylsilyl)oxy)methyl)-3-(2,2,2-trifluoro-N-(4-methoxybenzyl)acetamido)pyrrolidine-1-carboxylate (INTERMEDIATE C) (5.00 g, 8.00 mmol) in THF (40.0 ml))/MeOH (40.0 ml) was added 3.0M LiOH LiOH (5.34 ml, 16.01 mmol). The mixture stirred for 3 hours before quenching with H2O (50 mL), extracting with EtOAc (3×@ 50 mL), drying over Na2SO4, and concentrating to obtain the title compound. MS: 415.4 (M+H).

### Step 2: benzyl (2R,3S,5S)-2-(((tert-butyldimethylsilyl)oxy)methyl)-3-((4-methoxybenzyl)amino)-5-(methoxymethyl)pyrrolidine-1-carboxylate (D-2)

To a mixture of benzyl (2R,3S,5S)-2-(hydroxymethyl)-3-((4-methoxybenzyl)amino)-5-(methoxymethyl)pyrrolidine-1-carboxylate (D-1) (3.30 g, 7.96 mmol) in DCM (53.1 ml) at ambient temperature was added TBS-Cl (1.440 g, 9.55 mmol) and IMIDAZOLE (1.084 g, 15.92 mmol). The mixture stirred for 1 hour before concentrating and purifying the residue using silia column chromatography (2% to 60% 3:1 EtOAc:EtOH/hexanes) to obtain the title compound. MS: 529.5 (M+H).

### Step 3: benzyl (2R,3S,5S)-2-(((tert-butyldimethylsilyl)oxy)methyl)-3-(N-(4-methoxybenzyl)methylsulfonamido)-5-(methoxymethyl)pyrrolidine-1-carboxylate (Intermediate D)

To a mixture of 1-METHYLIMIDAZOLE (1.900 ml, 23.83 mmol) in DCM (39.7 ml) at 0° C. was added Ms-Cl (0.928 ml, 11.91 mmol). The mixture was allowed to stir for 20 min before adding a mixture of benzyl (2R,3S,5S)-2-(((tert-butyldimethylsilyl)oxy)methyl)-3-((4-methoxybenzyl)amino)-5-(methoxymethyl)pyrrolidine-1-carboxylate (D-2) (4.2 g, 7.94 mmol) and TRIETHYLAMINE (4.43 ml, 31.8 mmol) in 10 mL DCM. The mixture was warmed to ambient temperature and stirred for 4 hours. The mixture was quenched with a saturated solution of NaHCO3(50 mL), extracted with DCM (3×@ 50 mL), dried over Na2SO4, and concentrated. The resulting residue was purified using silica gel chromatography (5% to 90% 3:1 EtOAc:EtOH/hexanes) to obtain the title compound. MS: 607.5 (M+H).

### Intermediate E

### 2-(4-oxocyclohexyl)phenyl trifluoromethanesulfonate

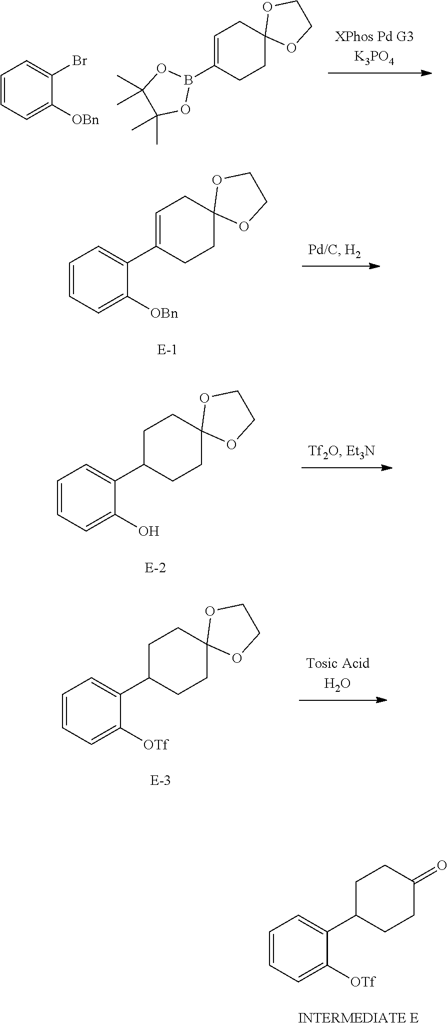

### Step 1: 8-(2-(benzyloxy)phenyl)-1,4-dioxaspiro[4.5]dec-7-ene (E-1)

To a mixture of 4,4,5,5-tetramethyl-2-(1,4-dioxaspiro[4.5]dec-7-en-8-yl)-1,3,2-dioxaborolane (3.25 g, 12.21 mmol) in Dioxane (24.42 ml) at ambient temperature was added 1-(benzyloxy)-2-bromobenzene (4.82 g, 18.32 mmol), Xphos Pd G3 (0.517 g, 0.611 mmol), and K3PO4 (7.78 g, 36.6 mmol) dissolved in Water (6.11 ml). The mixture was heated to 80° C. and stirred for 1 hour before cooling, take up in DCM (60 mL)/H2O (60 mL), extract with DCM (3×@ 60 mL), dried over Na2SO4, and concentrated. The resulting residue was purified using silica column chromatography (2% to 25% EtOAc/hexanes) to obtain the title compound. MS: 323.2 (M+H).

### Step 2: 2-(1,4-dioxaspiro[4.5]decan-8-yl)phenol (E-2)

To a mixture of 8-(2-(benzyloxy)phenyl)-1,4-dioxaspiro[4.5]dec-7-ene (E-1) (3.94 g, 12.22 mmol) in Ethyl acetate (61.1 ml) was added Pd/C (1.301 g, 1.222 mmol). A balloon of H2 was added (vacuum purge 3×) and the resulting mixture stirred for 2 days. The mixture was filtered through a pad of celite and the resulting filtrate was concentrated. The resulting residue was purified using silica column chromatography (5% to 60% EtOAc/hexanes) to obtain the title compound. MS: 235.3 (M+H).

### Step 3: 2-(1,4-dioxaspiro[4.5]decan-8-yl)phenyl trifluoromethanesulfonate (E-3)

To a mixture of 2-(1,4-dioxaspiro[4.5]decan-8-yl)phenol (E-2) (1.00 g, 4.27 mmol) in DCM (17.07 ml) at −78° C. was added TRIETHYLAMINE (1.190 ml, 8.54 mmol) followed by TriflicAnhydride (5.12 ml, 5.12 mmol) in DCM dropwise. The mixture stirred for 2 hours before warming to 0° C. where it was quenched with a saturated solution of NaHCO₃(50 mL), extract with DCM (3×@ 50 mL), dry over Na2SO4, and concentrate. The resulting residue was purified using silica column chromatography (2% to 40% EtOAc/hexanes) to obtain the title compound. MS: 367.2 (M+H).

### Step 4: 2-(4-oxocyclohexyl)phenyl trifluoromethanesulfonate (Intermediate E)

To a mixture of 2-(1,4-dioxaspiro[4.5]decan-8-yl)phenyl trifluoromethanesulfonate (E-3) (1.50 g, 4.09 mmol) in THF (13.65 ml) at ambient temperature was added TosicAcid (0.234 g, 1.228 mmol) and H2O (0.738 ml, 40.9 mmol). The mixture was heated to 50° C. and stirred for 7 hours. The mixture was cooled and concentrated. The mixture was taken up in DCM (10 mL) and H2O (10 mL), extract with DCM (3×@ 10 mL), dry over Na2SO4, and concentrate. The resulting residue was purified using silica column chromatography (2% to 30% EtOAc/hexanes) to obtain the title compound. MS: 323.1 (M+H).

### Intermediate F

### 2-Fluoro-6-(4-oxocyclohexyl)phenyl trifluoromethanesulfonate

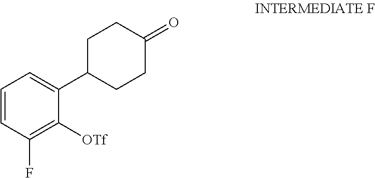 [SMILES: O=C1CCC(c2cccc(F)c2OS(=O)(=O)C(F)(F)F)CC1]

2-Fluoro-6-(4-oxocyclohexyl)phenyl trifluoromethanesulfonate (INTERMEDIATE F) was prepared according to the same procedure provided in INTERMEDIATE E by substituting with the appropriate coupling partner.

### Intermediate G

### 4-(3-(benzyloxy)phenyl)cyclohexan-1-one

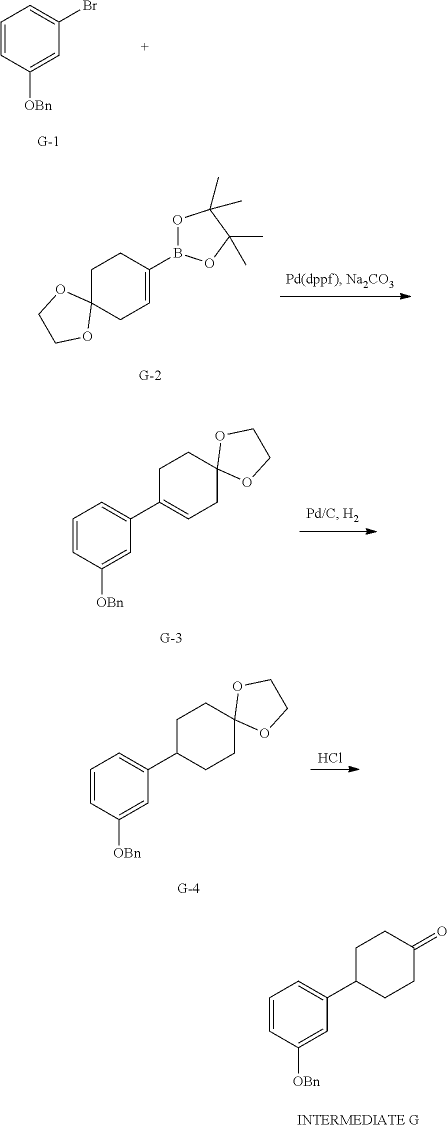 [SMILES: Brc1cccc(OCc2ccccc2)c1 ; CC1(C)OB(C2=CCC3(CC2)OCCO3)OC1(C)C ; C1=C(c2cccc(OCc3ccccc3)c2)CCC2(C1)OCCO2 ; c1ccc(COc2cccc(C3CCC4(CC3)OCCO4)c2)cc1 ; Cl ; O=C1CCC(c2cccc(OCc3ccccc3)c2)CC1]

### Step 1: 8-(3-(benzyloxy)phenyl)-1,4-dioxaspiro[4.5]dec-7-ene (G-3)

A 250 ml of RBF fitted a condenser was charged a suspension of 1-(benzyloxy)-3-bromobenzene (G-1) (6 g, 22.80 mmol), 4,4,5,5-tetramethyl-2-(1,4-dioxaspiro[4.5]dec-7-en-8-yl)-1,3,2-dioxaborolane (G-2) (6.13 g, 23.03 mmol),1,1bis(diphenyl-phosphino)ferrocene-palladium(II)dichloride dichloromethane complex (1.862 g, 2.280 mmol) and sodium carbonate (7.25 g, 68.4 mmol) in DME (100 mL)/Water (30 mL) was bubbled through N2 for 5 min. The mixture was heated to 80° C. After 15 hrs, most of solvent was removed under reduced pressure. The mixture was added aqueous sodium hydrogen carbonate (saturated, 100 mL) and the mixture was extracted with ethyl acetate (3×150 mL). The combined organic fractions were washed with brine (saturated, 100 mL), dried (Na₂SO₄), filtered and the solvent was evaporated under reduced pressure. The residue was purified by silica column chromatography (0-30% EtOAc/hexanes) to give the title compound. MS: 323(M+H).

### Step 2: 8-(3-(benzyloxy)phenyl)-1,4-dioxaspiro[4.5]decane (G-4)

A solution of 8-(3-(benzyloxy)phenyl)-1,4-dioxaspiro[4.5]dec-7-ene (G-3) (6.05 g, 18.77 mmol) in MeOH (75 ml) was added Pd/C (200 mg, 0.188 mmol). The mixture was shaked in a parr shaker under H2 at 40 psi. After 8 hrs, the mixture was filtered through a pad of celite and washed with methanol. The combined filtrates were concentrated under reduced pressure to give the title compound. MS: 325 (M+H).

### Step 3: 4-(3-(benzyloxy)phenyl)cyclohexan-1-one (Intermediate G)

A 250 ml of RBF was charged a solution of 8-(3-(benzyloxy)phenyl)-1,4-dioxaspiro[4.5]decane (G-4) (5.9 g, 18.19 mmol) in Acetone (100 ml)/Water (50 ml). Hydrogen chloride (7.0 M aq, 7.79 ml, 54.6 mmol) was added to above solution, After 5 hrs, most of solvents were removed under reduced pressure and diluted with 100 ml of water. The mixture was extracted with ethyl acetate (3×100 mL). The combined organic fractions were washed with brine (saturated, 100 mL), dried (Na₂SO₄), filtered and the solvent was evaporated under reduced pressure. The residue was purified by silica column chromatography (0-50% EtOAc/hexanes) to give the title compound. MS: 281(M+H).

### Intermediate H

### tert-butyl (2R,3S)-2-[[(tert-butyldiphenylsilyl)oxy]methyl]-3-[(dimethylsulfamoyl)[(4-methoxyphenyl)methyl]amino]pyrrolidine-1-carboxylate

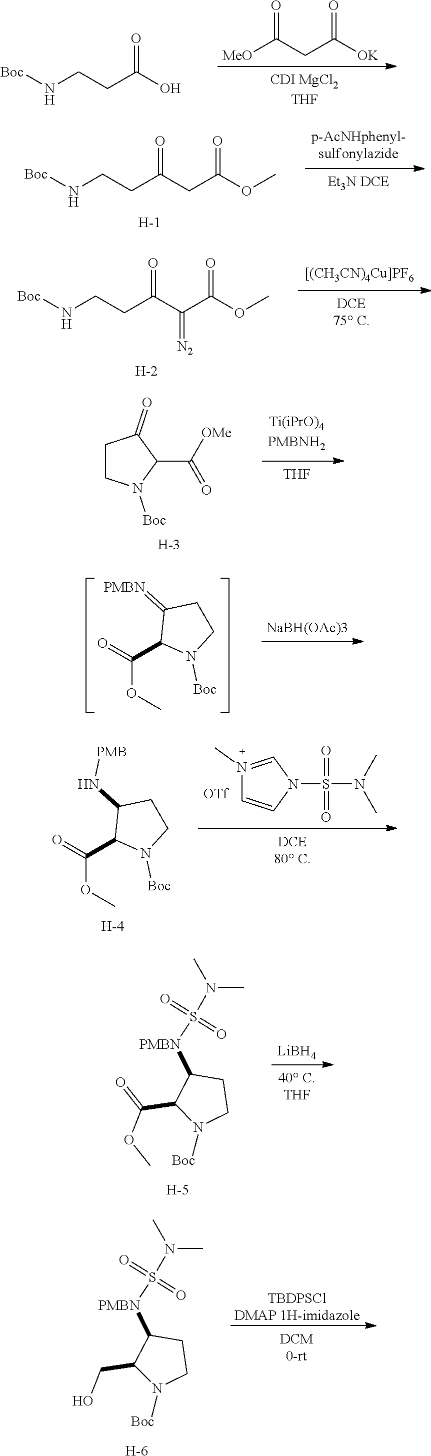

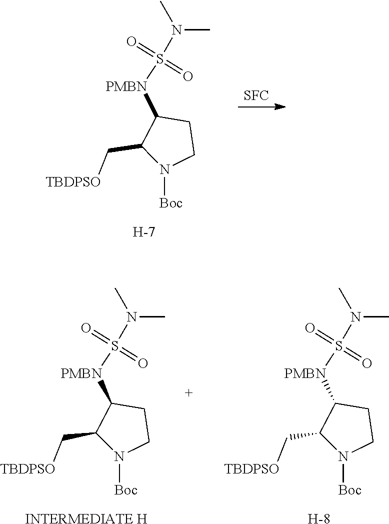

### Step 1: methyl 5-[(tert-butoxycarbonyl)amino]-3-oxopentanoate (H-1)

Into a 10-L 4-necked round-bottom flask purged and maintained with an inert atmosphere of nitrogen, was placed CDI (391 g, 1.2 eq, 2.412 mol), tetrahydrofuran (3800.00 mL). This was followed by the addition of 3-[(tert-butoxycarbonyl)amino]propanoic acid (380.00 g, 1.0 eq, 2.01 mol). The mixture was stirred for 3 h at r.t. To this was added 1-methyl 3-potassium propanedioate (627.00 g, 2 eq, 4.02 mol), MgCl2 (97.50 g, 1 eq, 2.01 mol). The resulting solution was stirred overnight at RT. The mixture diluted with 4 L of EA. The pH value of the solution was adjusted to 4 with KHSO4 (5%). The resulting solution was extracted with 4 L of ethyl acetate and the organic layers combined. The organic phase was washed with 1×1 L of NaHCO₃ and 1×500 mL of H2O. The resulting mixture was washed with 500 mL of brine. The mixture was dried over anhydrous sodium sulfate and concentrated to give the title compound.

### Step 2: methyl 5-[(tert-butoxycarbonyl)amino]-2-diazo-3-oxopentanoate (H-2)

Into a 10-L 4-necked round-bottom flask purged and maintained with an inert atmosphere of nitrogen, was placed a solution of methyl 5-[(tert-butoxycarbonyl)amino]-3-oxopentanoate (H-1) (475.00 g, 1.940 mmol, 1.00 equiv) in DCE (4750 mL). Then 4-acetamidobenzenesulfonyl azide (257.0 g, 1.05 eq.) and TEA (309.0 g, 3.0 eq.) was dropwise added with stirring in 1 h at 5° C. The resulting solution was stirred for 2 h at RT. The reaction was then quenched by the addition of 1 L of HCl. The pH value of the solution was adjusted to 2 with HCl (1 mol/L). The resulting solution was extracted with 3×1 L of dichloromethane, the organic phase was dried over anhydrous sodium sulfate and concentrated under vacuum to afford the title compound.

### Step 3: 1-tert-butyl 2-methyl 3-oxopyrrolidine-1,2-dicarboxylate (H-3)

Into a 5-L 4-necked round-bottom flask purged and maintained with an inert atmosphere of nitrogen, was placed [(CH3CN)4Cu]PF6 (8.23 g, 0.012 eq.) in DCE (500 mL). The mixture was warmed to75° C. The solution of methyl 5-[[(tert-butoxy)carbonyl]amino]-2-diazo-3-oxopentanoate (H-2) (500 g, 1 eq.) in DCE (2 L) was added dropwise with stirring. The resulting solution was stirred for 3 h at 75° C. The reaction mixture was cooled to 25° C. with a water/ice bath. The reaction was then quenched by the addition of 1 L of water. The resulting solution was extracted with 2×1 L of dichloromethane. The resulting mixture was washed with 1×500 mL of brine. The organic phase was dried over anhydrous sodium sulfate and concentrated under vacuum. The residue was applied onto a silica gel column (1:6 ethyl acetate/petroleum ether) to afford the title compound.

### Step 4: 1-(tert-butyl) 2-methyl (CIS)-3-((4-methoxybenzyl)amino) pyrrolidine-1,2-dicarboxylate (H-4)

Into a 3-L 3-necked round-bottom flask purged and maintained with an inert atmosphere of nitrogen, was placed 1-tert-butyl 2-methyl 3-oxopyrrolidine-1,2-dicarboxylate (H-3) (140.00 g, 636.000 mmol,1.0 eq.), tetrahydrofuran (1.4 L), PMBNH2 (79 g, 636.000 mmol, 1.0 eq.). This was followed by the addition of Ti(Oi-Pr)₄ (235 g, 827.000 mmol, 1.3 eq.) at 0° C. The resulting solution was stirred for 3 h at RT. The reaction was then quenched by the addition of water. The solids were filtered out. The resulting solution was extracted with 100 mL of THF and the organic layers combined. The resulting mixture was washed with 1×1 L of brine. The mixture was dried over anhydrous sodium sulfate. To this was added NaBH(OAc)3 (202 g, 954.000 mmol, 1.5 eq.). The resulting solution was stirred for 16 h at RT. The reaction was then quenched by the addition of water. The resulting solution was extracted with 3×1 L of EA and the organic layers combined. The resulting mixture was washed with 1×1 L of brine, The organic phase was dried over anhydrous sodium sulfate and concentrated. The residue was applied onto a silica gel column (1:3 ethyl acetate/petroleum ether) to afford the title compound.

### Step 5: 1-tert-butyl 2-methyl (CIS)-3-[(dimethylsulfamoyl)[(4-methoxyphenyl)methyl]-amino]pyrrolidine-1,2-dicarboxylate (H-5)

Into a 1-L 4-necked round-bottom flask purged and maintained with an inert atmosphere of nitrogen, was placed a solution of 1-tert-butyl 2-methyl (CIS)-3-[[(4-methoxyphenyl)-methyl]amino]pyrrolidine-1,2-dicarboxylate (H-4) (144.00 g, 396.000 mmol, 1.00 equiv) in DCE (432 mL), 3-(dimethylsulfamoyl)-1-methyl-1-(trifluoromethanesulfonyloxy)-1lambda4-imidazol-1-ium (201.00 g, 593.000 mmol, 1.50 equiv). The resulting solution was stirred overnight at 80 degrees C. The reaction mixture was cooled to 25 degree C. with a water/ice bath. The resulting mixture was concentrated under vacuum. The residue was applied onto a silica gel column (1:5 ethyl acetate/petroleum ether) to afford the title compound.

### Step 6: tert-butyl (CIS)-3-[(dimethylsulfamoyl)[(4-methoxyphenyl)methyl]amino]-2-(hydroxymethyl)pyrrolidine-1-carboxylate (H-6)

Into a 2-L 4-necked round-bottom flask purged and maintained with an inert atmosphere of nitrogen, was placed a solution of 1-tert-butyl 2-methyl (CIS)-3-[(dimethylsulfamoyl)[(4-methoxyphenyl)methyl]amino]pyrrolidine-1,2-dicarboxylate (H-5) (85.00 g, 180.000 mmol, 1.00 equiv) in THF (850 mL). This was followed by the addition of LiBH4 (361.00 mL, 720.000 mmol, 4.00 equiv) dropwise with stirring at 0 degrees C. The resulting solution was stirred for 4 h at 40 degrees C. The reaction mixture was cooled to 20 degree C. with a water/ice bath. The reaction was then quenched by the addition of 1 L of water/ice. The resulting solution was extracted with 3×1 L of ethyl acetate and the organic layers combined. The resulting mixture was washed with 1×500 mL of NH₄Cl. The mixture was dried over anhydrous sodium sulfate and concentrated under vacuum. The residue was applied onto a silica gel column (1:3 ethyl acetate/petroleum ether) to afford the title compound.

### Step 7: tert-butyl (CIS)-2-[[(tert-butyldiphenylsilyl)oxy]methyl]-3-[(dimethylsulfamoyl)[(4-methoxyphenyl)methyl]amino]pyrrolidine-1-carboxylate (H-7)

Into a 2-L 4-necked round-bottom flask purged and maintained with an inert atmosphere of nitrogen, was placed a solution of tert-butyl (CIS)-3-[(dimethylsulfamoyl)[(4-methoxy-phenyl)methyl]amino]-2-(hydroxymethyl)pyrrolidine-1-carboxylate (H-6) (60.00 g, 135.000 mmol, 1.00 equiv) in DCM (600 mL), imidazole (12.00 g, 149.000 mmol, 1.30 equiv). This was followed by the addition of DMAP (1.65 g, 13.500 mmol, 0.10 equiv) at 0 degrees C. To this was added TBDPSCl (40.82 g, 176.000 mmol, 1.10 equiv) at 0 degrees C. The resulting solution was stirred for 2 h at 25 degrees C. The reaction was then quenched by the addition of 300 mL of water/ice. The resulting solution was extracted with 2×600 mL of dichloromethane and the organic layers combined. The resulting mixture was washed with 1×500 mL of brine. The mixture was dried over anhydrous sodium sulfate and concentrated under vacuum. The residue was applied onto a silica gel column (1:3 ethyl acetate/petroleum ether) to afford the title compound.

### Step 8: tert-butyl (2R,3S)-2-[[(tert-butyldiphenylsilyl)oxy]methyl]-3-[(dimethylsulfamoyl)[(4-methoxyphenyl)methyl]amino]pyrrolidine-1-carboxylate (Intermediate H) and tert-butyl (2S,3R)-2-[[(tert-butyldiphenylsilyl)oxy]methyl]-3-[(dimethylsulfamoyl)[(4-methoxyphenyl)methyl]amino]pyrrolidine-1-carboxylate (H-8)

This obtained a mixture of tert-butyl (CIS)-2-[[(tert-butyldiphenylsilyl)oxy]methyl]-3-[(dimethylsulfamoyl)[(4-methoxyphenyl)methyl]amino]pyrrolidine-1-carboxylate (H-7) was then purified by Prep-SFC with the following conditions (Column, CHIRALPAK AD-33.0*100 mm, 3 um 001Lot No. AD3SCK-SB010; mobile phase, IPA(0.1% TEA)) to obtain: tert-butyl (2R,3S)-2-[[(tert-butyldiphenylsilyl)oxy]methyl]-3-[(dimethylsulfamoyl)[(4-methoxyphenyl)methyl]amino]pyrrolidine-1-carboxylate (INTERMEDIATE H): MS: (ES, m/z): 682 [M+H]. 1HMR: (300 MHz, DMSO-d₆, ppm): δ 7.69-7.32 (m, 10H), 7.09 (dd, J=16.3, 8.2 Hz, 2H), 6.85 (t, J=8.1 Hz, 2H), 4.62 (dd, J=25.5, 17.0 Hz, 1H), 4.27 (t, J=18.4 Hz, 2H), 4.00 (dd, J=34.1, 11.1 Hz, 1H), 3.73 (s, 5H), 3.37 (s, 2H), 2.52 (d, J=6.0 Hz, 6H), 2.25 (d, J=12.7 Hz, 1H), 2.04 (s, 1H), 1.44 (s, 5H), 1.27 (s, 4H), 0.99 (s, 9H). tert-butyl (2S,3R)-2-[[(tert-butyldiphenylsilyl)oxy]methyl]-3-[(dimethylsulfamoyl)[(4-methoxyphenyl)methyl]amino]pyrrolidine-1-carboxylate (H-8): (ES, m z): MS: 682 [M+H]. 1HNMR: (300 MHz, DMSO-d₆, ppm): δ 7.49 (ddq, J=27.4, 14.4, 7.2 Hz, 10H), 7.09 (dd, J=16.5, 8.2 Hz, 2H), 6.93-6.74 (m, 2H), 4.62 (dd, J=25.3, 16.9 Hz, 1H), 4.27 (t, J=18.1 Hz, 2H), 4.00 (dd, J=34.4, 10.7 Hz, 1H), 3.91-3.54 (m, 5H), 3.35 (s, 2H), 2.52 (d, J=6.0 Hz, 6H), 2.25 (d, J=12.7 Hz, 1H), 2.02 (d, J=13.9 Hz, 1H), 1.44 (s, 5H), 1.27 (s, 4H), 0.99 (s, 9H).

### Intermediate I

### benzyl (2R,3S)-3-((N,N-dimethylsulfamoyl)(4-methoxybenzyl)amino)-2-(((triethylsilyl) oxy)methyl)pyrrolidine-1-carboxylate

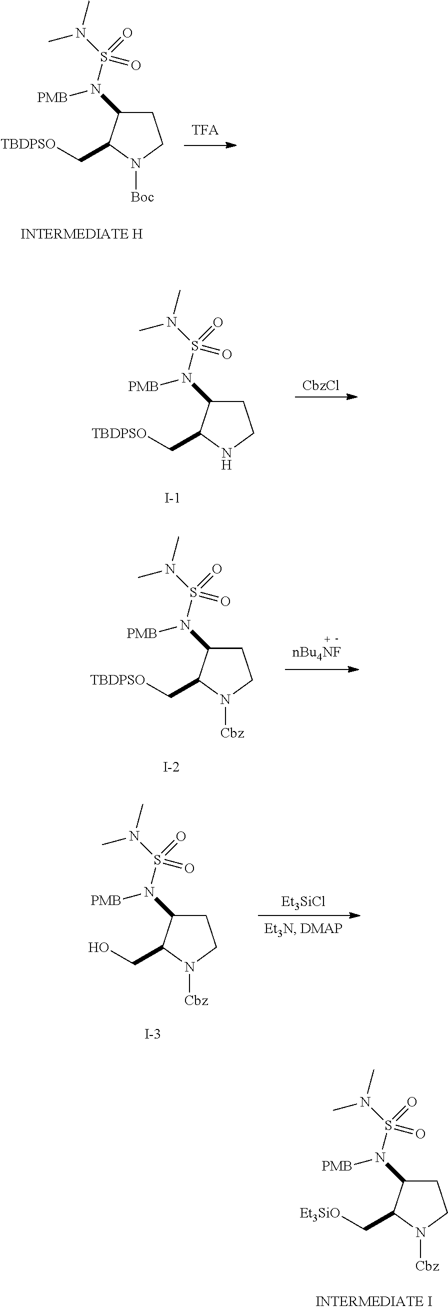

### Step 1: (2R,3S)-2-(((tert-butyldiphenylsilyl)oxy)methyl)-3-((N,N-dimethylsulfamoyl)(4-meth oxybenzyl)amino)pyrrolidine (I-1)

A solution of tert-butyl (2R,3S)-2-(((tert-butyldiphenylsilyl)oxy)methyl)-3-((N,N-dimethylsulfamoyl)(4-methoxybenzyl)amino)pyrrolidine-1-carboxylate (INTERMEDIATE H) (2000 mg, 2.93 mmol) in DCM (40 ml) in a ice bath was added TFA (8000 μL, 104 mmol) and stirred at 0 C. After 40 min, most of TFA and DCM were removed under reduced pressure and the residue was added aqueous sodium hydrogen carbonate (saturated, 100 mL) and the mixture was extracted with ethyl acetate (3×100 mL). The combined organic phases were combined and washed with brine (saturated, 100 mL), dried (Na₂SO₄), filtered and the solvent was evaporated under reduced pressure gave the title compound. MS: 582 (M+H).

### Step 2: benzyl (2R,3S)-2-(((tert-butyldiphenylsilyl)oxy)methyl)-3-((N,N-dimethylsulfamoyl)(4-methoxybenzyl)amino)pyrrolidine-1-carboxylate (I-2)

A solution of (2R,3S)-2-(((tert-butyldiphenylsilyl)oxy)methyl)-3-((N,N-dimethylsulfamoyl)(4-methoxybenzyl)amino)pyrrolidine (I-1) (592 mg, 1.017 mmol) in DCM (7000 μl) was added Et₃N (425 μl, 3.05 mmol), DMAP (62.2 mg, 0.509 mmol) followed by Cbz-Cl (218 μl, 1.526 mmol) at rt and stirred at rt for 1 hr. The reaction mixture was directly purified by silica column chromatography (0-50% EtOAc/hexanes) to give the title compound. MS: 716 (M+H).

### Step 3: benzyl (2R,3S)-3-((N,N-dimethylsulfamoyl)(4-methoxybenzyl)amino)-2-(hydroxy methyl)pyrrolidine-1-carboxylate (I-3)

A solution of benzyl (2R,3S)-2-(((tert-butyldiphenylsilyl)oxy)methyl)-3-((N,N-dimethylsulfamoyl)(4-methoxybenzyl)amino)pyrrolidine-1-carboxylate (I-2) (220 mg, 0.307 mmol) in THF (2000 μl) was added TETRABUTYLAMMONIUM FLUORIDE in THF (1.0 M, 369 μl, 0.369 mmol). After stirring at rt for 15 hrs, the mixture was added aqueous sodium hydrogen carbonate (saturated, 10 mL) and the mixture was extracted with ethyl acetate (3×20 mL). The combined organic fractions were washed with brine (saturated, 10 mL), dried (Na₂SO₄), filtered and the solvent was evaporated under reduced pressure to give the title compound. MS: 478 (M+H).

### Step 4: benzyl (2R,3S)-3-((N,N-dimethylsulfamoyl)(4-methoxybenzyl)amino)-2-(((triethylsilyl)oxy)methyl)pyrrolidine-1-carboxylate (Intermediate I)

A 40 mL vial containing benzyl (2R,3S)-3-((N,N-dimethylsulfamoyl)(4-methoxy benzyl)amino)-2-(hydroxymethyl)pyrrolidine-1-carboxylate (I-3) (344 mg, 0.720 mmol) in DCM (5 ml) was charged with DMAP (17.60 mg, 0.144 mmol). The vial was purged with N2, followed by addition of dry DCM (5 ml) and Et₃N (0.301 ml, 2.161 mmol). The resulting solution was then cooled in a ice bath and CHLOROTRIETHYLSILANE (0.145 ml, 0.864 mmol) was added dropwise. The resulting cloudy mixture was stirred at room temperature. After 15 hrs, the reaction was added aqueous sodium hydrogen carbonate (saturated, 50 mL) and the mixture was extracted with ethyl acetate (3×70 mL). The combined organic fractions were washed with brine (saturated, 50 mL), dried (Na₂SO₄), filtered and the solvent was evaporated under reduced pressure. The residue was purified by silica column chromatography (0-50% EtOAc/hexanes) to give the title compound. MS: 592 (M+H).

### Intermediate J

### tert-butyl (2R,3S,5R)-3-((N,N-dimethylsulfamoyl)(4-methoxybenzyl)amino)-5-methyl-2-(((4-(3-(((trifluoromethyl)sulfonyl)oxy)phenyl)cyclohexyl)oxy)methyl)pyrrolidine-1-carboxylate

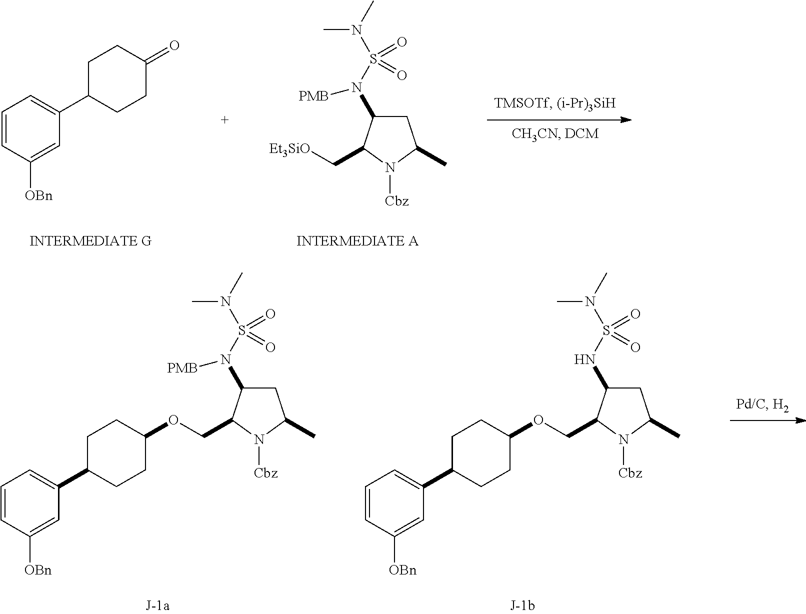

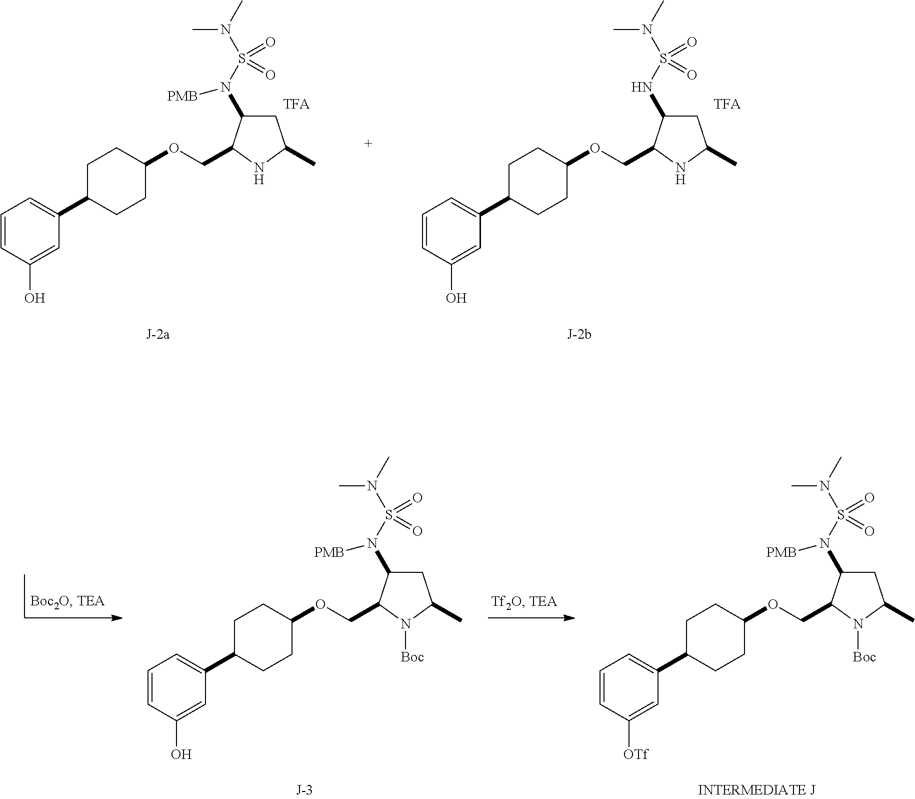

### Step 1: Benzyl (2R,3S,5R)-2-(((4-(3-(benzyloxy)phenyl)cyclohexyl)oxy)methyl)-3-((N,N-dimethylsulfamoyl)(4-methoxybenzyl)amino)-5-methylpyrrolidine-1-carboxylate (J-1a) and benzyl (2R,3S,5R)-2-(((4-(3-(benzyloxy)phenyl)cyclohexyl)oxy)methyl)-3-((N,N dimethylsulfamoyl)amino)-5-methylpyrrolidine-1-carboxylate (J-1b)

To a solution of benzyl (2R,3S,5R)-3-((N,N-dimethylsulfamoyl)-(4-methoxybenzyl)-amino)-5-methyl-2-(((triethylsilyl)oxy)methyl)pyrrolidine-1-carboxylate (INTERMEDIATE A) (2.5 g, 4.13 mmol) and 4-(3-(benzyloxy)phenyl)cyclohexan-1-one (1.17 g, 4.19 mmol) (INTERMEDIATE G) in CH₃CN (35 ml) was cooled in an ice bath and added triisopropylsilane (1693 μL, 8.25 mmol) followed by trimethylsilyl trifluoromethanesulfonate (900 μL, 4.98 mmol) as a solution in DCM (400 μl) dropwise under N₂. The resulting mixture was stirred at 0° C. for 30 min. The reaction was quenched with aqueous sodium hydrogen carbonate (saturated, 100 mL) and the mixture was extracted with ethyl acetate (3×150 mL). The combined organic fractions were washed with brine (saturated, 100 mL), dried (Na₂SO₄), filtered and the solvent was evaporated under reduced pressure. The residue was purified by silica column chromatography (0-50% EtOAc/hexanes) to give a mixture of the title compounds J-1a (MS: 636 (M+H)) and J-1b (MS: 756 (M+H)).

### Step 2: (2R,3S,5R)-2-(((4-phenyl)cyclohexyl)oxy)methyl)-3-((N,N-dimethylsulfamoyl)(4-methoxybenzyl)amino)-5-methylpyrrolidine, trifluroacetic acid salt (J-2a) and (2R,3S,5R)-2-(((4-(3-(benzyloxy)phenyl)cyclohexyl)oxy)methyl)-3-((N,N-dimethylsulfamoylamino)-5-methylpyrrolidine, trifluroacetic acid salt (J-2b)

To a mixture of both Benzyl (2R,3S,5R)-2-(((4-(3-(benzyloxy)phenyl)-cyclohexyl)oxy)methyl)-3-((N,N-dimethylsulfamoyl)(4-methoxybenzyl)amino)-5-methylpyrrolidine-1-carboxylate (J-1a) and benzyl (2R,3S,5R)-2-(((4-(3-(benzyloxy)phenyl)-cyclohexyl)oxy)methyl)-3-((N,N-dimethylsulfamoyl)amino)-5-methylpyrrolidine-1-carboxylate (J-1b) (2.74 g, 3.62 mmol) in EtOH (20 ml)/Ethyl acetate (15 ml) was added palladium on carbon (0.386 g, 0.362 mmol). The reaction was stirred under a H₂ balloon. After 5 hrs, the reaction went to completion and the mixture was filtered through a pad of celite and washed with methanol. The combined filtrates were concentrated under reduced pressure. The residue was purified by C18 column chromatography (10-100% Water in Acetonitrile with 0.05% TFA) to obtain separately J-2a (MS: 532 (M+H)) and J-2b (MS: 412 (M+H)) as TFA salts.

### Step 3: tert-butyl (2R,3S,5R)-3-((N,N-dimethylsulfamoyl)(4-methoxybenzyl)amino)-2-(((4-(3-hydroxyphenyl)cyclohexyl)oxy)methyl)-5-methylpyrrolidine-1-carboxylate (J-3)

A solution of (2R,3S)-3-((N,N-dimethylsulfamoyl)(4-methoxybenzyl)amino)-2-(((4-(3-hydroxyphenyl)cyclohexyl)oxy)methyl)pyrrolidine, TFA salt (J-2a) (430 mg, 0.683 mmol) and Et₃N (0.3 ml, 2.152 mmol) in DCM (7 ml) was added di-tert-butyl dicarbonate (156 mg, 0.717 mmol) at rt. After stirring at rt for 30 min, the reaction went to completion. The solvent was removed under reduced pressure to give the title compound. MS: 632 (M+H).

### Step 4: tert-butyl (2R,3S,5R)-3-((N,N-dimethylsulfamoyl)(4-methoxybenzyl)amino)-5-methyl-2-(((4-(3-(((trifluoromethyl)sulfonyl)oxy)phenyl)cyclohexyl)oxy)methyl)pyrrolidine-1-carboxylate (Intermediate J)

A solution of tert-butyl (2R,3S,5R)-3-((N,N-dimethylsulfamoyl)(4-methoxybenzyl)-amino)-2-(((4-(3-hydroxyphenyl)cyclohexyl)oxy)methyl)-5-methylpyrrolidine-1-carboxylate (J-3) (205 mg, 0.324 mmol) in DCM (7.0 ml) was cooled to OC and added triethylamine (0.3 ml, 2.152 mmol) followed by trifluoromethanesulfonic anhydride (0.751 ml, 0.751 mmol). The ice bath was removed and the mixture was stirred at rt for 30 min. The reaction mixture was directly purified by silica column chromatography (0-50% EtOAc/hexanes) to give the title compound. MS: 764 (M+H).

### Intermediate K

### (2R,3S,5R)-2-(((4-phenyl)cyclohexyl)oxy)methyl)-3-((N,N-dimethylsulfamoyl)(4-methoxy benzyl)amino)-5-methylpyrrolidine

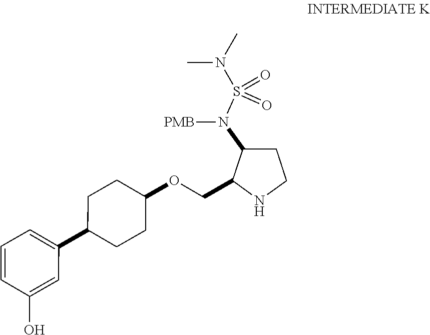

(2R,3S,5R)-2-(((4-phenyl)cyclohexyl)oxy)methyl)-3-((N,N-dimethylsulfamoyl)(4-methoxy benzyl)amino)-5-methylpyrrolidine (INTERMEDIATE K) was prepared according to the same procedure provided in preparation of J-2a by using the appropriate reagents and INTERMEDIATE I. MS: 518 (M+H).

### Intermediate L

### N-((2R,3S,5R)-2-(((4-(3-hydroxyphenyl)cyclohexyl)oxy)methyl)-5-methylpyrrolidin-3-yl)-N-(4-methoxybenzyl)methanesulfonamide

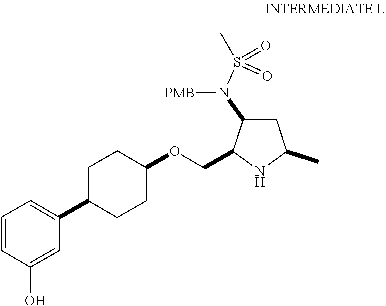

N-((2R,3S,5R)-2-(((4-(3-hydroxyphenyl)cyclohexyl)oxy)methyl)-5-methylpyrrolidin-3-yl)-N-(4-methoxybenzyl)methanesulfonamide (INTERMEDIATE L) was prepared according to the same procedure provided in preparation of J-2a by using the appropriate reagents and INTERMEDIATE B. MS: 503 (M+H).

### Intermediate M

### 2R,3S,5R-3-((N,N-dimethylsulfamoyl)(4-methoxybenzyl)amino)-2-(((4-(3-hydroxyphenyl)cyclohexyl)oxy)methyl)-5-methylpyrrolidine

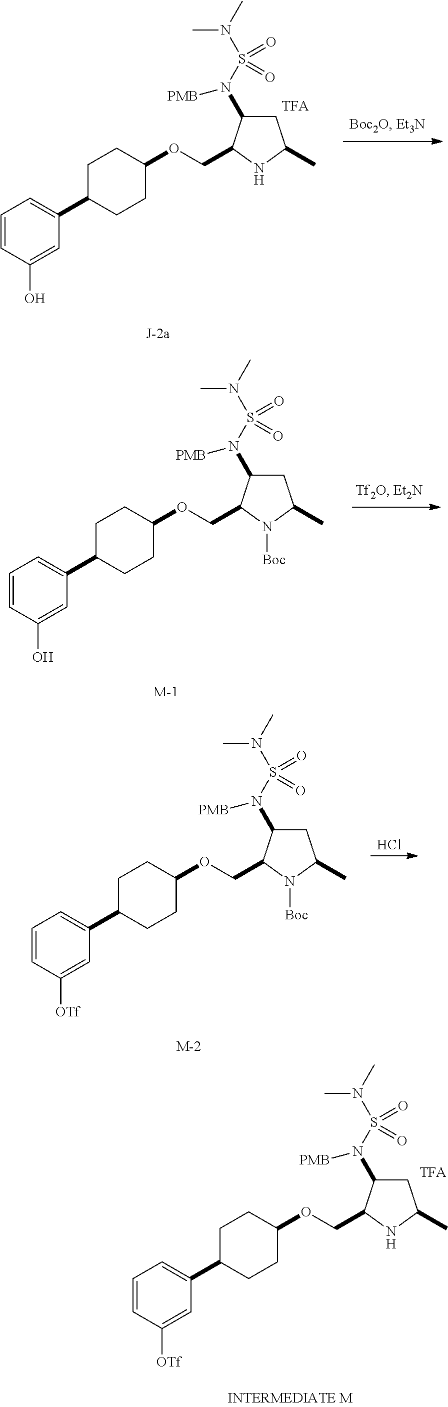

### Step 1: tert-butyl (2R,3S,5R)-3-((N,N-dimethylsulfamoyl)(4-methoxybenzyl)amino)-2-(((4-(3-hydroxyphenyl)cyclohexyl)oxy)methyl)-5-methylpyrrolidine-1-carboxylate (M-1)

A solution of 2R,3S,5R-3-((N,N-dimethylsulfamoyl)(4-methoxybenzyl)amino)-2-(((4-(3-hydroxyphenyl)cyclohexyl)oxy)methyl)-5-methylpyrrolidine (J-2a) (800 mg, 1.505 mmol) in DCM (15 ml) was added Et₃N (629 μl, 4.51 mmol) followed by di-tert-butyl dicarbonate (380 μl, 1.655 mmol). After stirring at rt for 2 hrs, the reaction mixture was directly purified by silica column chromatography (0-70% EtOAc:EtOH (3:1 v/v)/Hexanes) to give the title compound. MS: 632 (M+H).

### Step 2: tert-butyl (2R,3S,5R)-3-((N,N-dimethylsulfamoyl)(4-methoxybenzyl)amino)-5-methyl-2-(((4-(3-(((trifluoromethyl)sulfonyl)oxy)phenyl)cyclohexyl)oxy)methyl)pyrrolidine-1-carboxylate (M-2)

A solution of tert-butyl (2R,3S,5R)-3-((N,N-dimethylsulfamoyl)(4-methoxybenzyl)amino)-2-(((4-(3-hydroxyphenyl)cyclohexyl)oxy)methyl)-5-methylpyrrolidine-1-carboxylate (M-1) (431 mg, 0.324 mmol) was added Et₃N (0.3 ml, 2.152 mmol) followed by trifluoromethanesulfonic anhydride in DCM (1.0 M, 0.751 ml, 0.751 mmol). After stirring at rt for 1 hr, the reaction mixture was directly purified by silica column chromatography (0-50% EtOAc/hexanes) to give the title compound. MS: 764 (M+H).

### Step 3: 3-(4-(((2R,3S,5R)-3-((N,N-dimethylsulfamoyl)(4-methoxybenzyl)amino)-5-methylpyrrolidin-2-yl)methoxy)cyclohexyl)phenyl trifluoromethanesulfonate TFA salt (Intermediate M)

Tert-butyl (2R,3S,5R)-3-((N,N-dimethylsulfamoyl)(4-methoxybenzyl)amino)-5-methyl-2-(((4-(3-(((trifluoromethyl)sulfonyl)oxy)phenyl)cyclohexyl)oxy)methyl)pyrrolidine-1-carboxylate (M-2) (67 mg, 0.088 mmol) was stirred in DCM (1000 μl) and TFA (1000 μl) at rt. After 1 hr, the solvent and excess TFA were removed under reduced pressure. The residue was purified by C18 column chromatography (10-100% Water in Acetonitrile with 0.05% TFA) to give the title compound as a TFA salt. MS: 664 (M+H).

### Intermediate N

### tert-butyl (2R,3S)-3-((N,N-dimethylsulfamoyl)(4-methoxybenzyl)amino)-2-(((4-(3-(((trifluoromethyl)sulfonyl)oxy)phenyl)cyclohexyl)oxy)methyl)pyrrolidine-1-carboxylate

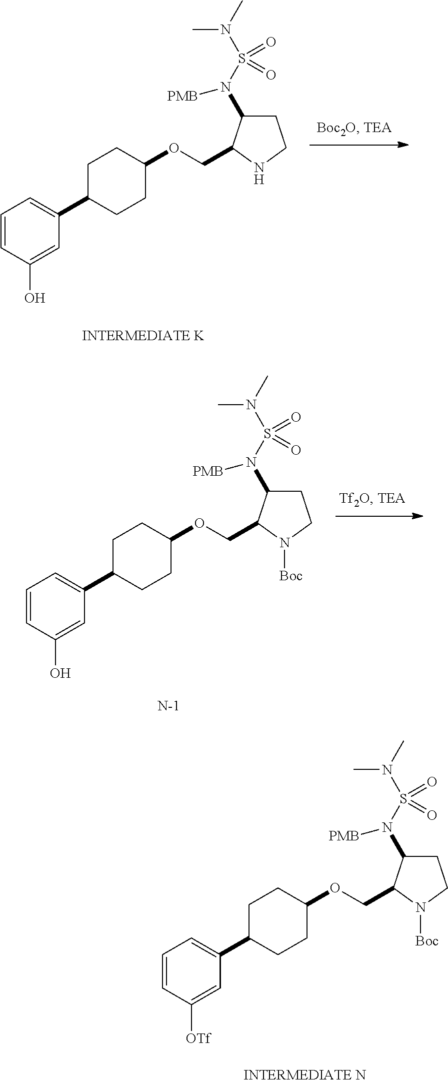

### Step 1: tert-butyl (2R,3S)-3-((N,N-dimethylsulfamoyl)(4-methoxybenzyl)amino)-2-(((4-(3-hydroxyphenyl)cyclohexyl)oxy)methyl)pyrrolidine-1-carboxylate (N-1)

A solution of (2R,3S)-2-(((4-(3-phenyl)cyclohexyl)oxymethyl)-3-((N,N-dimethylsulfamoyl)(4-methoxybenzyl)amino)pyrrolidine (INTERMEDIATE K) (533 mg, 1.030 mmol) in DCM (10 ml) was added Et₃N (0.431 ml, 3.09 mmol) followed by di-tert-butyl dicarbonate (225 mg, 1.030 mmol). Stirred at rt for 15 hrs. The reaction mixture was directly purified by silica column chromatography (0-70% EtOAc/hexane) to give the title compound. MS: 618 (M+H).

### Step 2: tert-butyl (2R,3S)-3-((N,N-dimethylsulfamoyl)(4-methoxybenzyl)amino)-2-(((4-(3-(((trifluoromethyl)sulfonyl)oxy)phenyl)cyclohexyl)oxy)methyl)pyrrolidine-1-carboxylate (Intermediate N)

To a stirred solution of tert-butyl (2R,3S)-3-((N,N-dimethylsulfamoyl)(4-methoxybenzyl)amino)-2-(((4-(3-hydroxyphenyl)cyclohexyl)oxy)methyl)pyrrolidine-1-carboxylate (N-1) (636 mg, 1.03 mmol) in DCM (10 ml) cooled in an ice bath was added triethylamine (0.431 ml, 3.09 mmol) followed by trifluoromethanesulfonic anhydride in DCM (1.0 M,1.133 ml, 1.133 mmol) slowly. After addition, the ice bath was removed and the mixture was stirred at rt for 1 hr. The mixture was directly purified by silica column chromatography (0-50% EtOAc/Hexanes) to give the title compound. MS: 750 (M+H).

### Intermediate O

### N-((2R,3S,5R)-2-(((4-(3-fluoro-5-hydroxyphenyl)cyclohexyl)oxy)methyl)-5-methylpyrrolidin-3-yl)-N-(4-methoxybenzyl)methanesulfonamide

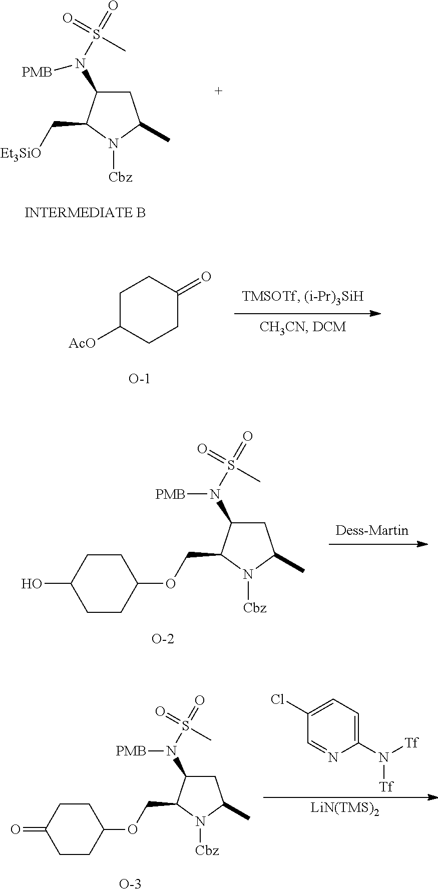

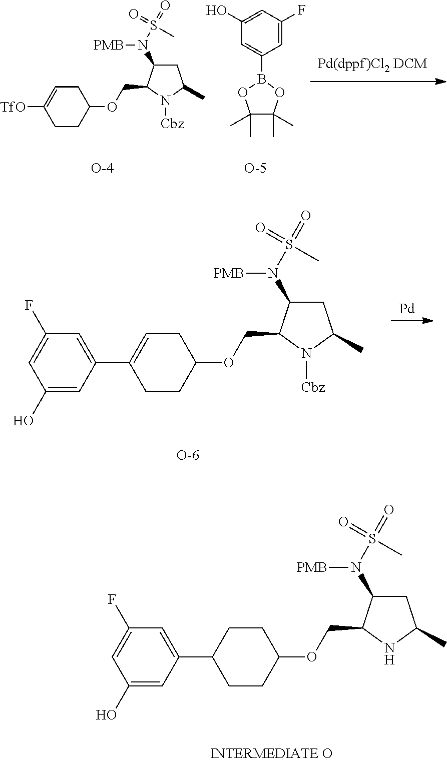

### Step 1: Benzyl (2R,3S,5R)-2-(((4-hydroxycyclohexyl)oxy)methyl)-3-(N-(4-methoxybenzyl)methylsulfonamido)-5-methylpyrrolidine-1-carboxylate (0-2)

A solution of benzyl (2R,3S,5R)-3-(N-(4-methoxybenzyl)methylsulfonamido)-5-methyl-2-(((triethylsilyl)oxy)methyl)pyrrolidine-1-carboxylate (INTERMEDIATE B)(1.98 g, 3.43 mmol) and 4-oxocyclohexyl acetate (590 mg, 3.78 mmol) (0-1) in Acetonitrile (16 ml) cooled in an ice bath was added TRIISOPROPYLSILANE (1.408 ml, 6.87 mmol) followed by TRIMETHYLSILYL TRIFLUOROMETHANESULFONATE (0.682 ml, 3.78 mmol) as a solution in CH₂Cl₂ (2 ml) dropwise. The resulting solution was stirred at 0° C. After 1 hr, to the mixture was added MeOH (15 ml) and aq LiOH (1.0 M, 12 ml, 12.00 mmol). After stirring at rt for 60 min, the mixture was concentrated to remove most of solvent and the residue was added water (100 mL) and extracted with ethyl acetate (3×150 mL). The combined organic fractions were washed with brine (saturated, 100 mL), dried (Na₂SO₄), filtered and the solvent was evaporated under reduced pressure to give the title compound. MS: 561 [M+H]⁺.

### Step 2: benzyl (2R,3S,5R)-3-(N-(4-methoxybenzyl)methylsulfonamido)-5-methyl-2-(((4-oxocyclohexyl)oxy)methyl)pyrrolidine-1-carboxylate (0-3)

A solution of benzyl (2R,3S,5R)-2-(((4-hydroxycyclohexyl)oxy)methyl)-3-(N-(4-methoxybenzyl)methylsulfonamido)-5-methylpyrrolidine-1-carboxylate (0-2)(1.9 g, 3.39 mmol) in CH₂Cl₂ (30 ml) cooed in an ice bath was added Dess-Martin Periodinane (2.156 g, 5.08 mmol). After stirring at 0° C. for 4 min, the ice bath was removed and the resulting solution was stirred at rt. After 1 hr, the mixture was added water (100 mL) and extracted with CH₂Cl₂ (3×100 mL). The combined organic fractions were washed with brine (saturated, 100 mL), dried (Na₂SO₄) and filtered. The solvent was evaporated under reduced pressure. The residue was purified by silica column chromatography (0-70% EtOAc/isohexane) to give the title compounds. MS: 559 [M+H]⁺.

### Step 3: benzyl (2R,3S,5R)-3-(N-(4-methoxybenzyl)methylsulfonamido)-5-methyl-2-(((4-(((trifluoromethyl)sulfonyl)oxy)cyclohex-3-en-1-yl)oxy)methyl)pyrrolidine-1-carboxylate (0-4)

A solution of benzyl (2R,3S,5R)-3-(N-(4-methoxybenzyl)methylsulfonamido)-5-methyl-2-(((4-oxocyclohexyl)oxy)methyl)pyrrolidine-1-carboxylate (0-3)(1096 mg, 1.962 mmol) and and 2-[N,N-BIS(TRIFLUOROMETHANESULFONYL)AMINO]-5-CHLOROPYRIDINE (847 mg, 2.158 mmol) cooled at −78° C. was added LITHIUM BIS(TRIMETHYLSILYL)AMIDE (2.158 ml, 2.158 mmol) slowly. The resulting solution was stirred at −78° C. After 2 hrs, the mixture was quenched with water (100 mL) and brine 50 ml. The mixture was extracted with ethyl acetate (3×100 mL). The combined organic fractions were washed with brine (saturated, 100 mL), dried (Na₂SO₄), filtered and the solvent was evaporated under reduced pressure. The residue was purified by silica column chromatography (0-60% EtOAc/isohexane). The desired fractions were combined and concentrated under reduced pressure to isolate the desired product with some impurities. Second purification was conducted using C18 column chromatography (10-100% Water in Acetonitrile) to give the title compound. MS: 691 [M+H]+.

### Step 4: benzyl (2R,3S,5R)-2-(((3′-fluoro-5′-hydroxy-2,3,4,5-tetrahydro-[1,1′-biphenyl]-4-yl)oxy)methyl)-3-(N-(4-methoxybenzyl)methylsulfonamido)-5-methylpyrrolidine-1-carboxylate (O-6)

A mixture of benzyl (2R,3S,5R)-3-(N-(4-methoxybenzyl)methylsulfonamido)-5-methyl-2-(((4-(((trifluoromethyl)sulfonyl)oxy)cyclohex-3-en-1-yl)oxy)methyl)pyrrolidine-1-carboxylate (160 mg, 0.232 mmol) (O-4), 3-fluoro-5-hydroxybenzeneboronic acid pinacol ester (0-5)(71.7 mg, 0.301 mmol),[1,1′bis(diphenylphosphino)ferrocene]dichloropalladium(II) (33.9 mg, 0.046 mmol) and Cs₂CO₃ (226 mg, 0.695 mmol) in THF (2 ml) charged in a 10 ml of microwave vial was bubbled with N₂ for 3 min, then capped the vial and heated to 90° C. After 1 hr, the reaction mixture was cooled to rt and concentrated under reduced pressure to remove the solvent. The resulting residue was purified by silica column chromatography (0-60% EtOAc/isohexane) to give the title compound. MS: 653 [M+H]⁺.

### Step 5: N-((2R,3S,5R)-2-(((4-(3-fluoro-5-hydroxyphenyl)cyclohexyl)oxy)methyl)-5-methylpyrrolidin-3-yl)-N-(4-methoxybenzyl)methanesulfonamide (Intermediate O)

A solution of benzyl (2R,3S,5R)-2-(((3′-fluoro-5′-hydroxy-2,3,4,5-tetrahydro-[1,1′-biphenyl]-4-yl)oxy)methyl)-3-(N-(4-methoxybenzyl)methylsulfonamido)-5-methylpyrrolidine-1-carboxylate (0-6)(146 mg, 0.224 mmol) in THF (3 mL) was added Pd/C (23.80 mg, 0.022 mmol) and acetic acid (150 μL, 2.62 mmol). The resulting suspension was degassed and refilled with H₂ from a balloon for three times, then was stirred under a H₂ balloon for 4 hrs. The mixture was filtered, washing with ethyl acetate. The combined filtrates were concentrated under reduced pressure to give the title compound. MS: 521 [M+H]⁺.

### Intermediate P-U

### N-((2R,3S,5R)-2-(((4-(2-fluoro-3-hydroxyphenyl)cyclohexyl)oxy)methyl)-5-methylpyrrolidin-3-yl)-N-(4-methoxybenzyl)methanesulfonamide (Intermediate P), N-((2R,3S,5R)-2-(((4-(2,5-difluoro-3-hydroxyphenyl)cyclohexyl)oxy)methyl)-5-methylpyrrolidin-3-yl)-N-(4-methoxybenzyl)methanesulfonamide (Intermediate O), N-((2R,3S,5R)-2-(((4-(4-cyano-3-hydroxyphenyl)cyclohexyl)oxy)methyl)-5-methylpyrrolidin-3-yl)-N-(4-methoxybenzyl)methanesulfonamide (Intermediate R), N-((2R3S,5R)-2-(((4-(2,5-difluoro-3-hydroxyphenyl)cyclohexyl)oxy)methyl)-5-methylpyrrolidin-3-yl)-N-(4-methoxybenzyl)methanesulfonamide (Intermediate S), N-((2R,3S,5R)-2-(((4-(3-hydroxy-5-(trifluoromethyl)phenyl)cyclohexyl)oxy)methyl)-5-methylpyrrolidin-3-yl)-N-(4-methoxybenzyl)methanesulfonamide (Intermediate T), and N-((2R,3S,5R)-2-(((4-(3-hydroxy-5-(trifluoromethoxy)phenyl)cyclohexyl)oxy)methyl)-5-methylpyrrolidin-3-yl)-N-(4-methoxybenzyl)methanesulfonamide (Intermediate U)

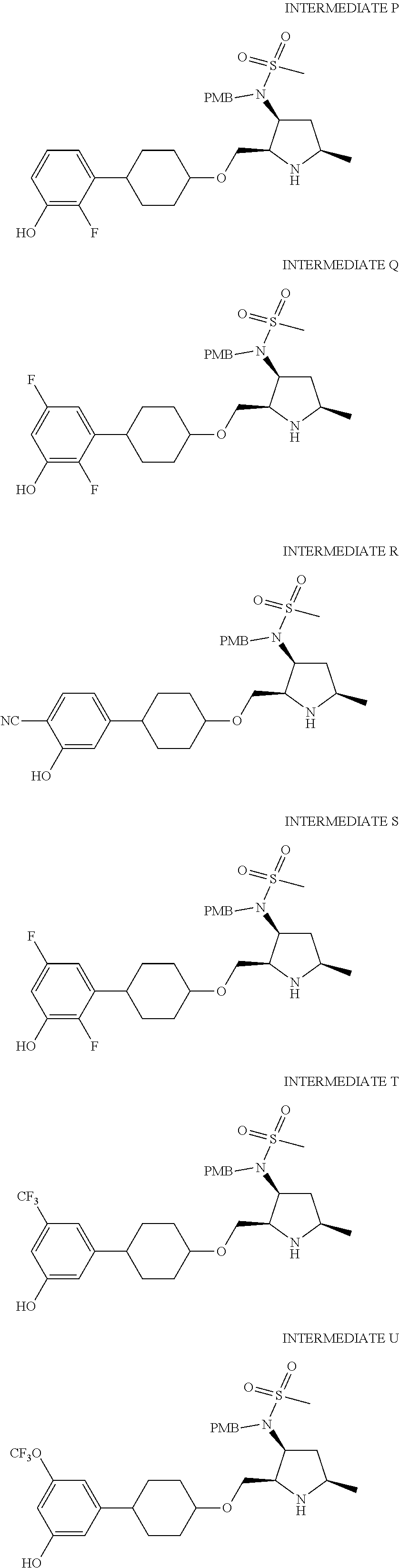

The following INTERMEDIATES P through U were prepared according to the general procedures herein and in an analogous manner to that used to synthesize INTERMEDIATE O using the appropriate boronic ester starting materials.

### Intermediate V

### 2-chloroethyl (2R,3S,5R)-3-(N-(4-methoxybenzyl)methylsulfonamido)-5-methyl-2-(((triethylsilyl)oxy)methyl)pyrrolidine-1-carboxylate

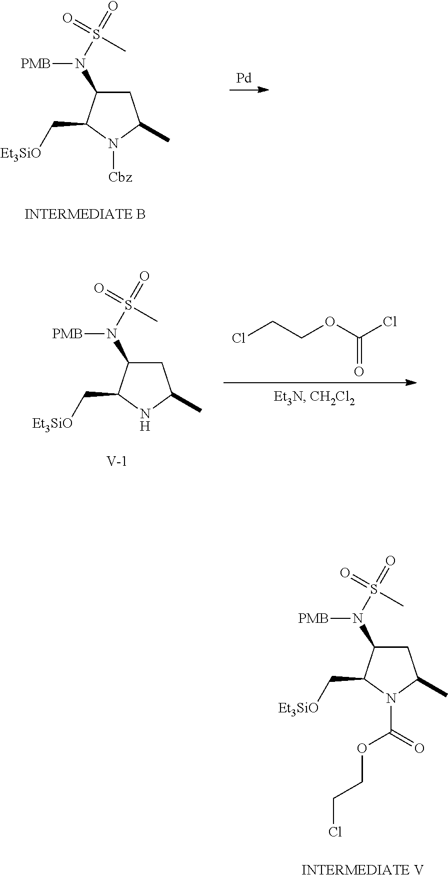

### Step 1: N-(4-methoxybenzyl)-N-((2R,3S,5R)-5-methyl-2-(((triethylsilyl)oxy)methyl)pyrrolidin-3-yl)methanesulfonamide (V-1)

A mixture of benzyl (2R,3S,5R)-3-(N-(4-methoxybenzyl)methylsulfonamido)-5-methyl-2-(((triethylsilyl)oxy)methyl)pyrrolidine-1-carboxylate (INTERMEDIATE B)(4.0 g, 6.93 mmol) and palladium (0.590 g, 0.555 mmol) in THF (45 ml) was degassed and refilled with H₂ from a balloon for three times. The suspension was stirred under a H₂ balloon for 5 hrs. The mixture was filtered via a celite cake, washing with methanol. The combined filtrates were concentrated under reduced pressure to give the title compound. MS: 443 [M+H]⁺.

### Step 2: 2-chloroethyl (2R,3S,5R)-3-(N-(4-methoxybenzyl)methylsulfonamido)-5-methyl-2-(((triethylsilyl)oxy)methyl)pyrrolidine-1-carboxylate (Intermediate V)

To a solution of N-(4-methoxybenzyl)-N-((2R,3S,5R)-5-methyl-2-(((triethylsilyl)oxy) methyl)pyrrolidin-3-yl)methanesulfonamide (V-1) (1000 mg, 2.259 mmol) in CH₂CL₂ (22 mL) was added Et₃N (0.409 mL, 2.94 mmol) followed by 2-chloroethyl carbonochloridate (339 mg, 2.372 mmol) at rt. After stirring at rt for 15 min, the reaction mixture was added water (100 ml) and the mixture was extracted with dichloromethane (3×150 mL). The combined organic fractions were washed with brine (saturated, 100 mL), dried (Na₂SO₄), filtered and the solvent was evaporated under reduced pressure. The residue was purified by silica column chromatography (0-50% EtOAc/isohexane) to give the title compound. MS: 549 [M+H]+.

### Intermediate W

### 3-chloropropyl (2R,3S,5R)-3-(N-(4-methoxybenzyl)methylsulfonamido)-5-methyl-2-(((triethylsilyl)oxy)methyl)pyrrolidine-1-carboxylate

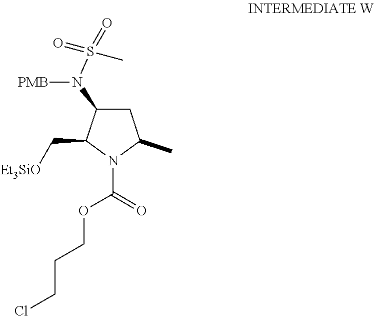

INTERMEDIATE W was prepared according to the procedures used to synthesize INTERMEDIATE V using 3-chloropropyl carbonochloridate.

### Intermediate X

### 2-(benzyloxy)-6-chloro-4-ethoxypyridine

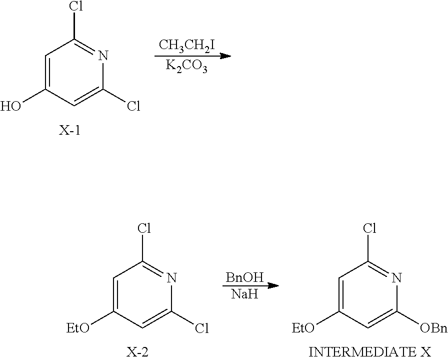

### Step 1: 2,6-dichloro-4-ethoxypyridine (X-2)

A solution of 2,6-dichloropyridin-4-ol (X-1)(1500 mg, 9.15 mmol) in DMF (60 mL) was added iodoethane (2.206 ml, 27.4 mmol) and potassium carbonate (3793 mg, 27.4 mmol). The resulting suspension was stirred at rt for 2 hrs. The mixture was filtered via a celite cake, washing with ethyl acetate. The combined filtrates were concentrated under reduced pressure. The residue was purified by silica column chromatography (0-20% EtOAc/isohexane) to give the title compound. MS: 193 [M+H]⁺

### Step 2: 2-(benzyloxy)-6-chloro-4-ethoxypyridine (Intermediate X)

A solution of phenylmethanol (0.988 ml, 9.50 mmol) in THF (5 mL) at 0° C. was added NaH (0.608 g, 15.21 mmol). The suspension was stirred at 0° C. for 10 min before adding a solution of 2,6-dichloro-4-ethoxypyridine (X-1)(1.46 g, 7.60 mmol) in THF (60 mL). Ice bath was removed and the reaction was stirred at rt. After 4 hrs, the mixture was quenched with water (150 mL) and the mixture was extracted with ethyl acetate (3×100 mL). The combined organic fractions were washed with brine (saturated, 100 mL), dried (Na₂SO₄), filtered and the solvent was evaporated under reduced pressure. The residue was purified by silica column chromatography (0-40% EtOAc/isohexane) to give the title compound. MS: 264 [M+H]⁺.

### Intermediate Y

### 2-(benzyloxy)-6-chloro-3-fluoro-4-methoxypyridine

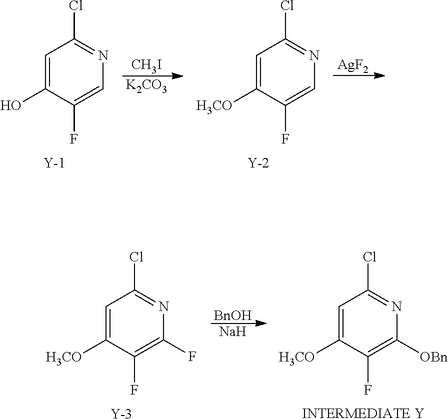

### Step 1: 2-chloro-5-fluoro-4-methoxypyridine (Y-2)

Y-2 was prepared according to the procedures used to synthesize X-2 using the appropriate iodomethane starting material. MS: 162 [M+H]⁺.

### Step 2: 6-chloro-2,3-difluoro-4-methoxypyridine (Y-3)

An oven dried flask was charged a solution of 2-chloro-5-fluoro-4-methoxypyridine (Y-2) (1.39 g, 8.60 mmol) in anhydrous CH₃CN (44 mL). To this was added SILVER(II) FLUORIDE (3.76 g, 25.8 mmol) under N₂ at rt. The suspension was stirred at rt, After stirring for 4 hrs, another portion of SILVER(II) FLUORIDE (1.83 g, 12.9 mmol) was added to the reaction and stirred overnight at rt. The mixture was filtered via a celite cake, washing with dichloromethane. The combined filtrates were added 2.0 N aqueous sodium hydroxide (100 mL) and brine (100 mL). The mixture was extracted with dichloromethane (3×100 mL). The combined organic fractions were dried (MgSO₄), filtered and the solvent was evaporated under reduced pressure in a cold bath product to give the title compound. MS: 180 [M+H]⁺.

### Step 3: 2-(benzyloxy)-6-chloro-3-fluoro-4-methoxypyridine (Intermediate Y)

INTERMEDIATE Y was prepared according to the procedures used to synthesize INTERMEDIATE X. MS: 268 [M+H]+.

### Intermediate Z

### 6-chloro-N-(4-methoxybenzyl)-N-methyl-5-(trifluoromethyl)pyridin-2-amine

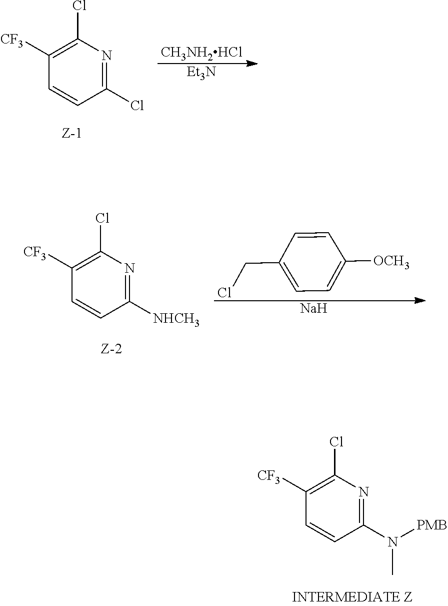

### Step 1: 6-chloro-N-methyl-5-(trifluoromethyl)pyridin-2-amine (Z-2)

To a solution of 2,6-dichloro-3-(trifluoromethyl)pyridine (Z-1) (1600 mg, 7.41 mmol) and Et₃N (3.10 mL, 22.22 mmol) in DMF (15 ml) charged in a 40 ml vial was added METHYLAMINE HYDROCHLORIDE (750 mg, 11.11 mmol) at rt. The mixture was stirred at rt. After 2 hrs, the suspension was added EtOAc and filtered via a celite cake, washing with ethyl acetate. The combined filtrates were concentrated under reduced pressure to give the title compound. MS: 212 [M+H]⁺.

### Step 2: 6-chloro-N-(4-methoxybenzyl)-N-methyl-5-(trifluoromethyl)pyridin-2-amine (Intermediate Z)

To a solution of 6-chloro-N-methyl-5-(trifluoromethyl)pyridin-2-amine (Z-2) (1.56 g, 7.41 mmol) and 1-(chloromethyl)-4-methoxybenzene (2.0 mL, 13.92 mmol) in DMF (50 mL) was carefully added NaH (1.185 g, 29.6 mmol) in portions at rt. The mixture was stirred at rt. After 30 min, the reaction mixture was carefully poured to 100 ml of stirring ice water and the mixture was extracted with 1:1 EtOAc/Hexane (3×150 mL). The combined organic fractions were washed with brine (saturated, 100 mL), dried (Na₂SO₄), filtered and the solvent was evaporated under reduced pressure. The residue was purified by silica column chromatography (0-20% EtOAc/isohexane) to give the title compound. MS: 331 [M+H]⁺.

### Intermediate AA

### 6-chloro-4-methoxy-N,N-bis(4-methoxybenzyl)pyridin-2-amine

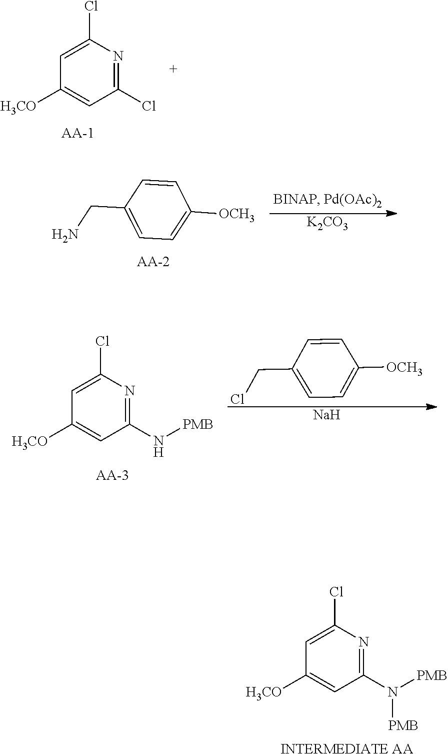

### Step 1: 6-chloro-4-methoxy-N-(4-methoxybenzyl)pyridin-2-amine (AA-3)

A suspension of 2,6-dichloro-4-methoxypyridine (AA-1)(600 mg, 3.37 mmol), (R)-(+)-2,2′-bis(diphenylphosphino)-1,1′-binaphthyl (BINAP) (210 mg, 0.337 mmol), (4-methoxyphenyl)methanamine (AA-2) (0.528 ml, 4.04 mmol), palladium(II) Acetate (76 mg, 0.337 mmol) and potassium carbonate (1630 mg, 11.80 mmol) in Toluene (16 mL) in a sealed ml of microwave vial was bubbled with N₂ for 3 min. Heat to 100° C. for 2.5 hrs, then the mixture was added water (150 mL) and was extracted with ethyl acetate (3×100 mL). The combined organic fractions were washed with brine (saturated, 100 mL), dried (Na₂SO₄), filtered and the solvent was evaporated under reduced pressure. The residue was purified by silica column chromatography (0-20% EtOAc/isohexane) to give the title compound. MS: 279 [M+H]⁺.

### Step 2: 6-chloro-4-methoxy-N,N-bis(4-methoxybenzyl)pyridin-2-amine (Intermediate AA)

6-chloro-4-methoxy-N-(4-methoxybenzyl)pyridin-2-amine (AA-3)(130 mg, 0.466 mmol) in DMF (4000 μl) was added NaH (37.3 mg, 0.933 mmol). The suspension was stirred at rt. After 1 hr, the reaction mixture was cooled in an ice bath and quenched with water (30 mL) and the mixture was extracted with ethyl acetate (3×30 mL). The combined organic fractions were washed with brine (saturated, 30 mL), dried (Na₂SO₄), filtered and the solvent was evaporated under reduced pressure. The residue was purified by silica column chromatography (0-20% EtOAc/isohexane) to give the title compound. MS: 399 [M+H]⁺.

### Intermediate BB

### 6-chloro-4-methoxy-N-(4-methoxybenzyl)-N-methylpyridin-2-amine

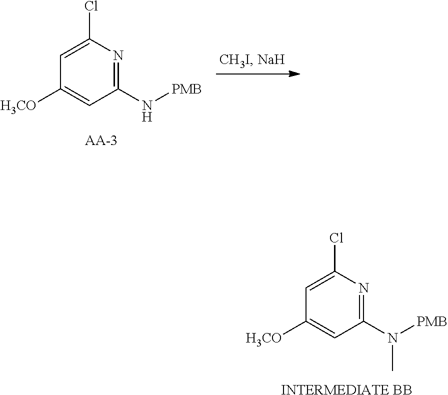

To a solution of 6-chloro-4-methoxy-N-(4-methoxybenzyl)pyridin-2-amine (AA-3)(139 mg, 0.499 mmol) and iodomethane (0.094 ml, 1.496 mmol) in DMF (5 ml) was added NaH (23.93 mg, 0.997 mmol) at rt. After stirring at rt for 1 hr, reaction was quenched with water (1 ml) was and the mixture was concentrated under reduced pressure. The residue was added water (20 mL) and extracted with ethyl acetate (3×50 mL). The combined organic fractions were washed with brine (saturated, 20 mL), dried (Na₂SO₄), filtered and the solvent was evaporated under reduced pressure to give the title compound. MS: 293 [M+H]⁺.

### Intermediate CC-GG

### 6-chloro-N,N-bis(4-methoxybenzyl)pyridin-2-amine (Intermediate CC), 6-chloro-N-(4-methoxybenzyl)-N-methylpyridin-2-amine (Intermediate DD), 6-chloro-N-(4-methoxybenzyl)-N-methyl-5-(trifluoromethyl)pyridin-2-amine (Intermediate EE), 6-chloro-3-fluoro-N,N-bis(4-methoxybenzyl)pyridin-2-amine (Intermediate FF), and 6-chloro-4-methoxy-N-(4-methoxybenzyl)-N-methylpyridin-2-amine (Intermediate GG)

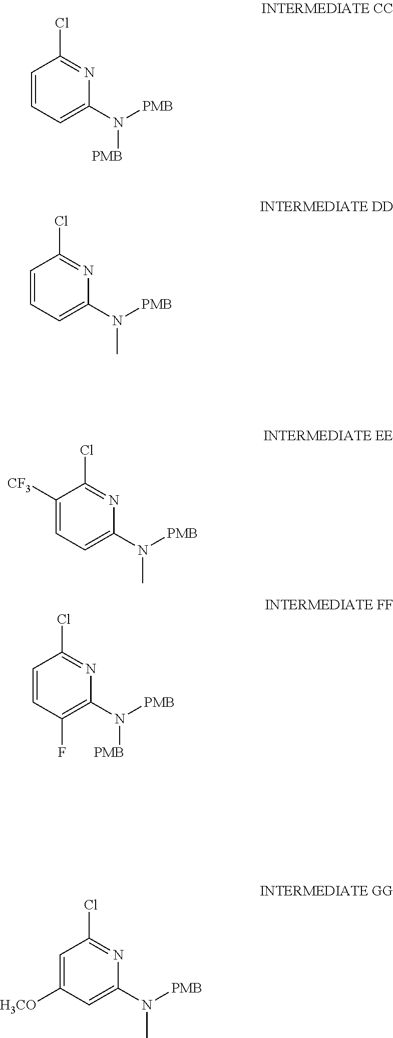

The following INTERMEDIATES CC through GG were prepared according to the general procedures herein and in an analogous manner to that used to synthesize INTERMEDIATE Z, INTERMEDIATE AA, and INTERMEDIATE BB using the appropriate starting materials.

### Intermediate HH

### 2-(benzyloxy)-6-bromo-3,5-difluoropyridine

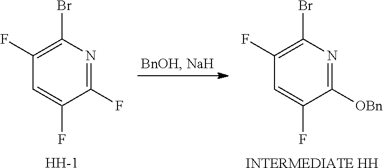

To a mixture of BENZYL ALCOHOL (0.981 ml, 9.44 mmol) in THF (14.30 ml) at 0° C. was added NaH (0.377 g, 9.44 mmol) and the mixture stirred for 10 min before adding 2-bromo-3,5,6-trifluoropyridine (HH-1) (1.00 g, 4.72 mmol). The mixture was allowed to slowly warm to ambient temperature and stirred for another hour. The reaction was quenched with H2O (50 mL), extract with DCM (3×@ 50 mL), dry over Na2SO4, and concentrate. The resulting residue was purified using silica column chromatography (2% to 55% EtOAc/hexanes) to obtain the title compound. MS: 300.2 [M+H]+.

### Intermediate II-MM

### 2-(benzyloxy)-6-chloro-4-(difluoromethyl)pyridine (Intermediate II), 2-(benzyloxy)-6-chloro-3-(trifluoromethoxy)pyridine (Intermediate JJ), 6-(benzyloxy)-2-chloro-3-(trifluoromethoxy)pyridine (Intermediate KK), 6-(benzyloxy)-2-bromo-3-methylpyridine (Intermediate LL), and 2-(benzyloxy)-6-chloro-3-methoxypyridine (Intermediate MM)

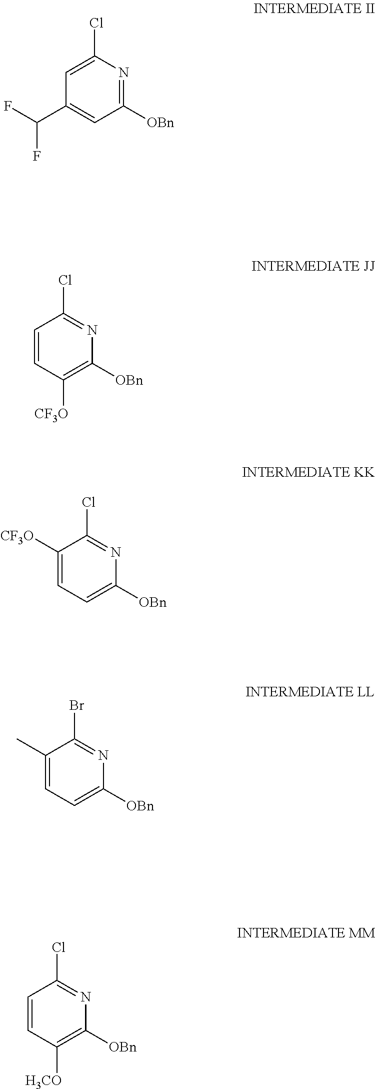

The following INTERMEDIATES II through MM were prepared according to the general procedures herein and in an analogous manner to that used to synthesize INTERMEDIATE HH using the appropriate starting materials.

### Intermediate NN and Intermediate OO

### 6-chloro-3-(trifluoromethyl)pyridin-2-ol (Intermediate NN) and 6-chloro-5-(trifluoromethyl)pyridin-2-ol (Intermediate 00)

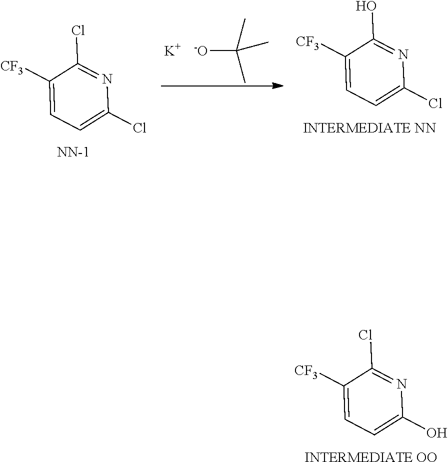

To a mixture of 2,6-dichloro-3-(trifluoromethyl)pyridine (NN-1) (1200 mg, 5.56 mmol), 2,6-dichloro-3-(trifluoromethyl)pyridine (1200 mg, 5.56 mmol) and POTASSIUM TERT-BUTOXIDE (2494 mg, 22.22 mmol) in t-BuOH (20 ml) in 20 ml of microwave vial was heated to 80° C. for 10 hours. The reaction mixture was concentrated under reduced pressure and the residue was dissolved MeOH (16 ml) and Water (8 ml) and added HCl (8 ml, 56.0 mmol). Stirred at rt overnight. The suspension mixture was filtered and the filtrate was loaded and purified by C18 column chromatography (10-80% Water in Acetonitrile with 0.05% TFA) to give the title compounds separately. INTERMEDIATE NN: MS: 198.0 [M+H]⁺. INTERMEDIATE OO: MS: 198.1 [M+H]+.

### Intermediate PP

### 4-(6-hydroxy-4-(trifluoromethyl)pyridin-2-yl)cyclohexan-1-one

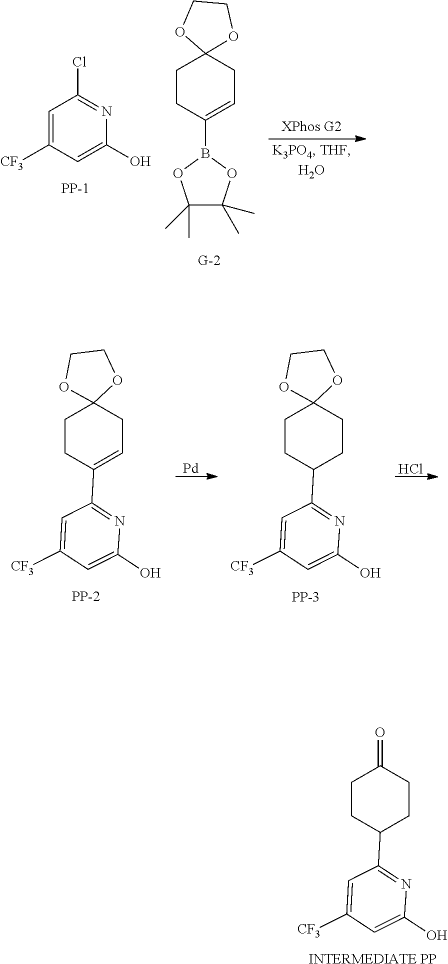

### Step 1: 6-(1,4-dioxaspiro[4.5]dec-7-en-8-yl)-4-(trifluoromethyl)pyridin-2-ol (PP-2)

To a mixture of 6-chloro-4-(trifluoromethyl)pyridin-2-ol (PP-1)(200 mg, 1.012 mmol), 4,4,5,5-tetramethyl-2-(1,4-dioxaspiro[4.5]dec-7-en-8-yl)-1,3,2-dioxaborolane (G-2) (323 mg, 1.215 mmol), 1,1′-bis(diphenylphosphino)ferrocene-palladium(II)dichloride dichloromethane complex (83 mg, 0.101 mmol) and Cs₂CO₃ (990 mg, 3.04 mmol) in DIOXANE (6.0 mL) and water (1.5 mL) charged in 20 mL of microwave vial was capped and bubbled with N2 for 3 min. The resulting solution was heated at 80° C. for 90 minutes. The mixture was quenched with water (50 mL) and extracted by ethyl acetate (3×50 mL). The combined organic fractions were washed with brine (saturated, 50 mL), dried (Na₂SO₄), filtered and the solvent was evaporated under reduced pressure. The residue was purified by silica column chromatography (0-100% EtOAc/CH₂Cl₂) to give the title compound. MS: 302 [M+H]⁺.

Step 2: 6-(1,4-dioxaspiro[4.5]decan-8-yl)-4-(trifluoromethyl)pyridin-2-ol (PP-3) A suspension of 6-(1,4-dioxaspiro[4.5]dec-7-en-8-yl)-4-(trifluoromethyl)pyridin-2-ol (PP-2)(300 mg, 0.996 mmol) and Pd/C (126 mg, 0.118 mmol) in MeOH (13 mL) was degassed and refilled with H₂ from a balloon for three times. The mixture was then stirred under a H₂ balloon for 2 hrs at rt. The mixture was filtered through a celite cake, washing with methanol. The combined filtrates were concentrated under reduced pressure to give the title compound. MS: 304 [M+H]⁺.

### Step 3: 4-(6-hydroxy-4-(trifluoromethyl)pyridin-2-yl)cyclohexan-1-one (Intermediate PP)

To a solution of 6-(1,4-dioxaspiro[4.5]decan-8-yl)-4-(trifluoromethyl)pyridin-2-ol (PP-3) (302 mg, 0.996 mmol) in acetone (8 mL) and water (2.0 mL) was added aq. HCl (7.0 M, 0.427 ml, 2.99 mmol). The resulting suspension was stirred at rt. After 5 hrs, the mixture was diluted with water (50 mL) and the mixture was extracted with ethyl acetate (3×50 mL). The combined organic fractions were washed with brine (saturated, 50 mL), dried (MgSO₄), filtered and the solvent was evaporated under reduced pressure. The residue was purified by silica column chromatography (0-100% EtOAc/CH₂Cl₂) to give the title compound. MS: 260 [M+H]+.

The following intermediates were prepared according to the general procedures herein and in an analogous manner to that used to synthesize INTERMEDIATE PP using the appropriate intermediates. The starting materials were either prepared as described in the intermediates section, commercially available, or prepared from commercially available reagents using conventional reactions well known in the art.

| | | | Observed |
|---|---|---|---|
| | | | Mass |
| INTERMEDIATE | Structure | Name | [M + H]⁺ |
| INTERMEDIATE QQ | 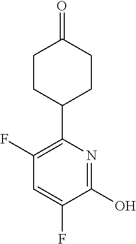 [SMILES: O=C1CCC(c2nc(O)c(F)cc2F)CC1] | 4-(3,5-difluoro-6- hydroxypyridin-2- yl)cyclohexan-1-one | 228.1 |
| INTERMEDIATE RR | 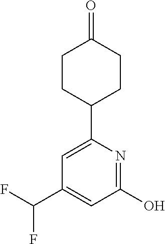 [SMILES: O=C1CCC(c2cc(C(F)F)cc(O)n2)CC1] | 4-(4-(difluoromethyl)-6- hydroxypyridin-2- yl)cyclohexan-1-one | 241.9 |
| INTERMEDIATE SS | 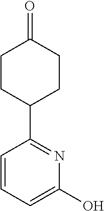 [SMILES: O=C1CCC(c2cccc(O)n2)CC1] | 4-(6-hydroxypyridin-2- yl)cyclohexan-1-one | 192.1 |
| INTERMEDIATE TT | 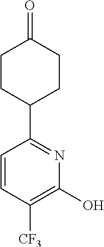 [SMILES: O=C1CCC(c2ccc(C(F)(F)F)c(O)n2)CC1] | 4-(6-hydroxy-5- (trifluoromethyl)pyridin- 2-yl)cyclohexan-1-one | 260.4 |
| INTERMEDIATE UU | 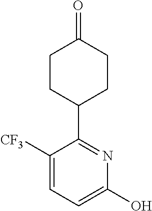 [SMILES: O=C1CCC(c2nc(O)ccc2C(F)(F)F)CC1] | 4-(5-hydroxy-2- (trifluoromethyl)phenyl) cyclohexan-1-one | 260.2 |
| INTERMEDIATE VV | 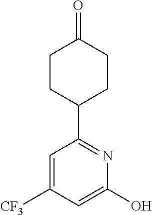 [SMILES: O=C1CCC(c2cc(C(F)(F)F)cc(O)n2)CC1] | 4-(6-hydroxy-4- (trifluoromethyl)pyridin- 2-yl)cyclohexan-1-one | 260.2 |
| INTERMEDIATE WW | 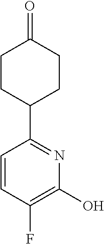 [SMILES: O=C1CCC(c2ccc(F)c(O)n2)CC1] | 4-(5-fluoro-6- hydroxypyridin-2- yl)cyclohexan-1-one | 210.1 |
| INTERMEDIATE XX | 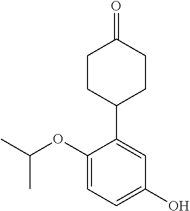 [SMILES: CC(C)Oc1ccc(O)cc1C1CCC(=O)CC1] | 4-(5-hydroxy-2- isopropoxyphenyl) cyclohexan-1-one | 249.2 |
| INTERMEDIATE YY | 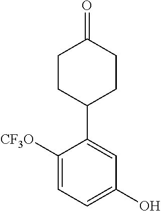 | 4-(5-hydroxy-2- (trifluoromethyl)phenyl) cyclohexan-1-one | 275.2 |
| INTERMEDIATE ZZ | 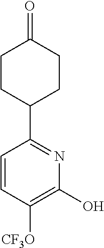 | 4-(6-hydroxy-5- (trifluoromethyl)pyridin- 2-yl)cyclohexan-1-one | 276.0 |
| INTERMEDIATE AAA | 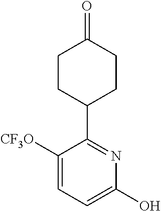 | 4-(6-hydroxy-3- (trifluoromethoxy)pyridin- 2-yl)cyclohexan-1-one | 276.3 |
| INTERMEDIATE BBB | 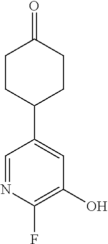 [SMILES: O=C1CCC(c2cnc(F)c(O)c2)CC1] | 4-(2-fluoro-6- hydroxypyridin-4- yl)cyclohexan-1-one | 210.1 |
| INTERMEDIATE CCC | 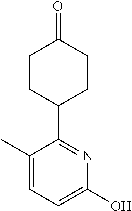 | 4-(6-hydroxy-3- methylpyridin-2- yl)cyclohexan-1-one | 206.2 |
| INTERMEDIATE DDD | 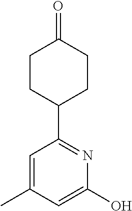 | 4-(6-hydroxy-4- methylpyridin-2- yl)cyclohexan-1-one | 206.1 |
| INTERMEDIATE EEE | 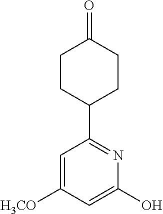 [SMILES: COc1cc(O)nc(C2CCC(=O)CC2)c1] | 4-(6-hydroxy-4- methoxypyridin-2- yl)cyclohexan-1-one | 222.2 |
| INTERMEDIATE FFF | 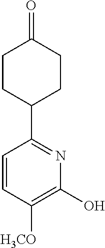 [SMILES: COc1ccc(C2CCC(=O)CC2)nc1O] | 4-(6-hydroxy-5- methoxypyridin-2- yl)cyclohexan-1-one | 222.3 |
| INTERMEDIATE GGG | 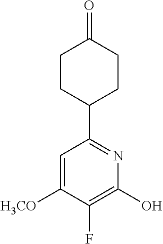 [SMILES: COc1cc(C2CCC(=O)CC2)nc(O)c1F] | 4-(5-fluoro-6-hydroxy-4- methoxypyridin-2- yl)cyclohexan-1-one | 240.2 |
| INTERMEDIATE HHH | 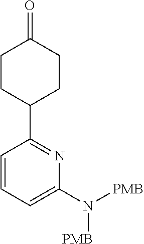 | 4-(6-(bis(4- methoxybenzyl)amino) pyridin-2-yl)cyclohexan-1-one | 431.5 |
| INTERMEDIATE III | 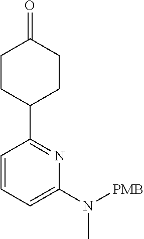 | 4-(6-((4- methoxybenzyl)(methyl) amino)pyridin-2- yl)cyclohexan-1-one | 325.4 |
| INTERMEDIATE JJJ | 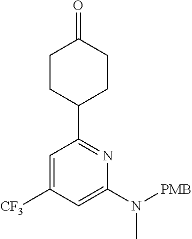 | 4-(6-((4- methoxybenzyl)(methyl) amino)-4- (trifluoromethyl)pyridin- 2-yl)cyclohexan-1-one | 393.4 |
| INTERMEDIATE KKK | 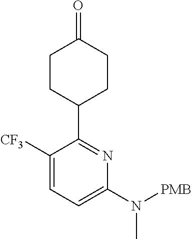 | 4-(6-((4- methoxybenzyl)(methyl) amino)-3- (trifluoromethyl)pyridin- 2-yl)cyclohexan-1-one | 393.3 |
| INTERMEDIATE LLL | 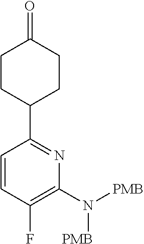 | 4-(6-(bis(4- methoxybenzyl)amino)-5- fluoropyridin-2- yl)cyclohexan-1-one | 449.4 |
| INTERMEDIATE MMM | 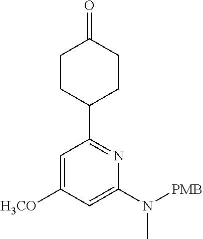 | 4-(4-methoxy-6-((4- methoxybenzyl)(methyl) amino)pyridin-2- yl)cyclohexan-1-one | 355.4 |
| INTERMEDIATE NNN | 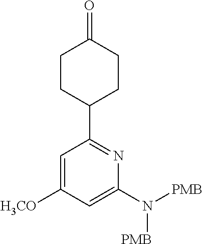 | 4-(6-(bis(4- methoxybenzyl)amino)-4- methoxypyridin-2- yl)cyclohexan-1-one | 461.6 |
| INTERMEDIATE OOO | 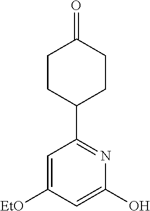 [SMILES: CCOc1cc(O)nc(C2CCC(=O)CC2)c1] | 4-(4-ethoxy-6- hydroxypyridin-2- yl)cyclohexan-1-one | 236.2 |

### Intermediate PPP and Intermediate QQQ

### 2-chloroethyl (2R,3S,5S)-2-(((tert-butyldimethylsilyl)oxy)methyl)-3-(N-(4-methoxybenzyl)methylsulfonamido)-5-(methoxymethyl)pyrrolidine-1-carboxylate (Intermediate PPP) and 3-chloropropyl (2R,3S,5S)-2-(((tert-butyldimethylsilyl)oxy)methyl)-3-(N-(4-methoxybenzyl)methylsulfonamido)-5-(methoxymethyl)pyrrolidine-1-carboxylate (Intermediate QQQ)

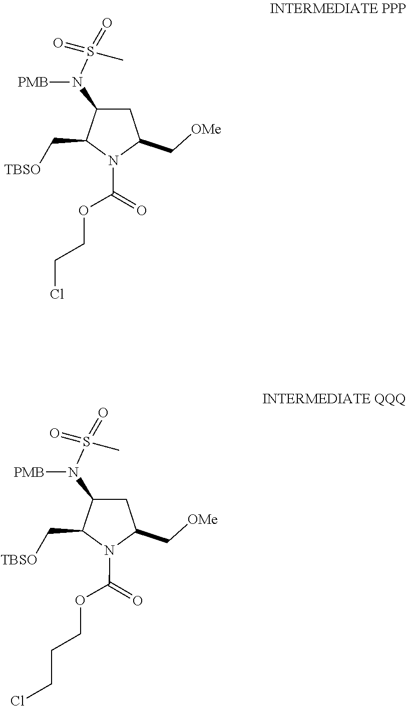

INTERMEDIATE PPP and INTERMEDIATE QQQ was prepared according to the procedures used to synthesize INTERMEDIATE V using INTERMEDIATE D and the correct corresponding chloroalkyl carbonochloridate.

### Intermediate RRR

### 1-benzyl 2-methyl (2R,3S,5S)-5-(((tert-butyldiphenylsilyl)oxy)methyl)-3-((4-methoxybenzyl)amino)pyrrolidine-1,2-dicarboxylate

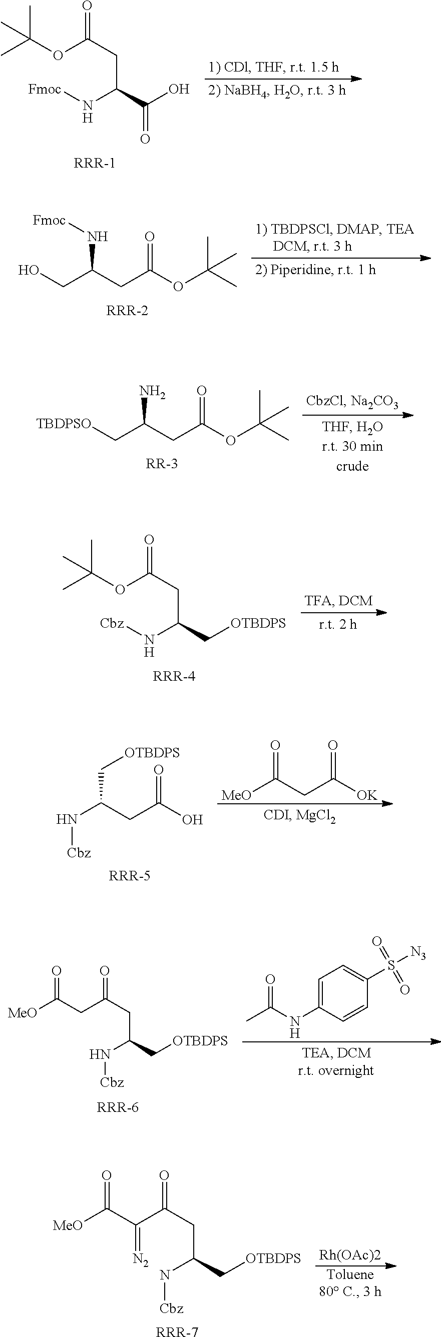

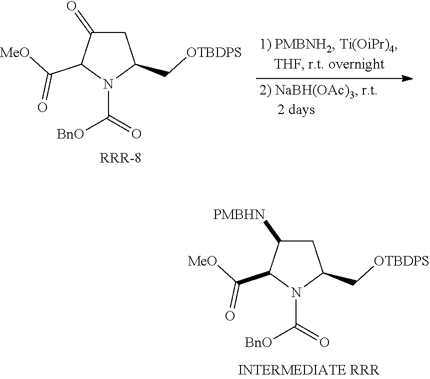

### Step 1: tert-butyl (3S)-3-[[(9H-fluoren-9-ylmethoxy) carbonyl]amino]-4-hydroxybutanoate (RRR-2)

Into a 5-L 3-necked round-bottom flask purged and maintained with an inert atmosphere of nitrogen, was placed a solution of (2S)-4-(tert-butoxy)-2-[[(9H-fluoren-9-ylmethoxy)carbonyl]amino]-4-oxobutanoic acid (RRR-1) (350.00 g, 850.650 mmol, 1.00 equiv) in THF (3.5 L). This was followed by the addition of CDI (239.76 g, 1701.300 mmol, 2.00 equiv) at 0 C. The resulting solution was stirred for 1.5 h at room temperature. After that the mixture was added to a solution of NaBH4 (64.65 g, 1701.300 mmol, 2.00 equiv) in H2O (1500 mL) at 10 C. The resulting solution was allowed to react, with stirring, for an additional 3 h at room temperature. The resulting mixture was concentrated. The resulting solution was extracted with EA (2×1 L). The combined organic layers were dried over anhydrous Na2SO4. After filtration, the filtrate was concentrated under reduced pressure to give the title compound.

### Step 2: tert-butyl (3S)-3-amino-4-[(tert-butyldiphenylsilyl)oxy]butanoate (RRR-3)

Into a 3-L 3-necked round-bottom flask purged and maintained with an inert atmosphere of nitrogen, was placed a solution of tert-butyl-(3S)-3-[[(9H-fluoren-9-ylmethoxy)carbonyl]amino]-4-hydroxybutanoate (RRR-2) (413.00 g, 1039.070 mmol, 1 equiv) in DCM (1.24 L). This was followed by the addition of DMAP (12.69 g, 103.907 mmol, 0.10 equiv) and TEA (136.96 g, 1350.790 mmol, 1.30 equiv) dropwise with stirring. To this was added tert-butyl(chloro)diphenylsilane (314.16 g, 1142.976 mmol, 1.10 equiv) at a temperature lower than 40 C. The resulting solution was allowed to react, with stirring, for an additional 3 h at room temperature. To the mixture was added piperidine (265.43 g, 3117.209 mmol, 3.00 equiv). The resulting solution was stirred for 1 h at room temperature. The reaction was then quenched by the addition of 700 mL of NaHCO₃. The resulting solution was extracted with 500 mL of DCM. The combined organic layers were washed with NaCl (1×700 mL) and dried over anhydrous Na2SO4. After filtration, the filtrate was concentrated under reduced pressure to give the title compound.

### Step 3: tert-butyl-(3S)-3-[[(benzyloxy)carbonyl]amino]-4-[(tert-butyldiphenylsilyl)oxy]butanoate (RRR-4)

Into a 3-L 3-necked round-bottom flask purged and maintained with an inert atmosphere of nitrogen, was placed a solution of tert-butyl (3S)-3-amino-4-[(tert-butyldiphenylsilyl)oxy]butanoate (RRR-3) (170 g, 410.995 mmol, 1.00 equiv) in THF (1.7 L). This was followed by the addition of CbzCl (77.12 g, 452.095 mmol, 1.10 equiv) dropwise with stirring at room temperature. To this was added a solution of Na2CO3 (87.13 g, 821.990 mmol, 2.00 equiv) in H2O (800 mL) dropwise with stirring at room temperature. The resulting solution was stirred for 30 min at room temperature. The resulting solution was extracted with 2×800 mL of EA. The combined organic layers were washed with NaCl (2×800 mL) and dried over anhydrous Na2SO4. After filtration, the filtrate was concentrated under reduced pressure to give the title compound.

### Step 4: (3S)-3-[[(benzyloxy)carbonyl]amino]-4-[(tert-butyldiphenylsilyl)oxy]butanoic acid (RRR-5)

Into a 2-L 4-necked round-bottom flask purged and maintained with an inert atmosphere of nitrogen, was placed a solution of tert-butyl(3S)-3-[[(benzyloxy)carbonyl]amino]-4-[(tert-butyldiphenylsilyl) oxy]butanoate (RRR-4) (200.00 g, 365.119 mmol, 1.00 equiv) in DCM (1 L). This was followed by the addition of TFA (199.88 mL, 1752.932 mmol, 7.37 equiv) dropwise with stirring at room temperature. The resulting solution was stirred for 2 h at room temperature. The reaction was then quenched by the addition of 500 L of water/ice. The resulting mixture was washed with H2O (3×500 mL) and dried over anhydrous Na2SO4. After filtration, the filtrate was concentrated under reduced pressure. The residue was applied onto a silica gel column with EA/petroleum ether (0%-25%) to give the title compound.

### Step 5: methyl (5S)-5-[[(benzyloxy)carbonyl]amino]-6-[(tert-butyldiphenylsilyl)oxv]-3-oxohexanoate (RRR-6)

Into a 2-L 3-necked round-bottom flask purged and maintained with an inert atmosphere of nitrogen, was placed a solution of (3S)-3-[[(benzyloxy)carbonyl]amino]-4-[(tert-butyldiphenylsilyl)oxy]butanoic acid (RRR-5) (80.00 g, 162.714 mmol, 1.00 equiv) in THF (800 mL). This was followed by the addition of CDI (39.58 g, 244.072 mmol, 1.50 equiv) dropwise with stirring at room temperature. The resulting solution was stirred for 3 h at room temperature. This was followed by the addition of MgCl2 (30.98 g, 325.429 mmol, 2.00 equiv) and 1-methyl 3-potassium propanedioate (50.82 g, 325.429 mmol, 2.00 equiv) dropwise with stirring at room temperature. The resulting solution was stirred for additional 2 days at room temperature. The resulting solution was diluted with 400 mL of EA. The reaction was then quenched by the addition of 500 mL of water/ice. The resulting mixture was washed with NaHCO₃(2×400 mL). The resulting solution was extracted with EA (2×400 mL). The combined organic layers were washed with NaCl (1×400 mL) and dried over anhydrous Na2SO4. After filtration, the filtrate was concentrated under reduced pressure to give the title compound.

### Step 6: methyl (5S)-5-[[(benzyloxy)carbonyl]amino]-6-[(tert-butyldiphenylsilyl)oxy]-2-diazo-3-oxohexanoate (RRR-7)

Into a 2-L 3-necked round-bottom flask purged and maintained with an inert atmosphere of nitrogen, was placed a solution of methyl (5S)-5-[[(benzyloxy)carbonyl]amino]-6-[(tert-butyldiphenylsilyl)oxy]-3-oxohexanoate (RRR-6) (80.00 g, 146.059 mmol, 1.00 equiv) in DCM (800 mL). This was followed by the addition of TEA (44.34 g, 438.178 mmol, 3.00 equiv) and 4-acetamidobenzenesulfonyl azide (35.09 g, 146.059 mmol, 1.00 equiv) dropwise with stirring at room temperature. The resulting solution was stirred overnight at room temperature. The solids were filtered out. The filtrate was washed with 2×500 mL of H2O. The resulting mixture was washed with 1×500 mL of citric acid and 1×500 mL of NaCl. The mixture was dried over anhydrous sodium sulfate and concentrated to give the title compound.

### Step 7: 1-benzyl 2-methyl (5S)-5-[[(tert-butyldiphenylsilyl)oxy]methyl]-3-oxopyrrolidine-1,2-dicarboxylate (RRR-8)

Into a 2-L 3-necked round-bottom flask purged and maintained with an inert atmosphere of nitrogen, was placed a solution of methyl (5S)-5-[[(benzyloxy)carbonyl]amino]-6-[(tert-butyldiphenylsilyl)oxy]-2-diazo-3-oxohexanoate (RRR-7) (80.00 g, 139.441 mmol, 1.00 equiv) in Toluene (800 mL), Rh2(OAc)4 (6.16 g, 13.944 mmol, 0.10 equiv). The resulting solution was stirred for 3 h at 80 C in an oil bath. The reaction mixture was cooled with a water bath. The solids were filtered out. The filtrate was concentrated. The residue was applied onto a silica gel column with EA/petroleum ether (0%-20%) to give the title compound.

### Step 8: 1-benzyl 2-methyl (2R,3S,5S)-5-(((tert-butyldiphenylsilyl)oxy)methyl)-3-((4-methoxybenzyl)amino)pyrrolidine-1,2-dicarboxylate (Intermediate RRR)

Into a 1000-mL 3-necked round-bottom flask purged and maintained with an inert atmosphere of nitrogen, was placed a solution of 1-benzyl 2-methyl (5S)-5-[[(tert-butyldiphenylsilyl)oxy]methyl]-3-oxopyrrolidine-1,2-dicarboxylate (RRR-8) (50.00 g, 91.624 mmol, 1.00 equiv) in THF (500 mL), (4-methoxyphenyl)methanamine (15.08 g, 109.949 mmol, 1.20 equiv). This was followed by the addition of Ti(Oi-Pr)4 (26.04 g, 91.624 mmol, 1.00 equiv) dropwise with stirring at 0 C. The resulting solution was stirred overnight at room temperature. To this was added NaBH(OAc)3 (135.93 g, 641.370 mmol, 7.00 equiv) in several batches. The resulting solution was allowed to react, with stirring, for an additional 2 days at room temperature. The resulting solution was diluted with 200 mL of EA. The reaction was then quenched by the addition of 400 mL of NaHCO₃. The solids were filtered out. The filtrate was extracted with 2×300 mL of EA and the organic layers combined. The resulting mixture was washed with 2×500 mL of NaCl. The mixture was dried over anhydrous sodium sulfate and concentrated. The residue was applied onto a silica gel column with EA/petroleum ether (0%-45%). Obtained the crude product was purified by Prep-SFC with the following conditions (Column: CHIRAL ART Cellulose-SB S-5 um 50*250 mm, 50 mm*250 mm 5 um; Mobile Phase A: CO2, Mobile Phase B: MEOH (2 mM NH3-MEOH); Flow rate: 180 mL/min; Gradient:50% B; UV220 nm; RT1: 3.92; RT2: 4.78; Injection Volume: 2 mL; Number of Runs: 75) to obtain the title compound. LC-MS: (ES, m/z): 667 [M+1]+. H-NMR: (300 MHz, Chloroform-d, ppm): δ 7.63 (t, J=12.5 Hz, 4H), 7.49-7.29 (m, 8H), 7.28-7.06 (m, 5H), 6.89 (d, J=8.5 Hz, 2H), 5.13-4.90 (m, 2H), 4.78-4.58 (m, 1H), 4.51-4.22 (m, 2H), 3.98-3.41 (m, 10H), 2.51 (s, 1H), 2.15-1.93 (m, 1H), 1.15-0.99 (m, 9H).

### Intermediate SSS

### benzyl (2R,3S,5S)-5-(acetoxymethyl)-2-(((tert-butyldimethylsilyl)oxy)methyl)-3-(N-(4-methoxybenzyl)methylsulfonamido)pyrrolidine-1-carboxylate

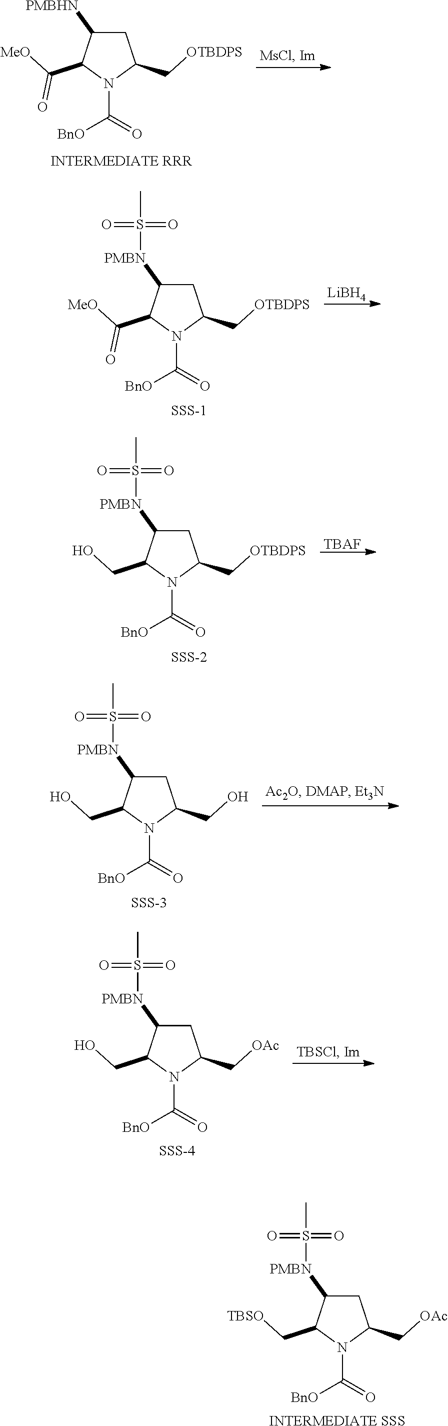

### Step 1: 1-benzyl 2-methyl (2R,3S,5S)-5-(((tert-butyldiphenylsilyl)oxy)methyl)-3-(N-(4-methoxybenzyl)methylsulfonamido)pyrrolidine-1,2-dicarboxylate (SSS-1)

To a mixture of 1-METHYLIMIDAZOLE (2.391 ml, 30.0 mmol) in DCM (22.72 ml) at 0° C. was added METHANESULFONYL CHLORIDE (1.168 ml, 14.99 mmol) dropwise. The mixture stirred for 15 min before adding a mixture of 1-benzyl 2-methyl (2R,3S,5S)-5-(((tert-butyldiphenylsilyl)oxy)methyl)-3-((4-methoxybenzyl)amino)pyrrolidine-1,2-dicarboxylate (INTERMEDIATE RRR) (5.00 g, 7.50 mmol), TRIETHYLAMINE (4.18 ml, 30.0 mmol), and DCM 10 mL dropwise. The mixture was warmed to ambient temperature and stirred overnight. The mixture was quench with H2O (50 mL), extract with DCM (3×@ 50 mL), wash with 1.0 M HCl ((2×@ 50 mL), dry over Na2SO4, and concentrate. The resulting residue was purified using silica column chromatography (5% to 75% 3:1 EtOAc:EtOH/hexanes) to obtain the title compound. MS: 745.7 [M+H]⁺.

### Step 2: benzyl (2R,3S,5S)-5-(((tert-butyldiphenylsilyl)oxy)methyl)-2-(hydroxymethyl)-3-(N-(4-methoxybenzyl)methylsulfonamido)pyrrolidine-1-carboxylate (SSS-2)

To a mixture of 1-benzyl 2-methyl (2R,3S,5S)-5-(((tert-butyldiphenylsilyl)oxy)methyl)-3-(N-(4-methoxybenzyl)methylsulfonamido)pyrrolidine-1,2-dicarboxylate (SSS-1) (8.00 g, 10.74 mmol) in THF (32.5 ml) at 0° C. was added LiBH4 (8.05 ml, 16.11 mmol) in THF slowly. The mixture was warmed to ambient temperature and stirred for 20 hours. The mixture was quenched with H2O (50 mL), extracted with EtOAc (3×@ 50 mL), dried over Na2SO4, and concentrated. The resulting residue was purified using silica column chromatography (5% to 90% 3:1 EtOAc/hexanes) to obtain the title compound. MS: 718.3 [M+H]⁺.

### Step 3: benzyl (2R,3S,5S)-2,5-bis(hydroxymethyl)-3-(N-(4-methoxybenzyl)methylsulfonamido)pyrrolidine-1-carboxylate (SSS-3)

To a mixture of benzyl (2R,3S,5S)-5-(((tert-butyldiphenylsilyl)oxy)methyl)-2-(hydroxymethyl)-3-(N-(4-methoxybenzyl)methylsulfonamido)pyrrolidine-1-carboxylate (SSS-2) (7.70 g, 10.74 mmol) in THF (53.7 ml) at ambient temperature was added TBAF (12.89 ml, 12.89 mmol) in THF. The mixture stirred for 1 hour before quenching with a saturated solution of NH4Cl (100 mL), extracting with EtOAc (3×@ 100 mL), drying over Na2SO4, and concentrating. The resulting solution was purified using silica column chromatography (3:1 EtOAc:EtOH/hexanes) to obtain the title compound. MS: 479.4 [M+H]⁺.

### Step 4: benzyl (2R,3S,5S)-5-(acetoxymethyl)-2-(hydroxymethyl)-3-(N-(4-methoxybenzyl)methylsulfonamido)pyrrolidine-1-carboxylate (SSS-4)

A flask equipped with stir bar was charged with benzyl (2R,3S,5S)-2,5-bis(hydroxymethyl)-3-(N-(4-methoxybenzyl)methylsulfonamido)pyrrolidine-1-carboxylate (SSS-3) (5.14 g, 10.74 mmol) followed by DMAP (0.262 g, 2.148 mmol). The flask was sealed with a septum, purged with N2 and dry DCM (64.9 ml) was added followed by dry TRIETHYLAMINE (2.246 ml, 16.11 mmol). The resulting solution was cooled to −22° C. in a dry ice/acetone bath and a solution of ACETIC ANHYDRIDE (1.064 ml, 11.28 mmol) in dry DCM (12 mL) was added dropwise, maintaining an internal temperature of less than −21° C. The reaction was stirred at less than −20° C. for 1 hour then diluted with DCM, 1M citric acid, and water and the layers separated. The aqueous layer was extracted with additional DCM (×2) and the combined organic layers were dried over magnesium sulfate, filtered, and concentrated under reduced pressure. The resulting solution was purified using silica column chromatography (2-90% 3:1 ethyl acetate:EtOH in hexane) to obtain the title compound. MS: 543.0 [M+Na]+.

### Step 5: benzyl (2R,3S,5S)-5-(acetoxymethyl)-2-(((tert-butyldimethylsilyl)oxy)methyl)-3-(N-(4-methoxybenzyl)methylsulfonamido)pyrrolidine-1-carboxylate (Intermediate SSS)

To a mixture of benzyl (2R,3S,5S)-5-(acetoxymethyl)-2-(hydroxymethyl)-3-(N-(4-methoxybenzyl)methylsulfonamido)pyrrolidine-1-carboxylate (SSS-4) (4.74 g, 9.10 mmol) in DCM (27.6 ml) at ambient temperature was added TBS-Cl (2.058 g, 13.66 mmol) and IMIDAZOLE (1.860 g, 27.3 mmol). The mixture stirred for 2 hours before quenching with H2O (50 mL), extracting with DCM (3×@ 50 mL), drying over Na2SO4, and concentrating. The resulting residue was purified using silica column chromatography (2% to 70% 3:1 EtOAc:EtOH/hexanes) to obtain the title compound. MS: 657.2 [M+Na]⁺.

### Intermediate TTT and Intermediate UUU

### 2-chloroethyl (2R,3S,5S)-5-(acetoxymethyl)-2-(((tert-butyldimethylsilyl)oxy)methyl)-3-(N-(4-methoxybenzyl)methylsulfonamido)pyrrolidine-1-carboxylate (Intermediate TTT) and 3-chloropropyl (2R,3S,5S)-5-(acetoxymethyl)-2-(((tert-butyldimethylsilyl)oxy)methyl)-3-(N-(4-methoxybenzyl)methylsulfonamido)pyrrolidine-1-carboxylate (Intermediate UUU)

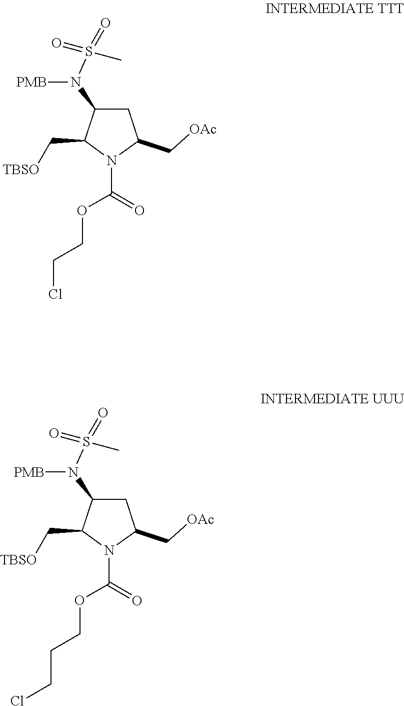

INTERMEDIATE TTT and INTERMEDIATE UUU was prepared according to the procedures used to synthesize INTERMEDIATE V using INTERMEDIATE SSS and the correct corresponding chloroalkyl carbonochloridate.

### Intermediate VVV

### benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate

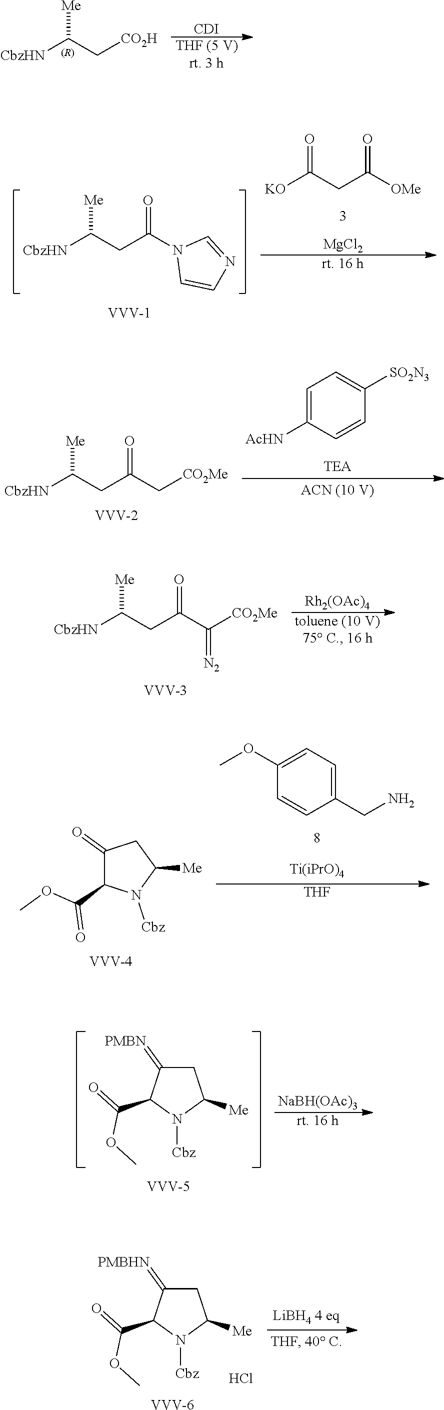

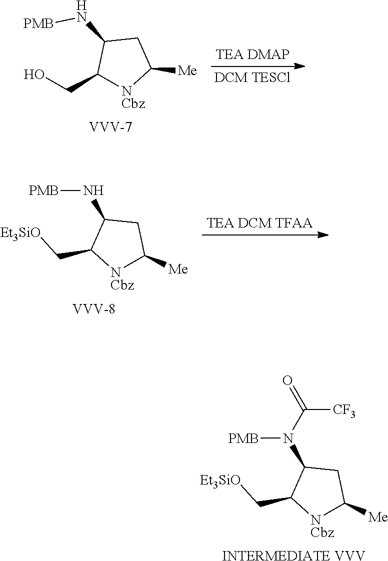

### Step 1: benzyl (R)-(4-(1H-imidazol-1-yl)-4-oxobutan-2-yl)carbamate (VVV-1)

Into a 5-L 4-necked round-bottom flask, was placed CDI (107.64 g, 663.83 mmol, 1.05 equiv), THF (750.00 mL). To this was added (3R)-3-[[(benzyloxy)carbonyl]amino]butanoic acid (150.00 g, 632.23 mmol, 1.00 equiv), in portions at 0-5° C. in 30 min. The resulting solution was stirred for 3 h at room temperature. Which was used in the next step without further purification.

### Step 2: methyl (5R)-5-[[(benzyloxy)carbonyl]amino]-3-oxohexanoate (VVV-2)

Into a 3-L 4-necked round-bottom flask, was placed 1-methyl 3-potassium propanedioate (147 g, 941.23 mmol, 1.50 equiv), THF (750 mL). This was followed by the addition of MgCl₂ (45 g, 470.61 mmol, 0.75 equiv), in portions at 25° C. The resulting solution was stirred for 4 h at 40° C. To this was added the solution in the step 1 at 25° C. The resulting solution was stirred for 16 h at 25° C. The reaction was then quenched by the addition of 1 L of water/ice. The pH value of the solution was adjusted to 4 with HCl (2 mol/L). The resulting solution was extracted with 2×2 L of ethyl acetate and the organic layer was combined. The resulting mixture was washed with 1×1 L of H₂O and 1×1 L of brine. The mixture was dried over anhydrous sodium sulfate and concentrated to give the title compound.

### Step 3: methyl (5R)-5-[[(benzyloxy)carbonyl]amino]-2-diazo-3-oxohexanoate (VVV-3)

Into a 5-L 4-necked round-bottom flask, was methyl (5R)-5-[[(benzyloxy)carbonyl]amino]-3-oxohexanoate (VVV-2) (167.00 g, 569.34 mmol, 1.00 equiv), ACN (1.70 L) and 4-acetamidobenzenesulfonyl azide (136.78 g, 569.34 mmol, 1.00 equiv) at 0° C. Dropwise TEA (11.52 g, 113.84 mmol, 0.20 equiv) at 0° C. The flask was wrapped with aluminum foil and the resulting solution was stirred for 3 h at room temperature in a water/ice bath. The solids were filtrated out by filtration. The reaction was quenched with 2 L of water. The filtrate was extracted with EA (2×2.7 L) and the organic layers combined. The mixture was dried over anhydrous sodium sulfate and concentrated under vacuum to give the title compound.

### Step 4: 1-benzyl 2-methyl (2R,5R)-5-methyl-3-oxopyrrolidine-1,2-dicarboxylate (VVV-4)

Into a 5-L round-bottom flask, was placed methyl (5R)-5-[[(benzyloxy)carbonyl]amino]-2-diazo-3-oxohexanoate (VVV-3) (140 g, 438.40 mmol, 1.00 equiv), toluene (1.40 L), 1,1,1-tris(acetyloxy)dirhodium-1-yl acetate (19.4 g, 43.84 mmol, 0.1 equiv). The resulting solution was stirred for 16 h at 75° C. The residue was applied onto a silica gel column (PE:THF=1:1) to obtain the title compound.

### Step 5: 1-benzyl 2-methyl (2R,5R)-3-((4-methoxybenzyl)imino)-5-methylpyrrolidine-1,2-dicarboxylate (VVV-5)

Into a 5-L 4-necked round-bottom flask, was placed 1-benzyl 2-methyl 5-methyl-3-oxopyrrolidine-1,2-dicarboxylate (VVV-4) (95.00 g, 326.12 mmol, 1.00 equiv), THF (1.80 L), 4-methoxy-benzenemethanamine (44.74 g, 326.12 mmol, 1.00 equiv) and tetraisopropoxy(methyl)titanium (97.59 g, 326.12 mmol, 1.00 equiv). The resulting solution was stirred for overnight at r.t. The resulting solution was directly in the next step.

### Step 6: benzyl 2-methyl (2R,3S,5R)-3-[[(4-methoxyphenyl)methyl]amino]-5-methylpyrrolidine-1,2-dicarboxylate hydrochloride (VVV-6)

Into the step 5 mixture, was added bis(acetyloxy)(sodio)-lambda4-boranyl acetate (483 g, 2.28 mol, 7.00 equiv), once every half an hour, a total of 7 times are added. The resulting solution was stirred for overnight at 30° C. The reaction was then quenched by the addition of 5 L of water/ice and stir for 1 h. The resulting solution was extracted with 1×1.8 L of ethyl acetate. The organic layer was dried over anhydrous sodium sulfate and concentrated. Dissolve the product in 4 L of MTBE and added HCl (4 N in dioxane, 45 mL). After stirring for 5 h, the white solid collected by suction filtration to obtain the title compound.

### Step 7: benzyl (2R,3S,5R)-2-(hydroxymethyl)-3-[[(4-methoxyphenyl)methyl]amino]-5-methylpyrrolidine-1-carboxylate (VVV-7)

Into a 2-L round-bottom flask, added benzyl 2-methyl (2R,3S,5R)-3-[[(4-methoxyphenyl)methyl]amino]-5-methylpyrrolidine-1,2-dicarboxylate hydrochloride (VVV-6) (72 g, 0.16 mol, 1.00 equiv) and EA (1 L), to this was added 40 mL of HCl in dioxane (4 N). The resulting solution was stirred for 16 h at r.t. Then the reaction was filtered, the filtrate was concentrated. This resulted in 60 g of 1-benzyl 2-methyl (2R,3S,5R)-3-[[(4-methoxyphenyl)methyl]amino]-5-methylpyrrolidine-1,2-dicarboxylate. Then into a 2-L 4-necked round-bottom flask, was placed 1-benzyl 2-methyl (2R,3S,5R)-3-[[(4-methoxyphenyl)methyl]amino]-5-methylpyrrolidine-1,2-dicarboxylate (60.00 g, 145.45 mmol, 1.00 equiv), THF (1200 mL). This was followed by the addition of lithio-lambda5-borane (12.67 g, 581.83 mmol, 4.00 equiv) in batches. The resulting solution was stirred for 16 h at 40° C. The reaction was then quenched by the addition of 3 L of water/ice. The resulting solution was extracted with EA (2×1.2 L), the organic layer was dried over anhydrous sodium sulfate and concentrated. The residue was applied onto a silica gel column (PE:EA=3:1) to obtain the title compound.

### Step 8: benzyl (2R,3S,5R)-3-[[(4-methoxyphenyl)methyl]amino]-5-methyl-2-[[(triethylsilyl)oxy]methyl]pyrrolidine-1-carboxylate (VVV-8)

Into a 1-L 3-necked round-bottom flask purged and maintained with an inert atmosphere of nitrogen, was placed benzyl (2R,3S,5R)-2-(hydroxymethyl)-3-[[(4-methoxyphenyl)methyl]amino]-5-methylpyrrolidine-1-carboxylate (VVV-7) (40.00 g, 104.03 mmol, 1.00 equiv), triethanolamine (18.63 g, 124.87 mmol, 1.20 equiv.), DCM (300 mL), 4-dimethylaminopyridine (2.54 g, 20.79 mmol, 0.20 equiv.). To the mixture was added chlorotriethylsilane (18.82 g, 124.86 mmol, 1.20 equiv) dropwised at 0° C. The resulting solution was stirred for 1 h at room temperature. The reaction was then quenched by the addition of 1 L of water/ice. The resulting solution was extracted with DCM (2×200 mL), the organic layer was dried over anhydrous sodium sulfate and concentrated. The residue was applied onto a silica gel column (PE:THF=5:1-3:1) to obtain the title compound.

### Step 9: benzyl (2R,3S,5R)-5-methyl-2-[[(triethylsilyl)oxy]methyl]-3-[2,2,2-trifluoro-N-[(4-methoxyphenyl)methyl]acetamido]pyrrolidine-1-carboxylate (Intermediate VVV)

Into a 1-L 3-necked round-bottom flask, was placed benzyl (2R,3S,5R)-3-[[(4-methoxyphenyl)methyl]amino]-5-methyl-2-[[(triethylsilyl)oxy]methyl]pyrrolidine-1-carboxylate (VVV-8) (42.0 g, 84.20 mmol, 1.0 equiv), triethanolamine (18.9 g, 88.40 mmol, 1.05 equiv) and methylene chloride (400 mL). To this was added trifluoro acetic acid (10.08 g, 88.4 mmol, 1.05 equiv) at 0° C. The resulting solution was stirred for 30 min at room temperature. The resulting mixture was washed with 1×200 mL of sat NaHCO₃(aq). The resulting solution was extracted with DCM (2×400 mL), the organic layer was dried over anhydrous sodium sulfate and concentrated. The residue was applied onto a silica gel column (PE:EA=10:1) to obtain the title compound. LC-MS: (ES, m z): 595 [M+H]+. ¹H-NMR: (300 MHz, CDCl₃, ppm) δ 7.36 (s, 5H), 7.03 (d, J=8.6 Hz, 2H), 6.91 (d, J=8.7 Hz, 2H), 5.28-4.99 (m, 2H), 4.98-4.80 (m, 2H), 4.63-4.38 (m, 2H), 4.08-3.63 (m, 5H), 3.56-3.43 (m, 1H), 2.06-1.91 (m, 1H), 1.77-1.63 (m, 1H), 1.37-1.22 (m, 3H), 0.98 (t, J=7.5 Hz, 10H), 0.64 (t, J=8.2 Hz, 7H).

### Intermediate WWW and Intermediate XXX

### 2-chloroethyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Intermediate WWW) and 3-chloropropyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Intermediate XXX)

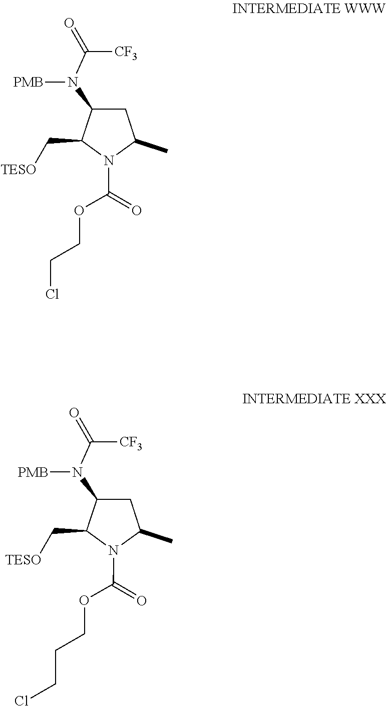

INTERMEDIATE WWW and INTERMEDIATE XXX was prepared according to the procedures used to synthesize INTERMEDIATE V using INTERMEDIATE VVV and the correct corresponding chloroalkyl carbonochloridate.

### Intermediate YYY

### 6-(4-oxocyclohexyl)-4-(trifluoromethyl)pyridin-2-yl trifluoromethanesulfonate

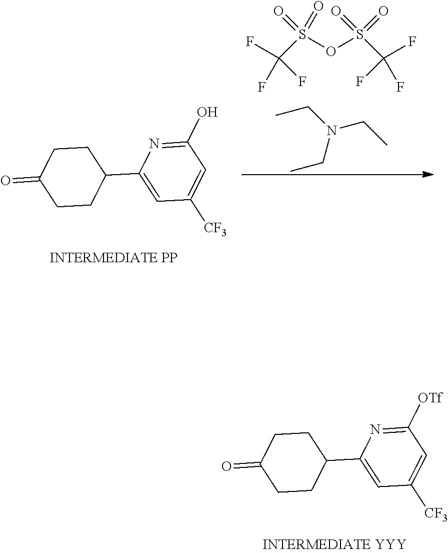

To a mixture of INTERMEDIATE PP (80 mg, 0.309 mmol) in DCM (3.086 ml) at −78° C. was added triethylamine (0.086 ml, 0.617 mmol) followed by trifluoromethanesulfonic anhydride (0.370 ml, 0.370 mmol) dropwise. The mixture was stirred for 1 hr. The reaction was quenched with a saturated solution of NaHCO₃(15 mL), extracted with DCM (3×@ 20 mL), dried over Na2SO4, and concentrated. The resulting residue was purified using silica column chromatography (2% to 40% EtOAc/hexanes) to afford the title compound. MS: 392.1 [M+H]+.

The following intermediates were prepared according to the general procedures herein and in an analogous manner to that used to synthesize INTERMEDIATE YYY using the appropriate intermediates. The starting materials were either prepared as described in the intermediates section described earlier.

| | | | Observed |
|---|---|---|---|
| | | | Mass |
| INTERMEDIATE | Structure | Name | [M + H]⁺ |
| INTERMEDIATE ZZZ | 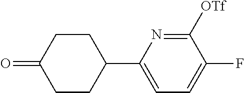 [SMILES: O=C1CCC(c2ccc(F)c(OS(=O)(=O)C(F)(F)F)n2)CC1] | 3-fluoro-6-(4- oxocyclohexyl)pyridin-2- yl trifluoromethanesulfonate | 342.1 |
| INTERMEDIATE AAAA | 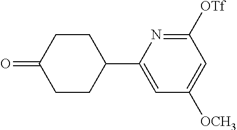 [SMILES: COc1cc(OS(=O)(=O)C(F)(F)F)nc(C2CCC(=O)CC2)c1] | 4-methoxy-6-(4- oxocyclohexyl)pyridin-2- yl trifluoromethanesulfonate | 354.6 |

### Intermediate BBBB

### but-3-en-1-yl (4-nitrophenyl) carbonate

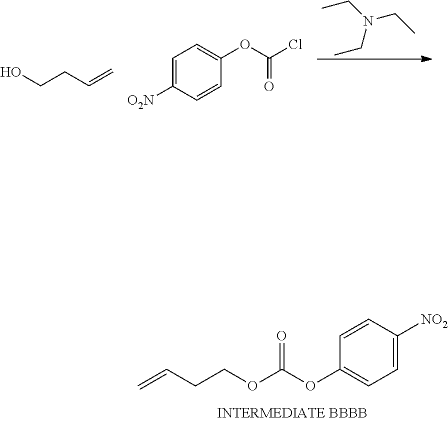

To a mixture of 3-BUTEN-1-OL (2.56 ml, 29.8 mmol) was added CH2Cl2 (49.6 ml). The mixture was cooled to 0° C. and 4-nitrophenyl carbonochloridate (3 g, 14.88 mmol) and triethylamine (6.22 ml, 44.7 mmol) were added dropwise. The mixture was stirred overnight and the crude title compound was used in situ without further purification.

### Intermediate CCCC

### but-3-en-1-yl (2R,3S,5R)-3-(N-(4-methoxybenzyl)methylsulfonamido)-5-methyl-2-(((triethylsilyl)oxy)methyl)pyrrolidine-1-carboxylate

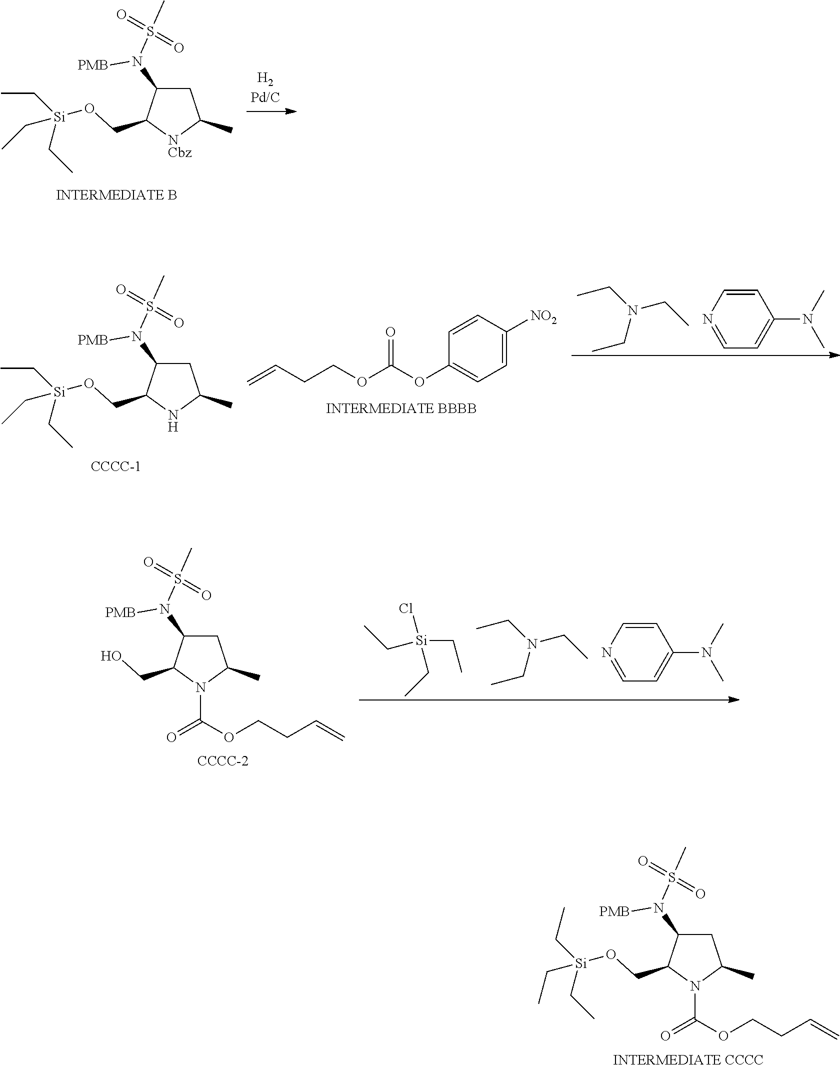

### Step 1: N-(4-methoxybenzyl)-N-((2R,3S,5R)-5-methyl-2-(((triethylsilyl)oxy)methyl)pyrrolidin-3-yl)methanesulfonamide (CCCC-1)

To a mixture of INTERMEDIATE B (4 g, 6.93 mmol) in Methanol (46.2 ml) was added Palladium on carbon (1.476 g, 1.387 mmol) and fitted with a H2 balloon. After 2 hours, the mixture was filtered through a pad of celite and the filtrate concentrated to afford the title compound which will be used directly for the next step.

### Step 2: but-3-en-1-yl (2R,3S,5R)-2-(hydroxymethyl)-3-(N-(4-methoxybenzyl)methylsulfonamido)-5-methylpyrrolidine-1-carboxylate (CCCC-2)

To a mixture of CCCC-1 (3.43 g, 7.75 mmol) and INTERMEDIATE BBBB (51.7 ml, 15.50 mmol) was added triethylamine (3.24 ml, 23.24 mmol) and 4-DIMETHYLAMINOPYRIDINE (0.473 g, 3.87 mmol). The reaction was stirred at room temperature overnight. The mixture was quenched with H2O (50 mL), extracted with DCM (3×50 mL), dried over Na2SO4, and concentrated. The resulting residue was purified using silica column chromatography (2% to 50% EtOAc:EtOH (3:1 v/v)/Hexanes) to afford the title compound. MS: 427.3 [M+H]+.

### Step 3: but-3-en-1-yl (2R,3S,5R)-3-(N-(4-methoxybenzyl)methylsulfonamido)-5-methyl-2-(((triethylsilyl)oxy)methyl)pyrrolidine-1-carboxylate (Intermediate CCCC)

To a mixture of CCCC-2 (2.82 g, 6.61 mmol) in DCM (13.22 ml) was added triethylamine (2.76 ml, 19.83 mmol), chlorotriethylsilane (2.219 ml, 13.22 mmol) and N,N-dimethylpyridin-4-amine (0.242 g, 1.983 mmol). The reaction was stirred for 16 h and was concentrated and purified by silica column chromatography (0-60% EtOAc:EtOH (3:1 v/v)/Hexanes) to afford the title compound. MS: 541.5 [M+H]⁺.

### Example 1

### N′-((2¹R,2⁴R⁵²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)-N,N-dimethyl-sulfamide

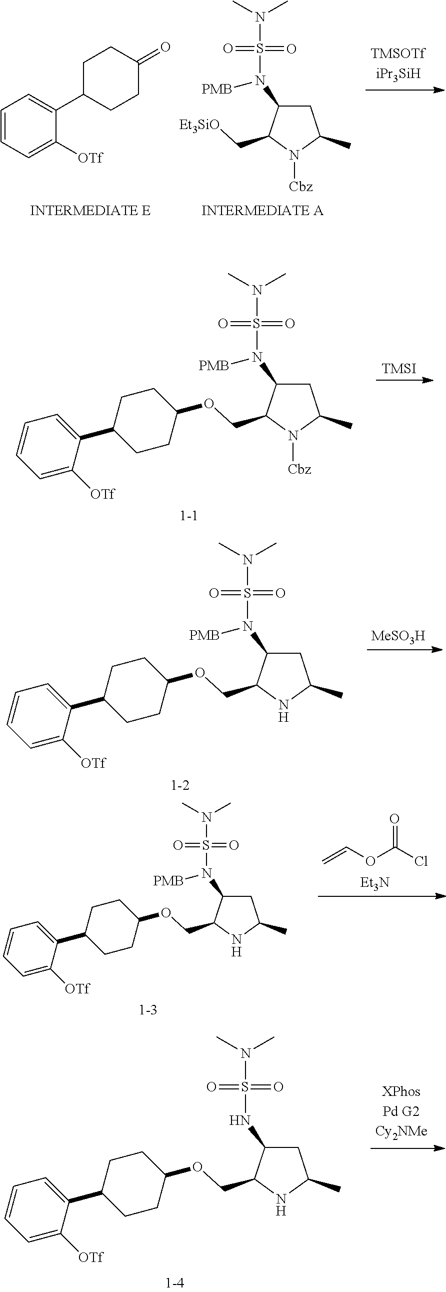

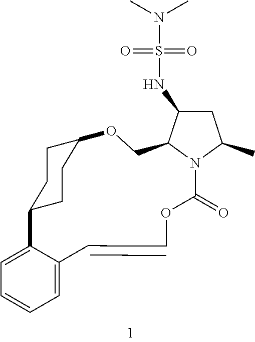

### Step 1: benzyl (2R,3S,5R)-3-((N,N-dimethylsulfamoyl)(4-methoxybenzyl)amino)-5-methyl-2-((((1s,4S)-4-(2-(((trifluoromethyl)sulfonyl)oxy)phenyl)cyclohexyl)oxy)methyl)pyrrolidine-1-carboxylate (1-1)

To a mixture of benzyl (2R,3S,5R)-3-((N,N-dimethylsulfamoyl)(4-methoxy-benzyl)amino)-5-methyl-2-(((triethylsilyl)oxy)methyl)pyrrolidine-1-carboxylate (INTERMEDIATE A) (1000 mg, 1.651 mmol) in MeCN (13.800 mL)/DCM (2.76 mL) at ambient temperature was added 2-(4-oxocyclohexyl)phenyl trifluoromethanesulfonate (INTERMEDIATE E) (532 mg, 1.651 mmol) and triisopropylsilane (0.676 mL, 3.30 mmol). The mixture was cooled to −20° C. and TMS-OTf (0.298 mL, 1.651 mmol) was added dropwise. The mixture was warmed to 0° C. and stirred for 10 min before quenching with a saturated solution of NaHCO₃(25 mL), extract with DCM (3×@ 50 mL), dry over Na2SO4, and concentrate. The resulting residue was purified using silica column chromatography (5% to 65% EtOAc/hexanes) to obtain the title compound. MS: 798.3 (M+H).

### Step 2: 2-((1S,4s)-4-(((2R,3S,5R)-3-((N,N-dimethylsulfamoyl)(4-methoxybenzyl)amino)-5-methylpyrrolidin-2-yl)methoxy)cyclohexyl)phenyl trifluoromethanesulfonate (1-2)

To a mixture of benzyl (2R,3S,5R)-3-((N,N-dimethylsulfamoyl)(4-methoxybenzyl)-amino)-5-methyl-2-((((1s,4S)-4-(2-(((trifluoromethyl)sulfonyl)oxy)phenyl)-cyclohexyl)oxy)-methyl)pyrrolidine-1-carboxylate (1-1) (900 mg, 1.128 mmol) in MeCN (5640 μl) at 0° C. was added TMS-I (184 μl, 1.354 mmol). The mixture stirred for 30 min before adding MeOH (6 mL) and the mixture was stirred for another 15 min. The mixture was concentrated and the resulting residue was purified using C₁₈ column chromatography (5% to 100% MeCN (0.05% TFA)/H2O (0.05% TFA)) to obtain the title compound. MS: 664.4 (M+H).

### Step 3: 2-((1S,4s)-4-(((2R,3S,5R)-3-((N,N-dimethylsulfamoyl)amino)-5-methylpyrrolidin-2-yl)methoxy)cyclohexyl)phenyl trifluoromethanesulfonate (1-3)

To a mixture of 2-((1S,4s)-4-(((2R,3S,5R)-3-((N,N-dimethylsulfamoyl)(4-methoxybenzyl)amino)-5-methylpyrrolidin-2-yl)methoxy)cyclohexyl)phenyl trifluoromethanesulfonate (1-2) (275 mg, 0.414 mmol) in DCM (2072 μl) at ambient temperature was added methanesulfonic acid (269 μl, 4.14 mmol). The mixture was stirred for 1 hour before concentrating. The resulting residue was purified using C18 column chromatography (5% to 100% MeCN (0.05% TFA)/H2O (0.05% TFA)) to obtain the title compound. MS: 544.2 (M+H).

### Step 4: vinyl (2R,3S,5R)-3-((N,N-dimethylsulfamoyl)amino)-5-methyl-2-((((1s,4S)-4-(2-(((trifluoromethyl)sulfonyl)oxy)phenyl)cyclohexyl)oxy)methyl)pyrrolidine-1-carboxylate (1-4)

To a mixture of 2-((1S,4s)-4-(((2R,3S,5R)-3-((N,N-dimethylsulfamoyl)amino)-5-methylpyrrolidin-2-yl)methoxy)cyclohexyl)phenyl trifluoromethanesulfonate (1-3) (85 mg, 0.156 mmol) in DCM (521 μl) at ambient temperature was added vinyl carbonochloridate (18.32 mg, 0.172 mmol) and TRIETHYLAMINE (43.6 μl, 0.313 mmol). The mixture stirred for one hour before concentrating and purifying using silica column chromatography (70% 3:1 EtOAc:EtOH/hexanes) to obtain the title compound. MS: 614.2 (M+H).

### Step 5: N′-((2¹R,2⁴R,5²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)-N,N-dimethyl-sulfamide (1)

To a mixture of vinyl (2R,3S,5R)-3-((N,N-dimethylsulfamoyl)amino)-5-methyl-2-((((1s,4S)-4-(2-(((trifluoromethyl)sulfonyl)oxy)phenyl)cyclohexyl)oxy)methyl)pyrrolidine-1-carboxylate (1-4) (350 mg, 0.570 mmol) in DMF (2.85E+04 μl) at ambient temperature was added N-cyclohexyl-N-methylcyclohexanamine (611 μl, 2.85 mmol) and XPhos Pd G2 (112 mg, 0.143 mmol). The mixture was purged with N2, sealed, and heated to 100° C. for two hours. The mixture was cooled, concentrated, and purified using column chromatography (5% to 100% 3:1 EtOAc:EtOH/hexanes). Another C₁₈ column was used (5% to 100% MeCN (0.05% TFA)/H2O (0.05% TFA)) to obtain the title compound. MS: 464.2 (M+H). 1H NMR (500 MHz, Chloroform-d) δ 7.45 (d, J=6.3 Hz, 1H), 7.17 (bs, 1H), 7.12 (s, 2H), 7.05 (d, J=12.7 Hz, 1H), 6.92 (d, J=12.8 Hz, 1H), 6.11 (bs, 1H), 4.69 (s, 1H), 4.29 (s, 1H), 4.22 (s, 2H), 3.94 (s, 1H), 3.50 (d, J=8.9 Hz, 1H), 2.84 (s, 6H), 2.78-2.70 (in, 1H), 2.35-2.15 (m, 4H), 2.00-1.85 (m, 2H), 1.71-1.53 (m, 4H), 1.45 (d, J=5.6 Hz, 3H).

The following examples were prepared according to the general procedures herein and in an analogous manner to that used to synthesize EXAMPLE 1 using the appropriate intermediates. The starting materials were either prepared as described in the intermediates section, commercially available, or prepared from commercially available reagents using conventional reactions well known in the art.

| | | | Observed |
|---|---|---|---|
| Example | | | Mass |
| Number | Structure | Name | [M + H]⁺ |
| 2 | 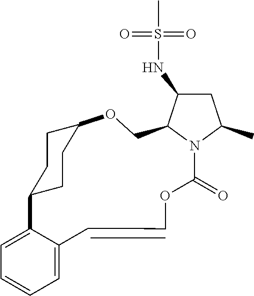 | N- ((2¹R,2⁴R,5²R,5³S,5⁵R,E)- 5⁵-methyl-6-oxo-3,7- dioxa-5(2,1)-pyrrolidina- 1(1,2)-benzena-2(1,4)- cyclohexanacyclonaphan- 8-en-5³- yl)methanesulfonamide | 435.3 |
| 3 | 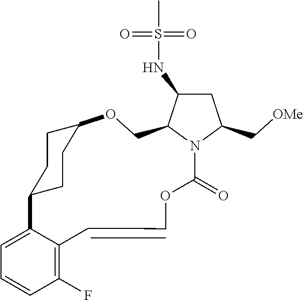 | N- ((2¹R,2⁴R,5²R,5³S,5⁵S,E)- 1³-fluoro-5⁵- (methoxymethyl)-6-oxo- 3,7-dioxa-5(2,1)- pyrrolidina-1(1,2)- benzena-2(1,4)- cyclohexanacyclonaphan- 8-en-5³- yl)methanesulfonamide | 483.4 |

### Example 4

### N′-((2′R,2⁴R,5²R,5³S,5⁵R)-5⁵-methyl-6-oxo-3,7-dioxa-5(2,1)-pyrrolidina-1(1,2)-benzena-2(1,4)- cyclohexanacyclononaphane-5³-yl)-N,N-dimethyl-sulfamide

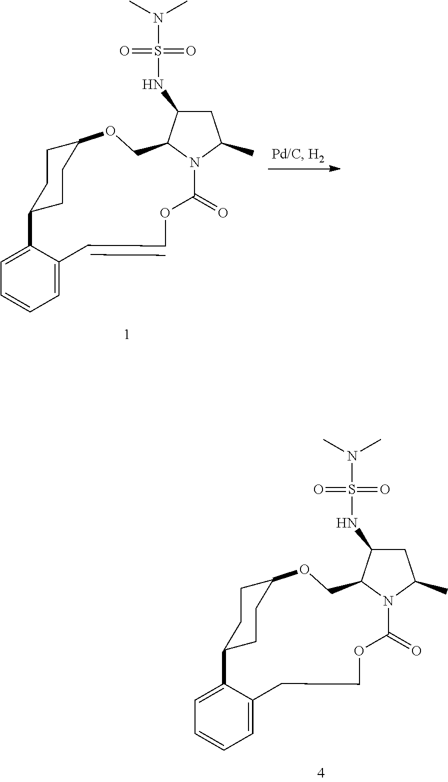

To a mixture of N′-((2¹R,2⁴R,5²R,5³S,5⁵R,E)-5-methyl-6-oxo-3,7-dioxa-5(2,1)-pyrrolidina-1(1,2)-benzena-2(1,4)-cyclohexanacyclononaphan-8-en-5³-yl)-N,N-dimethyl-sulfamide (1) (8 mg, 0.017 mmol) in MeOH (173 μl)/THF (173 μl) at ambient temperature was added Pd/C (3.67 mg, 3.45 μmol). A balloon of H2 was added and the mixture stirred for 20 hours. The resulting mixture was filtered through a pad of celite and the resulting filtrate was concentrated to give the title compound. MS: 466.3 (M+H). 1H NMR (500 MHz, Chloroform-d) δ 7.19-7.10 (m, 3H), 7.05 (d, J=7.0 Hz, 1H), 4.40-4.35 (m, 1H), 4.23 (bs, 1H), 4.13-4.05 (m, 2H), 3.85 (s, 1H), 3.45 (d, J=9.2 Hz, 1H), 3.27 (s, 1H), 2.84 (s, 6H), 2.69 (d, J=7.9 Hz, 1H), 2.56-2.47 (m, 1H), 2.38-2.18 (m, 4H), 1.97 (d, J=11.5 Hz, 2H), 1.75-1.56 (m, 4H), 1.51 (d, J=5.4 Hz, 3H), 1.38-1.23 (m, 3H).

The following examples were prepared according to the general procedures herein and in an analogous manner to that used to synthesize EXAMPLE 4 using the appropriate intermediates. The starting materials were either prepared as described in the intermediates section, commercially available, or prepared from commercially available reagents using conventional reactions well known in the art.

| | | | Observed |
|---|---|---|---|
| Example | | | Mass |
| Number | Structure | Name | [M + H]⁺ |
| 5 | 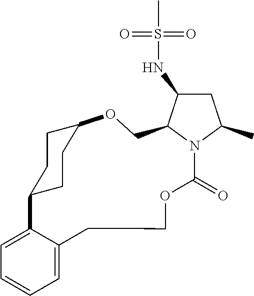 | N-((2¹R,2⁴R,5²R,5³S, 5⁵R)-5⁵-methyl-6-oxo- 3,7-dioxa-5(2,1)- pyrrolidina-1(1,2)- benzena-2(1,4)-cyclo- hexanacyclonaphane- 5³-yl)methane sulfonamide | 437.4 |
| 6 | 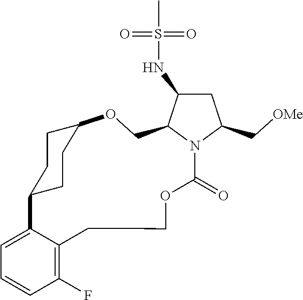 | N-((2¹R,2⁴R,5²R,5³S, 5⁵S)-1³-fluoro-5⁵- (methoxymethyl)-6-oxo- 3,7-dioxa-5(2,1)- pyrrolidina-1(1,2)- benzena-2(1,4)-cyclo- hexanacyclonaphane- 5³-yl)methane sulfonamide | 485.5 |

### Example 7

N′-((2'S,2⁴S,5²R,5³S,5⁵R)-6-oxo-3,11-dioxa-5(2,1)-pyrrolidina-1(1,3)-benzena-2(1,4) cyclohexanacycloundecaphane-5³-yl)-N,N-dimethylsulfamide

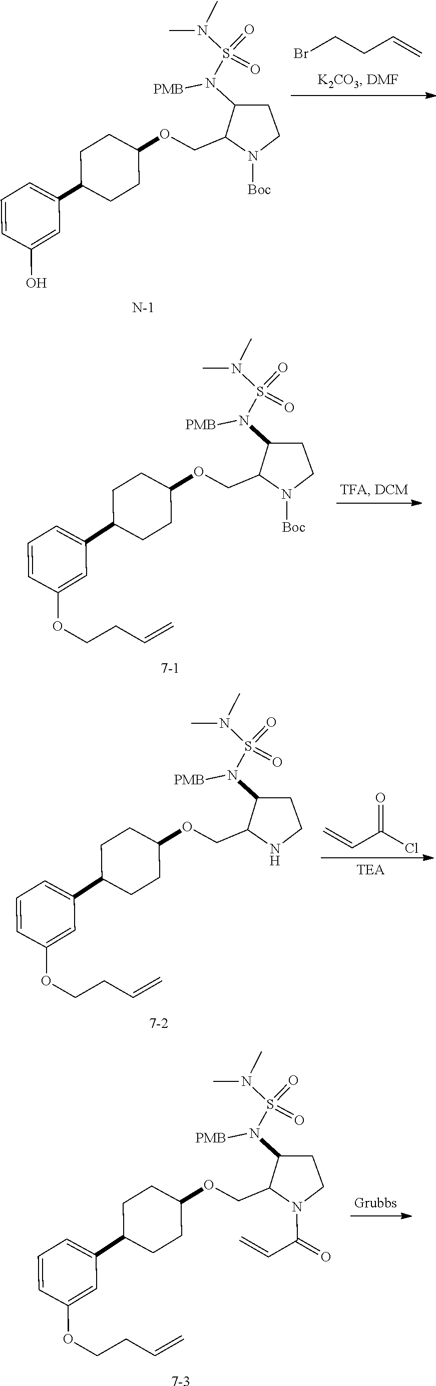

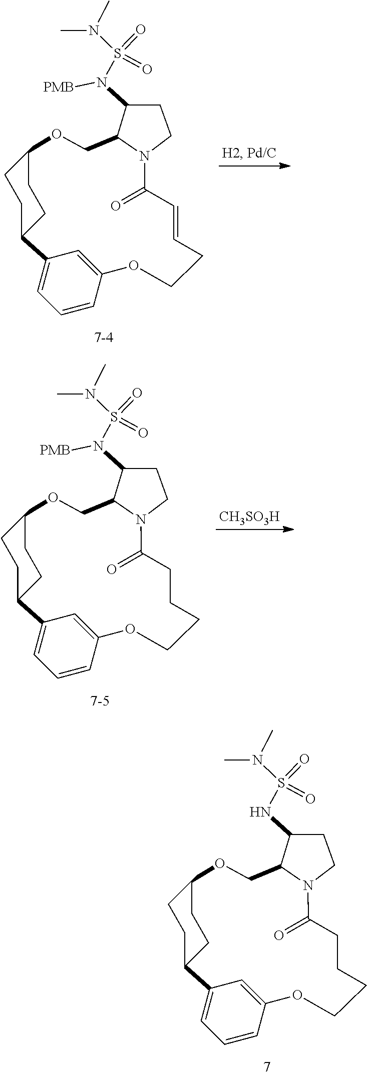

### Step 1: tert-butyl (2R,3S)-2-(((4-(3-(but-3-en-1-yloxy)phenyl)cyclohexyl)oxy)methyl)-3-((N,N-dimethylsulfamoyl)(4-methoxybenzyl)amino)pyrrolidine-1-carboxylate (7-1)

A solution of tert-butyl (2R,3S)-3-((N,N-dimethylsulfamoyl)(4-methoxybenzyl)amino)-2-(((4-(3-hydroxyphenyl)cyclohexyl)oxy)methyl)pyrrolidine-1-carboxylate (N-1) (340 mg, 0.550 mmol) in DMF (5000 μl) was added 4-bromobut-1-ene (279 μl, 2.75 mmol) followed by Cs₂CO₃ (897 mg, 2.75 mmol). The reaction was stirred at 60° C. for 15 hrs. The mixture was added water (250 mL) and extracted with ethyl acetate (3×50 mL). The combined organic fractions were washed with brine (saturated, 50 mL), dried (Na₂SO₄), filtered and the solvent was evaporated under reduced pressure. The residue was purified by silica column chromatography (0-60% EtOAc/hexane) to give the title compound. MS: 672 (M+H).

### Step 2: (2R,3S)-2-(((4-(3-(but-3-en-1-yloxy)phenyl)cyclohexyl)oxy)methyl)-3-((N,N-dimethylsulfamoyl)(4-methoxybenzyl)amino)pyrrolidine (7-2)

A mixture of tert-butyl (2R,3S)-2-(((4-(3-(but-3-en-1-yloxy)phenyl)cyclohexyl)oxy) methyl)-3-((N,N-dimethylsulfamoyl)(4-methoxybenzyl)amino)pyrrolidine-1-carboxylate (7-1) (215 mg, 0.320 mmol) in DCM (4000 μl) and TFA (1000 μl) was stirred at rt for 15 hrs. The reaction was concentrated to give to give the title compound. MS: 572 (M+H).

### Step 3: (2R,3S)-1-acryloyl-2-(((4-(3-(but-3-en-1-yloxy)phenyl)cyclohexyl)oxy)methyl)-3-((N,N-dimethylsulfamoyl)(4-methoxybenzyl)amino)pyrrolidine (7-3)

A mixture of (2R,3S)-2-(((4-(3-(but-3-en-1-yloxy)phenyl)cyclohexyl)oxy)methyl)-3-((N,N-dimethylsulfamoyl)(4-methoxybenzyl)amino)pyrrolidine (7-2) (147 mg, 0.257 mmol) in DCM (4000 μl) was added Et₃N (358 μl, 2.57 mmol) followed by ACRYLOYL CHLORIDE (62.3 μl, 0.771 mmol). After stirring at rt for 60 min, the reaction mixture was directly purified by silica column chromatography (0-50% EtOAc/hexane) to give the title compound. MS: 626 (M+H).

### Step 4: N′-((2¹S,2⁴S,5²R,5³S)-5³-((4-methoxybenzyl)amino)-6-oxo-3,11-dioxa-5(2,1)-pyrrolidina-1(1,3)-benzena-2(1,4)-cyclohexanacycloundecaphan-7-en-5³-yl)-N,N-dimethyl-sulfamide (7-4)

To a mixture of (2R,3S)-1-acryloyl-2-(((4-(3-(but-3-en-1-yloxy)phenyl)cyclohexyl) oxy)methyl)-3-((N,Ndimethylsulfamoyl)(methoxybenzyl)amino)pyrrolidine (7-3)(100 mg, 0.160 mmol) in DCE (16 ml) at ambient temperature was added GRUBBS CATALYST C571 (30.1 mg, 0.053 mmol) and N2 was bubbled through the solution for 3 min. The mixture was heated to 60° C. and stirred overnight. The mixture was cooled and concentrated. The resulting residue was purified using silica column chromatography (5%-100% 3:1 EtOAc:EtOH/hexanes) to obtain the title compound. MS: 598 (M+H).

### Step 5: N′-((2¹S,2⁴S,5²R,5³S)-5³-((4-methoxybenzyl)amino)-6-oxo-3,11-dioxa-5(2,1)-pyrrolidina-1(1,3)-benzena-2(1,4)-cyclohexanacycloundecaphane-5³-yl)-N,N-dimethylsulfamide (7-5)

To a mixture of N′-((2¹S,2⁴S,5²R,5³S)-5³-((4-methoxybenzyl)amino)-6-oxo-3,11-dioxa-5(2,1)-pyrrolidina-1(1,3)-benzena-2(1,4)-cyclohexanacycloundecaphan-7-en-5³-yl)-N,N-dimethyl-sulfamide (7-4) (40 mg, 0.067 mmol) in Methanol (335 μl)/THF (335 μl) was added Pd/C (7.12 mg, 6.69 μmol) and a hydrogen balloon was added (vacuum purged 3 times). The mixture stirred for one hour before the mixture was filtered through a pad of celite and concentrated to give the title compound. MS: 600 (M+H).

### Step 6: N′-((2¹S,2⁴S,5²R,5³S)-6-oxo-3,11-dioxa-5(2,1)-pyrrolidina-1(1,3)-benzena-2(1,4)-cyclohexanacycloundecaphane-5³-yl)-N,N-dimethyl-sulfamide (7)

To a mixture of N′-((2¹S,2⁴S,5²R,5³S)-5³-((4-methoxybenzyl)amino)-6-oxo-3,11-dioxa-5(2,1)-pyrrolidina-1(1,3)-benzena-2(1,4)-cyclohexanacycloundecaphane-5³-yl)-N,N-dimethyl-sulfamide (7-5) (30 mg, 0.050 mmol) in DCM (500 μl) at ambient temperature was added methanesulfonic acid (32.5 μl, 0.500 mmol). The mixture stirred for 1 hour before concentrating. The resulting residue was purified using C₁₈ reverse phase HPLC (5%-100% MeCN (0.05% TFA)/H2O (0.05% TFA)) to obtain the title compound. MS: 480 (M+H). 1H NMR (500 MHz, DMSO-d6) δ 7.52 (d, J=6.9 Hz, 1H), 7.08 (t, J=7.9 Hz, 1H), 6.69 (d, J=7.1 Hz, 1H), 6.66-6.61 (m, 2H), 4.31-4.25 (m, 1H), 4.07-3.89 (m, 3H), 3.78-3.66 (m, 2H), 3.42 (dt, J=18.9, 10.2 Hz, 1H), 3.32 (d, J=9.0 Hz, 1H), 2.70 (s, 6H), 2.32-2.22 (m, 2H), 2.13-1.73 (m, 8H), 1.59-1.27 (m, 8H).

The following examples were prepared according to the general procedures herein and in an analogous manner to that used to synthesize EXAMPLE 7 using the appropriate intermediates. The starting materials were either prepared as described in the intermediates section, commercially available, or prepared from commercially available reagents using conventional reactions well known in the art.

| | | | Exact |
|---|---|---|---|
| Example | | | Mass |
| Number | Structure | Name | [M + H]⁺ |
| 8 | 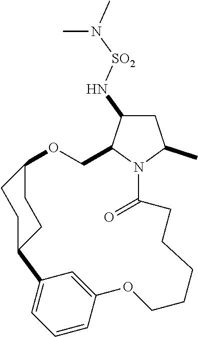 | N′- ((2¹S,2⁴S,5²R,5³S,5⁵R)- 5⁵-methyl-6-oxo-3,12- dioxa-5(2,1)-pyrrolidina- 1(1,3)-benzena-2(1,4)- cyclohexanacyclododecaphane- 5³-yl)-N,N- dimethyl-sulfamide | 508 |
| 9 | 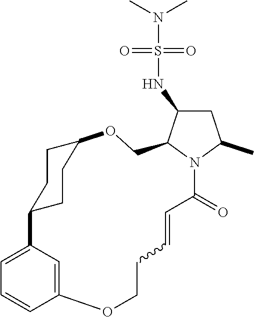 | N′-((2¹S,2⁴S,5²R,5³S, 5⁵R)-5⁵-methyl-6-oxo- 3,11-dioxa-5(2,1)- pyrrolidina-1(1,3)- benzena-2(1,4)- cyclohexanacycloundecaphan- 7-en-5³-yl)-N,N- dimethyl-sulfamide | 492 |
| 10 | 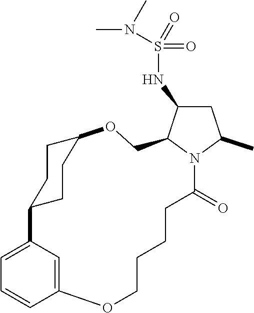 | N′-((2¹S,2⁴S,5²R,5³S, 5⁵R)-5⁵-methyl-6-oxo- 3,11-dioxa-5(2,1)- pyrrolidina-1(1,3)- benzena-2(1,4)- cyclohexanacycloundecaphane- 5³-yl)-N,N- dimethyl-sulfamide | 494 |
| 11 | 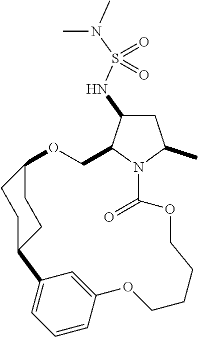 | N′- ((2¹S,2⁴S,5²R,5³S,5⁵R)- 5⁵-methyl-6-oxo-3,7,12- trioxa-5(2,1)-pyrrolidina- 1(1,3)-benzena-2(1,4)- cyclohexanacyclododecaphane- 5³-yl)-N,N- dimethyl-sulfamide | 510 |

### Example 12

### N′-((2'S,2⁴S,5²R,5³S,5⁵R)-5⁵-methyl-6-oxo-3-oxa-5(2,1)-pyrrolidina-1(1,3)-benzena-2(1,4) cyclohexanacycloundecaphane-5³-yl)-N,N-didimethyl-sulfamide

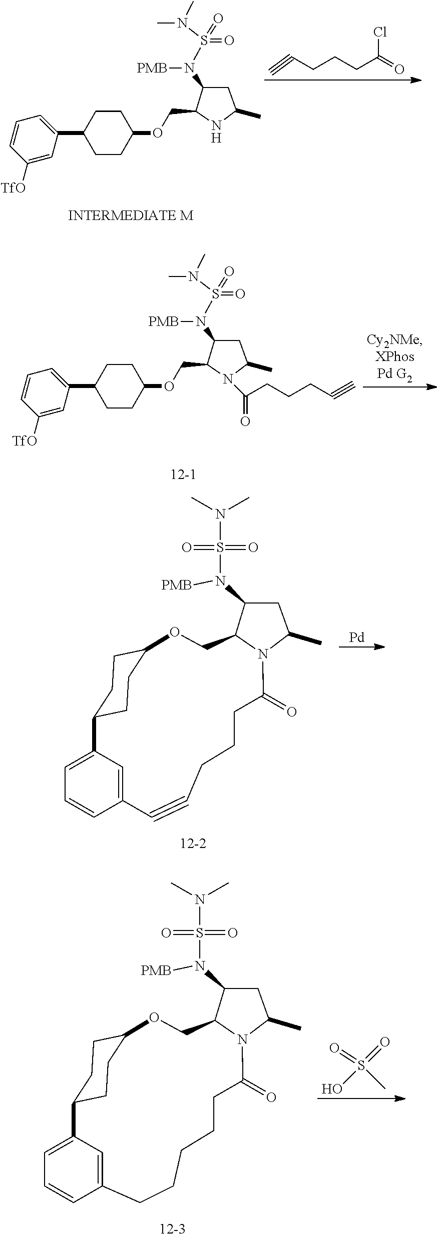

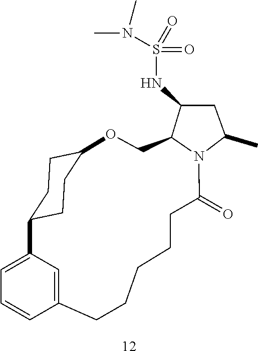

### Step 1: 3-(4-(((2R,3S,5R)-3-((N,N-dimethylsulfamoyl)(4-methoxybenzyl)amino)-1-(hex-5-ynoyl)-5-methylpyrrolidin-2-yl)methoxy)cyclohexyl)phenyl trifluoromethanesulfonate (12-1)

A suspension of 3-(4-(((2R,3S,5R)-3-((N,N-dimethylsulfamoyl)(4methoxybenzyl) amino)-5-methylpyrrolidin-2-yl)methoxy)cyclohexyl)phenyl trifluoromethanesulfonate TFA salt (INTERMEDIATE M) (60 mg, 0.079 mmol), Et₃N (54.9 μl, 0.394 mmol) and DMAP (4.81 mg, 0.039 mmol) in DCM (1000 μl) was added HEX-5-YNOYL CHLORIDE (18.70 μl, 0.158 mmol) stirred at rt. After 18 hrs, the reaction mixture was directly purified by silica column chromatography (0-50% EtOAc/hexanes) to give the title compound. MS: 758 (M+H).

### Step 2: N′-((2¹S,2⁴S,5²R,5³S,5⁵R)-5³-((4-methoxybenzyl)amino)-5⁵-methyl-6-oxo-3-oxa-5(2,1)-pyrrolidina-1(1,3)-benzena-2(1,4)-cyclohexanacycloundecaphane-10-yn-5³-yl)-N,N-didimethyl-sulfamide (12-2)

A mixture of 3-(4-(((2R,3S,5R)-3-((N,N-dimethylsulfamoyl)(4-methoxybenzyl)amino)-1-(hex-5-ynoyl)-5-methylpyrrolidin-2-yl)methoxy)cyclohexyl)phenyl trifluoromethanesulfonate (12-1) (28 mg, 0.037 mmol), XPhos Pd G2 (8.72 mg, 0.011 mmol) and N-cyclohexyl-N-methylcyclohexanamine (39.6 μl, 0.185 mmol) in DMF (3700 μl) was charged in 40 ml of microwave vial and bubbled with N₂ for 4 min. The vial was sealed and heated to 80° C. After 1 hr, reaction went to completion with formation of the desired product. Cooled to rt and concentrated under reduced pressure. The residue was purified by C₁₈ reverse phase HPLC (10-100% Water in Acetonitrile with 0.05% TFA) to give the title compound. MS: 608 (M+H).

### Step 3: N′-((2¹S,2⁴S,5²R,5³S,5⁵R)-5³-((4-methoxybenzyl)amino)-5⁵-methyl-6-oxo-3-oxa-5(2,1)-pyrrolidina-1(1,3)-benzena-2(1,4)-cyclohexanacycloundecaphane-5³-yl)-N,N-dimethyl-sulfamide (12-3)

A solution of N′-((2¹S,2⁴S,5²R,5³S,5⁵R)-53-((4-methoxybenzyl)amino)-5⁵-methyl-6-oxo-3-oxa-5(2,1)-pyrrolidina-1(1,3)-benzena-2(1,4)-cyclohexanacycloundecaphane-10-yn-5³-yl)-N,N-didimethyl-sulfamide (12-2) (8 mg, 0.013 mmol) in MeOH (1000 μl) was added palladium on carbon (1.401 mg, 0.013 mmol) at rt. The mixture was stirred under a H₂ balloon at rt. After 1 hr, the reaction was completed and the mixture was filtered through a pad of celite and washed with MeOH. The combined filtrates were concentrated under reduced pressure to give the title compound. MS: 612 (M+H).

### Step 4: N′-((2¹S,2⁴S,5²R,5³S,5⁵R)-5⁵-methyl-6-oxo-3-oxa-5(2,1)-pyrrolidina-1(1,3)-benzena-2(1,4)cyclohexanacycloundecaphane-5³-yl)-N,N-dimethyl-sulfamide (12)

The title compound 12 was prepared from N′-((2¹S,2⁴S,5²R,5³S,5⁵R)-5³-((4-methoxybenzyl)amino)-5⁵-methyl-6-oxo-3-oxa-5(2,1)-pyrrolidina-1(1,3)-benzena-2(1,4)-cyclohexanacycloundecaphane-5³-yl)-N,N-didimethyl-sulfamide (12-3) and appropriate reagents according to the same procedure provided in step 6 for the preparation of 7. MS: 492 (M+H).

### Example 13

### N′-((2¹S,2⁴S,5²R,5³S,5⁵R)-)-5⁵-methyl-6-oxo-3,7,11-trioxa-5(2,1)-pyrrolidina-1(1,3)-benzena-2(1,4)-cyclohexanacycloundecaphane-5³-yl)-N,N-dimethyl-sulfamide

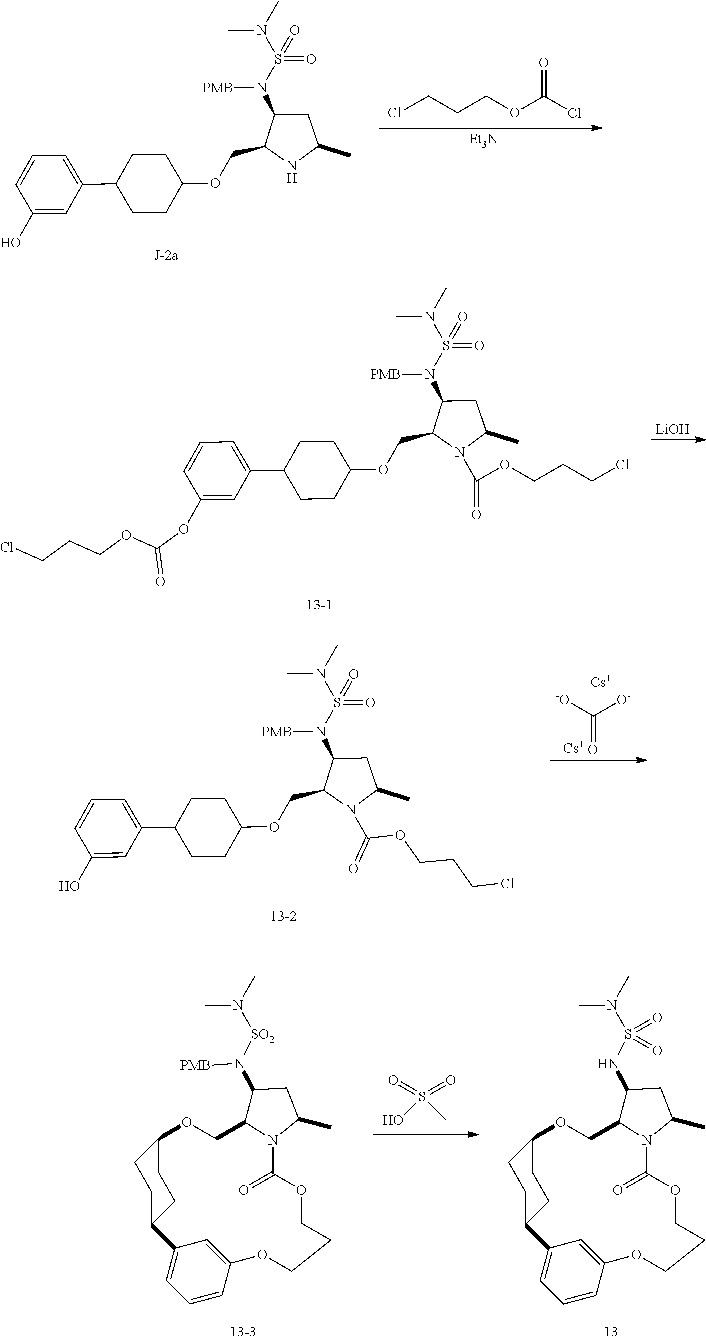

### Step 1: 3-chloropropyl (2R,3S,5R)-2-(((4-(3-(((3chloropropoxy)carbonyl)oxy)phenyl) cyclohexyl)oxy)methyl)-3-((N,N-dimethylsulfamoyl)(4-methoxybenzyl)amino)-5-methylpyrrolidine-1-carboxylate (13-1)

To a solution of (2R,3S,5R)-2-((4-(3-(phenylhydroxy)cyclohexyl)oxy)methyl)-3-((N,N-dimethylsulfamoyl)(4-methoxybenzyl)amino)-5-methylpyrrolidine (J-2a) (40 mg, 0.075 mmol) in DCM (1000 μl) was added Et₃N (62.9 μl, 0.451 mmol) followed by 3-chloropropyl carbonochloridate (27.2 μl, 0.226 mmol). After 30 min at rt, reaction was completed. Concentrated under reduced pressure to give the title compound. MS: 772 (M+H).

### Step 2: 3-chloropropyl-1-((2R,3S)-3-((N,N-dimethylsulfamoyl)amino)-2-(((4-(3-hydroxyphenyl)cyclohexyl)oxy)methyl)pyrrolidin-1-carboxylate (13-2)

To a solution of 3-chloropropyl (2R,3S,5R)-2-(((4-(3-(((3chloropropoxy)carbonyl)oxy) phenyl)cyclohexyl)oxy)methyl)-3-((N,N-dimethylsulfamoyl)(4-methoxybenzyl)amino)-5-methylpyrrolidine-1-carboxylate (13-1) (58 mg, 0.075 mmol) in MeOH (400 μl)/THF (400 μl) was added lithium hydroxide (aq 1.0M, 375 μl, 0.375 mmol). After 30 min at rt, hydrolysis reaction completed. Acidifed with HCl (aq 7.0 M,107 μl, 0.751 mmol). The mixture was added water (10 mL) and the mixture was extracted with ethyl acetate (3×10 mL). The combined organic fractions were washed with brine (saturated, 10 mL), dried (Na₂SO₄), filtered, and the solvent was evaporated under reduced pressure to give the title compound. MS: 652 (M+H).

### Step 3: N′-((2¹S,2⁴S,5²R,5³S,5⁵R)-)-5³-((4-methoxybenzyl)amino-5⁵-methyl-6-oxo-3,7,11-trioxa-5(2,1)-pyrrolidina-1(1,3)-benzena-2(1,4)-cyclohexanacycloundecaphane-5³-yl)-N,N-dimethyl-sulfamide (13-3)

To a solution of 3-chloropropyl (2R,3S,5R)-3-((N,N-dimethylsulfamoyl)(4-methoxybenzyl)amino)-2-(((4-(3-hydroxyphenyl)cyclohexyl)oxy)methyl)-5-methylpyrrolidine-1-carboxylate (13-2)(49 mg, 0.075 mmol) in DMF (8500 μl) was added Cs₂CO₃ (147 mg, 0.451 mmol). Stirred at 60° C. for 15 hrs. The reaction was filtered with help of EtOAc and the filtrates was concentrated under reduced pressure. The residue was purified by 1000 micron prep silica gel TLC plate [20×20 cm], eluting with 50% EtOAc/isohexane. The desired band was collected, filtered with EtOAc and concentrated under reduced pressure to give the title compound. MS: 616 (M+H).

### Step 4: N′-((2¹S,2⁴S,5²R,5³S,5⁵R)-)-5⁵-methyl-6-oxo-3,7,11-trioxa-5(2,1)-pyrrolidina-1(1,3)-benzena-2(1,4)-cyclohexanacycloundecaphane-5³-yl)-N,N-dimethyl-sulfamide (13)

13 was prepared from N′-((2¹S,2⁴S,5²R,5³S,5⁵R)-)-5³-((4-methoxybenzyl)amino-5⁵-methyl-6-oxo-3,7,11-trioxa-5(2,1)-pyrrolidina-1(1,3)-benzena-2(1,4)-cyclohexana-cycloundecaphane-5³-yl)-N,N-dimethyl-sulfamide (13-3) according to the same procedure provided in step 6 for the preparation of 7. MS: 496 (M+H). ¹H NMR (500 MHz, Methanol-d₄) δ 7.12-7.04 (m, 1H), 6.75-6.69 (m, 2H), 6.66 (d, J=6.7 Hz, 1H), 4.76-4.68 (m, 1H), 4.41-4.34 (m, 1H), 4.25 (d, J=15.1 Hz, 2H), 3.88 (d, J=28.7 Hz, 4H), 3.79 (s, 1H), 3.49 (s, 1H), 2.80 (s, 6H), 2.61-2.51 (m 1H), 2.48-2.32 (m, 2H), 2.10 (q, J=15.6, 14.4 Hz, 3H), 1.91-1.79 (m, 2H), 1.79-1.68 (m, 1H), 1.50 (d, J=29.7 Hz, 7H).

The following examples were prepared according to the general procedures herein and in an analogous manner to that used to synthesize EXAMPLE 13 using the appropriate intermediates. The starting materials were either prepared as described in the intermediates section, commercially available, or prepared from commercially available reagents using conventional reactions well known in the art.

| Ex- | | | |
|---|---|---|---|
| ample | | | Exact |
| Num- | | | Mass |
| ber | Structure | Name | [M + H]⁺ |
| 14 | 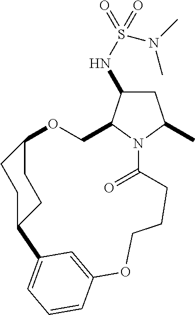 | N′- ((2¹S,2⁴S,5²R, 5³S,5⁵R)-5⁵- methyl-6-oxo- 3,10-dioxa- 5(2,1)-pyrrolidina- 1(1,3)- benzena-2(1,4)- cyclohexana- cyclodecaphane- 5³-yl)-N,N-methyl- sulfamide | 480 |
| 15 | 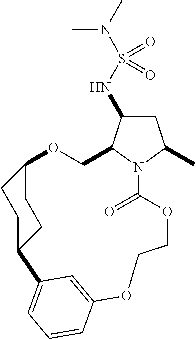 | N′- ((2¹S,2⁴S,5²R, 5³S,5⁵R)-5⁵- methyl-6- oxo-3,7,10- trioxa-5(2,1)- pyrrolidina- 1(1,3)- benzena-2(1,4)- cyclohexana- cyclodecaphane- 5³-yl)-N,N- dimethyl- sulfamide | 482 |
| 16 | 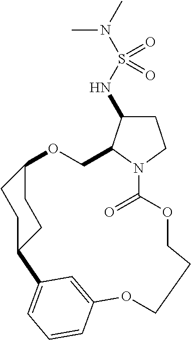 | N′-((2¹S,2⁴S, 5²R,5³S)-6- oxo-3,7,11- trioxa-5(2,1)- pyrrolidina-1(1,3)- benzena-2(1,4)- cyclohexana- cyclounde- caphane-5³- yl)-N,N- dimethyl-sulfamide | 482 |
| 17 | 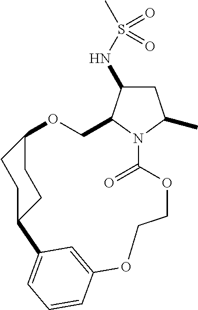 | N-((2¹S,2⁴S, 5²R,5³S,5⁵R)- 5⁵-methyl-6- oxo-3,7,10- trioxa-5(2,1)- pyrrolidina- 1(1,3)- benzena-2(1,4)- cyclohexana- cyclodecaphane- 5³-yl) methanesulfonamide | 453 |
| 18 | 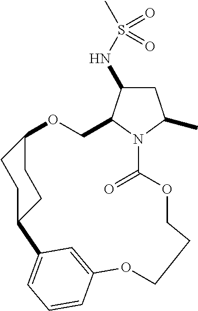 | N-((2¹S,2⁴S, 5²R,5³S,5⁵R)- 5⁵-methyl-6- oxo-3,7,11- trioxa-5(2,1)- pyrrolidina- 1(1,3)- benzena-2(1,4)- cyclohexana- cyclounde- caphane-5³-yl) methanesulfonamide | 467 |
| 19 | 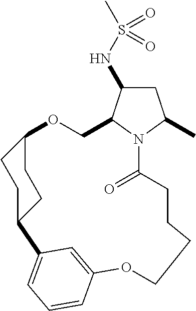 | N-((2¹S,2⁴S, 5²R,5³S,5⁵R)- 5⁵-methyl- 6-oxo-3,11- dioxa-5(2,1)- pyrrolidina- 1(1,3)- benzena-2(1,4)- cyclohexana- cyclounde- caphane-5³-yl) methanesulfonamide | 465 |
| 20 | 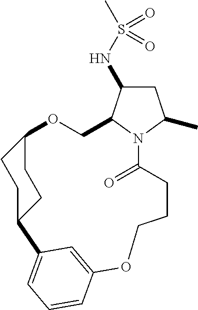 | N-((2¹S,2⁴S, 5²R,5³S,5⁵R)- 5⁵-methyl-6- oxo-3,10- dioxa-5(2,1)- pyrrolidina- 1(1,3)- benzena-2(1,4)- cyclohexana- cyclodecaphane- 5³-yl) methanesulfonamide | 451 |

### Example 21

### N′-((2¹S,2⁴S,5²R,5³S)-6-oxo-3,7-dioxa-5(2,1)-pyrrolidina-1(1,3)-benzena-2(1,4)-cyclo hexanacycloundecaphane-5³-yl)-N,N-dimethyl-sulfamide

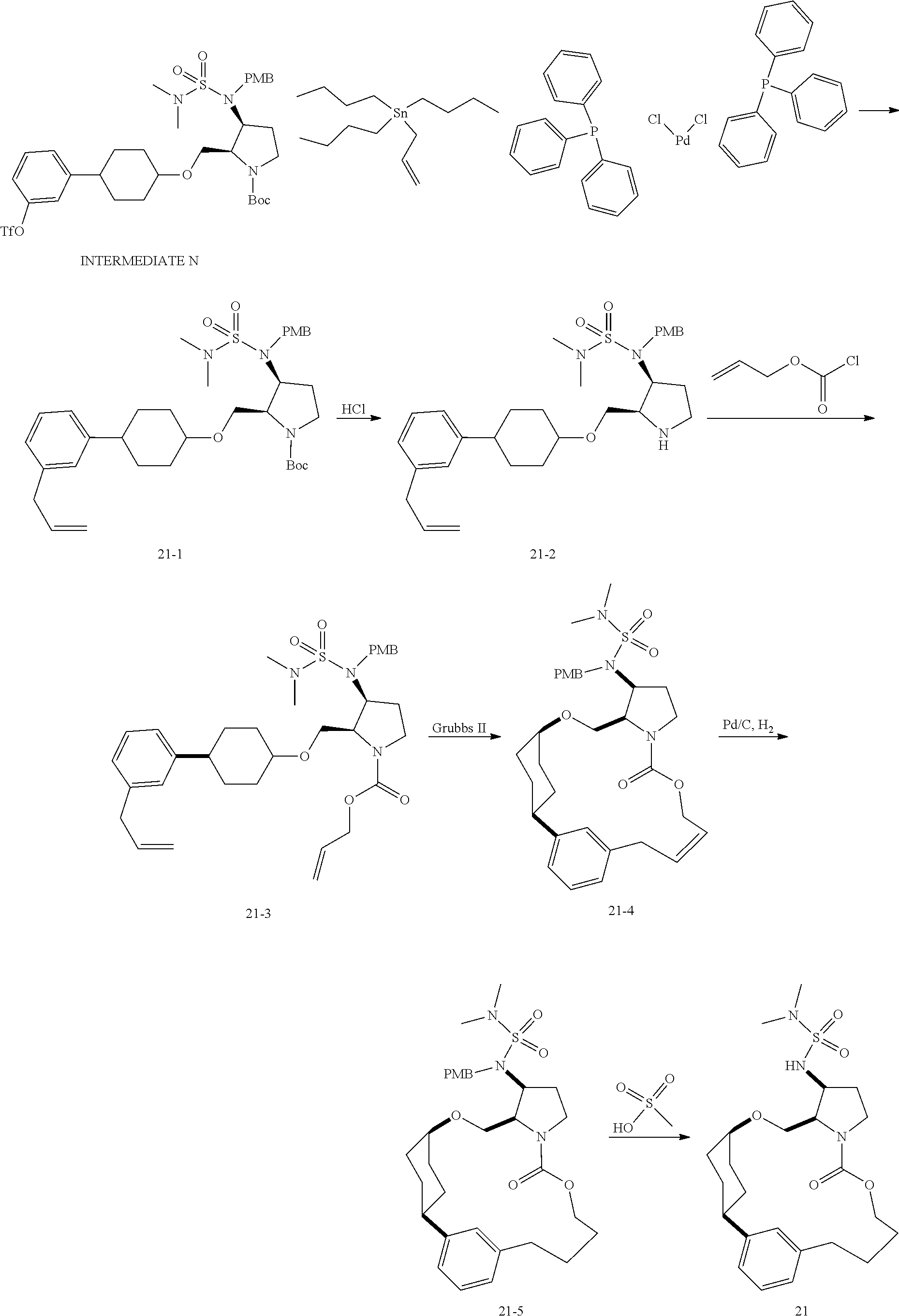

### Step 1: tert-butyl (2R,3S)-2-(((4-(3-allylphenyl)cyclohexyl)oxy)methyl)-3-((N,N-dimethylsulfamoyl)(4-methoxybenzyl)amino)pyrrolidine-1-carboxylate (21-1)

A 10 ml of microwave vial charged a mixture of tert-butyl (2R,3S)-3-((N,N-dimethylsulfamoyl)(4-methoxybenzyl)amino)-2-(((4-(3(((trifluoromethyl)sulfonyl) oxy)phenyl)cyclohexyl)oxy)methyl)pyrrolidine-1-carboxylate (INTERMEDIATE N) (96 mg, 0.128 mmol), allyltributyltin (63.5 μl, 0.205 mmol), lithium chloride (109 mg, 2.56 mmol) and bis(triphenylphosphine)palladium(II) dichloride (8.99 mg, 0.013 mmol) in DMF (1500 μl) was purged with N2 for 3 min. Stirred at 90° C. for 2 hrs. The reaction was concentrated under reduced pressure and the residue was purified by 2×1000 micron prep silica gel TLC plate [20×20 cm], eluting with 50% EtOAc/hexanes. The desired band was collected, filtered with ethyl acetate and concentrated under reduced pressure to give the title compound. MS: 642 (M+H).

### Step 2: (2R,3S)-2-(((4-(3-allylphenyl)cyclohexyl)oxy)methyl)-3-((N,N-dimethylsulfamoyl)(4-methoxybenzyl)amino)pyrrolidine (21-2)

A solution of tert-butyl (2R,3S)-2-(((4-(3-allylphenyl)cyclohexyl)oxy)methyl)-3-((N,N-dimethylsulfamoyl)(4-methoxybenzyl)amino)pyrrolidine-1-carboxylate (21-1) (90 mg, 0.140 mmol) in dioxane (700 μl) was added HCl in dioxane (701 μl, 2.80 mmol) at rt. After stirring at for 0.5 hr, reaction was concentrated to give the title compounds. MS: 542 (M+H).

### Step 3: allyl (2R,3S)-2-(((4-(3-allylphenyl)cyclohexyl)oxy)methyl)-3-((N,N-dimethylsulfamoyl)(4-methoxybenzyl)amino)pyrrolidine-1-carboxylate (21-3)

A solution of (2R,3S)-2-(((4-(3-allylphenyl)cyclohexyl)oxy)methyl)-3-((N,N-dimethylsulfamoyl)(4-methoxybenzyl)amino)pyrrolidine (21-2) (75 mg, 0.138 mmol) in DCM (2000 μl) was added Et₃N (193 μl, 1.384 mmol) followed by allyl carbonochloridate (17.72 μl, 0.166 mmol) at rt. After stirring at rt for 40 min, the reaction mixture was directly purified by silica column chromatography (0-50% EtOAc/hexanes) to give the title compound. MS: 626 (M+H).

### Step 4: N′-((2¹S,2⁴S,5²R,5³S)-5³-((4-methoxybenzyl)amino)-6-oxo-3,7-dioxa-5(2,1)-pyrrolidina-1(1,3)-benzena-2(1,4)-cyclohexanacycloundecaphan-9-en-5³-yl)-N,N-dimethyl-sulfamide (21-4)

A solution of allyl (2R,3S)-2-((((1s,4S)-4-(3-allylphenyl)cyclohexyl)oxy)methyl)-3-((N,N-dimethylsulfamoyl)(4-methoxybenzyl)amino)pyrrolidine-1-carboxylate (21-3) (61 mg, 0.097 mmol) in DCE (10 ml) in 10 ml of microwave vial was added Grubbs II (55.6 mg, 0.097 mmol), purged with N2 for 4 min, capped and then heated to 45° C. After 2 hrs, the reaction mixture was purified by 1000 micron prep silica gel TLC plate [20×20 cm], eluting with 50% EtOAc/hexanes. The desired band was collected, filtered with ethyl acetate and concentrated under reduced pressure to give the title compound. MS: 598 (M+H).

### Step 5: N′-((2¹S,2⁴S,5²R,5³S)-5³-((4-methoxybenzyl)amino)-6-oxo-3,7-dioxa-5(2,1)-pyrrolidina-1(1,3)-benzena-2(1,4)-cyclohexanacycloundecaphan-9-en-5³-yl)-N,N-dimethyl-sulfamide (21-5)

21-5 was prepared from N-((2¹S,2⁴S,5²R,5³S)-5³-((4-methoxybenzyl)amino)-6-oxo-3,7-dioxa-5(2,1)-pyrrolidina-1(1,3)-benzena-2(1,4)cyclohexana-cycloundecaphan-9-en-5³-yl)-N,N-dimethyl-sulfamide (21-4) according to the same procedure provided in step 5 for the preparation of 7. MS: 600 (M+H).

### Step 6: N′-((2¹S,2⁴S,5²R,5³S)-6-oxo-3,7-dioxa-5(2,1)-pyrrolidina-1(1,3)-benzena-2(1,4)-cyclo hexanacycloundecaphane-5³-yl)-N,N-dimethyl-sulfamide (21)

21 was prepared by using the appropriate reagents and N-((2¹S,2⁴S,5²R,5³S)-5³-((4-methoxybenzyl)amino)-6-oxo-3,7-dioxa-5(2,1)-pyrrolidina-1(1,3)-benzena-2(1,4)-cyclohexanacycloundecaphan-9-en-5³-yl)-N,N-dimethyl-sulfamide (21-5) according to the same procedure provided in step 6 for the preparation of 7. MS: 480 (M+H). ¹H NMR (400 MHz, Methanol-d₄) δ 7.16-7.06 (m, 2H), 6.92 (t, J=7.7 Hz, 2H), 4.16-3.92 (m, 2H), 3.82-3.62 (m, 2H), 3.56-3.43 (m, 2H), 2.93 (d, J=15.9 Hz, 1H), 2.79 (s, 6H), 2.74-2.59 (m, 2H), 2.50 (d, J=12.4 Hz, 2H), 2.31 (dt, J=20.7, 10.0 Hz, 2H), 2.18 (d, J=11.4 Hz, 3H), 2.07-1.81 (m, 3H), 1.71 (m, 3H), 1.66-1.47 (m, 3H), 1.42 (d, J=11.9 Hz, 2H).

### Example 22

### N′-((2¹S,2⁴S,5²R,5³S,5⁵R)-5⁵-methyl-6-oxo-3,7-dioxa-5(2,1)-pyrrolidina-1(1,3)-benzena-2(1,4)- cyclohexanacycloundecaphane-5³-yl)-N,N-dimethysulfamide

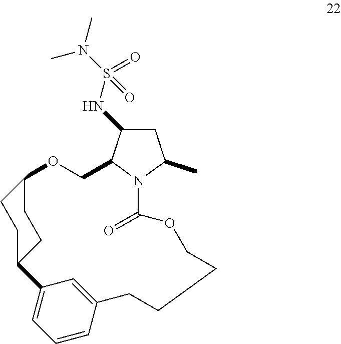

The title compound of EXAMPLE 22 was prepared by using the appropriate reagents and tert-butyl (2R,3S)-3-((N,N-dimethylsulfamoyl)(4-methoxybenzyl)amino)-2-(((4-(3-(((trifluoromethyl)sulfonyl)oxy)phenyl)cyclohexyl)-oxy)methyl)pyrrolidine-1-carboxylate (M-2) according to the same procedure provided in preparation of EXAMPLE 21. MS: 494 (M+H). ¹H NMR (500 MHz, Methanol-d₄) δ 7.17-7.08 (m, 2H), 6.94 (d, J=7.6 Hz, 2H), 4.92 (s, 1H), 4.25 (dt, J=7.9, 4.7 Hz, 1H), 3.99-3.84 (m, 3H), 3.76 (d, J=16.4 Hz, 2H), 3.53-3.44 (m, 1H), 2.89 (d, J=15.3 Hz, 1H), 2.81 (s, 6H), 2.74-2.62 (m, 1H), 2.55 (s, 1H), 2.42 (dt, J=14.1, 7.3 Hz, 1H), 2.17-1.95 (m, 4H), 1.95-1.72 (m, 4H), 1.52 (dd, J=21.7, 8.8 Hz, 8H), 1.35 (m, 1H).

### Example 23

### N′-((2¹S,2⁴S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide

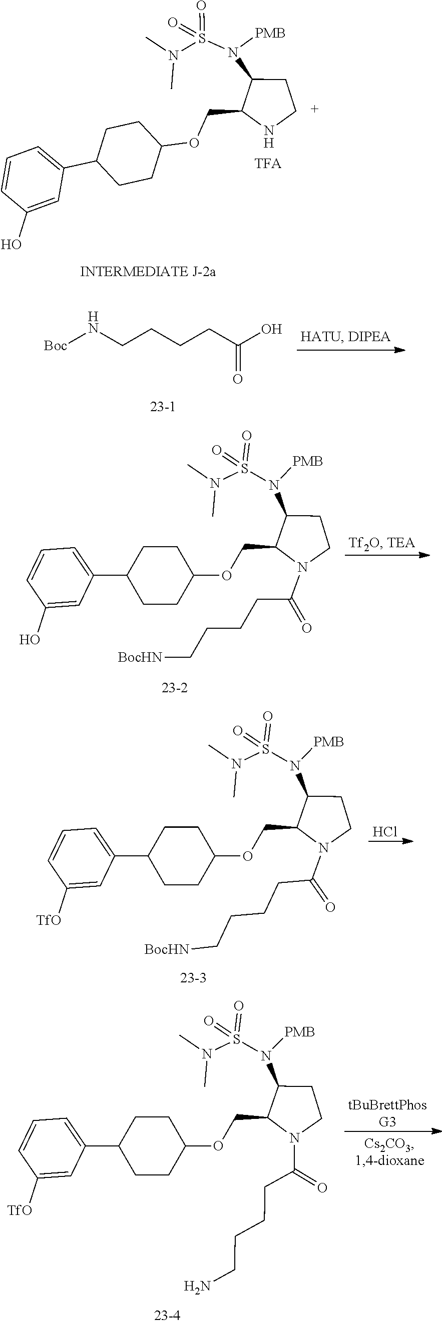

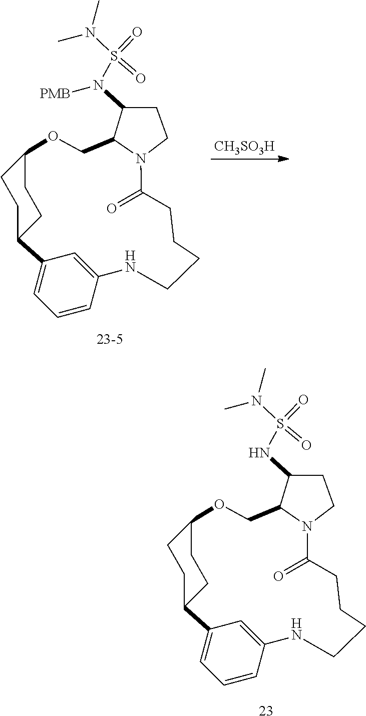

### Step 1: tert-butyl (5-((2R,3S)-3-((N,N-dimethylsulfamoyl)(4-methoxybenzyl)amino)-2-(((4-(3-hydroxyphenyl)cyclohexyl)oxy)methyl)pyrrolidin-1-yl)-5-oxopentyl)carbamate (23-2)

A solution of (5-((2R,3S)-3-((N,N-dimethylsulfamoyl)(4-methoxybenzyl)amino)-2-(((4-(3-hydroxyphenyl)cyclohexyl)oxy)methyl)pyrrodine (INTERMEDIATE J-2a) (100 mg, 0.162 mmol), HATU (74.1 mg, 0.195 mmol) and 5-((tert-butoxycarbonyl)amino)pentanoic acid (23-1) (52.9 mg, 0.244 mmol) in DMF (2.0 ml) was added DIPEA (0.085 ml, 0.487 mmol) at rt. Stirred at rt for 60 min, then concentrating under reduced pressure to remove DMF, the residue was purified by C18 column chromatography (10-100% Water in Acetonitrile with 0.05% TFA) to afford the title compound. MS: 717 (M+H).

### Step 2: 3-(4-(((2R,3S)-1-(5-((tert-butoxycarbonyl)amino)pentanoyl)-3-(N,Ndimethylsulfamoyl) (4-methoxybenzyl)amino)pyrrolidin-2-yl)methoxy)cyclohexyl)phenyltrifluoromethane sulfonate (23-3)

A solution of tert-butyl (5-((2R,3S)-3-((N,N-dimethylsulfamoyl)(4-methoxybenzyl)-amino)-2-(((4-(3-hydroxyphenyl)cyclohexyl)oxy)methyl)pyrrolidin-1-yl)-5-oxopentyl)carbamate (23-2) (111 mg, 0.155 mmol) in DCM (2 ml) cooled in a ice bath was added triethylamine (0.065 ml, 0.464 mmol) followed by 1.0 M trifluoromethanesulfonic anhydride in DCM (0.186 ml, 0.186 mmol). The reaction was stirred at rt overnight. The mixture was directly purified by silica column chromatography (0-70% EtOAc/hexanes) to give the title compound. MS: 849 (M+H).

### Step 3: 3-(4-(((2R,3S)-1-(5-aminopentanoyl)-3-((N,N-dimethylsulfamoyl)(4-methoxybenzyl) amino)pyrrolidin-2-yl)methoxy)cyclohexyl)phenyl trifluoromethanesulfonate (23-4)

A solution of 3-(4-(((2R,3S)-1-(5-((tert-butoxycarbonyl)amino)pentanoyl)-3-((N,N-dimethylsulfamoyl)(4-methoxybenzyl)amino)pyrrolidin-2-yl)methoxy)cyclohexyl)phenyl trifluoromethanesulfonate (23-3) (83 mg, 0.098 mmol) in Dioxane (0.7 ml) was added HCl in dioxane (0.7 ml, 2.80 mmol). After stirring at rt for 1 hr, the reaction was concentrated and the residue was purified by C18 column chromatography (10-100% Water in Acetonitrile with 0.05% TFA) to give the title compound as TFA salt. MS: 749 (M+H).

### Step 4: N′-(4-methoxybenzyl)-N-((2¹S,2⁴S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide (23-5)

A 10 ml of microwave vial was charged a mixture of 3-(4-(((2R,3S)-1-(5-aminopentanoyl)-3-((N,N-dimethylsulfamoyl)(4-methoxybenzyl)amino)pyrrolidin-2-yl)methoxy)cyclohexyl)phenyl trifluoromethanesulfonate, TFA salt (23-4) (28 mg, 0.033 mmol), tBuBrettPhos Pd G3, 8.47 mg, 9.92 μmol) and Cs₂CO₃ (43.1 mg, 0.132 mmol) in dioxane (3.5 ml). The mixture was purged with N2 for 4 min, then caped and heated to 75° C. for 10 hrs. The reaction was concentrated and the residue was purified by 1000 micron prep silica gel TLC plate [20×20 cm], eluting with 80% EtOAc/hexanes. The desired band was collected, filtered with ethyl acetate and concentrated under reduced pressure to give the title compound. MS: 599 (M+H).

### Step 5: N′-((2¹S,2⁴S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide (23)

The title compound 23 was prepared by using the appropriate reagents and N′-(4-methoxybenzyl)-N-((2¹S,2⁴S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide (23-5) according to the same procedure provided in step 6 for the preparation of 7. MS: 479 (M+H). ¹H NMR (500 MHz, Methanol-d₄) δ: 7.36 (t, J=7.8 Hz, 1H), 7.16 (d, J=7.1 Hz, 1H), 7.10 (s, 1H), 7.03 (d, J=7.9 Hz, 1H), 4.30 (dd, J=4.8, 2.8 Hz, 1H), 4.12 (dd, J=9.4, 3.4 Hz, 1H), 3.93 (dt, J=11.5, 7.6 Hz, 1H), 3.80 (s, 1H), 3.64-3.58 (m, 1H), 3.56-3.48 (m, 2H), 3.48-3.37 (m, 3H), 2.81 (d, J=6.3 Hz, 6H), 2.73-2.64 (m, 1H), 2.47-2.41 (m, 1H), 2.38 (t, J=7.0 Hz, 1H), 2.29-2.15 (m, 3H), 2.10 (tt, J=13.7, 7.2 Hz, 1H), 1.97-1.92 (m, 1H), 1.84 (dq, J=14.5, 7.2 Hz, 2H), 1.77-1.61 (m, 5H), 1.59-1.46 (m, 3H).

### Example 24

### N-((2¹S,2⁴S,5²R,5³S)-6-oxo-3,1′-dioxa-5(2,1)-piperidina-1(1,2)-benzena-2(1,4)-cyclohexanacycloundecaphane-5³-1 methanesulfonamide

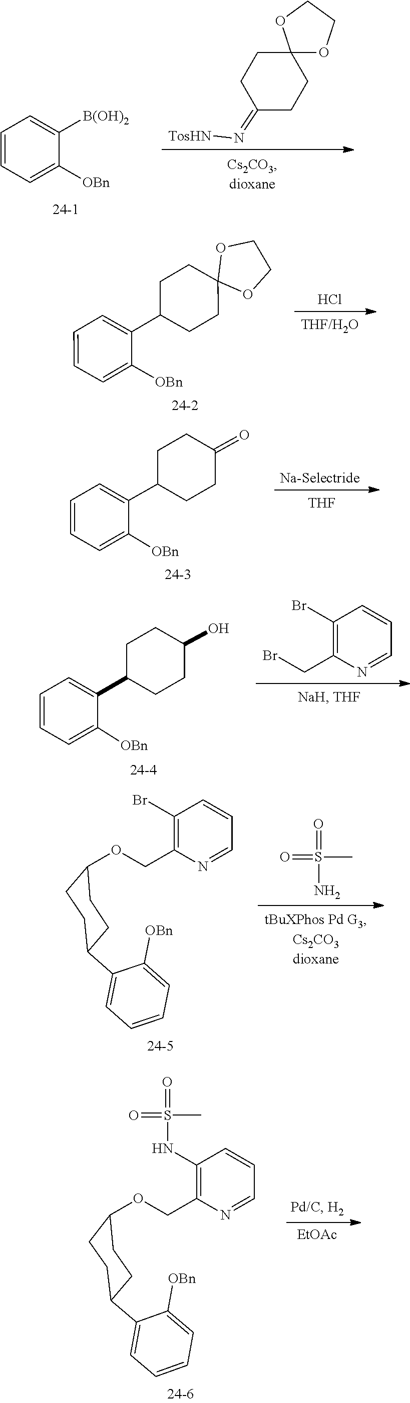

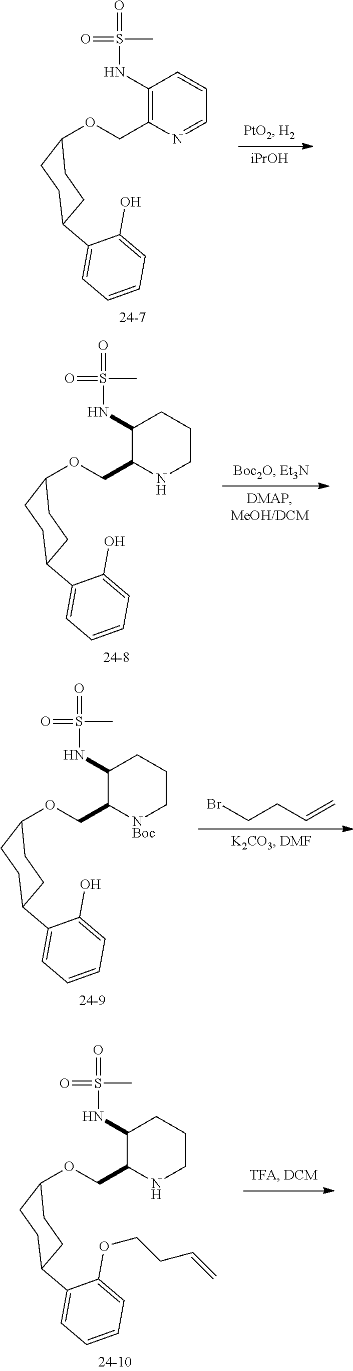

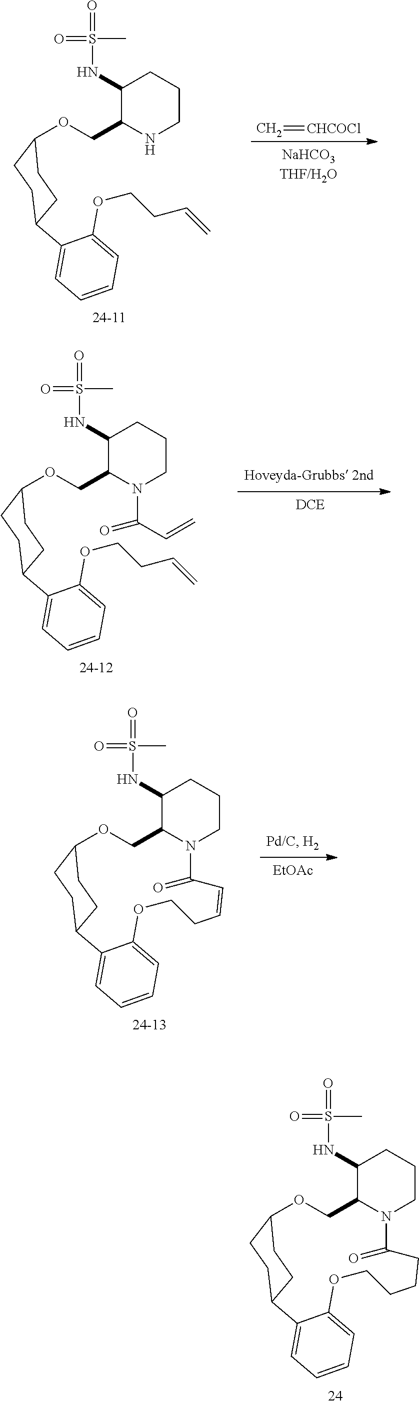

### Step 1: 8-(2-(benzyloxy)phenyl)-1,4-dioxaspiro[4.5]decane (24-2)

To a mixture of 4-methyl-N′-(1,4-dioxaspiro[4.5]decan-8-ylidene)benzenesulfono-hydrazide (25.6 g, 79 mmol) and 24-1 (15 g, 65.8 mmol) in 1,4-Dioxane (300 mL) was added cesium carbonate (64.3 g, 197 mmol). The reaction mixture was stirred at 120° C. for 16 h under N₂. Then the mixture was diluted with water (100 mL), extracted with EtOAc (100 mL×3). The combined organic phases were washed with brine (100 mL), and dried over Na₂SO₄. After filtration and concentration, the residue was purified by flash silica gel chromatography (10% EtOAc/Pet.ether gradient) to give the title compound. LCMS m/z (M+H): 325.17 required. 325.3 found.

### Step 2: 4-(2-(benzyloxy)phenyl)cyclohexan-1-one (24-3)

To a mixture of 24-2 (11 g, 33.9 mmol) in THF (150 mL) was added hydrogen chloride (5.65 mL, 33.9 mmol) (6 M in water) and the solution was stirred at 25° C. for 3 h. Then the mixture was basified with NaOH (1M in Water) (100 mL). The acidic aqueous phase was extracted with EtOAc (3×100 mL). The mixture was washed with brine (100 mL). The organic layer dried over Na₂SO₄. After filtration and concentration, the residue was purified by flash silica gel chromatography (10% EtOAc/Pet.ether gradient) to give the title compound. LCMS m/z (M+H): 281.15 required. 281.2 found.

### Step 3: (1s,4s)-4-(2-(benzyloxy)phenyl)cyclohexan-1-ol (24-4)

To a solution of 24-3 (7 g, 24.97 mmol) in THF (70 mL) were added sodium tri-sec-butylhydroborate (30.0 mL, 30.0 mmol) (1 M in THF) dropwise over 15 minutes at 0° C., and stirred at 0° C. for 30 minutes. The reaction mixture was quenched by the careful addition of H₂O (100 mL). The mixture was extracted with EtOAc (50 mL×3) and aq.NH₄Cl (50 mL×3). The combined organic layers were washed with brine (50 mL), dried over Na₂SO₄, filtered and the filtrate was concentrated. The crude product was purified by flash silica gel chromatography (16% EtOAc gradient) to give the title compound.

### Step 4: 2-((((1s,4s)-4-(2-(benzyloxy)phenyl)cyclohexyl)oxy)methyl)-3-bromopyridine (24-5)

To a solution of 3-bromo-2-(bromomethyl)pyridine (1 g, 2.79 mmol) and 24-4 (1.260 g, 4.46 mmol) in anhydrous THF (20 mL) was added NaH (0.156 g, 3.91 mmol) at 0° C. The resulting mixture was stirred at 60° C. for 12 h. The mixture was quenched with water (10 mL) and extracted with EtOAc (10 mL×3), separated. The organic solution was dried (MgSO₄), filtered and evaporated in vacuo. The residue was purified by flash silica gel chromatography (3% EtOAc/Pet.ether gradient) to give the title compound.

### Step 5: N-(2-((((1s,4s)-4-(2-(benzyloxy)phenyl)cyclohexyl)oxy)methyl)pyridin-3-yl)methanesulfonamide (24-6)

To a mixed solution of 24-5 (790 mg, 1.746 mmol), methanesulfonamide (257 mg, 2.62 mmol), tBuXPhos Pd G3 (139 mg, 0.175 mmol) and Cs₂CO₃ (1707 mg, 5.24 mmol) in Dioxane (15 mL) was added and stirred at 100° C. under N₂ for 12 h. The mixture was filtered and purified by flash silica gel chromatography (5-20% EtOAc/Pet.ether gradient) to give the title compound. LCMS m/z (M+H): 467.2 required, 467.6 found.

### Step 6: N-(2-((((1s,4s)-4-(2-hydroxyphenyl)cyclohexyl)oxy)methyl)pyridin-3-yl)methanesulfonamide (24-7)

To a solution of 24-6 (550 mg, 1.179 mmol) in EtOAc (10 mL) was added palladium (125 mg, 0.118 mmol) (10% wt) and the solution was stirred at 20° C. for 16 h under 15 psi. The mixture was filtered and concentrated to give the title compound.

### Step 7: N-((2R,3S)-2-((((1s,4S)-4-(2-hydroxyphenyl)cyclohexyl)oxy)methyl)piperidin-3-yl)methanesulfonamide (24-8)

To a solution of 24-7 (190 mg, 0.472 mmol, 89% yield) in i-PrOH (5 mL) was added platinum(IV) oxide (60.3 mg, 0.266 mmol), the solution was stirred at 20° C. for 8 h under H₂ (excess) (15 Psi). The mixture was filtered and concentrated to give the title compound. LCMS m/z (M+H): 383.3 required, 383.3 found.

### Step 8: tert-butyl (2R,3S)-2-((((1s,4S)-4-(2-hydroxyphenyl)cyclohexyl)oxy)methyl)-3-(methylsulfonamido)piperidine-1-carboxylate (24-9)

To a solution of 24-8 (210 mg, 0.549 mmol) in CH₂Cl₂ (1 mL) and MeOH (1 mL) was added BOC-Anhydride (0.191 mL, 0.823 mmol), TEA (0.230 mL, 1.647 mmol) and DMAP (6.71 mg, 0.055 mmol). Then the solution was stirred at 30° C. for 12 h under N₂. The mixture was filtered, concentrated and purified by prep-TLC (Pet. ether:EtOAc=1:1) to give the title compound.

### Step 9: tert-butyl (2R,3S)-2-((((1s,4S)-4-(2-(but-3-en-1-yloxy)phenyl)cyclohexyl)oxy)methyl)-3-(methylsulfonamido)piperidine-1-carboxylate (24-10)

To a mixture of 24-9 (150 mg, 0.311 mmol) and K₂CO₃ (129 mg, 0.932 mmol) in DMF (3 mL) was added dropwise 4-bromobut-1-ene (126 mg, 0.932 mmol) at 25° C. and the mixture was stirred at 90° C. for 15 hours. The mixture was quenched with water (10 mL) and extracted with EtOAc (10 mL×3), separated. The organic solution was dried (Na₂SO₄), filtered and evaporated in vacuo. The residue was purified by prep-TLC (Pet. ether:EtOAc=1:1) to give the title compound. LCMS m/z [M-100+H]: 437.3 found, 437.3 required.

### Step 10: N-((2R,3S)-2-((((1s,4S)-4-(2-(but-3-en-1-yloxy)phenyl)cyclohexyl)oxy)-methyl)piperidin-3-yl)methanesulfonamide (24-11)

To a mixture of 24-10 (100 mg, 0.186 mmol) in DCM (3 mL) was added dropwise TFA (1 mL) at 25° C. and the mixture was stirred for 1 h. The mixture was concentrated to give the title compound. LCMS m/z [M+H]: 437.3 found, 437.3 required

### Step 11: N-((2R,3S)-1-acryloyl-2-((((1s,4S)-4-(2-(but-3-en-1-yloxy)phenyl)cyclohexyl)oxy)-methyl)piperidin-3-yl)methanesulfonamide (24-12)

To a mixture of 24-11 (70 mg, 0.160 mmol) and aq. NaHCO₃(1 mL) in THF (2 mL) was added dropwise acryloyl chloride (17.41 mg, 0.192 mmol) at 0° C. and the mixture was stirred at 25° C. for 1 hours. The mixture was quenched with EtOAc (3 mL×3), separated. The organic solution was dried (MgSO₄), filtered and evaporated in vacuo to give the title compound.

### Step 12: N-((2¹S,2⁴S,5²R,5³S,Z)-6-oxo-3,11-dioxa-5(2,1)-piperidina-1(1,2)-benzena-2(1,4)-cyclohexanacycloundecaphan-7-en-5³-yl)methanesulfonamide (24-13)

To a solution of 24-12 (40 mg, 0.082 mmol) in DCE (40 mL) was added [1,3-bis(2,4,6-trimethylphenyl)imidazolidin-2-ylidene](([2-(propan-2-yloxy)phenyl]methylidene))ruthenium-bis(ylium) dichloride (5.11 mg, 8.15 μmol) and stirred at 50° C. for 15 hours. The reaction mixture was concentrated and purified by prep-TLC (EtOAc) to give the title compound. LCMS m/z (M+H): 491.2 required, 491.2 found.

### Step 13: N-((2¹S,2⁴S,5²R,5³S)-6-oxo-3,1′-dioxa-5(2,1)-piperidina-1(1,2)-benzena-2(1,4)-cyclohexanacycloundecaphane-5³-yl)methanesulfonamide (24)

To a solution of 24-13 (20 mg, 0.043 mmol) in EtOAc (10 mL) was added Pd—C(5.12 mg, 0.043 mmol). The mixture was stirred at 20° C. under H₂ (excess) for 1 hour. The reaction mixture was filtered to give the crude product and it was purified by prep-HPLC (water (0.1% TFA)-ACN) to give the title compound. LCMS m/z (M+H): 465.3 required, 465.3 found. ¹H NMR (400 MHz, CD₃OD) δ 7.13-7.00 (m, 2H), 6.89-6.82 (m, 1H), 6.80-6.74 (m, 1H), 4.52-4.39 (m, 2H), 4.19-4.02 (m, 1H), 3.89 (dd, J=4.0, 9.7 Hz, 1H), 3.84-3.73 (m, 2H), 3.65-3.43 (m, 2H), 3.04-2.97 (m, 3H), 2.85-2.56 (m, 2H), 2.53-2.35 (m, 3H), 2.32-2.16 (m, 1H), 2.11 (br d, J 13.9 Hz, 1H), 1.89 (br d, J=12.0 Hz, 2H), 1.85-1.68 (m, 4H), 1.66-1.38 (m, 4H), 1.35-1.27 (m, 2H)

### Example 25

### N′-((2¹S,2⁴S,5²R,5³S)-6-oxo-3,7,11-trioxa-5(2,1)-piperidina-1(1,3)-benzena-2(1,4)-cyclohexanacycloundecaphane-5³-yl)-N,N-dimethyl-sulfamide

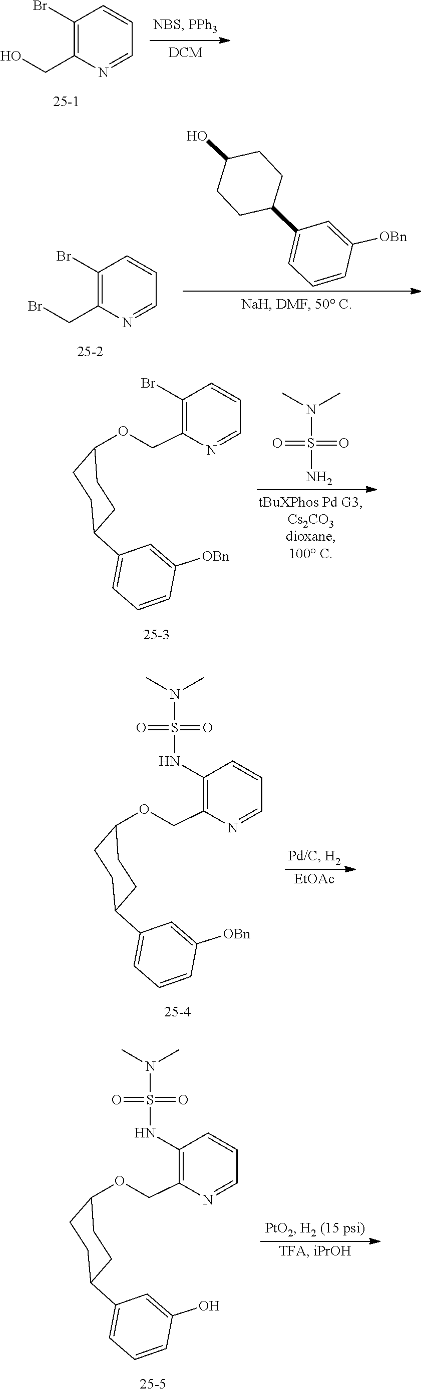

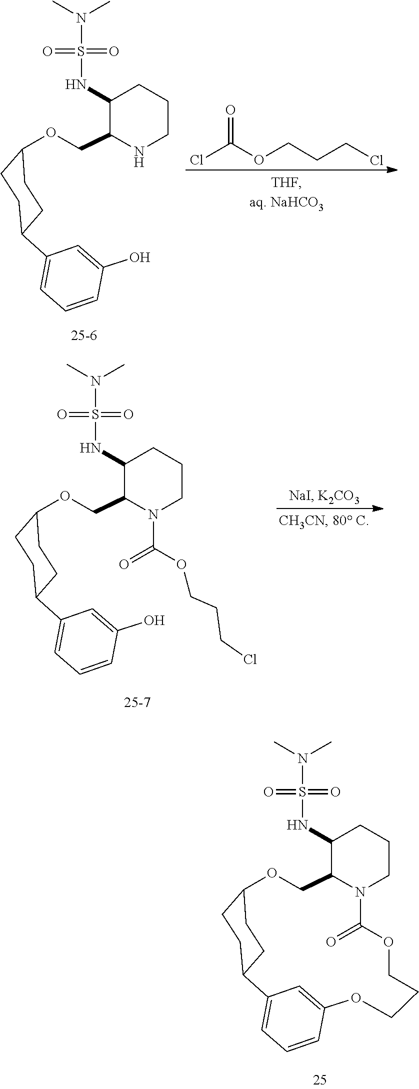

### Step 1: 3-bromo-2-(bromomethyl)pyridine (25-2)

To a mixture of 25-1 (20 g, 106 mmol) and triphenylphosphine (33.5 g, 128 mmol) in DCM (300 mL) was added NBS (22.72 g, 128 mmol) at 0° C. The reaction mixture was stirred at 0° C. to 15° C. for 2 h. The reaction mixture was poured into water (200 mL) and extracted with DCM (200 mL×3). The combined organic layers were washed with brine (200 mL), dried over Na₂SO₄, filtered and the filtrate was concentrated. The crude product was purified by flash silica gel chromatography (15% EtOAc/Pet. ether gradient) to give the title compound. LCMS m/z (M+H): 252.0 required, 252.0 found. ¹H NMR 1013349-006 (400 MHz, CDCl₃) δ 8.60-8.43 (m, 1H), 7.88 (dd, J=1.5, 8.1 Hz, 1H), 7.18-7.01 (m, 1H), 4.71 (d, J=2.2 Hz, 2H)

### Step 2: 2-((((1s,4s)-4-(3-(benzyloxy)phenyl)cyclohexyl)oxy)methyl)-3-bromopyridine (25-3)

To a solution of 25-2 (5.33 g, 21.25 mmol) and (1s,4s)-4-(3-(benzyloxy)phenyl)-cyclohexanol (5 g, 17.71 mmol) in anhydrous DMF (100 mL) was added NaH (1.062 g, 26.6 mmol) (60% wt) at 0° C. The resulting mixture was stirred at 0° C. for 0.5 h and stirred at 50° C. for 12 h. The mixture was quenched with water (300 mL) and extracted with EtOAc (100 mL×3). The combined organic layers were washed with brine (200 mL), dried over Na₂SO₄, filtered and the filtrate was concentrated. The crude product was purified by flash silica gel chromatography (12% EtOAc/Pet. ether gradient) to give the title compound. LCMS m/z (M+H): 454.1 required. 454.1 found. ¹H NMR 1013439-017 (400 MHz, CDCl3) δ 8.53 (dd, J=1.5, 4.6 Hz, 1H), 7.86 (dd, J=1.5, 8.1 Hz, 1H), 7.44-7.28 (m, 5H), 7.18 (t, J=7.9 Hz, 1H), 7.13-7.09 (m, 1H), 6.90-6.75 (m, 3H), 5.03 (s, 2H), 4.72 (s, 2H), 3.81 (br s, 1H), 2.56-2.46 (m, 1H), 2.18-2.07 (m, 2H), 1.91 (dq, J=3.1, 12.8 Hz, 2H), 1.65-1.49 (m, 4H)

### Step 3: N′-(2-((((1s,4s)-4-(3-(benzyloxy)phenyl)cyclohexyl)oxy)methyl)pyridin-3-yl)-N,N-dimethyl-sulfamide (25-4)

To a solution of 25-3 (2.1 g, 4.64 mmol) in Dioxane (20 mL) were added Cs₂CO₃ (4.54 g, 13.93 mmol), N,N-dimethylsulfamide (0.634 g, 5.11 mmol) and TBUXPHOS PD G3 (0.369 g, 0.464 mmol). The resulting mixture was stirred at 100° C. for 3 h. The mixture was filtered and the filtrate was concentrated. The crude product was purified by flash silica gel chromatography (50% EtOAc/Pet. ether gradient) to give the title compound. LCMS m/z (M+H): 496.3 required. 496.3 found. ¹H NMR 1013439-028 (400 MHz, CDCl₃) δ 8.73 (s, 1H), 8.22 (dd, J=1.2, 4.6 Hz, 1H), 7.82 (dd, J=1.2, 8.3 Hz, 1H), 7.45-7.17 (m, 6H), 6.90-6.77 (m, 3H), 5.05 (s, 2H), 4.84 (s, 2H), 3.80 (br s, 1H), 2.88-2.79 (m, 6H), 2.60-2.45 (m, 1H), 2.12 (br d, J=14.4 Hz, 2H), 1.86-1.66 (m, 4H), 1.61 (br d, J=13.9 Hz, 2H).

### Step 4: N′-(2-((((1s,4s)-4-(3-hydroxyphenyl)cyclohexyl)oxy)methyl)pyridin-3-yl)-N,N-dimethyl-sulfamide (25-5)

To a solution of 25-4 (1.2 g, 2.421 mmol) in EtOAc (100 mL) was added palladium (0.258 g, 0.242 mmol) (10% wt) and the solution was stirred at 20° C. for 18 h under H₂ (excess) 15 psi. The mixture was filtered and concentrated to give the title compound. LCMS m/z (M+H): 406.2 required, 406.2 found.

### Step 5: N′-((2R,3S)-2-((((1s,4S)-4-(3-hydroxyphenyl)cyclohexyl)oxy)methyl)piperidin-3-yl)-N,N-dimethyl-sulfamide (25-6)

To a solution of 25-5 (0.98 g, 2.417 mmol) in i-PrOH (50 mL) were added platinum(IV) oxide (0.274 g, 1.208 mmol) and trifluoroacetic acid (0.198 mL, 2.66 mmol). The solution was stirred at 20° C. for 12 h under H₂ (15 psi). The mixture was filtered and concentrated to give the title compound. LCMS m/z (M+H): 412.2 required, 412.2 found.

### Step 6: 3-chloropropyl (2R,3S)-3-((N,N-dimethylsulfamoyl)amino)-2-((((1s,4S)-4-(3-hydroxyphenyl)cyclohexyl)oxy)methyl)piperidine-1-carboxylate (25-7)

To a solution of 25-6 (200 mg, 0.486 mmol) in THF (4 mL) and aq. NaHCO₃(2 mL) was added 3-chloropropyl carbonochloridate (76 mg, 0.486 mmol). The mixture was stirred at 0° C. for 2 h. The mixture was quenched with water (30 mL) and extracted with EtOAc (20 mL×3), washed with brine (20 mL), dried over Na₂SO₄, filtered and concentrated under reduced pressure to give the title compound. LCMS m/z (M+H): 532.3 required, 532.3 found.

### Step 7: N′-((2¹S,2⁴S,5²R,5³S)-6-oxo-3,7,11-trioxa-5(2,1)-piperidina-1(1,3)-benzena-2(1,4)-cyclohexanacycloundecaphane-5³-yl)-N,N-dimethyl-sulfamide (25)

To a solution of 25-7 (80 mg, 0.150 mmol) in CH₃CN (2 mL) were added K₂CO₃ (62.3 mg, 0.451 mmol) and sodium iodide (11.27 mg, 0.075 mmol). The mixture was stirred at 80° C. for 6 h. The mixture was quenched with water (20 mL) and extracted with EtOAc (20 mL×3), washed with brine (20 mL), dried over Na₂SO₄, filtered and concentrated under reduced pressure. The residue was purified by prep-HPLC (water (0.1% TFA)-ACN). The cyclized product (20 mg) was separated by SFC to give the desired product.

SFC Condition
Column: Chiralcel OD-3 100×4.6 mm I.D., 3 um
Mobile phase: A: CO₂ B: ethanol (0.05% DEA)
Gradient: from 5% to 40% of B in 4 min and hold 40% for 2.5 min, then 5% of B for 1.5 min
Flow rate: 2.8 mL/min
LCMS m/z (M+H): 496.2 required, 496.2 found. ¹H NMR (25) (400 MHz, CD₃OD) δ 7.07 (t, J=7.8 Hz, 1H), 6.78 (s, 1H), 6.73-6.63 (m, 2H), 4.84-4.70 (m, 1H), 4.68-4.49 (m, 1H), 4.37-4.12 (m, 3H), 4.05-3.91 (m, 2H), 3.69 (br s, 1H), 3.58 (dd, J=3.5, 9.4 Hz, 1H), 3.44-3.34 (m, 1H), 3.10 (br t, J=13.3 Hz, 1H), 2.82-2.76 (m, 6H), 2.59-2.47 (m, 1H), 2.28-2.04 (m, 2H), 1.98-1.34 (m, 12H)

### Example 26 & 27

### N′-((2¹S,2⁴S,5²R,5³S)-6-oxo-3,7,12-trioxa-5(2,1)-piperidina-1(1,3)-benzena-2(1,4)-cyclohexanacyclododecaphane-5³-yl)-N,N-dimethy-sulfamide (26) & N′-((2¹R,2⁴R,5²S,5³R)-6-oxo-3,7,12-trioxa-5(2,1)-piperidina-1(1,3)-benzena-2(1,4)-cyclohexanacyclododecaphane-5³-yl)-N,N-dimethyl-sulfamide (27)

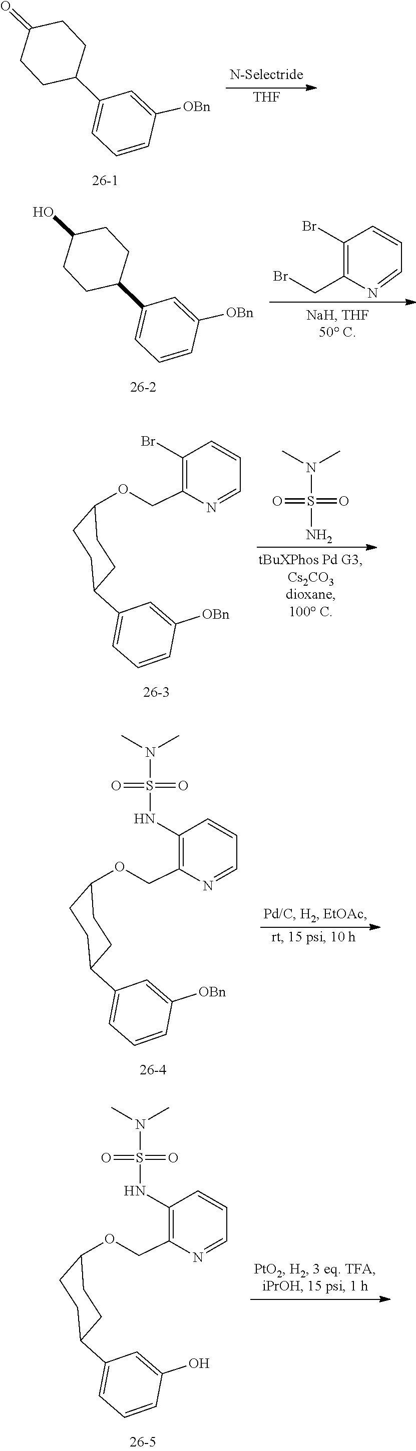

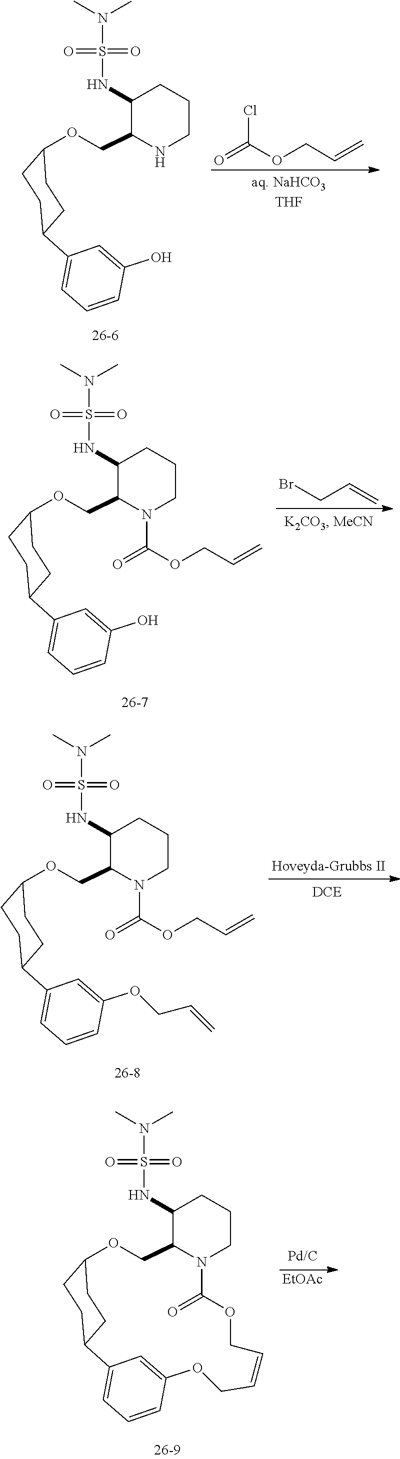

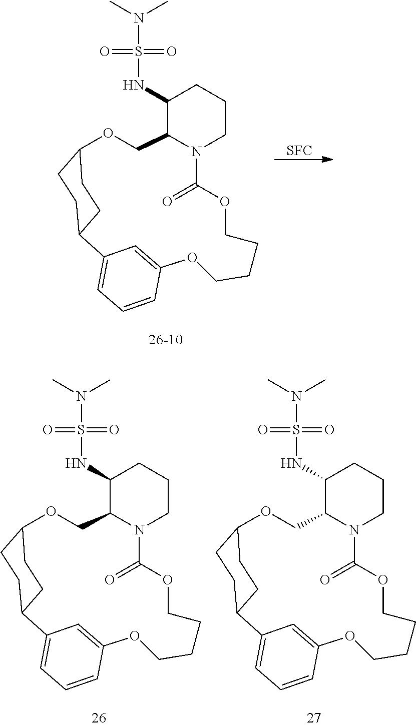

### Step 1: (1s,4s)-4-(3-(benzyloxy)phenyl)cyclohexan-1-ol (26-2)

To a solution of 26-1 (18 g, 64.2 mmol) in THF (200 mL) was added sodium tri-sec-butylhydroborate (77 mL, 77 mmol) (1 M in THF) dropwise over 15 minutes at 0° C., and stirred at 0° C. for 1 h. The reaction mixture was quenched by the careful addition of H₂O (1000 mL) and NH₄Cl (100 mL). The mixture was extracted with EtOAc (200 mL×3). The combined organic layers were washed with brine (200 mL), dried over Na₂SO₄, filtered and the filtrate was concentrated. The crude product was purified by flash silica gel chromatography (10% EtOAc/Pet.ether gradient) to give the title compound.

### Step 2: 2-((((1s,4s)-4-(3-(benzyloxy)phenyl)cyclohexyl)oxy)methyl)-3-bromopyridine (26-3)

To a solution of 3-bromo-2-(bromomethyl)-pyridine (4.44 g, 17.71 mmol) and compound 26-2 (5 g, 17.71 mmol) in anhydrous THF (100 mL) was added NaH (1.062 g, 26.6 mmol) (60% wt) at 0° C. The resulting mixture was stirred at 0° C. for 0.5 h and then stirred at 50° C. for 12 h. The mixture was quenched with water (300 mL) and extracted with EtOAc (100 mL×3). The combined organic layers were washed with brine (200 mL), dried over Na₂SO₄, filtered and the filtrate was concentrated. The crude product was purified by flash silica gel chromatography (5% EtOAc/Pet. ether gradient) to give the title compound. LCMS m/z (M+H): 453.9 required. 453.9 found.

### Step 3: N′-(2-((((1s,4s)-4-(3-(benzyloxy)phenyl)cyclohexyl)oxy)methyl)pyridin-3-yl)-N,N-dimethyl-sulfamide (26-4)

To a solution of 26-3 (4.3 g, 9.51 mmol) in Dioxane (50 mL) were added Cs₂CO₃ (9.29 g, 28.5 mmol), N,N-dimethylsulfamide (1.298 g, 10.46 mmol) and TBUXPHOS PD G3 (0.378 g, 0.475 mmol). The resulting mixture was stirred at 100° C. for 3 h. The mixture was filtered and the filtrate was concentrated. The crude product was purified by flash silica gel chromatography (50% EtOAc/Pet. ether gradient) to give the title compound. LCMS m/z (M+H): 496.3 required. 496.3 found. ¹H NMR 1014062-028 (400 MHz, CDCl₃) δ 8.73 (s, 1H), 8.22 (dd, J=1.2, 4.6 Hz, 1H), 7.82 (dd, J=1.2, 8.3 Hz, 1H), 7.45-7.17 (m, 6H), 6.90-6.77 (m, 3H), 5.05 (s, 2H), 4.84 (s, 2H), 3.80 (br s, 1H), 2.88-2.79 (m, 6H), 2.60-2.45 (m, 1H), 2.12 (br d, J=14.4 Hz, 2H), 1.86-1.66 (m, 4H), 1.61 (br d, J=13.9 Hz, 2H)

### Step 4: N′-(2-((((1s,4s)-4-(3-hydroxyphenyl)cyclohexyl)oxy)methyl)pyridin-3-yl)-N,N-dimethyl-sulfamide (26-5)

To a solution of 26-4 (2 g, 4.04 mmol) in EtOAc (50 mL) was added Pd—C(0.859 g, 0.807 mmol) (10% wt) and the solution was stirred at 20° C. for 12 h under H₂ (excess) 15 psi. The mixture was filtered and concentrated to give the title compound. LCMS m/z (M+H): 406.3 required, 406.3 found.

### Step 5: N′-((2R,3S)-2-((((1s,4S)-4-(3-hydroxyphenyl)cyclohexyl)oxy)methyl)piperidin-3-yl)-N,N-dimethyl-sulfamide (26-6)

To a solution of 26-5 (1.5 g, 3.7 mmol) in i-PrOH (100 mL) were added platinum(IV) oxide (1.260 g, 5.55 mmol) and trifluoroacetic acid (0.827 mL, 11.10 mmol). The solution was stirred at 20° C. for 3 h under H₂ (excess) 15 psi. The mixture was filtered and concentrated to give the title compound. LCMS m/z (M+H): 412.2 required, 412.2 found.

### Step 6: allyl (2R,3S)-3-((N,N-dimethylsulfamoyl)amino)-2-((((1s,4S)-4-(3-hydroxyphenyl)-cyclohexyl)oxy)methyl)piperidine-1-carboxylate (26-7)

To a solution of 26-6 (500 mg, 1.215 mmol) in THF (8 mL) and saturated NaHCO₃(4.00 mL) was added allyl chloroformate (0.132 mL, 1.239 mmol). The mixture was stirred at 0° C. for 1 h. The mixture was quenched with water (30 mL) and extracted with EtOAc (20 mL×3), washed with brine (20 mL), dried over Na₂SO₄, filtered and concentrated under reduced pressure. The residue was purified by flash silica gel chromatography (60% ethyl acetate/pet. ether gradient) to give the title compound. LCMS m/z (M+H): 496.3 required, 496.3 found.

### Step 7: allyl (2R,3S)-2-((((1s,4S)-4-(3-(allyloxy)phenyl)cyclohexyl)oxy)methyl)-3-((N,N-dimethylsulfamoyl)amino)piperidine-1-carboxylate (26-8)

To a solution of 26-7 (220 mg, 0.444 mmol) in MeCN (5 mL) were added K₂CO₃ (184 mg, 1.332 mmol) and allyl bromide (59.1 mg, 0.488 mmol). The solution was stirred at 20° C. for 13 h. The reaction mixture was poured into water (20 mL), extracted with EtOAc (20 mL×3). The combined organic phases were washed with brine (20 mL), and dried over Na2SO4. After filtration and concentration, the residue was purified by pre-TLC (Pet. ether:EtOAc=2:1) to give the title compound. LCMS m/z (M+H): 536.3 required, 536.3 found.

### Step 8: N′-((2¹S,2⁴S,5²R,5³S,Z)-6-oxo-3,7,12-trioxa-5(2,1)-piperidina-1(1,3)-benzena-2(1,4)-cyclohexanacyclododecaphan-9-en-5³-yl)-N,N-dimethyl-sulfamide (26-9)

To a solution of 26-8 (130 mg, 0.243 mmol) in DCE (130 mL) was added (1,3-dimesitylimidazolidin-2-ylidene)(2-isopropoxybenzylidene)ruthenium(vi) chloride (30.4 mg, 0.049 mmol). The mixture was stirred at 50° C. for 12 h. The reaction mixture was concentrated to give the crude. The residue was purified by flash silica gel chromatography (90% ethyl acetate/pet. ether gradient) to give the title compound. LCMS m/z (M+H): 508.3 required, 508.3 found.

### Step 9: N′-((2¹S,2⁴S,5²R,5³S)-6-oxo-3,7,12-trioxa-5(2,1)-piperidina-1(1,3)-benzena-2(1,4)-cyclohexanacyclododecaphane-5³-yl)-N,N-dimethy-sulfamide (26-10)

To a solution of 26-9 (80 mg, 0.158 mmol) in EtOAc (5 mL) was added Pd—C(84 mg, 0.079 mmol) (10% wt) and the solution was stirred at 20° C. for 2 h under hydrogen (excess) 15 psi. The mixture was filtered and concentrated to give the crude. The crude was purified by pre-TLC (Pet. ether:EtOAc=1:1) to give the title compound.

### Step 10: N′-((2¹S,2⁴S,5²R,5³S)-6-oxo-3,7,12-trioxa-5(2,1)-piperidina-1(1,3)-benzena-2(1,4)-cyclohexanacyclododecaphane-5³-yl)-N,N-dimethy-sulfamide (26) & N′-((2¹R,2⁴R,5²S,5³R)-6-oxo-3,7,12-trioxa-5(2,1)-piperidina-1(1,3)-benzena-2(1,4)-cyclohexanacyclododecaphane-5³-yl) N,N-dimethyl-sulfamide (27)

26-10 was separated by SFC to give 26 and 27.

Sfc Condition:
Column: ChiralPak AD-3 150×4.6 mm I.D., 3 um
Mobile phase: A: CO₂ B: Ethanol (0.05% DEA)
Gradient: from 5% to 40% of B in 4.5 min, then 5% of B for 1.5 min
Flow rate: 2.5 mL/min
Column temp.:40° C.
Back pressure: 100 bar
LCMS m/z (M+H): 510.3 required, 510.3 found

¹H NMR (26) (400 MHz, CD₃OD) δ 7.08 (t, J=7.8 Hz, 1H), 6.75-6.62 (m, 3H), 4.85-4.77 (m, 1H), 4.35-4.28 (m, 1H), 4.22-4.09 (m, 2H), 4.06-3.89 (m, 3H), 3.69 (br s, 1H), 3.57 (dd, J=4.1, 9.6 Hz, 1H), 3.44-3.34 (m, 1H), 3.14-3.04 (m, 1H), 2.79 (s, 6H), 2.55 (br t, J=11.7 Hz, 1H), 2.18-1.98 (m, 3H), 1.96-1.77 (m, 5H), 1.77-1.49 (m, 8H)

¹H NMR (27) (400 MHz, CD₃OD) δ 7.08 (t, J=7.8 Hz, 1H), 6.74-6.68 (m, 2H), 6.64 (br d, J 7.8 Hz, 1H), 4.83-4.77 (m, 1H), 4.35-4.27 (m, 1H), 4.22-3.97 (m, 4H), 3.92 (t, J=9.6 Hz, 1H), 3.69 (br s, 1H), 3.56 (dd, J=3.9, 9.4 Hz, 1H), 3.43-3.33 (m, 1H), 3.09 (br t, J=13.1 Hz, 1H), 2.79 (s, 6H), 2.61-2.51 (m, 1H), 2.18-1.98 (m, 3H), 1.95-1.78 (m, 5H), 1.77-1.48 (m, 8H).

### Example 28 & 29

### N-((2¹S,2⁴S,5²R,5³S,E)-6-oxo-3,11-dioxa-5(2,1)-piperidina-1(1,3)-benzena-2(1,4)-cyclohexanacycloundecaphan-7-en-5³-yl)methanesulfonamide (28) & N-((2¹S,2⁴S,5²R,5³S)-6-oxo-3,11-dioxa-5(2,1)-piperidina-1(1,3)-benzena-2(1,4)-cyclohexanacycloundecaphane-5³-yl)methanesulfonamide (29)

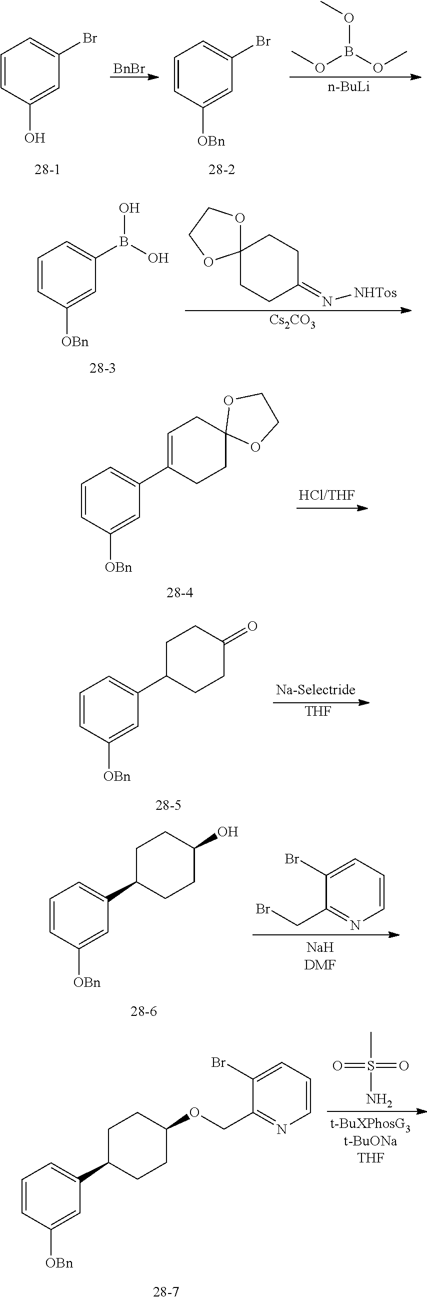

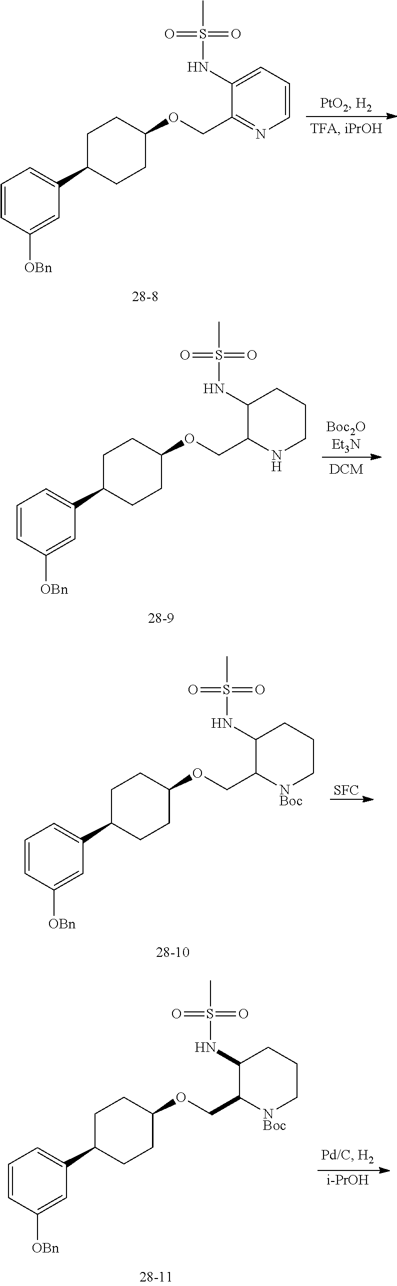

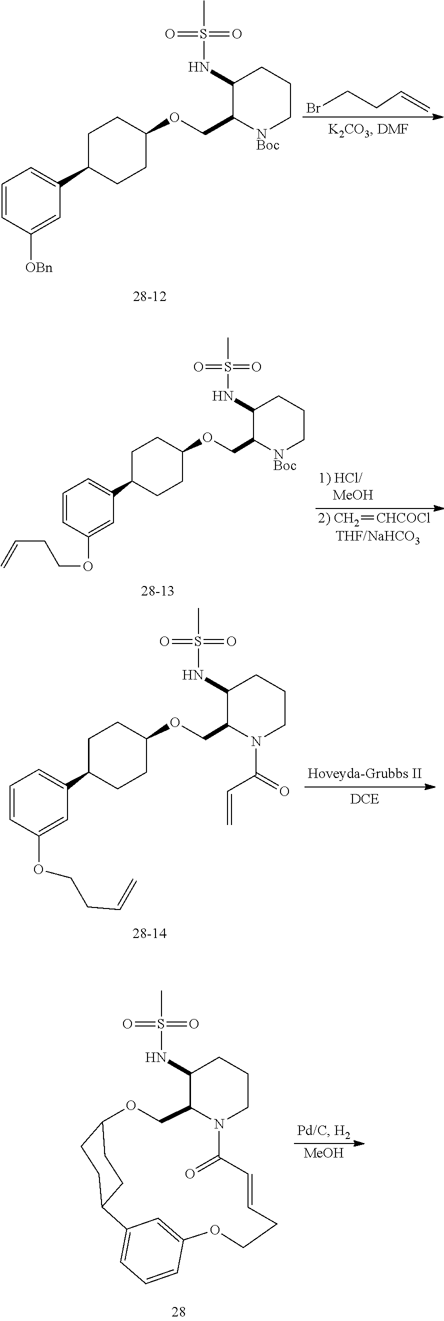

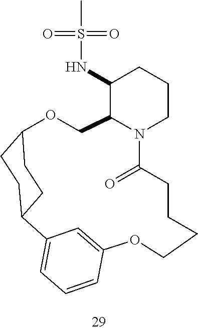 [SMILES: CS(=O)(=O)N[C@H]1CCCN2C(=O)CCCCOc3cccc(c3)C3CCC(CC3)OC[C@@H]12]

### Step 1: 1-(benzyloxy)-3-bromobenzene (28-2)

To a mixture of 28-1 (25 g, 145 mmol) and K₂CO₃ (59.9 g, 434 mmol) in DMF (150 mL) was added 3-bromophenol (25 g, 145 mmol) at 25° C., the mixture was stirred at 50° C. for 15 h. The mixture was quenched with water (500 mL) and extracted with EtOAc (300 mL*3). The organic solution was dried over Na₂SO₄, filtered and evaporated in vacuo. The residue was purified by flash silica gel chromatography (10% EtOAc/Pet.ether gradient) to give the title compound. ¹H NMR (500 MHz, CDCl₃) δ 7.30-7.17 (m, 5H), 7.03-6.92 (m, 3H), 6.76 (ddd, J 0.9, 2.4, 8.2 Hz, 1H), 4.97-4.81 (m, 2H).

### Step 2: (3-(benzyloxy)phenyl)boronic acid (28-3)

To a solution of 28-2 (38 g, 144 mmol) in THF (400 mL) was added n-butyllithium (69.3 mL, 173 mmol) (2M in hexane) at −78° C. and the solution was stirred at −78° C. for 1 h. Then trimethyl borate (32.3 mL, 289 mmol) was added to the above solution at −78° C. The solution was stirred at −78° C. for 1 h and 15° C. for 16 h. Then HCl (6N, 50 mL) was added and the mixture was stirred at 15° C. for 1 h. The mixture was quenched with water (200 mL) and extracted with EtOAc (200 mL*3). The combined organic layers were washed with brine (200 mL), dried over Na₂SO₄, filtered and concentrated in vacuo. The residue was dissolved in Pet.ether (100 mL)/EtOAc (10 mL) and stirred for 10 minutes. The solid appeared and filtered to give the title compound. ¹H NMR (500 MHz, CDCl₃) δ 7.86-7.77 (m, 2H), 7.55-7.33 (m, 7H), 5.18 (s, 2H).

### Step 3: 8-(3-(benzyloxy)phenyl)-1,4-dioxaspiro[4.5]decane (28-4)

To a mixture of 4-methyl-N′-(1,4-dioxaspiro[4.5]decan-8-ylidene)benzenesulfono-hydrazide (41.0 g, 126 mmol) and 28-3 (24 g, 105 mmol) in 1,4-Dioxane (400 mL) was added cesium carbonate (103 g, 316 mmol). The reaction mixture was stirred at 120° C. for 3 h under N₂. Then the mixture was diluted with water (500 mL), extracted with EtOAc (300 mL×3). The combined organic phases were washed with brine (500 mL), and dried over Na₂SO₄. After filtration and concentration, the residue was purified by flash silica gel chromatography (10% EtOAc/Pet.ether gradient) to give the title compound. LRMS m/z (M+H): 325.1 required, 325.1 found. ¹H NMR (400 MHz, CDCl₃) δ 7.52-7.26 (m, 5H), 7.19 (t, J=7.8 Hz, 1H), 6.92-6.71 (m, 3H), 5.03 (s, 2H), 3.97 (s, 4H), 2.63-2.46 (m, 1H), 1.93-1.62 (m, 8H).

### Step 4: 4-(3-(benzyloxy)phenyl)cyclohexan-1-one (28-5)

To a solution of 28-4 (12 g, 37.0 mmol) in THF (120 mL) was added HCl (40 mL, 160 mmol) (4 M in water) and the solution was stirred at 20° C. for 16 h. The mixture was concentrated in vacuo. The acidic aqueous phase was extracted with EtOAc (2×100 mL). The combined EtOAc phase was washed with water (2.x.100 mL), brine (100 mL), dried over Na₂SO₄ and filtered. The filtrate was concentrated to give the title compound. LRMS m/z (M+H): 281 required. 281 found.

### Step 5: (1s,4s)-4-(3-(benzyloxy)phenyl)cyclohexan-1-ol (28-6)

To a solution of 28-5 (10 g, 35.7 mmol) in THF (150 mL) were added sodium tri-sec-butylhydroborate (42.8 mL, 42.8 mmol) (1 M in THF) dropwise over 15 minutes at 0° C., and stirred at 0° C. for 30 minutes. The reaction mixture was quenched by the careful addition of H₂O (50 mL). The mixture was extracted with EtOAc (200 mL×3) and aq.NH₄Cl (50 mL). The combined organic layers were washed with brine (200 mL), dried over Na₂SO₄, filtered and the filtrate was concentrated. The crude product was purified by flash silica gel chromatography (10% EtOAc/Pet.ether gradient) to give the title compound. LRMS m/z (M-18): 265.1 found, 265.1 required.

### Step 6: 2-((((1s,4s)-4-(3-(benzyloxy)phenyl)cyclohexyl)oxy)methyl)-3-bromopyridine (28-7)

To a solution of 3-bromo-2-(bromomethyl) pyridine (7.78 g, 31.0 mmol) and 28-6 (7.3 g, 25.9 mmol) in anhydrous DMF (100 mL) was added NaH (1.241 g, 31.0 mmol) at 0° C. The resulting mixture was stirred at 0° C. for 0.5 h and stirred at 50° C. for 2 h. The mixture was quenched with water (300 mL) and extracted with EtOAc (300 mL*3). The organic solution was dried over Na₂SO₄ filtered and evaporated in vacuo. The residue was purified by flash silica gel chromatography (20% EtOAc/Pet.ether gradient) to give the title compound. LRMS m/z (M+H): 454.0 required. 454.0 found.

### Step 7: N-(2-((((1s,4s)-4-(3-(benzyloxy)phenyl)cyclohexyl)oxy)methyl)pyridin-3-yl)methanesulfonamide (28-8)

To a solution of 28-7 (3.8 g, 8.40 mmol), methanesulfonamide (0.959 g, 10.08 mmol) and sodium 2-methylpropan-2-olate (1.615 g, 16.80 mmol) in THF (60 mL) was added t-BuXPhos Pd G₃ (0.667 g, 0.840 mmol). The mixture was stirred at 70° C. under N₂ for 16 h. The mixture was quenched with water (60 mL) and extracted with EtOAc (60 mL*3). The organic solution was dried over Na₂SO₄, filtered and evaporated in vacuo. The residue was purified by flash silica gel chromatography (50% EtOAc/Pet.ether gradient) to give the title compound. LRMS (M+H): 467.2 required, 467.2 found.

### Step 8: N-(2-((((1s,4s)-4-(3-(benzyloxy)phenyl)cyclohexyl)oxy)methyl)piperidin-3-yl)methanesulfonamide (28-9)

To a solution of 28-8 (1 g, 2.143 mmol) in i-PrOH (30 mL) was added platinum(IV) oxide (0.097 g, 0.429 mmol) and the solution was stirred at 30° C. for 3 h under H₂ (50 Psi). The mixture was filtered and concentrated to give the title compound. LRMS m/z (M+H): 473.3 required, 473.3 found.

### Step 9: tert-butyl 2-((((1s,4s)-4-(3-(benzyloxy)phenyl)cyclohexyl)oxy)methyl)-3-(methylsulfonamido)piperidine-1-carboxylate (28-10)

To a solution of 28-9 (700 mg, 1.481 mmol) in DCM (20 mL) was added BOC₂O (0.688 mL, 2.96 mmol), TEA (0.619 mL, 4.44 mmol). The solution was stirred at 20° C. for 3 h. The reaction mixture was poured into water (20 mL), extracted with DCM (20 mL×3). The organic layer was washed with brine (10 mL), dried over Na₂SO₄. After filtration and concentration, the residue was purified by flash silica gel chromatography (50% EtOAc/Pet.ether gradient) to give the title compound. LRMS m/z (M+H⁺-100): 473.3 required, 473.3 found. ¹H NMR (500 MHz, CDCl₃) δ 7.50-7.28 (m, 5H), 7.21 (t, J=7.9 Hz, 1H), 6.92-6.78 (m, 3H), 5.09-5.05 (m, 2H), 4.72-4.51 (m, 1H), 4.01-3.88 (m, 2H), 3.73-3.52 (m, 3H), 3.04-2.92 (m, 3H), 2.71 (br t, J=12.7 Hz, 1H), 2.59-2.46 (m, 1H), 1.82-1.68 (m, 6H), 1.62-1.55 (m, 4H), 1.47 (s, 9H).

### Step 10: tert-butyl (2R,3S)-2-((((1s,4S)-4-(3-(benzyloxy)phenyl)cyclohexyl)oxy)methyl)-3-(methylsulfonamido)piperidine-1-carboxylate (28-11)

28-10 (690 mg, 1.205 mmol) was separated by SFC to give the undesired isomer and 28-11. LRMS m/z (M+H-100): 473.1 required, 473.1 found. ¹H NMR (400 MHz, CDCl₃) δ 7.49-7.42 (m, 2H), 7.42-7.28 (m, 3H), 7.21 (t, J=8.0 Hz, 1H), 6.91-6.78 (m, 3H), 5.07 (s, 4H), 4.58 (br d, J=1.2 Hz, 1H), 3.98 (br t, J=9.4 Hz, 3H), 3.71-3.47 (m, 4H), 2.99 (s, 3H), 2.77-2.66 (m, 1H), 2.53 (br s, 1H), 2.04 (br s, 3H), 1.76-1.69 (m, 5H), 1.60 (br s, 4H), 1.46 (s, 9H). SFC condition: Column: Chiralpak AD-3 150×4.6 mm I.D., 3 um Mobile phase: A: CO₂ B:iso-propanol (0.05% DEA) Gradient: from 5% to 40% of B in 5 min and from 40% to 5% of B in 0.5 min, hold 5% of B for 1.5 min Flow rate: 2.5 mE/min Column temp.: 35° C. ABPR: 1500 psi.

### Step 11: tert-butyl (2R,3S)-2-((((1s,4S)-4-(3-hydroxyphenyl)cyclohexyl)oxy)methyl)-3-(methylsulfonamido)piperidine-1-carboxylate (28-12)

To a solution of 28-12 (200 mg, 0.349 mmol) in i-PrOH (5 mL) was added Pd—C(37.2 mg, 0.035 mmol) (10%, dry) and the solution was stirred at 20° C. for 1 h under H2 (15 Psi). The mixture was filtered and concentrated to give the title compound. LRMS m/z (M+H): 483.1 required. 483.1 found.

### Step 12: tert-butyl (2R,3S)-2-((((1s,4S)-4-(3-(but-3-en-1-yloxy)phenyl)cyclohexyl)oxy)methyl)-3-(methylsulfonamido)piperidine-1-carboxylate (28-13)

To a mixture of 28-12 (169 mg, 0.350 mmol) and K₂CO₃ (145 mg, 1.050 mmol) in DMF (5 mL) was added dropwise 4-bromobut-1-ene (142 mg, 1.050 mmol) at 25° C. and the mixture was stirred at 80° C. for 15 h. The mixture was quenched with water (20 mL) and extracted with EtOAc (20 mL*3). The organic solution was dried over Na₂SO₄, filtered and evaporated in vacuo. The residue was purified by TLC to give the title compound. LRMS m/z [M−100+H⁺]: 437.5 found, 437.5 required.

### Step 13: N-((2R,3S)-1-acryloyl-2-((((1s,4S)-4-(3-(but-3-en-1-yloxy)phenyl)cyclohexyl)oxy)methyl)piperidin-3-yl)methanesulfonamide (28-14)

28-13 (150 mg, 0.279 mmol) was dissolved in HCl/MeOH (2 mL) and stirred at 20° C. for 1 h. The mixture was concentrated. Then it was dissolved in aq.NaHCO₃(1.5 mL) in THF (3 mL) and acryloyl chloride (30.3 mg, 0.335 mmol) was added dropwise at 0° C. and the mixture was stirred at 25° C. for 1 h. The mixture was quenched with EtOAc (3 mL*3) The organic solution was dried over Na2SO4, filtered and concentrated to give the title compound. LRMS m/z (M+H): 491.2 required. 491.2 found.

### Step 14: N-((2¹S,2⁴S,5²R,5³S,E)-6-oxo-3,11-dioxa-5(2,1)-piperidina-1(1,3)-benzena-2(1,4)-cyclohexanacycloundecaphan-7-en-5³-yl)methanesulfonamide (28)

To a solution of 28-14 (119 mg, 0.243 mmol) in DCE (25 mL) was added (1,3-dimesitylimidazolidin-2-ylidene)(2-isopropoxybenzylidene)ruthenium(vi) chloride (60.8 mg, 0.097 mmol), and the mixture was stirred at 60° C. for 16 h. TMT (5 mL) was added and stirred at 25° C. for 0.5 h. The mixture was concentrated and purified by TLC (DCM/MeOH=10:1) to give the title compound. LRMS m/z (M+H): 463.2 required, 463.2 found.

### Step 15: N-((2¹S,2⁴S,5²R,5³S)-6-oxo-3,11-dioxa-5(2,1)-piperidina-1(1,3)-benzena-2(1,4)-cyclohexanacycloundecaphane-5³-yl)methanesulfonamide (29)

To a solution of 28 (40 mg, 0.086 mmol) in MeOH (5 mL) was added Pd—C(9.20 mg, 8.65 μmol) (10%, dry) and the solution was stirred at 20° C. for 1 h under H₂ (15 Psi). The mixture was filtered and purified by HPLC ((0.1% TFA)-ACN) to give the title compound. LRMS m/z (M+H): 465.1 required. 465.1 found. ¹H NMR (500 MHz, CD₃OD) δ 7.08 (t, J=7.8 Hz, 1H), 6.93-6.54 (m, 3H), 5.35-5.23 (m, 0.5H), 4.64-4.42 (m, 1H), 4.28-4.10 (m, 2H), 3.99 (t, J 10.2 Hz, 0.5H), 3.93-3.85 (m, 1.5H), 3.74-3.63 (m, 1.5H), 3.54-3.39 (m, 1H), 3.30-3.20 (m, 0.5H), 3.01 (d, J=1.2 Hz, 3H), 2.93-2.65 (m, 2H), 2.62-2.44 (m, 1.5H), 2.20-1.41 (m, 16H).

### Example 30, 31, & 32

### N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (30), N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (31), & N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (32)

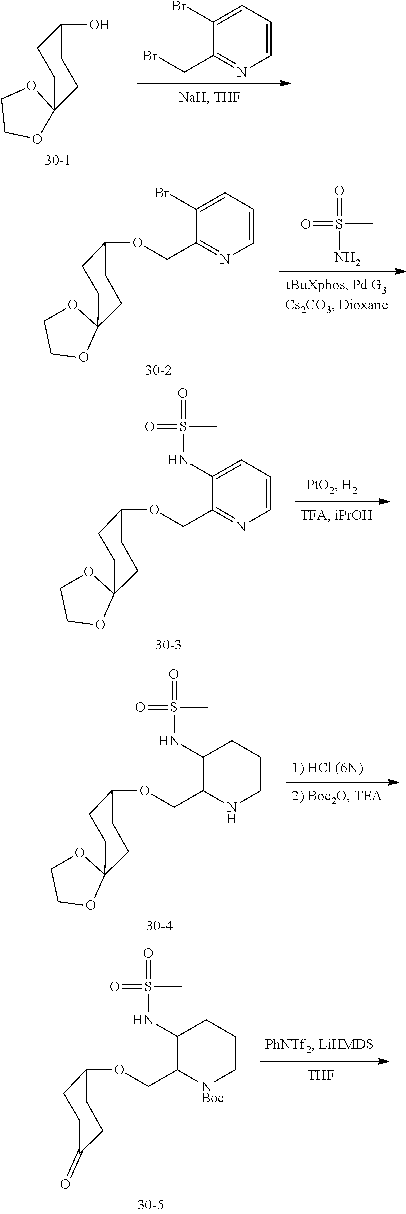

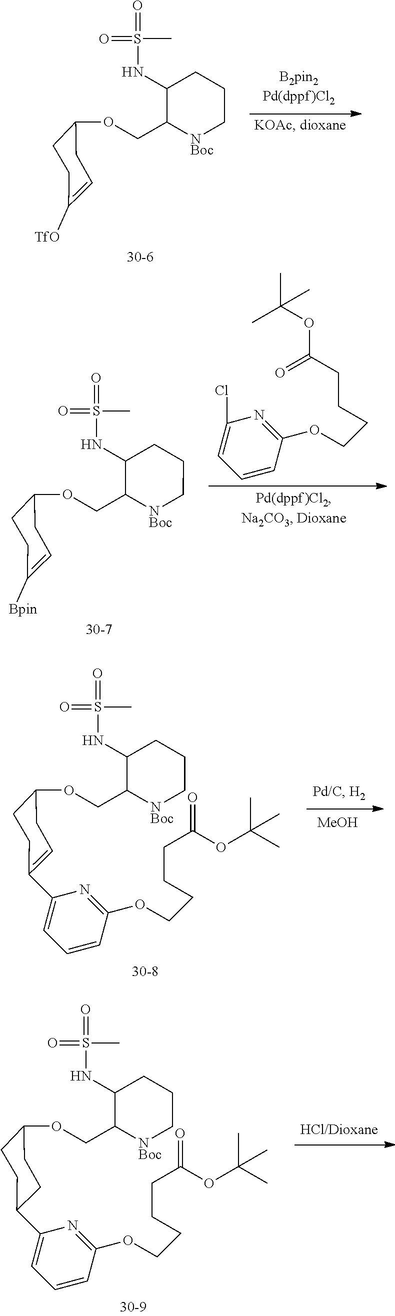

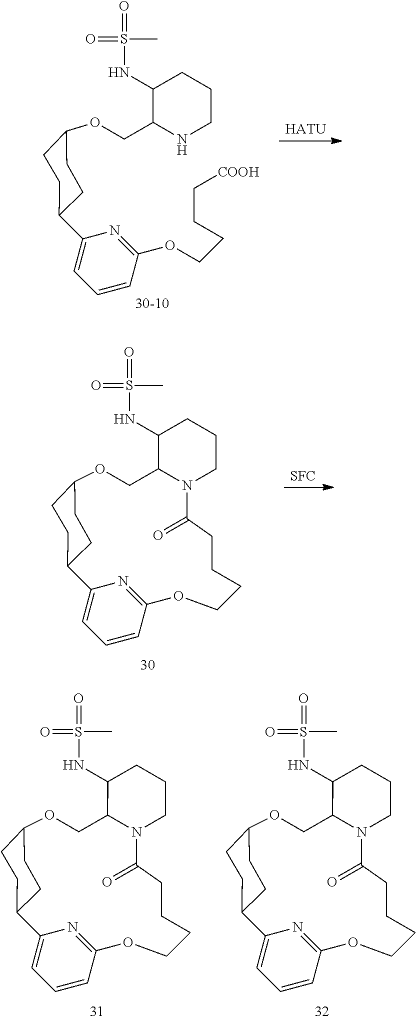 [SMILES: CS(=O)(=O)NC1CCCNC1COC1CCC(c2cccc(OCCCCC(=O)O)n2)CC1 ; "HATU" ; CS(=O)(=O)NC1CCCN2C(=O)CCCCOc3cccc(n3)C3CCC(CC3)OCC12 ; "SFC" ; CS(=O)(=O)NC1CCCN2C(=O)CCCCOc3cccc(n3)C3CCC(CC3)OCC12 ; CS(=O)(=O)NC1CCCN2C(=O)CCCCOc3cccc(n3)C3CCC(CC3)OCC12]

### Step 1: 2-(((1,4-dioxaspiro[4.5]decan-8-yl)oxy)methyl)-3-bromopyridine (30-2)

To a solution of 30-1 (3.78 g, 23.91 mmol) in THF (100 mL) was added NaH (0.956 g, 23.91 mmol) at 0° C. and stirred at 0° C. for 30 mins. 3-bromo-2-(bromomethyl) pyridine (5 g, 19.93 mmol) was added to the above mixture and stirred at 30° C. for 16 h. The reaction mixture was poured into water (100 mL), extracted with EtOAc (100 mL×3). The organic layer was washed with brine (100 mL), dried over Na₂SO₄ and filtered. The filtrate was concentrated and the residue was purified by flash silica gel chromatography (60% ethyl acetate/pet. ether gradient) to give the title compound. LCMS m/z (M+H): 328.0 required, 328.0 found.

### Step 2: N-(2-(((1,4-dioxaspiro[4.5]decan-8-yl)oxy)methyl)pyridin-3-yl)methanesulfonamide (30-3)

To a solution of 30-2 (2 g, 6.09 mmol), methanesulfonamide (0.696 g, 7.31 mmol) and Cs₂CO₃ (5.96 g, 18.28 mmol) in Dioxane (40 mL) was added t-Buxphos pd G₃ (0.484 g, 0.609 mmol). The mixture was stirred at 100° C. under N₂ for 16 h. Then the reaction was filtered and concentrated in vacuo. The residue was purified by flash silica gel chromatography (10% MeOH/DCM gradient) to give the title compound. LCMS (M+H): 343.2 required, 343.2 found.

### Step 3: N-(2-(((1,4-dioxaspiro[4.5]decan-8-yl)oxy)methyl)piperidin-3-yl)methanesulfonamide (30-4)

To a solution of 30-3 (2.2 g, 6.43 mmol) in i-PrOH (40 mL) was added TFA (0.527 mL, 7.07 mmol) and platinum(IV) oxide (0.146 g, 0.643 mmol). The solution was stirred at 30° C. for 16 h under H₂ (50 Psi). The mixture was filtered to give the title compound. LCMS m/z (M+H): 349.0 required, 349.0 found.

### Step 4: tert-butyl 3-(methylsulfonamido)-2-(((4-oxocyclohexyl)oxy)methyl)piperidine-1-carboxylate (30-5)

A mixture of 30-4 (2.1 g, 6.03 mmol) in THF (20 mL) and HCl (6N) (20 mL) was stirred at 20° C. for 3 h. The mixture was concentrated to give N-(2-(((4-oxocyclohexyl)oxy)methyl)-piperidin-3-yl)methanesulfonamide (LCMS m/z (M+H): 305.1 required, 305.1 found). To a solution of N-(2-(((4-oxocyclohexyl)oxy)methyl)piperidin-3-yl)methanesulfonamide (1.83 g, 6.01 mmol) in DCM (40 mL) was added BOC₂O (2.79 mL, 12.02 mmol), TEA (2.51 mL, 18.04 mmol). The solution was stirred at 20° C. for 3 h. The reaction mixture was poured into water (20 mL), extracted with DCM (20 mL×3). The organic layer was washed with brine (20 mL), dried over Na₂SO₄ and filtered. After filtration was concentrated to give crude product, the residue was purified by flash silica gel chromatography (100% EtOAc/Pet.ether gradient) to give the title compound. MS (ESI) m/z: 305.2 [M+H-Boc]. ¹H NMR (400 MHz, CDCl₃) δ 4.02-3.91 (m, 2H), 3.79 (br d, J=3.1 Hz, 1H), 3.69 (dd, J=4.5, 9.6 Hz, 1H), 3.63-3.53 (m, 1H), 3.03-2.95 (m, 3H), 2.78-2.65 (m, 1H), 2.57-2.45 (m, 2H), 2.36-2.26 (m, 2H), 2.04 (s, 5H), 1.72 (br d, J=9.8 Hz, 2H), 1.58 (s, 2H), 1.46 (s, 9H).

### Step 5: tert-butyl 3-(methylsulfonamido)-2-((((R)-4-(((trifluoromethyl)sulfonyl)oxy)cyclohex-3-en-1-yl)oxy)methyl)piperidine-1-carboxylate (30-6)

To a solution of 30-5 (1.3 g, 3.21 mmol) and n,n-bis(trifluoromethylsulfonyl)aniline (2.296 g, 6.43 mmol) in THF (25 mL) at −78° C. was added lithium bis(trimethylsilyl)amide (8.03 mL, 8.03 mmol) (1 M in THF) under N2 at −78° C. The mixture was stirred at −78° C. for 30 minutes and warmed to 25° C. for 3 h. The reaction mixture was poured into water (20 mL), extracted with EtOAc (20 mL×3), the combined organic phases were washed with brine (20 mL), dried over Na₂SO₄. After filtration and concentration. The residue was purified by flash silica gel chromatography (100% EtOAc/Pet.ether gradient) to give the title compound. MS (ESI) m/z: 437.1 [M+H-Boc].

### Step 6: tert-butyl 3-(methylsulfonamido)-2-((((R)-4-(4,4,5,5-tetramethyl-1,3,2-dioxaborolan-2-yl)cyclohex-3-en-1-yl)oxy)methyl)piperidine-1-carboxylate (30-7)

To a solution of 30-6 (700 mg, 1.305 mmol) in Dioxane (10 mL) were added 4,4,4′,4′,5,5,5′,5′-octamethyl-2,2′-bi(1,3,2-dioxaborolane) (497 mg, 1.957 mmol), potassium acetate (384 mg, 3.91 mmol) and Pd(dppf)Cl₂ (95 mg, 0.130 mmol). The mixture was stirred at 100° C. under N₂ for 12 h. The mixture of 30-7 was used next step directly. MS (ESI) m/z: 415.2 [M+H-Boc].

### Step 7: tert-butyl 2-((((R)-4-(6-((5-(tert-butoxy)-5-oxopentyl)oxy)pyridin-2-yl)cyclohex-3-en-1-yl)oxy)methyl)-3-(methylsulfonamido)piperidine-1-carboxylate (30-8)

To a solution of 30-7 (660 mg, 1.283 mmol) in Dioxane (10 mL) and water (3 mL) was added tert-butyl 5-((6-chloropyridin-2-yl)oxy)pentanoate (440 mg, 1.539 mmol), Na2CO₃ (408 mg, 3.85 mmol) and Pd(dppf)Cl₂ (94 mg, 0.128 mmol), the mixture was stirred at 90° C. for 16 h. The mixture was poured into water (10 mL), extracted with EtOAc (10 mL×3). The combined organic phases were washed with brine (20 mL), and dried over Na₂SO₄. After filtration and concentration, the residue was purified by fash silica gel chromatography (100% EtOAc/Pet.ether gradient) to give the title compound. LCMS m/z (M+H): 638.5 required, 638.5 found.

### Step 8: tert-butyl 2-((((1s,4s)-4-(6-((5-(tert-butoxy)-5-oxopentyl)oxy)pyridin-2-yl)cyclohexyl)oxy)methyl)-3-(methylsulfonamido)piperidine-1-carboxylate (30-9)

To a solution of 30-8 (350 mg, 0.549 mmol) in MeOH (10 mL) was added Pd—C(58.4 mg, 0.549 mmol) (10%, dry) and the solution was stirred at 20° C. for 1 h under H2 (15 Psi). The mixture was filtered and concentrated to give the title compound. LCMS m/z (M+H): 640.6 required, 640.6 found.

### Step 9: 5-((6-((1s,4s)-4-((3-(methylsulfonamido)piperidin-2-yl)methoxy)cyclohexyl)pyridin-2-yl)oxy)pentanoic acid (30-10)

To a solution of 30-9 (245 mg, 0.383 mmol) in 4M HCl/dioxane (10 mL) was stirred at 25° C. for 2 h. LCMS showed the reaction was completed. The reaction mixture was concentrated to give the title compound. LCMS m/z (M+H+MeOH—H₂O): 498.2 required, 498.2 found.

### Step 10: N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (30)

To a solution of 30-10 (185 mg, 0.383 mmol) and HATU (218 mg, 0.574 mmol) in DMF (5 mL) were added and the resulting mixture was stirred at 20° C. for 16 h. The reaction mixture was poured into water (20 mL), extracted with EtOAc (20 mL×3). The organic layer was washed with brine (10 mL), dried over Na₂SO₄ and filtered. The filtrate was concentrated and the residue was purified by HPLC (water (0.1% TFA)-ACN) to give the title compound. LCMS m/z (M+H): 466.4 required, 466.4 found. ¹H NMR (500 MHz, CD₃OD) δ 7.46 (t, J=7.6 Hz, 1H), 6.74-6.62 (m, 1H), 6.46 (d, J=8.2 Hz, 1H), 4.57-4.45 (m, 2H), 4.08 (dt, J=4.5, 10.0 Hz, 1H), 3.91-3.81 (m, 1H), 3.68-3.59 (m, 3H), 3.01 (s, 3H), 2.95-2.78 (m, 3H), 2.67-2.57 (m, 1H), 2.34-2.23 (m, 1H), 2.14-1.68 (m, 10H), 1.62-1.39 (m, 6H).

### Step 11: N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (31) & N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide (32)

30 (66 mg, 0.142 mmol) was separated by SFC (1014048-086-1) to give 31 and 32.

Sfc Condition:
Column: Chiralpak AD-3 50×4.6 mm I.D., 3 um
Mobile phase: A: CO₂ B: ethanol (0.05% DEA)
Gradient: from 5% to 40% of B in 2 min and hold 40% for 1.2 min, then 5% of B for 0.8 min
Flow rate: 4 mL/min
Column temp.: 35° C.
ABPR: 1500 psi

¹H NMR (31) (500 MHz, CD₃OD) δ 7.45 (dd, J=7.2, 8.2 Hz, 1H), 6.73-6.62 (m, 1H), 6.51-6.39 (m, 1H), 4.86-4.79 (m, 1H), 4.59-4.40 (m, 2H), 4.15-4.07 (m, 1H), 3.91-3.79 (m, 2H), 3.67-3.60 (m, 1H), 3.49 (td, J=4.9, 11.9 Hz, 1H), 3.03-2.98 (m, 3H), 2.95-2.86 (m, 1H), 2.80-2.69 (m, 2H), 2.65-2.56 (m, 1H), 2.33-2.21 (m, 1H), 2.18-2.07 (m, 1H), 2.01-1.74 (m, 7H), 1.63-1.38 (m, 7H).

¹H NMR (32) (500 MHz, CD₃OD) δ 7.44 (dd, J=7.2, 8.2 Hz, 1H), 6.72-6.61 (m, 1H), 6.49-6.38 (m, 1H), 4.81 (br s, 1H), 4.64-4.41 (m, 2H), 4.15-4.03 (m, 1H), 3.92-3.77 (m, 2H), 3.68-3.58 (m, 1H), 3.54-3.43 (m, 1H), 3.03-2.97 (m, 3H), 2.95-2.86 (m, 1H), 2.79-2.67 (m, 2H), 2.64-2.55 (m, 1H), 2.32-2.21 (m, 1H), 2.18-2.07 (m, 1H), 2.01-1.73 (m, 7H), 1.63-1.38 (m, 7H).

### Example 33, 34, 35, & 36

### N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (33, Cis, Cis Isomer 1), N-((2¹S,2³R⁵²R,5³S)-6-oxo-3,10-dioxa-5(2,1)-piperidina-1(1,3)-benzena-2(1,3)-cyclopentanacyclodecaphane-5³-yl)methanesulfonamide (34, Cis, Cis Isomer 2), N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (35, Cis, Cis Isomer 3), & N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (36, Cis, Cis Isomer 4)

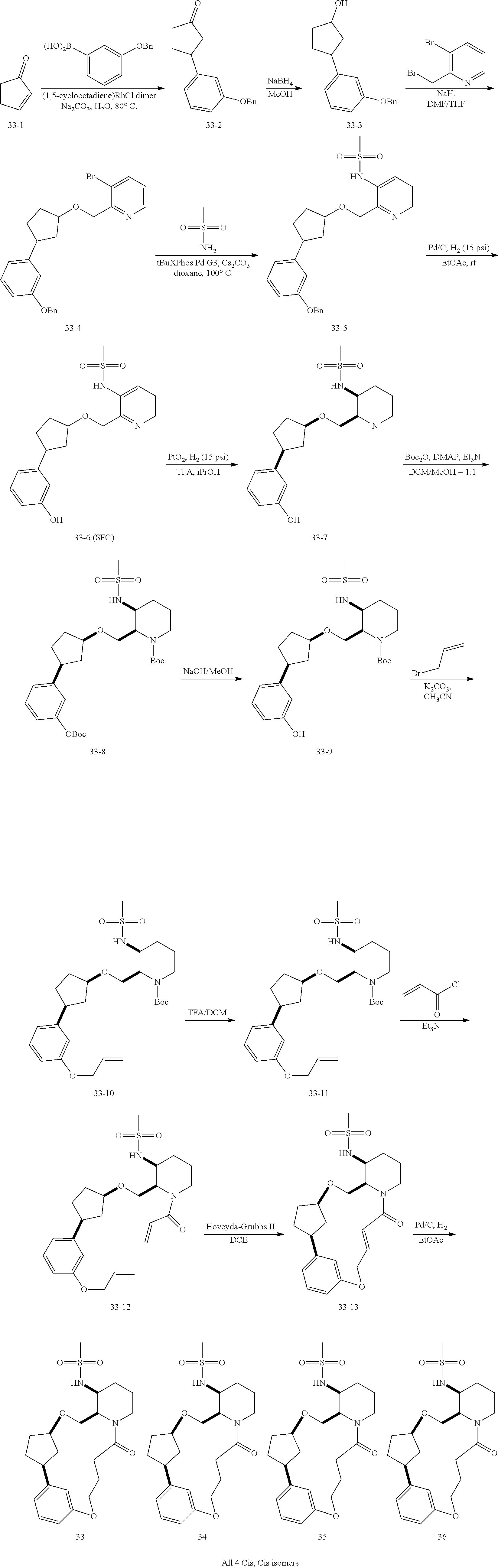

### Step 1: 3-(3-(benzyloxy)phenyl)cyclopentan-1-one (33-2)

To a solution of (3-(benzyloxy)phenyl)boronic acid (12 g, 52.6 mmol) in Water (150 mL) were added Na₂CO₃ (4.46 g, 42.1 mmol), chloro(1,5-cyclooctadiene)rhodium(i) dimer (0.311 g, 0.631 mmol) and Compound 33-1 (5.18 g, 63.1 mmol). The mixture was stirred at 80° C. for 2 h. The mixture was quenched with extracted with EtOAc (200 mL×3), washed with NaHCO₃(100 mL), dried over Na₂SO₄, filtered and concentrated under reduced pressure. The residue was purified by flash silica gel chromatography (10% ethyl acetate/pet. ether gradient) to give the title compound. LCMS m/z (M+H): 267.1 required, 267.3 found.

### Step 2: 3-(3-(benzyloxy)phenyl)cyclopentan-1-ol (33-3)

To a solution of 33-2 (10 g, 37.5 mmol) in MeOH (100 mL) was added NaBH₄ (2.131 g, 56.3 mmol) at 0° C., then the mixture was stirred at 25° C. for 3 h. The reaction was quenched with water (300 mL) and extracted with EtOAc (3×100 mL). The combined organic layers were washed with brine (200 mL), dried (Na₂SO₄), filtered and concentrated in vacuo. The filtrate was purified by Flash silica gel chromatography (30% EtOAc/Pet.ether gradient) to give the title compound. LCMS m/z (M+H-H₂O): 251.3 found, 251.3 required.

### Step 3: 2-(((3-(3-(benzyloxy)phenyl)cyclopentyl)oxy)methyl)-3-bromopyridine (33-4)

To a solution of 33-3 (4 g, 14.91 mmol) and 3-bromo-2-(bromomethyl)pyridine (6.80 g, 17.89 mmol) in THF (60 ml)/DMF (10 ml) was added sodium hydride (0.715 g, 17.89 mmol) (60% wt), and the mixture was stirred at 25° C. for 2 h. Then another batch of 3-bromo-2-(bromomethyl)pyridine (1.700 g, 4.47 mmol) and sodium hydride (0.179 g, 4.47 mmol) (60% wt) were added to the above solution. Then the reaction mixture was stirred at 25° C. for 12 h. The reaction mixture was poured into water (200 mL), extracted with EtOAc (100 mL×3). The organic layer was washed with brine (100 mL), dried over Na₂SO₄ and filtered. The filtrate was concentrated and the residue was purified by Flash silica gel chromatography (30% EtOAc/Pet.ether gradient) to the title compound. LCMS m/z (M+H): 440.2 required, 440.2 found.

### Step 4: N-(2-(((3-(3-(benzyloxy)phenyl)cyclopentyl)oxy)methyl)pyridin-3-yl)methanesulfonamide (33-5)

To a solution of 33-4 (2.2 g, 5.02 mmol), methanesulfonamide (0.716 g, 7.53 mmol) and Cs₂CO₃ (4.91 g, 15.06 mmol) in 1,4-Dioxane (30 mL) was added tBuXPhos Pd G₃ (0.399 g, 0.502 mmol) under N₂, and the mixture was stirred at 100° C. for 12 hours. The reaction was poured into water (10 mL), extracted with EtOAc (10 mL×3). The organic layer was washed with brine (10 mL), dried over Na₂SO₄, filtered and concentrated. The residue was purified by Flash silica gel chromatography (60% EtOAc/Pet.ether gradient) to give the title compound. LCMS m/z (M+H): 453.2 required, 453.2 found.

### Step 5: N-(2-((((1R,3S)-3-(3-hydroxyphenyl)cyclopentyl)oxy)methyl)pyridin-3-yl)methanesulfonamide (33-6)

To a solution of 33-5 (2.6 g, 5.74 mmol) in EtOAc (30 mL) was added Pd—C(1.223 g, 1.149 mmol) (10% wt) and the mixture was stirred at 25° C. under H₂ (15 psi) for 12 h. The mixture was filtered and the filtrate was concentrated. The crude product was separated by SFC to give the title compound and the undesired trans isomer. LCMS m/z (M+H): 363.2 required, 363.2 found.

Sfc Condition:
Column: ChiralPak AD-3 150×4.6 mm I.D., 3 um
Mobile phase: A: CO₂ B: Methanol (0.05% DEA)
Gradient: from 5% to 40% of B in 5.5 min and hold 40% for 3 min, then 5% of B for 1.5 min
Flow rate: 2.5 mL/min Column temp.:40° C.
Back pressure: 100 bar

### Step 6: N-((2R,3S)-2-((((1R,3S)-3-(3-hydroxyphenyl)cyclopentyl)oxy)methyl)piperidin-3-yl)methanesulfonamide (33-7)

To a solution of 33-6 (500 mg, 1.380 mmol) in i-PrOH (10 mL) were added trifluoroacetic acid (0.257 mL, 3.45 mmol) and platinum(IV) oxide (376 mg, 0.083 mmol) (5% wt). The solution was stirred at 30° C. for 3 h under H₂ (15 psi). The reaction mixture was filtered through a Celite pad and poured into TEA to adjust PH=7, filtered and concentrated to give the title compound. LCMS m/z (M+H): 369.2 required, 369.2 found.

### Step 7: tert-butyl (2R,3S)-2-((((1R,3S)-3-(3-((tert-butoxycarbonyl)oxy)phenyl)cyclopentyl)oxy)methyl)-3-(methylsulfonamido)piperidine-1-carboxylate (33-8)

To a mixture of 33-7 (400 mg, 1.086 mmol), in DCM (5 mL) and MeOH (5 mL) were added TEA (0.454 mL, 3.26 mmol), BOC-Anhydride (0.504 mL, 2.171 mmol) and DMAP (13.26 mg, 0.109 mmol) at 0° C., and the resulting mixture was stirred at 20° C. for 10 h. The reaction mixture was concentrated and quenched with water (30 mL), then extracted with EtOAc (10 mL×3), washed with brine (10 mL), dried over Na₂SO₄, filtered and the filtrate was concentrated. The residue was purified by Flash silica gel chromatography (5% EtOAc/Pet.ether gradient) to give the title compound. LCMS m/z (M+H-Boc): 469.2 required, 469.2 found.

### Step 8: tert-butyl (2R,3S)-2-((((1R,3S)-3-(3-hydroxyphenyl)cyclopentyl)oxy)methyl)-3-(methylsulfonamido)piperidine-1-carboxylate (33-9)

To a solution of 33-8 (100 mg, 0.176 mmol) in MeOH (2 mL) was added NaOH (0.879 mL, 1.758 mmol) (2 M in water). The solution was stirred at 20° C. for 13 h. The mixture was quenched with water (20 mL) and extracted with EtOAc (20 mL×3), washed with brine (20 mL), dried over Na₂SO₄, filtered and concentrated under reduced pressure to give the title compound. LCMS m/z (M+H-Boc): 369.2 required, 369.2 found.

### Step 9: tert-butyl (2R,3S)-2-((((1R,3S)-3-(3-(allyloxy)phenyl)cyclopentyl)oxy)methyl)-3-(methylsulfonamido)piperidine-1-carboxylate (33-10)

To a solution of 33-9 (250 mg, 0.533 mmol) in MeCN (8 mL) were added K₂CO₃ (221 mg, 1.600 mmol) and 3-bromoprop-1-ene (64.5 mg, 0.533 mmol) at 0° C. The reaction was stirred at 0° C. to 25° C. for 2 h. The mixture was quenched with H₂O (30 mL) and extracted with EtOAc (30 mL×3), washed with brine (30 mL), dried over Na₂SO₄, filtered and concentrated under reduced pressure. The residue was purified by flash silica gel chromatography (60% ethyl acetate/pet. ether gradient) to give the title compound. LCMS m/z (M-100+H): 409.2 required, 409.2 found.

### Step 10: N-((2R,3S)-2-((((1R,3S)-3-(3-(allyloxy)phenyl)cyclopentyl)oxy)methyl)piperidin-3-yl)methanesulfonamide (33-11)

To a solution of 33-10 (150 mg, 0.295 mmol) in DCM (2 mL) was added TFA (0.068 mL, 0.885 mmol). The solution was stirred at 20° C. for 1 h. The mixture was concentrated to give the title compound. LCMS m/z (M+H): 409.3 required, 409.3 found.

### Step 11: N-((2R,3S)-1-acryloyl-2-((((1R,3S)-3-(3-(allyloxy)phenyl)cyclopentyl)oxy)methyl)-piperidin-3-yl)methanesulfonamide (33-12)

To a solution of 33-11 (120 mg, 0.294 mmol) in DCM (3 mL) were added Et₃N (0.123 mL, 0.881 mmol) and acryloyl chloride (29.2 mg, 0.323 mmol) at 0° C. The mixture was stirred at 0° C. to 20° C. for 2 h. The mixture was quenched with water (30 mL) and extracted with DCM (20 mL×3), washed with brine (20 mL), dried over Na₂SO₄, filtered and concentrated under reduced pressure. The residue was purified by flash silica gel chromatography (60% ethyl acetate/pet. ether gradient) to give the title compound. LCMS m/z (M+H): 463.2 required, 463.2 found.

### Step 12: N-((2¹S,2³R,5²R,5³S,E)-6-oxo-3,10-dioxa-5(2,1)-piperidina-1(1,3)-benzena-2(1,3)-cyclopentanacyclodecaphan-7-en-5³-yl)methanesulfonamide (33-13)

To a solution of 33-12 in DCE (80 mL) was added (1,3-dimesitylimidazolidin-2-ylidene)(2-isopropoxybenzylidene)ruthenium(vi) chloride (18.96 mg, 0.030 mmol). The mixture was stirred at 50° C. for 12 h. The reaction mixture was concentrated to give the crude. The residue was purified by flash silica gel chromatography (60% ethyl acetate/pet. ether gradient) to give the title compound. LCMS m/z (M+H): 435.1 required, 435.1 found.

### Step 13: N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (33, Cis, Cis Isomer 1), N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (34, Cis, Cis Isomer 2), N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (35, Cis, Cis Isomer 3), & N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide (36, Cis, Cis Isomer 4)

To a solution of 33-13 (50 mg, 0.115 mmol) in EtOAc (5 mL) was added Pd—C(61.2 mg, 0.058 mmol) (10% wt) and the solution was stirred at 20° C. for 20 mins under dihydrogen (excess) 15 psi. The mixture was filtered and concentrated to give the crude. The crude was purified by pre-TLC (Pet. ether:EtOAc=1:1) and separated by SFC_1 to give Part 1 (1:1 by SFC) and part 2 (1:1 by SFC). Part 1 was separated by SFC_2 to give 33 and 34. Part 2 was separated by SFC_3 to give 35 and 36.

Sfc 1 Condition:
Column: Chiralpak IC-3 150×4.6 mm I.D., 3 um
Mobile phase: 40% of ethanol (0.05% DEA) in CO₂
Flow rate: 2.5 mL/min

Sfc 2 Condition:
Column: Chiralpak AD-3 150×4.6 mm I.D., 3 um
Mobile phase: A: CO₂ B: ethanol (0.05% DEA)
Gradient: from 5% to 40% of B in 5 min and from 40% to 5% of B in 0.5 min£¬, hold 5% of B for 1.5 min
Flow rate: 2.5 mL/min

Sfc 3 Condition:
Column: Chiralpak AD-3 150×4.6 mm I.D., 3 um
Mobile phase: A: CO₂ B: iso-propanol (0.05% DEA)
Gradient: from 5% to 40% of B in 5 min and hold 40% for 2.5 min, then 5% of B for 2.5 min
Flow rate: 2.5 mL/min

LCMS m/z (M+H): 437.2 required, 437.2 found.

¹H NMR (33) (400 MHz, CD₃OD) δ 7.12-7.01 (m, 2H), 6.74 (br d, J=7.4 Hz, 1H), 6.68-6.63 (m, 1H), 4.53 (td, J=4.7, 9.7 Hz, 0.5H), 4.42 (br dd, J=3.5, 13.7 Hz, 0.5H), 4.26-3.96 (m, 4H), 3.86-3.69 (m, 2H), 3.56-3.34 (m, 1H), 3.23-3.09 (m, 2H), 3.05-2.97 (m, 3H), 2.85-2.68 (m, 1H), 2.41-2.24 (m, 2H), 2.21-1.92 (m, 4H), 1.89-1.54 (m, 8H)

¹H NMR (34) (400 MHz, CD₃OD) δ 7.12-7.01 (m, 1H), 6.86 (s, 1H), 6.78-6.63 (m, 2H), 5.26 (td, J=4.8, 9.5 Hz, 0.5H), 4.60-4.40 (m, 0.5H), 4.19-3.93 (m, 4H), 3.86-3.74 (m, 1H), 3.63 (dd, J=4.3, 9.8 Hz, 1H), 3.53-3.43 (m, 1H), 3.25-3.04 (m, 1H), 3.01-2.92 (m, 3H), 2.85-2.55 (m, 1H), 2.38-2.14 (m, 3H), 2.11-1.94 (m, 3H), 1.91-1.77 (m, 4H), 1.70-1.49 (m, 3H)

¹H NMR (35) (400 MHz, CD₃OD) δ 7.12-7.01 (m, 1H), 6.86 (s, 1H), 6.78-6.63 (m, 2H), 5.26 (td, J=4.8, 9.5 Hz, 1H), 4.60-4.40 (m, 1H), 4.20-3.93 (m, 4H), 3.86-3.75 (m, 1H), 3.63 (dd, J=4.1, 10.0 Hz, 1H), 3.55-3.43 (m, 1H), 3.25-3.09 (m, 2H), 3.01-2.92 (m, 4H), 2.85-2.55 (m, 1H), 2.38-2.15 (m, 3H), 2.11-1.94 (m, 3H), 1.91-1.77 (m, 4H), 1.72-1.49 (m, 3H)

¹H NMR (36) (400 MHz, CD₃OD) δ 7.12-7.01 (m, 2H), 6.74 (br d, J=7.4 Hz, 1H), 6.68-6.63 (m, 1H), 4.53 (td, J=4.7, 9.7 Hz, 0.5H), 4.42 (br dd, J=3.5, 13.7 Hz, 0.5H), 4.26-3.96 (m, 4H), 3.86-3.69 (m, 2H), 3.56-3.34 (m, 1H), 3.23-3.09 (m, 2H), 3.05-2.97 (m, 3H), 2.85-2.68 (m, 1H), 2.41-2.24 (m, 2H), 2.21-1.92 (m, 4H), 1.89-1.54 (m, 8H)

### Example 37, 38, 39, & 40

### N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (37, Isomer 1), N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (38, Isomer 2), N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (39, Isomer 3), & N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (40, Isomer 4)

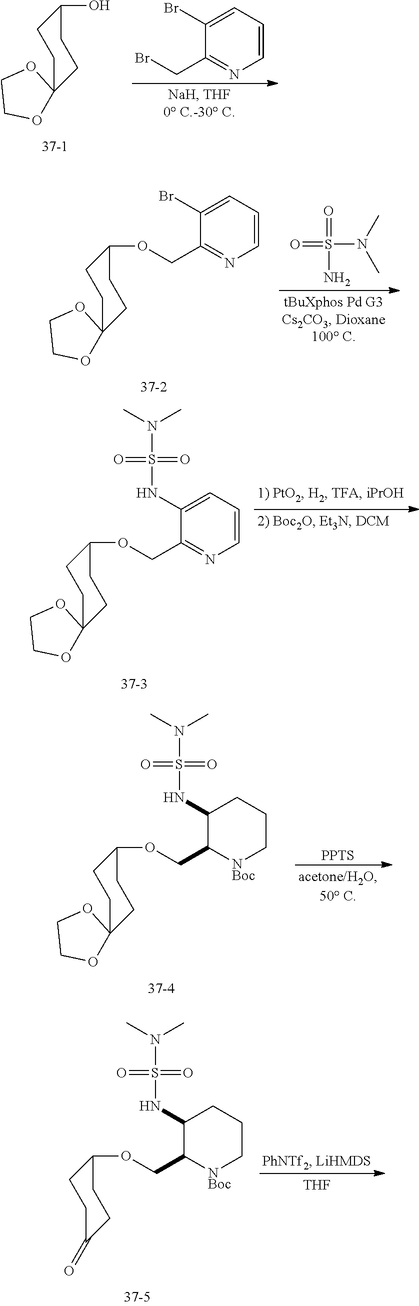

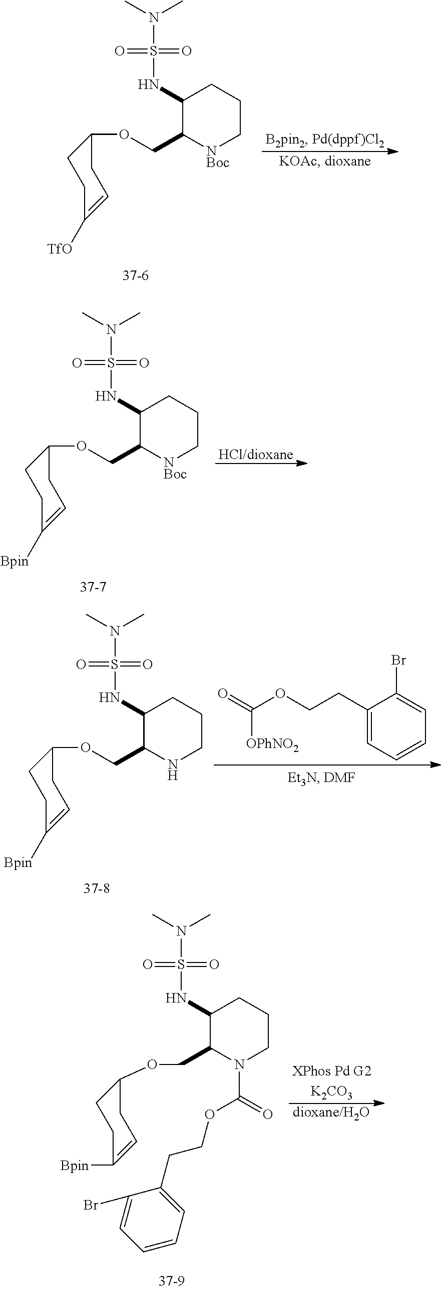

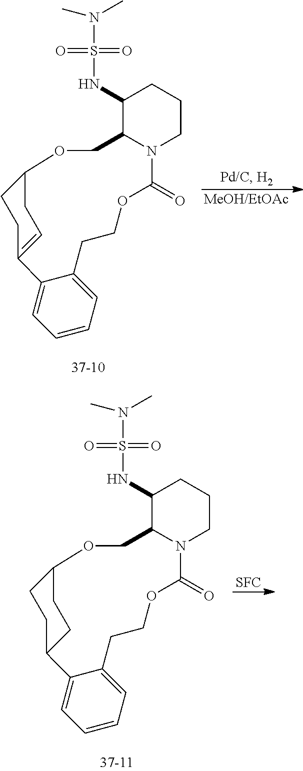

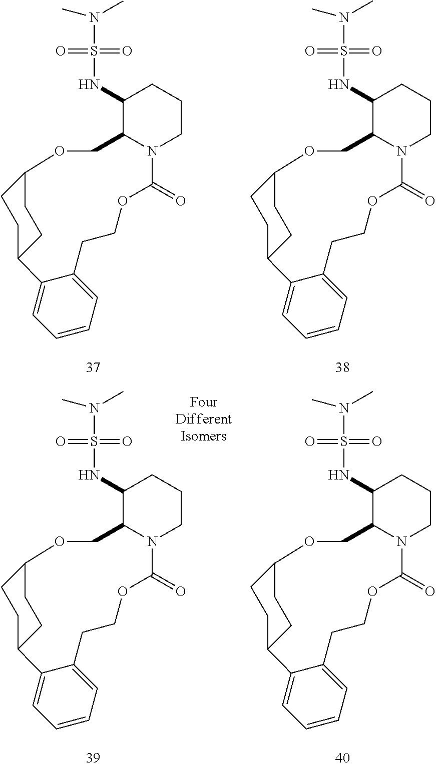

### Step 1: 2-(((1,4-dioxaspiro[4.5]decan-8-yl)oxy)methyl)-3-bromopyridine (37-2)

To a solution of 37-1 (15.25 g, 96 mmol) in THF (500 mL) was added NaH (3.86 g, 96 mmol) at 0° C. and stirred at 0° C. for 30 mins. 3-bromo-2-(bromomethyl)pyridine (24 g, 80 mmol) was added to the above mixture and stirred at 30° C. for 16 h. The reaction mixture was poured into water (500 mL), extracted with EtOAc (500 mL×3). The organic layer was washed with brine (100 mL), dried over Na₂SO₄ and filtered. The filtrate was concentrated and the residue was purified by flash silica gel chromatography (60% ethyl acetate/pet. ether gradient) to give the title compound.

### Step 2: N′-(2-(((1,4-dioxaspiro[4.5]decan-8-yl)oxy)methyl)pyridin-3-yl)N,N-dimethyl-sulfamide (37-3)

To a solution of 37-2 (10 g, 30.5 mmol), the sulfamide (4.54 g, 36.6 mmol) and Cs₂CO₃ (29.8 g, 91 mmol) in dioxane (200 mL) was added tBuXphos Pd G3 (2.420 g, 3.05 mmol). The mixture was stirred at 100° C. under N₂ for 16 h. Then the reaction was filtered and concentrated in vacuo. The residue was purified by flash silica gel chromatography (10% MeOH/DCM gradient) to give the title compound.

### Step 3: tert-butyl (2R,3S)-2-(((1,4-dioxaspiro[4.5]decan-8-yl)oxy)methyl)-3-((N,N-dimethylsulfamoyl)amino)piperidine-1-carboxylate (37-4)

To a solution of 37-3 (7 g, 18.84 mmol) in i-PrOH (120 mL) was added trifluoroacetic acid (1.545 ml, 20.73 mmol) and platinum(IV) oxide (0.856 g, 3.77 mmol). The solution was stirred at 30° C. for 16 h under H₂ (50 Psi). The mixture was filtered and concentrated to give reduced product, which was used next step directly (LCMS m/z (M+H): 378.0 required, 378.0 found). To a solution of reduced product (7 g, 18.54 mmol) in DCM (100 mL) was added Boc₂O (8.61 ml, 37.1 mmol), TEA (7.75 ml, 55.6 mmol). The solution was stirred at 20° C. for 16 hours. The reaction mixture was poured into water (50 mL), extracted with DCM (50 mL×3). The organic layer was washed with brine (50 mL), dried over Na₂SO₄ and filtered. After filtration was concentrated to give crude product, the residue was purified by flash silica gel chromatography (100% EtOAc/Pet.ether gradient) to give the title compound.

### Step 4: tert-butyl (2R,3S)-3-((N,N-dimethylsulfamoyl)amino)-2-(((4-oxocyclohexyl)oxy)-methyl)piperidine-1-carboxylate (37-5)

To a solution of 37-4 (6 g, 12.56 mmol) in acetone (60 mL) and Water (60 mL) was added PPTS (6.31 g, 25.1 mmol). The solution was stirred at 60° C. for 3 hours. The reaction mixture was poured into water (50 mL) and aq.NaHCO₃(25 mL), extracted with EtOAc (50 mL×3). The organic layer was washed with brine (30 mL), dried over Na₂SO₄, filtered and concentrated to give crude product, which was purified by flash silica gel chromatography (80% ethyl acetate/pet. ether gradient) to give the title compound.

### Step 5: tert-butyl (2R,3S)-3-((N,N-dimethylsulfamoyl)amino)-2-((((R)-4-(((trifluoromethyl)sulfonyl)oxy)cyclohex-3-en-1-yl)oxy)methyl)piperidine-1-carboxylate (37-6)

To a solution of 37-5 (4 g, 9.23 mmol) and N,N-bis(trifluoromethylsulfonyl)aniline (6.59 g, 18.45 mmol) in THF (80 mL) at −78° C. was added lithium bis(trimethylsilyl)amide (23.06 ml, 23.06 mmol) (1 M in THF) under N₂ at −78° C. The mixture was stirred at −78° C. for 30 minutes and warmed to 25° C. for 3 h. The reaction mixture was poured into water (50 mL), extracted with EtOAc (50 mL×3). The combined organic phases were washed with brine (50 mL), dried over Na₂SO₄. After filtration and concentration, the residue was purified by flash silica gel chromatography (100% EtOAc/Pet.ether gradient) to give the title compound.

### Step 6: tert-butyl (2R,3S)-3-((N,N-dimethylsulfamoyl)amino)-2-((((R)-4-(4,4,5,5-tetramethyl-1,3,2-dioxaborolan-2-yl)cyclohex-3-en-1-yl)oxy)methyl)piperidine-1-carboxylate (37-7)

To a solution of 37-6 (4 g, 7.07 mmol) in Dioxane (80 mL) were added 4,4,4′,4′,5,5,5′,5′-octamethyl-2,2′-bi(1,3,2-dioxaborolane) (2.335 g, 9.19 mmol), potassium acetate (2.082 g, 21.22 mmol) and Pd(dppf)Cl₂ (0.517 g, 0.707 mmol). The mixture was stirred at 100° C. under N₂ for 12 hours. The mixture was filtered and the filtrate was purified by flash silica gel chromatography (100% EtOAc/Pet.ether gradient) to give the title compound.

### Step 7: N′-((2R,3S)-2-((((R)-4-(4,4,5,5-tetramethyl-1,3,2-dioxaborolan-2-yl)cyclohex-3-en-1-yl)oxy)methyl)piperidin-3-yl)-N,N-dimethyl-sulfamide (37-8)

A solution of 37-7 (300 mg, 0.552 mmol) in 4M HCl/dioxane (10 mL) was stirred at 25° C. for 2 hours. The reaction mixture was concentrated to give the title compound.

### Step 8: 2-bromophenethyl (2R,3S)-3-((N,N-dimethylsulfamoyl)amino)-2-((((R)-4-(4,4,5,5-tetramethyl-1,3,2-dioxaborolan-2-yl)cyclohex-3-en-1-yl)oxy)methyl)piperidine-1-carboxylate (37-9)

To a mixture of 37-8 (200 mg, 0.451 mmol) in DMF (10 mL) was added TEA (0.314 mL, 2.255 mmol) and 2-bromophenethyl (4-nitrophenyl) carbonate (165 mg, 0.451 mmol). The reaction mixture was stirred at 80° C. for 10 h under N₂. The mixture was diluted with water (30 mL), extracted with EtOAc (30 mL×3). The combined organic phases were washed with brine (50 ml), and dried over Na₂SO₄. After filtration and concentration, the residue was purified by TLC (SiO₂, pet. ether:EtOAC=1:1) to give the title compound.

### Step 9: N′-((2⁴R,5²R,5³S)-6-oxo-3,7-dioxa-5(2,1)-piperidina-1(1,2)-benzena-2(1,4)-cyclohexanacyclononaphan-21-en-5³-yl)-N,N-dimethyl-sulfamide (37-10)

To a mixture of 37-9 (130 mg, 0.194 mmol) in dioxane (13 mL) was added K₂CO₃ (0.969 mL, 0.969 mmol, 1 M in H₂O) and chloro(2-dicyclohexylphosphino-2′,4′,6′-triisopropyl-1,1′-biphenyl)[2-(2′-amino-1,1′-biphenyl)]palladium(ii) (15.26 mg, 0.019 mmol). The reaction mixture was stirred at 80° C. for 10 h under N₂. The reaction mixture was poured into water (20 ml), extracted with EtOAc (20 mL×3). The combined organic phases were dired over Na₂SO₄, filtered. The filtrate was concentrated and purified by TLC (SiO₂, pet. ether:EtOAc=1:1) to give the title compound.

### Step 10: N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (37-11)

To a solution of 37-10 (60 mg, 0.129 mmol) in MeOH (2 mL) and EtOAc (2 mL) was added Pd—C(138 mg, 0.129 mmol) and stirred at 25° C. under H2 (15 psi) for 1 hour. The reaction mixture filtered and concentrated to give the title compound.

### Step 11: N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (37, Isomer 1), N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (38, Isomer 2), N′-((2¹S,2⁴S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide (39, Isomer 3), & N′-((2¹S,2⁴S,5²R,5³S)-6-oxo-3,7-dioxa-5(2,1)-piperidina-1(1,2)-benzena-2(1,A)-cyclohexanacyclononaphane-5³-yl)-N,N-dimethyl-sulfamide (40, Isomer 4)

37-11 (60 mg, 0.129 mmol) was separated by SFC to give the title compounds.

Sfc Condition:
Column: DAICEL CHIRALPAK AD (250 mm×30 mm, 10 um)
Condition: 0.1% NH₃H₂O ETOH
Begin B: 35
End B: 60
FlowRate (ml/min): 80

¹H NMR (37) (500 MHz, CD₃OD) δ 7.22-6.97 (m, 4H), 4.81-4.64 (m, 1H), 4.10-3.87 (m, 2H), 3.85-3.60 (m, 3H), 3.53-3.37 (m, 2H), 3.21-2.93 (m, 1H), 2.81 (d, J=9.6 Hz, 8H), 2.67-2.44 (m, 1H), 2.27-2.04 (m, 2H), 2.02-1.69 (m, 4H), 1.66-1.28 (m, 6H).

¹H NMR (38) (500 MHz, CD₃OD) δ 7.23-6.98 (m, 4H), 4.81-4.64 (in, TH), 4.10-3.86 (m, 2H), 3.83-3.61 (m, 3H), 3.54-3.37 (m, 2H), 3.22-2.93 (in, TH), 2.81 (br d, J=9.6 Hz, 8H), 2.67-2.41 (in, TH), 2.26-2.04 (m, 2H), 2.01-1.70 (m, 4H), 1.65-1.28 (in, 6H). ¹H NMR (39) (400 MHz, CD₃OD) δ 7.19-6.98 (m, 4H), 4.85-4.66 (m, 2H), 4.34-3.98 (in, 3H), 3.94-3.80 (in, TH), 3.77-3.65 (in, TH), 3.56-3.33 (m, 2H), 3.30-3.17 (m, 2H), 3.12-2.89 (in, TH), 2.86-2.71 (m, 6H), 2.27-1.93 (m, 5H), 1.91-1.70 (m, 3H), 1.66-1.19 (in, 4H). ¹H NMR (40) (400 MHz, CD₃OD) δ 7.17-6.99 (m, 4H), 4.85-4.71 (m, 2H), 4.32-4.00 (m, 3H), 3.94-3.80 (in, TH), 3.70 (br t, J=11.5 Hz, TH), 3.55-3.33 (m, 2H), 3.28-3.13 (in, TH), 3.11-2.89 (in, TH), 2.85-2.69 (m, 7H), 2.25-2.07 (m, 3H), 2.05-1.92 (m, 3H), 1.76 (br s, 2H), 1.63-1.50 (in, TH), 1.47-1.34 (m, 2H), 1.30-1.19 (in, TH).

### Example 41-50

The following EXAMPLES 41-50 were prepared according to the general procedures herein and in an analogous manner to that used to synthesize EXAMPLE 13 using the appropriate INTERMEDIATES O through U.

| Ex- | | | Exact |
|---|---|---|---|
| ample | | | Mass |
| Num- | | | [M + |
| ber | Structure | Name | H\|⁺ |
| 41 | 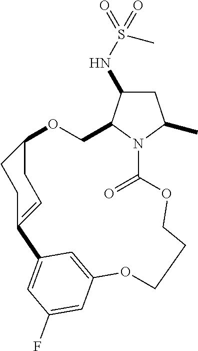 | N-(2⁴S,5²R, 5³S,5⁵R)- 1⁵fluoro-5⁵- methyl-6-oxo- 3,7,11- trioxa-5(2,1)- pyrrolidina- 1(1,3)- benzena-2(1,4)- cyclohexana- cyclounde- caphan-2¹-en-5³- yl)methane- sulfonamide | 483.4 |
| 42 | 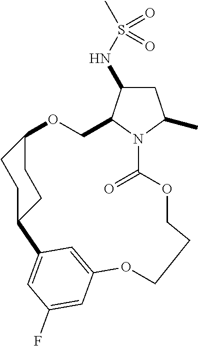 | N-((2¹S,2⁴S, 5²R,5³S,5⁵R)- 1⁵-fluoro-5⁵- methyl-6- oxo-3,7,11- trioxa-5(2,1)- pyrrolidina- 1(1,3)- benzena-2(1,4)- cyclohexana- cyclounde- caphane-5³- yl)methane- sulfonamide | 485.4 |
| 43 | 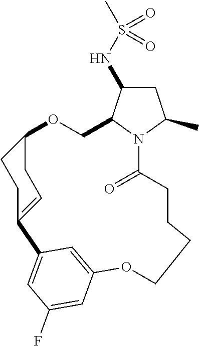 | N-((2¹S,2⁴S, 5²R,5³S,5⁵R)- 1⁵-fluoro-5⁵- methyl-6- oxo-3,11- dioxa-5(2,1)- pyrrolidina- 1(1,3)- benzena-2(1,4)- cyclohexana- cyclounde- caphan-2¹-en-5³- yl)methane- sulfonamide | 481.3 |
| 44 | 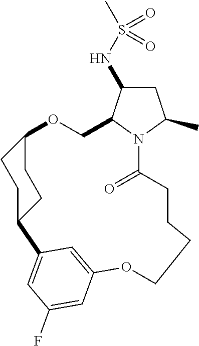 | N-((2¹S,2⁴S, 5²R,5³S,5⁵R)- 1⁵-fluoro-5⁵- methyl-6- oxo-3,11- dioxa-5(2,1)- pyrrolidina- 1(1,3)- benzena-2(1,4)- cyclohexana- cyclounde- caphane-5³- yl)methane- sulfonamide | 483.4 |
| 45 | 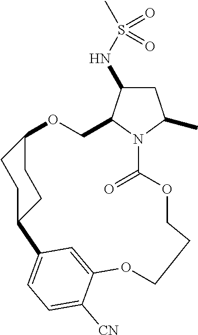 | N-((2¹S,2⁴S, 5²R,5³S,5⁵R)- 1⁴-cyano-5⁵- methyl-6- oxo-3,7,11- trioxa-5(2,1)- pyrrolidina- 1(1,3)- benzena-2(1,4)- cyclohexana- cyclounde- caphane-5³- yl)methane- sulfonamide | 492.3 |
| 46 | 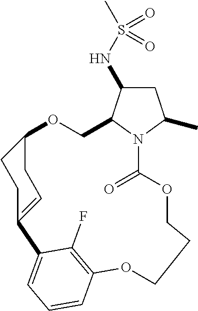 | N-((2¹S,2⁴S, 5²R,5³S,5⁵R)- 1²-fluoro-5⁵- methyl-6- oxo-3,7,11- trioxa-5(2,1)- pyrrolidina- 1(1,3)- benzena-2(1,4)- cyclohexana- cyclounde- caphan-2¹-en-5³- yl)methane- sulfonamide | 483.4 |
| 47 | 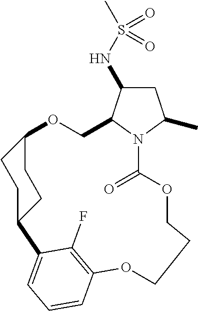 | N-((2¹S,2⁴S, 5²R,5³S,5⁵R)- 1²-fluoro-5⁵- methyl-6- oxo-3,7,11- trioxa-5(2,1)- pyrrolidina- 1(1,3)- benzena-2(1,4)- cyclohexana- cyclounde- caphane-5³- yl)methane- sulfonamide | 485.3 |
| 48 | 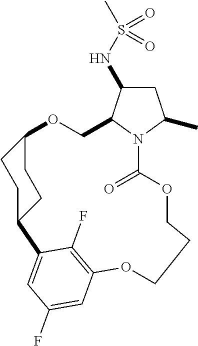 | N-((2¹S,2⁴S, 5²R,5³S,5⁵R)- 1²,1⁵-difluoro- 5⁵-methyl- 6-oxo- 3,7,11-trioxa- 5(2,1)- pyrrolidina- 1(1,3)- benzena-2(1,4)- cyclohexana- cyclounde- caphane-5³- yl)methane- sulfonamide | 503.2 |
| 49 | 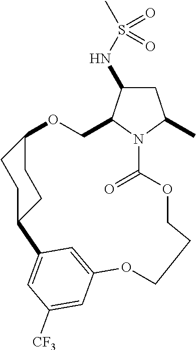 | N-((2¹S,2⁴S, 5²R,5³S,5⁵R)- 5⁵-methyl- 6-oxo-1⁵- (trifluoro- methyl)- 3,7,11- trioxa-5(2,1)- pyrrolidina- 1(1,3)-benzena- 2(1,4)- cyclohexana- cyclounde- caphane-5³- yl)methane- sulfonamide | 535.4 |
| 50 | 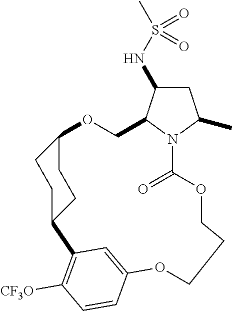 | N-((2¹S,2⁴S, 5²R,5³S,5⁵R)- 5⁵-methyl- 6-oxo-1⁶- (trifluoro- methoxy)- 3,7,11- trioxa-5(2,1)- pyrrolidina- 1(1,3)-benzena- 2(1,4)- cyclohexana- cyclounde- caphane-5³- yl)methane- sulfonamide | 551.3 |

### Example 51

### N-((2¹S,2⁴S,5²R,5³S,5⁵R)-5⁵-methyl-6-oxo-14-(trifluoromethyl)-3,7,11-trioxa-1(2,6)-pyridina-5(2,1)-pyrrolidina-2(1,4)-cyclohexanacycloundecaphane-5³-yl)methanesulfonamide

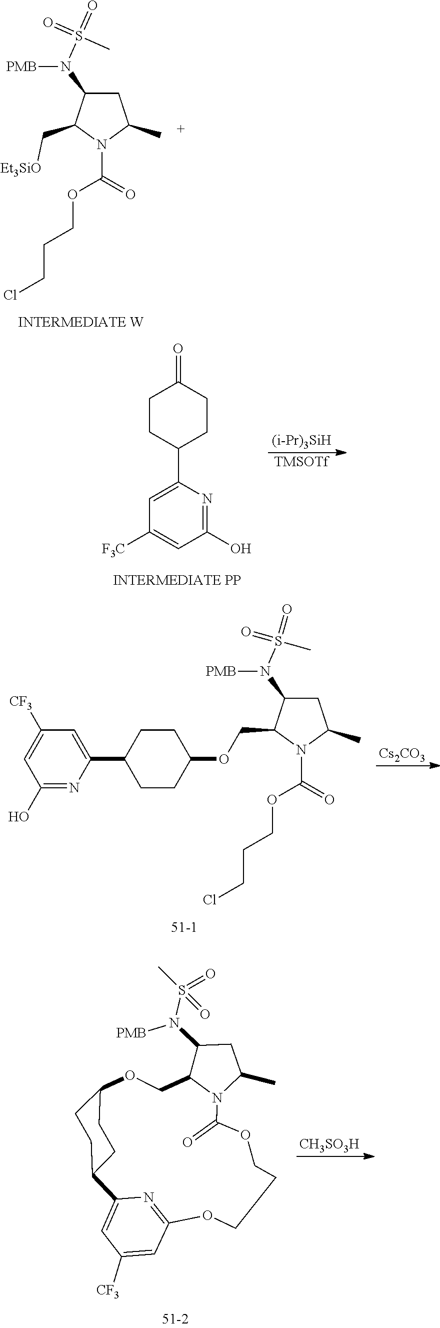

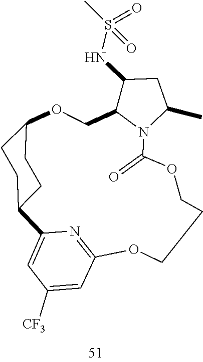 [SMILES: C[C@@H]1C[C@H](NS(C)(=O)=O)[C@@H]2CO[C@H]3CC[C@H](CC3)c3cc(C(F)(F)F)cc(n3)OCCCOC(=O)N12]

### Step 1: 3-chloropropyl (2R,3S,5R)-2-(((4-(6-hydroxy-4-(trifluoromethyl)pyridin-2yl)cyclohexyl) oxy)methyl)-3-(N-(4-methoxybenzyl)methylsulfonamido)-5-methylpyrrolidine-1-carboxylate (51-1)

To a solution of 3-chloropropyl (2R,3S,5R)-3-(N-(4-methoxybenzyl)methylsulfonamido)-5-methyl-2-(((triethylsilyl)oxy)methyl)pyrrolidine-1-carboxylate (INTERMEDIATE W)(61 mg, 0.108 mmol) and 4-(6-hydroxy-4-(trifluoromethyl)pyridin-2-yl)cyclohexan-1-one (INTERMEDIATE PP) (42.1 mg, 0.162 mmol) in acetonitrile (1 ml) at 0° C. was added triisopropylsilane (44.4 μl, 0.217 mmol) followed by trimethylsilyl trifluoromethanesulfonate (30 μL, 0.166 mmol) each as a solution in CH₂Cl₂ (0.5 ml) at 0° C. After 30 min, the reaction was quenched with 1 mL of sat. NaHCO₃ and the mixture was extracted with ethyl acetate (3×10 mL). The combined organic fractions were washed with brine (saturated, 10 mL), dried (Na₂SO₄), filtered and the solvent was evaporated under reduced pressure to give the title compound. MS: 692 [M+H]⁺.

### Step 2: N-(4-methoxybenzyl)-N-((2¹S,2⁴S,5²R,5³S,5⁵R)-5⁵-methyl-6-oxo-1⁴-(trifluoromethyl)-3,7,11-trioxa-1(2,6)-pyridina-5(2,1)-pyrrolidina-2(1,4)-cyclohexanacycloundecaphane-5³-yl)methanesulfonamide (51-2)

A suspension of 3-chloropropyl (2R,3S,5R)-2-((((1s,4S)-4-(6-hydroxy-4-(trifluoro methyl)pyridin-2-yl)cyclohexyl)oxy)methyl)-3-(N-(4methoxybenzyl)methylsulfonamido)-5-methylpyrrolidine-1-carboxylate (51-1) (62 mg, 0.090 mmol) and cesium carbonate (175 mg, 0.537 mmol) in DMF (8 mL) was heated at 60° C. After 2 hrs, the reaction mixture was filtered through a celite cake with ethyl acetate. The residue was purified by 2×1000 micron prep silica gel TLC plate [20×20 cm], eluting with 50% EtOAc/isohexane. The desired band was collected, filtered with ethyl acetate and concentrated under reduced pressure to give the title compound. MS: 656 [M+H]⁺.

### Step 3: N-((2¹S,2⁴S,5²R,5³S,5⁵R)-5⁵-methyl-6-oxo-1⁴-(trifluoromethyl)-3,7,11-trioxa-1(2,6)-pyridina-5(2,1)-pyrrolidina-2(1,4)-cyclohexanacycloundecaphane-5³-yl)methane Sulfonamide (51)

A solution of N-(4-methoxybenzyl)-N-((2¹S,2⁴S,5²R,5³S,5⁵R)-5⁵-methyl-6-oxo-14-(trifluoromethyl)-3,7,11-trioxa-1(2,6)-pyridina-5(2,1)-pyrrolidina-2(1,4)-cyclohexanacycloundecaphane-5³-yl)methanesulfonamide (51-2)(38 mg, 0.058 mmol) in dichloroethane (1 mL) was added methanesulfonic acid (0.075 ml, 1.159 mmol) at rt. After 10 min, the reaction mixture was concentrated under reduced pressure and the resulting residue was purified by preparative HPLC (C18, eluting MeCN/H2O with TFA modifier) to give the title compound. MS: 536 [M+H]+. ¹H NMR (400 MHz, Methanol-d4) δ 6.95 (s, 1H), 6.74 (s, 1H), 5.09 (d, J=5.3 Hz, 1H), 4.78 (d, J=11.2 Hz, 1H), 4.26-4.11 (m, 2H), 4.04-3.81 (m, 4H), 3.76 (s, 1H), 3.48-3.42 (m, 1H), 2.99 (s, 3H), 2.73 (t, J=11.3 Hz, 1H), 2.56 (s, 1H), 2.41 (dt, J=14.1, 7.2 Hz, 1H), 2.36-2.23 (m, 1H), 2.17 (d, J=14.9 Hz, 1H), 2.12-1.91 (m, 2H), 1.84 (q, J=11.5 Hz, 1H), 1.68 (s, 1H), 1.62-1.38 (m, 7H).

The following EXAMPLES 52-80 were prepared according to the general procedures herein and in an analogous manner to that used to synthesize EXAMPLE 51 using the appropriate intermediates. The intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H]⁺ |
| 52 | 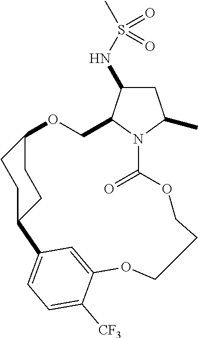 | N-((2¹S,2⁴S,5²R,5³S,5⁵R)-5⁵- methyl-6-oxo-1⁴- (trifluoromethyl)-3,7,11-trioxa- 5(2,1)-pyrrolidina-1(1,3)- benzena-2(1,4)- cyclohexanacycloundecaphane- 5³-yl)methanesulfonamide | 535.3 |
| 53 | 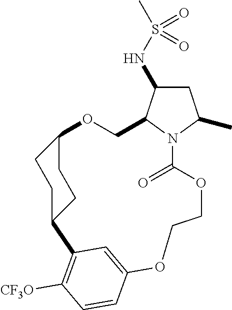 | N-((2¹S,2⁴S,5²R,5³S,5⁵R)-5⁵- methyl-6-oxo-16- (trifluoromethoxy)-3,7,10- trioxa-5(2,1)-pyrrolidina- 1(1,3)-benzena-2(1,4)- cyclohexanacyclodecaphane- 5³-yl)methanesulfonamide | 537.4 |
| 54 | 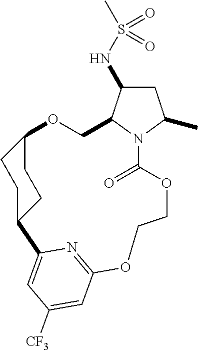 | N-((2¹S,2⁴S,5²R,5³S,5⁵R)-5⁵- methyl-6-oxo-1⁴- (trifluoromethyl)-3,7,10-trioxa- 1(2,6)-pyridina-5(2,1)- pyrrolidina-2(1,4)- cyclohexanacyclodecaphane- 5³-yl)methanesulfonamide | 522.4 |
| 55 | 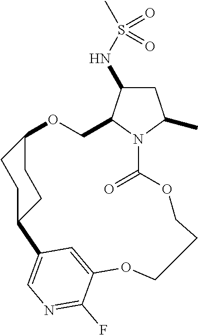 | N-((2¹S,2⁴S,5²R,5³S,5⁵R)-1⁶- fluoro-5⁵-methyl-6-oxo-3,7,11- trioxa-1(3,5)-pyridina-5(2,1)- pyrrolidina-2(1,4)- cyclohexanacycloundecaphane- 5³-yl)methanesulfonamide | 486.2 |
| 56 | 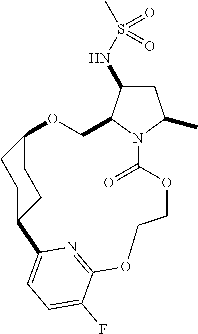 | N-((2¹S,2⁴S,5²R,5³S,5⁵R)-1⁵- fluoro-5⁵-methyl-6-oxo-3,7,10- trioxa-1(2,6)-pyridina-5(2,1)- pyrrolidina-2(1,4)- cyclohexanacyclodecaphane- 5³-yl)methanesulfonamide | 472.4 |
| 57 | 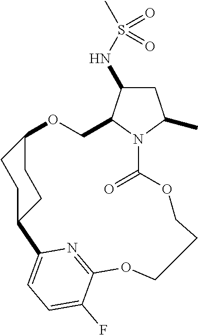 | N-((2¹S,2⁴S,5²R,5³S,5⁵R)-1⁵- fluoro-5⁵-methyl-6-oxo-3,7,11- trioxa-1(2,6)-pyridina-5(2,1)- pyrrolidina-2(1,4)- cyclohexanacycloundecaphane- 5³-yl)methanesulfonamide | 486.4 |
| 58 | 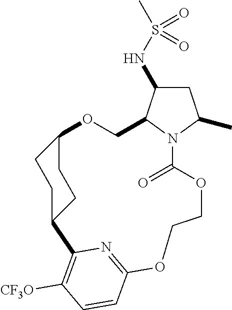 | N-((2¹S,2⁴S,5²R,5³S,5⁵R)-5⁵- methyl-6-oxo-1³- (trifluoromethoxy)-3,7,10- trioxa-1(2,6)-pyridina-5(2,1)- pyrrolidina-2(1,4)- cyclohexanacyclodecaphane- 5³-yl)methanesulfonamide | 538.5 |
| 59 | 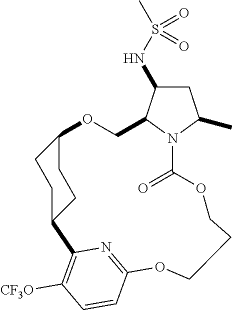 | N-((2¹S,2⁴S,5²R,5³S,5⁵R)-5⁵- methyl-6-oxo-1³- (trifluoromethoxy)-3,7,11- trioxa-1(2,6)-pyridina-5(2,1)- pyrrolidina-2(1,4)- cyclohexanacycloundecaphane- 5³-yl)methanesulfonamide | 552.5 |
| 60 | 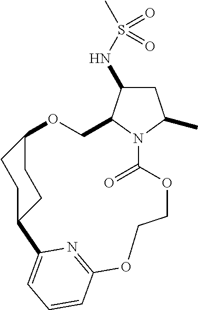 | N-((2¹S,2⁴S,5²R,5³S,5⁵R)-5⁵- methyl-6-oxo-3,7,10-trioxa- 1(2,6)-pyridina-5(2,1)- pyrrolidina-2(1,4)- cyclohexanacyclodecaphane- 5³-yl)methanesulfonamide | 454.4 |
| 61 | 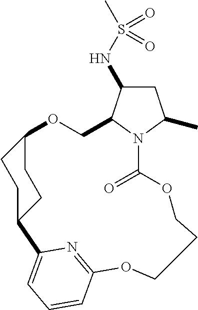 | N-((2¹S,2⁴S,5²R,5³S,5⁵R)-5⁵- methyl-6-oxo-3,7,11-trioxa- 1(2,6)-pyridina-5(2,1)- pyrrolidina-2(1,4)- cyclohexanacycloundecaphane- 5³-yl)methanesulfonamide | 468.4 |
| 62 | 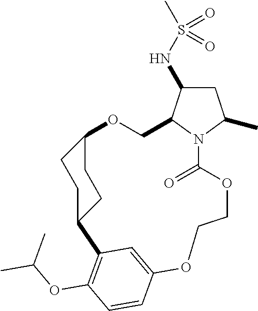 | N-((2¹S,2⁴S,5²R,5³S,5⁵R)-1⁶- isopropoxy-5⁵-methyl-6-oxo- 3,7,10-trioxa-5(2,1)- pyrrolidina-1(1,3)-benzena- 2(1,4)- cyclohexanacyclodecaphane- 5³-yl)methanesulfonamide | 511.4 |
| 63 | 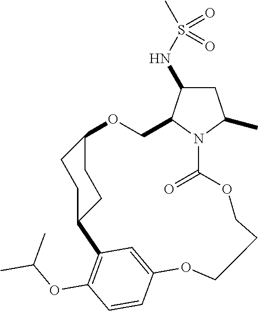 | N-((2¹S,2⁴S,5²R,5³S,5⁵R)-1⁶- isopropoxy-5⁵-methyl-6-oxo- 3,7,11-trioxa-5(2,1)- pyrrolidina-1(1,3)-benzena- 2(1,4)- cyclohexanacycloundecaphane- 5³-yl)methanesulfonamide | 525.4 |
| 64 | 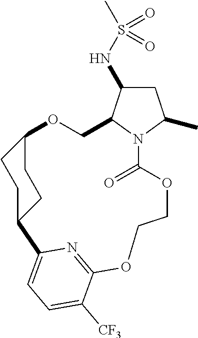 | N-((2¹S,2⁴S,5²R,5³S,5⁵R)-5⁵- methyl-6-oxo-1⁵- (trifluoromethyl)-3,7,10-trioxa- 1(2,6)-pyridina-5(2,1)- pyrrolidina-2(1,4)- cyclohexanacyclodecaphane- 5³-yl)methanesulfonamide | 522.4 |
| 65 | 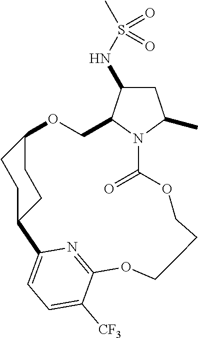 | N-((2¹S,2⁴S,5²R,5³S,5⁵R)-5⁵- methyl-6-oxo-1⁵- (trifluoromethyl)-3,7,11-trioxa- 1(2,6)-pyridina-5(2,1)- pyrrolidina-2(1,4)- cyclohexanacycloundecaphane- 5³-yl)methanesulfonamide | 536.4 |
| 66 | 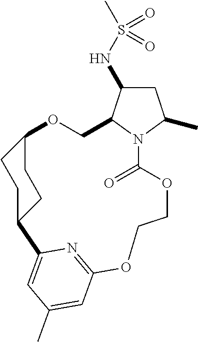 | N-((2¹S,2⁴S,5²R,5³S,5⁵R)- 1⁴,5⁵-dimethyl-6-oxo-3,7,10- trioxa-1(2,6)-pyridina-5(2,1)- pyrrolidina-2(1,4)- cyclohexanacyclodecaphane- 5³-yl)methanesulfonamide | 468.4 |
| 67 | 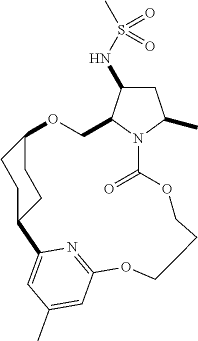 | N-((2¹S,2⁴S,5²R,5³S,5⁵R)- 1⁴,5⁵-dimethyl-6-oxo-3,7,11- trioxa-1(2,6)-pyridina-5(2,1)- pyrrolidina-2(1,4)- cyclohexanacycloundecaphane- 5³-yl)methanesulfonamide | 482.4 |
| 68 | 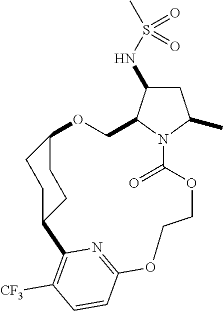 | N-((2¹S,2⁴S,5²R,5³S,5⁵R)-5⁵- methyl-6-oxo-1³- (trifluoromethyl)-3,7,10-trioxa- 1(2,6)-pyridina-5(2,1)- pyrrolidina-2(1,4)- cyclohexanacyclodecaphane- 5³-yl)methanesulfonamide | 522.4 |
| 69 | 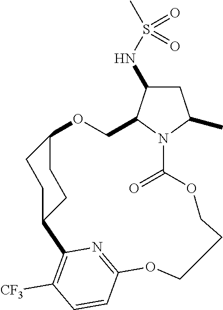 | N-((2¹S,2⁴S,5²R,5³S,5⁵R)-5⁵- methyl-6-oxo-1³- (trifluoromethyl)-3,7,11-trioxa- 1(2,6)-pyridina-5(2,1)- pyrrolidina-2(1,4)- cyclohexanacycloundecaphane- 5³-yl)methanesulfonamide | 536.4 |
| 70 | 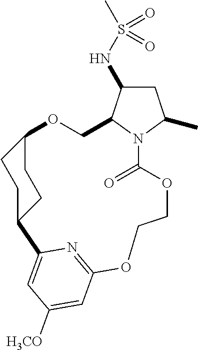 | N-((2¹S,2⁴S,5²R,5³S,5⁵R)-1⁴- methoxy-5⁵-methyl-6-oxo- 3,7,10-trioxa-1(2,6)-pyridina- 5(2,1)-pyrrolidina-2(1,4)- cyclohexanacyclodecaphane- 5³-yl)methanesulfonamide | 484.8 |
| 71 | 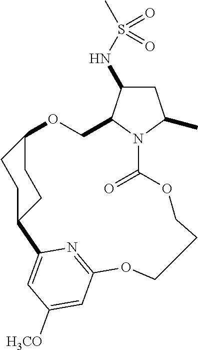 | N-((2¹S,2⁴S,5²R,5³S,5⁵R)-1⁴- methoxy-5⁵-methyl-6-oxo- 3,7,11-trioxa-1(2,6)-pyridina- 5(2,1)-pyrrolidina-2(1,4)- cyclohexanacycloundecaphane- 5³-yl)methanesulfonamide | 498.5 |
| 72 | 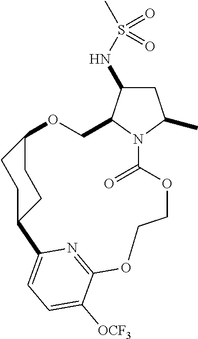 | N-((2¹S,2⁴S,5²R,5³S,5⁵R)-5⁵- methyl-6-oxo-1⁵- (trifluoromethoxy)-3,7,10- trioxa-1(2,6)-pyridina-5(2,1)- pyrrolidina-2(1,4)- cyclohexanacyclodecaphane- 5³-yl)methanesulfonamid | 538.4 |
| 73 | 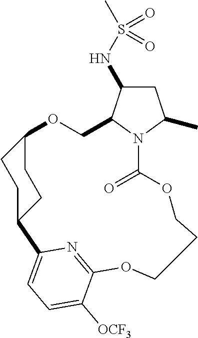 | N-((2¹S,2⁴S,5²R,5³S,5⁵R)-5⁵- methyl-6-oxo-1⁵- (trifluoromethoxy)-3,7,11- trioxa-1(2,6)-pyridina-5(2,1)- pyrrolidina-2(1,4)- cyclohexanacycloundecaphane- 5³-yl)methanesulfonamide | 552.4 |
| 74 | 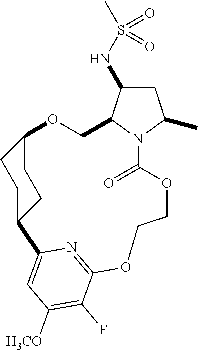 | N-((2¹S,2⁴S,5²R,5³S,5⁵R)-1⁵- fluoro-1⁴-methoxy-5⁵-methyl- 6-oxo-3,7,10-trioxa-1(2,6)- pyridina-5(2,1)-pyrrolidina- 2(1,4)-cyclohexanacyclode caphane-5³-yl)methane sulfonamide | 505.5 |
| 75 | 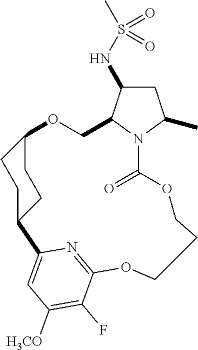 | N-((2¹S,2⁴S,5²R,5³S,5⁵R)-1⁴- fluoro-1³-methoxy-5⁵-methyl- 6-oxo-3,7,11-trioxa-1(2,6)- pyridina-5(2,1)-pyrrolidina- 2(1,4)-cyclohexanacyclounde caphane-5³-yl)methane sulfonamide | 516.5 |
| 76 | 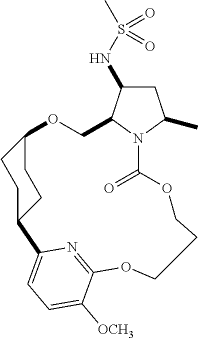 | N-((2¹S,2⁴S,5²R,5³S,5⁵R)-1⁵- methoxy-5⁵-methyl-6-oxo- 3,7,11-trioxa-1(2,6)-pyridina- 5(2,1)-pyrrolidina-2(1,4)- cyclohexanacycloundecaphane- 5³-yl)methanesulfonamide | 498.5 |
| 77 | 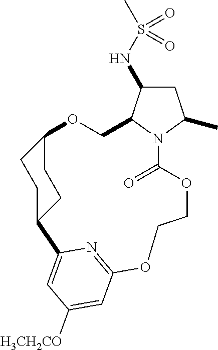 | N-((2¹S,2⁴S,5²R,5³S,5⁵R)-1⁴- ethoxy-5⁵-methyl-6-oxo- 3,7,10-trioxa-1(2,6)-pyridina- 5(2,1)-pyrrolidina-2(1,4)- cyclohexanacyclodecaphane- 5³-yl)methanesulfonamide | 498.6 |
| 78 | 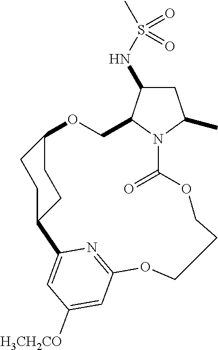 | N-((2¹S,2⁴S,5²R,5³S,5⁵R)-1⁴- ethoxy-5⁵-methyl-6-oxo- 3,7,11-trioxa-1(2,6)-pyridina- 5(2,1)-pyrrolidina-2(1,4)- cyclohexanacycloundecaphane- 5³-yl)methanesulfonamide | 511.5 |
| 79 | 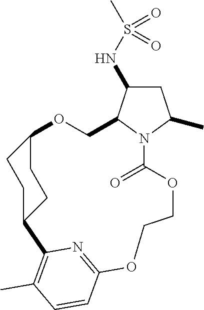 | N-((2¹S,2⁴S,5²R,5³S,5⁵R)- 1³,5⁵-dimethyl-6-oxo-3,7,10- trioxa-1(2,6)-pyridina-5(2,1)- pyrrolidina-2(1,4)- cyclohexanacyclodecaphane- 5³-yl)methanesulfonamide | 468.5 |
| 80 | 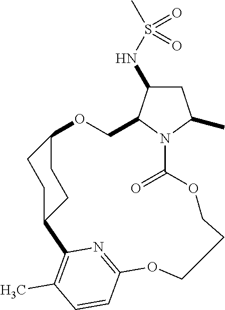 | N-((2¹S,2⁴S,5²R,5³S,5⁵R)- 1³,5⁵-dimethyl-6-oxo-3,7,11- trioxa-1(2,6)-pyridina-5(2,1)- pyrrolidina-2(1,4)- cyclohexanacycloundecaphane- 5³-yl)methanesulfonamide | 482.5 |
| 81 | 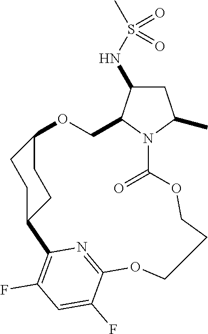 | N-((2¹S,2⁴S,5²R,5³S,5⁵R)- 1³,1⁵-difluoro-5⁵-methyl-6- oxo-3,7,11-trioxa-1(2,6)- pyridina-5(2,1)-pyrrolidina- 2(1,4)- cyclohexanacycloundecaphane- 5³-yl)methanesulfonamide | 504.4 |
| 82 | 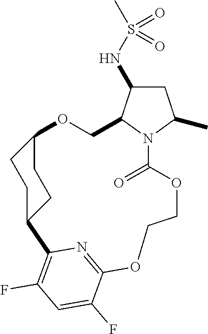 | N-((2¹S,2⁴S,5²R,5³S,5⁵R)- 1³,1⁵-difluoro-5⁵-methyl-6- oxo-3,7,10-trioxa-1(2,6)- pyridina-5(2,1)-pyrrolidina- 2(1,4)- cyclohexanacyclodecaphane- 5³-yl)methanesulfonamide | 490.4 |
| 83 | 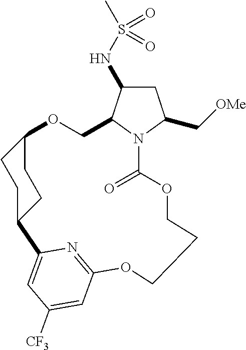 | N-((2¹S,2⁴S,5²R,5³S,5⁵S)-5⁵- (methoxymethyl)-6-oxo-1⁴- (trifluoromethyl)-3,7,11-trioxa- 1(2,6)-pyridina-5(2,1)- pyrrolidina-2(1,4)- cyclohexanacycloundecaphane- 5³-yl)methanesulfonamide | 566.4 |
| 84 | 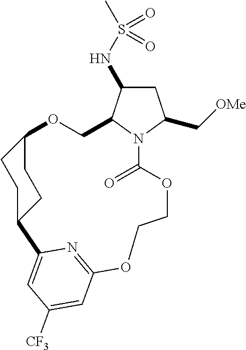 | N-((2¹S,2⁴S,5²R,5³S,5⁵S)-5⁵- (methoxymethyl)-6-oxo-1⁴- (trifluoromethyl)-3,7,10-trioxa- 1(2,6)-pyridina-5(2,1)- pyrrolidina-2(1,4)- cyclohexanacyclodecaphane- 5³-yl)methanesulfonamide | 552.4 |
| 85 | 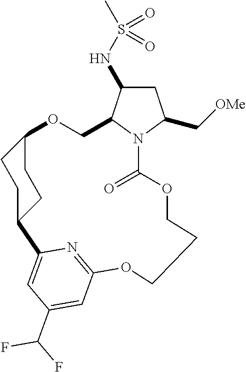 | N-((2¹S,2⁴S,5²R,5³S,5⁵S)-14- (difluoromethyl)-5⁵- (methoxymethyl)-6-oxo- 3,7,11-trioxa-1(2,6)-pyridina- 5(2,1)-pyrrolidina-2(1,4)- cyclohexanacycloundecaphane- 5³-yl)methanesulfonamide | 548.1 |
| 86 | 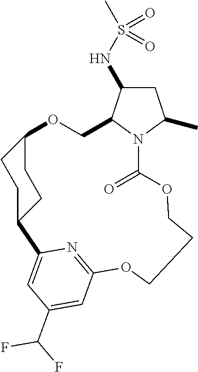 | N-((2¹S,2⁴S,5²R,5³S,5⁵R)-1⁴- (difluoromethyl)-5⁵-methyl-6- oxo-3,7,11-trioxa-1(2,6)- pyridina-5(2,1)-pyrrolidina- 2(1,4)- cyclohexanacycloundecaphane- 5³-yl)methanesulfonamide | 518.1 |
| 87 | 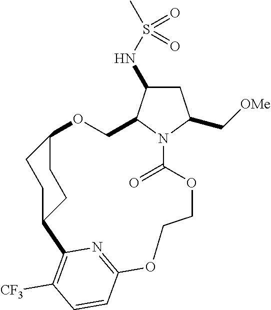 | N-((2¹S,2⁴S,5²R,5³S,5⁵S)-5⁵- (methoxymethyl)-6-oxo-1³- (trifluoromethyl)-3,7,10-trioxa- 1(2,6)-pyridina-5(2,1)- pyrrolidina-2(1,4)- cyclohexanacyclodecaphane- 5³-yl)methanesulfonamide | 552.1 |

### Example 88

### N-((2¹S,2⁴S,5²R,5³S,5⁵R)-1⁴-methoxy-5⁵-methyl-6-oxo-3,7-dioxa-11aza-1(2,6)-pyridina-5(2,1)-pyrrolidina-2(1,4)-cyclohexanacycloundecaphane-5³-yl)methanesulfonamide

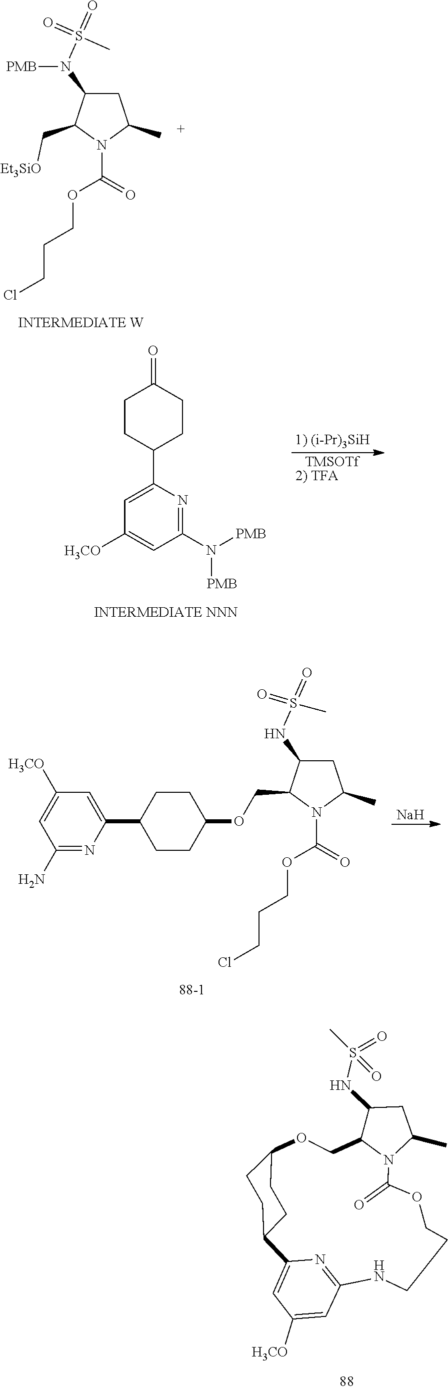

### Step 1: 3-chloropropyl (2R,3S,5R)-2-((((1s,4S)-4-(6-amino-4-methoxypyridin-2yl)cyclohexyl) oxy)methyl)-5-methyl-3-(methylsulfonamido)pyrrolidine-1-carboxylate (88-1)

To a solution of 3-chloropropyl (2R,3S,5R)-3-(N-(4-methoxybenzyl)methyl sulfonamido)-5-methyl-2-(((triethylsilyl)oxy)methyl)pyrrolidine-1-carboxylate (INTERMEDIATE W) (40 mg, 0.071 mmol) and 4-(6-(bis(4-methoxybenzyl)amino)-4-methoxypyridin-2-yl)cyclohexan-1-one (INTERMEDIATE NNN) (43.5 mg, 0.089 mmol) in acetonitrile (1 ml) charged in a 10 ml of microwave vial was cooled in an ice bath and added triisopropylsilane (30 μL, 0.146 mmol) as a solution of CH₂Cl₂ (300 μL) followed by trimethylsilyl trifluoromethanesulfonate (30 μL, 0.166 mmol) as a solution of CH₂Cl₂ (300 μL). The mixture was stirred at 0° C. After 70 min, TFA (109 μL, 1.43 mmol) was added to above mixture and stirred at rt. After 2 hrs, the reaction mixture was purified by preparative HPLC (C18, Water/Acetonitrile with TFA modifier) to obtain the title compound. MS: 533[M+H]⁺.

### Step 2: N-((2¹S,2⁴S,5²R,5³S,5⁵R)-1⁴-methoxy-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide (88)

To a solution of 3-chloropropyl (2R,3S,5R)-2-((((1s,4S)-4-(6-amino-4-methoxypyridin-2-yl)cyclohexyl)oxy)methyl)-5-methyl-3-(methylsulfonamido)pyrrolidine-1-carboxylate (88-1) (38 mg, 0.071 mmol) in DMF (8 ml) was added NaH (22.81 mg, 0.570 mmol). The resulting suspension was heated at 70° C. for 2 hrs. The mixture was quenched with water (10 mL) and the mixture was extracted with ethyl acetate (3×10 mL). The combined organic fractions were dried (Na₂SO₄), filtered and the solvent was evaporated under reduced pressure. The residue was purified by preparative HPLC (C18, Water/AcetonitExle with TFAmodifier) to obtain the title compound. MS: 497 [M+H]⁺. ¹H NMR (500 MHz, Methanol-d₄) δ 5.95 (s, 1H), 5.74 (s, 1H), 4.73 (s, 1H), 4.20 (s, 1H), 4.10 (s, 1H), 4.05-3.85 (m, 3H), 3.74 (d, J=13.2 Hz, 5H), 3.45 (d, J=16.7 Hz, 1H), 3.37 (s, 1H), 3.01 (s, 4H), 2.43 (s, 4H), 2.26-2.11 (m, 2H), 2.01 (d, J=21.1 Hz, 2H), 1.88 (d, J=9.5 Hz, 1H), 1.49-1.22 (in, 8H).

The following EXAMPLES 89-95 were prepared according to the general procedures herein and in an analogous manner to that used to synthesize EXAMPLE 88 using the appropriate intermediates. The ketone intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H]⁺ |
| 89 | 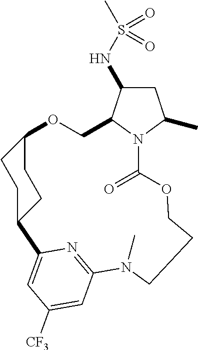 | N-((2¹S,2⁴S,5²R,5³S,5⁵R)- 5⁵,1¹-dimethyl-6-oxo-1⁴- (trifluoromethyl)-3,7-dioxa-11- aza-1(2,6)-pyridina-5(2,1)- pyrrolidina-2(1,4)- cyclohexanacycloundecaphane- 5³-yl)methanesulfonamide | 549.5 |
| 90 | 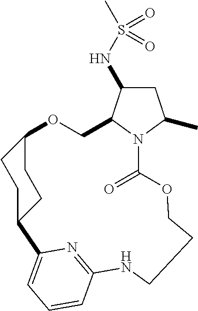 | N-((2¹S,2⁴S,5²R,5³S,5⁵R)-5⁵- methyl-6-oxo-3,7-dioxa-11- aza-1(2,6)-pyridina-5(2,1)- pyrrolidina-2(1,4)- cyclohexanacycloundecaphane- 5³-yl)methanesulfonamide | 467.4 |
| 91 | 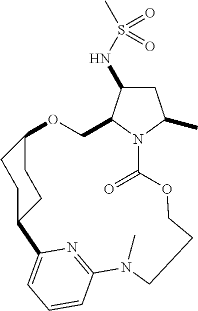 | N-((2¹S,2⁴S,5²R, 5³S,5⁵R)-5⁵,¹¹- dimethyl-6-oxo-3,7-dioxa-11- aza-1(2,6)-pyridina-5(2,1)- pyrrolidina-2(1,4)- cyclohexanacycloundecaphane- 5³-yl)methanesulfonamide | 481.4 |
| 92 | 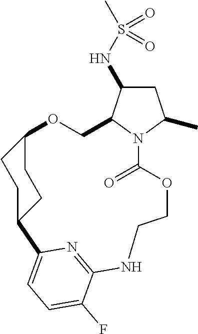 | N-((2¹S,2⁴S,5²R,5³S,5⁵R)-1⁵- fluoro-5⁵-methyl-6-oxo-3,7- dioxa-10-aza-1(2,6)-pyridina- 5(2,1)-pyrrolidina-2(1,4)- cyclohexanacyclodecaphane- 5³-yl)methanesulfonamide | 471.5 |
| 93 | 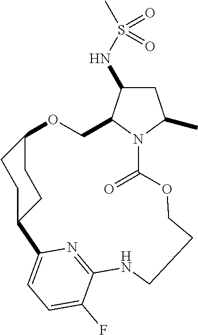 | N-((2¹S,2⁴S,5²R,5³S,5⁵R)-1⁵- fluoro-5⁵-methyl-6-oxo-3,7- dioxa-11-aza-1(2,6)-pyridina- 5(2,1)-pyrrolidina-2(1,4)- cyclohexanacycloundecaphane- 5³-yl)methanesulfonamide | 485.3 |
| 94 | 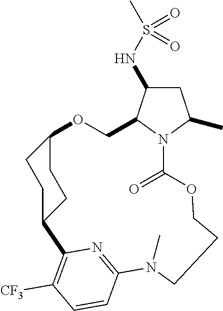 | N-((2¹S,2⁴S,5²R,5³S,5⁵R)- 5⁵,1¹-dimethyl-6-oxo-1³- (trifluoromethyl)-3,7-dioxa-11- aza-1(2,6)-pyridina-5(2,1)- pyrrolidina-2(1,4)- cyclohexanacycloundecaphane- 5³-yl)methanesulfonamid | 549.5 |
| 95 | 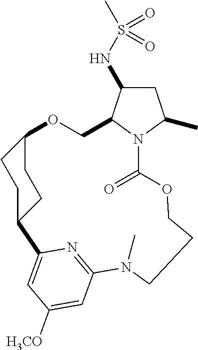 | N-((2¹S,2⁴S,5²R,5³S,5⁵R)-1⁴- methoxy-5⁵,1¹-dimethyl-6-oxo- 3,7-dioxa-11-aza-1(2,6)- pyridina-5(2,1)-pyrrolidina- 2(1,4)- cyclohexanacycloundecaphane- 5³-yl)methanesulfonamide | 511.5 |

### Example 96

### N-((2¹R,2⁴R,5²R,5³S,5⁵S)-5⁵-(hydroxymethyl)-6-oxo-1³-(trifluoromethyl)-3.7,10-trioxa-1(2,6)-pyridina-5 (2,1)-pyrrolidina-2(1,4)-cyclohexanacyclodecaphane-5³-yl)methanesulfonamide

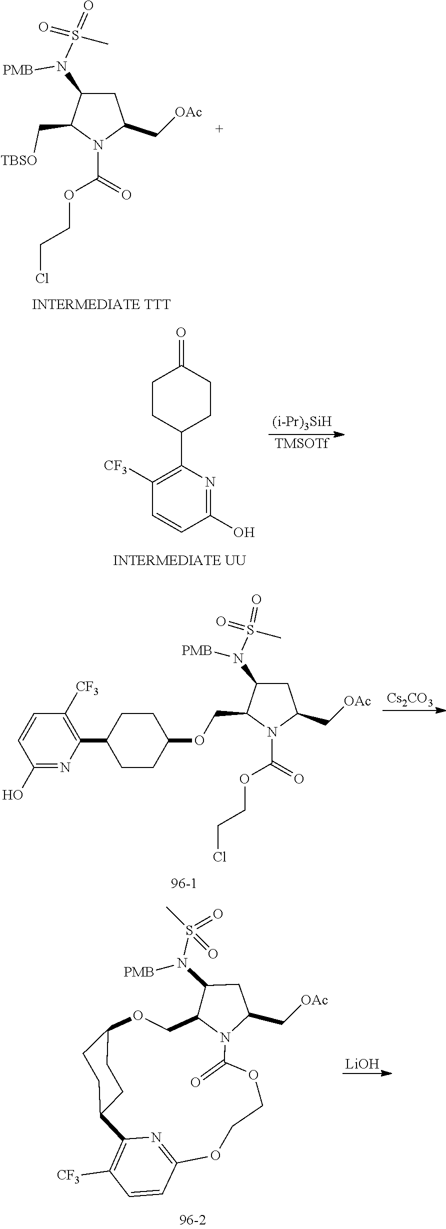

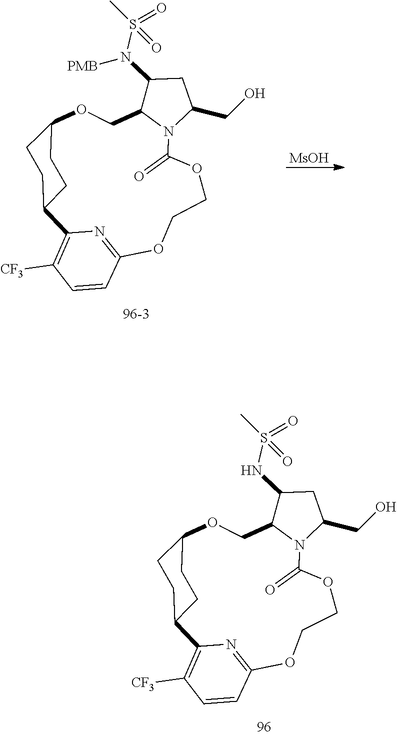

### Step 1: 2-chloroethyl (2R,3S,5S)-5-(acetoxymethyl)-2-((((1s,4S)-4-(6-hydroxy-3-(trifluoromethyl)pyridin-2-yl)cyclohexyl)oxy)methyl)-3-(N-(4-methoxybenzyl)methylsulfonamido)pyrrolidine-1-carboxylate (96-1)

To a mixture of 2-chloroethyl (2R,3S,5S)-5-(acetoxymethyl)-2-(((tert-butyldimethylsilyl)oxy)methyl)-3-(N-(4-methoxybenzyl)methylsulfonamido)pyrrolidine-1-carboxylate (INTERMEDIATE TTT) (150 mg, 0.247 mmol) in MeCN (2059 μl)/DCM (412 μl) at −20° C. was added 4-(6-hydroxy-3-(trifluoromethyl)pyridin-2-yl)cyclohexan-1-one (INTERMEDIATE UU) (77 mg, 0.296 mmol) and triisopropylsilane (102 μl, 0.494 mmol). TMS-OTf (44.6 μl, 0.247 mmol) was added dropwise and the mixture stirred for 10 min before quenching with a saturated solution of NaHCO₃(10 mL), extract with EtOAc (3×@ 10 mL), dry over Na2SO4, and concentrate to give the title compound. MS: 736.5 [M+H]+.

### Step 2: ((2¹R,2⁴R,5²R,5³S,5⁵S)-5³-(N-(4-methoxybenzyl)methylsulfonamido)-6-oxo-13-(trifluoromethyl)-3,7,10-trioxa-1(2,6)-pyridina-5(2,1)-pyrrolidina-2(1,4)-cyclohexanacyclodecaphane-5⁵-yl)methyl acetate (96-2)

To a mixture of 2-chloroethyl (2R,3S,5S)-5-(acetoxymethyl)-2-((((1s,4S)-4-(6-hydroxy-3-(trifluoromethyl)pyridin-2-yl)cyclohexyl)oxy)methyl)-3-(N-(4-methoxybenzyl)methylsulfonamido)pyrrolidine-1-carboxylate (96-1) (182 mg, 0.247 mmol) in DMF (16.500 ml) at ambient temperature was added cesium carbonate (242 mg, 0.742 mmol). The mixture was heated to 80° C. and stirred for 1 hour. The mixture was cooled, filtered, and concentrated. The resulting residue was purified using silica column chromatography (5% to 100% 3:1 EtOAc:EtOH/hexanes) to obtain the title compound. MS: 700.5 [M+H]+.

### Step 3: N-((2¹R,2⁴R⁵²R,5³S,5⁵S)-5⁵-(hydroxymethyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)-N-(4-methoxybenzyl)methanesulfonamide (96-3)

To a mixture of ((21R,24R,52R,53S,55S)-53-(N-(4-methoxybenzyl)methylsulfonamido)-6-oxo-13-(trifluoromethyl)-3,7,10-trioxa-1(2,6)-pyridina-5(2,1)-pyrrolidina-2(1,4)-cyclohexanacyclodecaphane-55-yl)methyl acetate (96-2) (130 mg, 0.186 mmol) in THF (464 μl)/Methanol (464 μl) was added LiOH (124 μl, 0.372 mmol). The mixture stirred for one hour before concentrating. The resulting residue was purified using C18 column chromatography (5% to 100% MeCN (0.05% TFA)/H2O (0.05% TFA)) to obtain the title compound. MS: 659.2 [M+H]⁺.

### Step 4: N-((2¹R,2⁴R,5²R,5³S,5⁵S)-5⁵-(hydroxymethyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide (96)

To a mixture of N-((21R,24R,52R,53S,55S)-55-(hydroxymethyl)-6-oxo-13-(trifluoromethyl)-3,7,10-trioxa-1(2,6)-pyridina-5(2,1)-pyrrolidina-2(1,4)-cyclohexanacyclodecaphane-53-yl)-N-(4-methoxybenzyl)methanesulfonamide (96-3) (15 mg, 0.023 mmol) in DCM (228 μl) at ambient temperature was added methanesulfonic acid (14.81 μl, 0.228 mmol). The mixture stirred for 1 hour before concentrating. The resulting residue was purified using C18 column chromatography (5% to 100% MeCN (0.05% TFA)/H2O (0.05% TFA)) to obtain the title compound. MS: 538.8 [M+H]⁺. 1H NMR (500 MHz, Chloroform-d) δ 7.78 (d, J=8.5 Hz, 1H), 6.63 (d, J=8.6 Hz, 1H), 6.46 (s, 1H), 4.97-4.83 (m, 2H), 4.68-4.56 (m, 1H), 4.49 (s, 1H), 4.28 (s, 2H), 4.15 (dd, J=18.3, 10.8 Hz, 2H), 4.08 (s, 1H), 3.93 (s, 1H), 3.54 (d, J=8.8 Hz, 1H), 3.49 (d, J=11.9 Hz, 1H), 3.08 (t, J=11.5 Hz, 1H), 3.02 (s, 3H), 2.58 (s, 2H), 2.28 (d, J=12.3 Hz, 2H), 2.09 (dt, J=25.9, 11.9 Hz, 3H), 1.95 (d, J=13.9 Hz, 2H), 1.76-1.56 (m, 2H), 1.47 (dd, J=35.5, 14.2 Hz, 2H).

The following EXAMPLE 97 were prepared according to the general procedures herein and in an analogous manner to that used to synthesize EXAMPLE 96 using the appropriate intermediates. The intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H]⁺ |
| 97 | 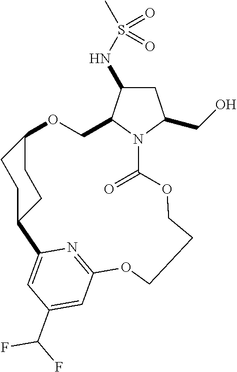 | N-((2¹S,2⁴S,5²R,5³S,5⁵S)-1⁴- (difluoromethyl)-5⁵- (hydroxymethyl)-6-oxo-3,7,11- trioxa-1(2,6)-pyridina-5(2,1)- pyrrolidina-2(1,4)- cyclohexanacycloundecaphane- 5³-yl)methanesulfonamide | 534.8 |

### Example 98

### N-((2¹R,2⁴R,5²R,5³S,5⁵S)-5⁵-((dimethylamino)methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide

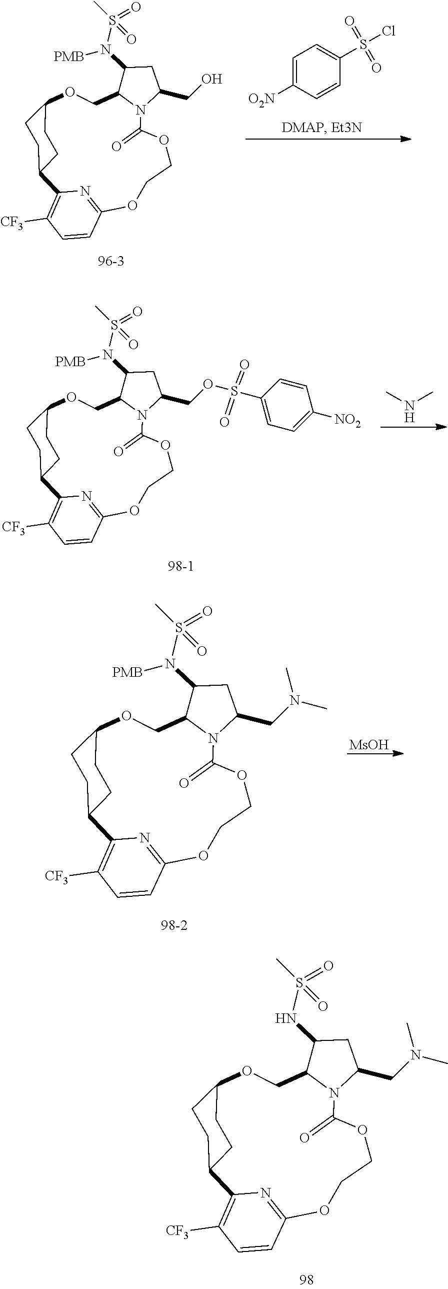

### Step 1: ((2¹R,2⁴R,5²R,5³S,55S)-5³-(N-(4-methoxybenzyl)methylsulfonamido)-6-oxo-13-(trifluoromethyl)-3,7,10-trioxa-1(2,6)-pyridina-5(2,1)-pyrrolidina-2(1,4)-cyclohexanacyclodecaphane-5⁵-yl)methyl 4-nitrobenzenesulfonate (98-1)

To a mixture of N-((21R,24R,52R,53S,55S)-55-(hydroxymethyl)-6-oxo-13-(trifluoromethyl)-3,7,10-trioxa-1(2,6)-pyridina-5(2,1)-pyrrolidina-2(1,4)-cyclohexanacyclodecaphane-53-yl)-N-(4-methoxybenzyl)methanesulfonamide (96-3) (90 mg, 0.137 mmol) in DCM (912 μl) at ambient temperature was added TRIETHYLAMINE (28.6 μl, 0.205 mmol), DMAP (3.34 mg, 0.027 mmol), and 4-nitrobenzenesulfonyl chloride (39.4 mg, 0.178 mmol). The mixture stirred for 3 hours before concentrating. The resulting mixture was purified using silica column chromatography (2% to 100% 3:1 EtOAc:EtOH/hexanes) to obtain the title compound. MS: 844.5 [M+H]⁺.

### Step 2: N-((2¹R,2⁴R,5²R,5³S,5⁵S)-5⁵-((dimethylamino) methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)-N-(4-methoxybenzyl)methanesulfonamide (98-2)

To a mixture of ((21R,24R,52R,53S,55S)-53-(N-(4-methoxybenzyl)methylsulfonamido)-6-oxo-13-(trifluoromethyl)-3,7,10-trioxa-1(2,6)-pyridina-5(2,1)-pyrrolidina-2(1,4)-cyclohexanacyclodecaphane-55-yl)methyl 4-nitrobenzenesulfonate (98-1) (35 mg, 0.042 mmol) in DMF (208 μl) at ambient temperature was added dimethylamine (104 μl, 0.208 mmol) in THF. The mixture was heated to 80° C. and stirred for 4 hours. The mixture was cooled and purified directly using C₁₈ column chromatography (5% to 100% MeCN (0.05% TFA)/H2O (0.05% TFA)) to obtain the title compound. MS: 685.5 [M+H]⁺.

### Step 3: N-((2¹R,2⁴R,5²R,5³S,55S)-5⁵-((dimethylamino)methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide (98)

To a mixture of N-((21R,24R,52R,53S,55S)-55-((dimethylamino)methyl)-6-oxo-13-(trifluoromethyl)-3,7,10-trioxa-1(2,6)-pyridina-5(2,1)-pyrrolidina-2(1,4)-cyclohexanacyclodecaphane-53-yl)-N-(4-methoxybenzyl)methanesulfonamide (98-2) (25 mg, 0.037 mmol) in DCM (365 μl) at ambient temperature was added methanesulfonic acid (23.71 μl, 0.365 mmol). The mixture stirred for 1 hour before concentrating. The resulting residue was purified using C₁₈ column chromatography (5% to 100% MeCN (0.05% TFA)/H2O (0.05% TFA)) to obtain the title compound. MS: 565.5 [M+H]⁺. 1H NMR (500 MHz, DMSO-d6) δ 7.96 (d, J=8.6 Hz, 1H), 7.48 (d, J=39.4 Hz, 1H), 6.81 (d, J=8.6 Hz, 1H), 4.59 (s, 1H), 4.28 (s, 2H), 4.23-3.82 (m, 7H), 3.66 (s, 3H), 3.42-3.07 (m, 3H), 2.99 (s, 3H), 2.92-2.76 (m, 3H), 2.76-2.56 (m, 3H), 2.51 (s, 7H), 2.18-1.81 (m, 4H), 1.56-1.19 (m, 5H).

The following EXAMPLES 99-102 were prepared according to the general procedures herein and in an analogous manner to that used to synthesize EXAMPLE 98 using the appropriate intermediates. The intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H]⁺ |
| 99 | 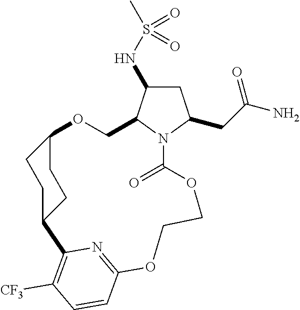 | 2-((2¹R,2⁴R,5²R,5³S,5⁵S)-5³- (methylsulfonamido)-6-oxo-1³- (trifluoromethyl)-3,7,10- trioxa- 1(2,6)-pyridina-5(2,1)- pyrrolidina-2(1,4)- cyclohexanacyclodecaphane- 5⁵-yl)acetamide | 565.4 |
| 100 | 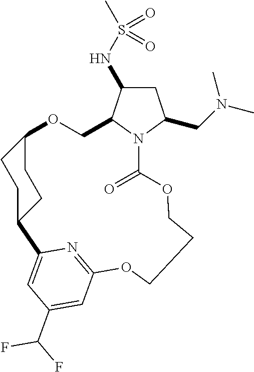 | N-((2¹S,2⁴S,5²R,5³S,5⁵S)-1⁴- (difluoromethyl)-5⁵- ((dimethylamino)methyl)-6- oxo-3,7,11-trioxa-1(2,6)- pyridina-5(2,1)-pyrrolidina- 2(1,4)- cyclohexanacycloundecaphane- 5³-yl)methanesulfonamide | 561.9 |
| 101 | 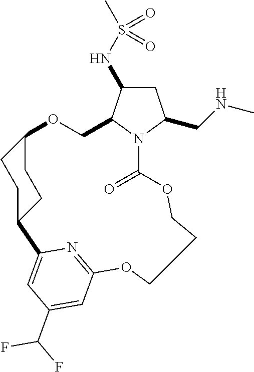 | N-((2¹S,2⁴S,5²R,5³S,5⁵S)-1⁴- (difluoromethyl)-5⁵- ((methylamino)methyl)-6-oxo- 3,7,11-trioxa-1(2,6)-pyridina- 5(2,1)-pyrrolidina-2(1,4)- cyclohexanacycloundecaphane- 5³-yl)methanesulfonamide | 547.9 |
| 102 | 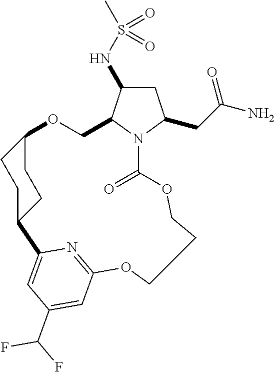 | 2-((2¹S,2⁴S,5²R,5³S,5⁵S)-1⁴- (difluoromethyl)-5³- (methylsulfonamido)-6-oxo- 3,7,11-trioxa-1(2,6)-pyridina- 5(2,1)-pyrrolidina-2(1,4)- cyclohexanacycloundecaphane- 5⁵-yl)acetamide | 561.9 |

### Example 103

### N-((2¹R,2⁴R,5²R,5³S,5⁵S)-5⁵-((dimethylamino)methyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide

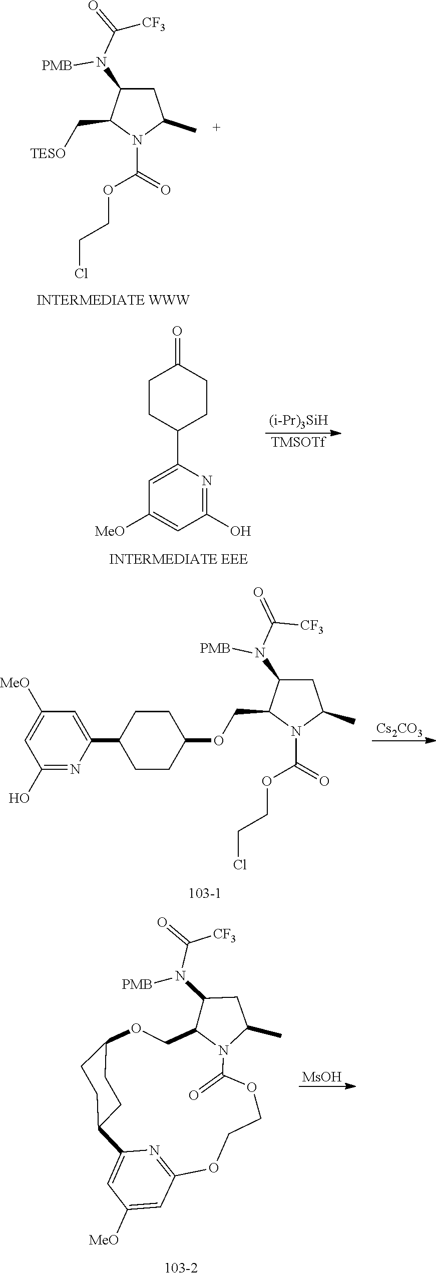

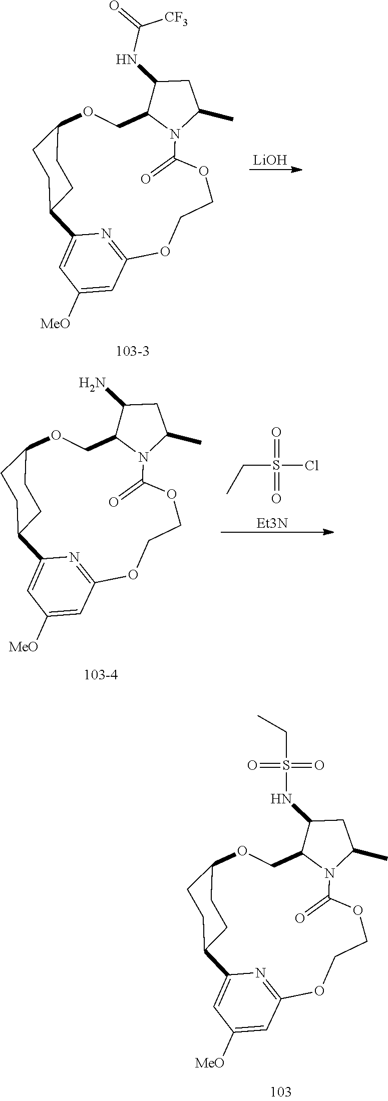

### Step 1: 2-chloroethyl (2R,3S,5R)-2-((((1s,4S)-4-(6-hydroxy-4-methoxypyridin-2-yl)cyclohexyl)oxy)methyl)-5-methyl-3-(2,2,2-trifluoro-N-(4-methoxybenzyl)acetamido)pyrrolidine-1-carboxylate (103-1)

To a mixture of 2-chloroethyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (INTERMEDIATE WWW) (250 mg, 0.441 mmol) in MeCN (3674 μl)/DCM (735 μl) at −20° C. was added 4-(6-hydroxy-4-methoxypyridin-2-yl)cyclohexan-1-one (INTERMEDIATE EEE) (127 mg, 0.573 mmol) and triisopropylsilane (181 μl, 0.882 mmol). TMS-OTf (80 μl, 0.441 mmol) was added dropwise and the mixture stirred for 10 min before quenching with a saturated solution of NaHCO₃(10 mL), extract with EtOAc (3×@ 10 mL), dry over Na2SO4, and concentrate to obtain the title compound. MS: 659.2 [M+H]+.

### Step 2: 2,2,2-trifluoro-N-((2¹R,2⁴R,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)-N-(4-methoxybenzyl)acetamide (103-2)

To a mixture of 2-chloroethyl (2R,3S,5R)-2-((((1s,4S)-4-(6-hydroxy-4-methoxypyridin-2-yl)cyclohexyl)oxy)methyl)-5-methyl-3-(2,2,2-trifluoro-N-(4-methoxybenzyl)acetamido)pyrrolidine-1-carboxylate (103-1) (290 mg, 0.441 mmol) in DMF (29.400 mL) at ambient temperature was added Cs2CO3 (431 mg, 1.322 mmol). The mixture was heated to 60° C. and stirred for 1 hour. The mixture was cooled, filtered, and concentrated. The resulting residue was purified using C₁₈ column chromatography (5% to 100% MeCN (0.05% TFA)/H2O (0.05% TFA)) to obtain the title compound. MS: 622.5 [M+H]+.

### Step 3: 2,2,2-trifluoro-N-((2¹R,2⁴R,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)acetamide (103-3)

To a mixture of 2,2,2-trifluoro-N-((21R,24R,52R,53S,55R)-14-methoxy-55-methyl-6-oxo-3,7,10-trioxa-1(2,6)-pyridina-5(2,1)-pyrrolidina-2(1,4)-cyclohexanacyclodecaphane-53-yl)-N-(4-methoxybenzyl)acetamide (103-2) (75 mg, 0.121 mmol) in DCM (1206 μl) at ambient temperature was added methanesulfonic acid (78 μl, 1.206 mmol). The mixture stirred for 1 hour before concentrating. The resulting residue was purified using C₁₈ column chromatography (5% to 100% MeCN (0.05% TFA)/H2O (0.05% TFA)) to obtain the title compound. MS: 502.7 [M+H]⁺.

### Step 4: (2¹R,2⁴R,5²R,5³S,5⁵R)-5³-amino-1⁴-methoxy-5⁵-methyl-3,7,10-trioxa-1(2,6)-pyridina-5(2,1)-pyrrolidina-2(1,4)-cyclohexanacyclodecaphan-6-one (103-4)

To a mixture of 2,2,2-trifluoro-N-((21R,24R,52R,53S,55R)-14-methoxy-55-methyl-6-oxo-3,7,10-trioxa-1(2,6)-pyridina-5(2,1)-pyrrolidina-2(1,4)-cyclohexanacyclodecaphane-53-yl)acetamide (103-3) (49 mg, 0.098 mmol) in THF (489 μl)/MeOH (489 μl) was added 3.0 M LiOH (147 μl, 0.442 mmol). The mixture was heated to 50° C. and stirred for 3 hours before cooling and concentrating. The resulting residue was filtered through a resin exchange column to capture the title compound. MS: 406.5 [M+H]⁺.

### Step 5: N-((2¹R,2⁴R,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)ethanesulfonamide (103)

To a mixture of (21R,24R,52R,53S,55R)-53-amino-14-methoxy-55-methyl-3,7,10-trioxa-1(2,6)-pyridina-5(2,1)-pyrrolidina-2(1,4)-cyclohexanacyclodecaphan-6-one (103-4) (8 mg, 0.020 mmol) in DCM (197 μl) was added TRIETHYLAMINE (13.75 μl, 0.099 mmol) and ethanesulfonyl chloride (5.07 mg, 0.039 mmol). The mixture was stirred for 1 hour before concentrating. The resulting residue was purified using C₁₈ column chromatography (5% to 100% MeCN (0.05% TFA)/H2O (0.05% TFA)) to obtain the title compound. MS: 498.5 [M+H]⁺. 1H NMR (500 MHz, Chloroform-d) δ 6.31 (s, 1H), 6.05 (s, 1H), 5.52 (s, 1H), 5.44 (d, J=7.2 Hz, 1H), 4.81 (s, 1H), 4.37 (s, 1H), 4.14 (d, J=32.1 Hz, 4H), 3.85 (d, J=6.4 Hz, 2H), 3.82 (d, J=12.6 Hz, 4H), 3.40 (d, J=7.9 Hz, 1H), 3.07 (q, J=7.3 Hz, 2H), 2.42-2.29 (m, 2H), 2.22 (d, J=13.3 Hz, 2H), 2.01 (dt, J=27.2, 9.2 Hz, 3H), 1.85 (d, J=11.6 Hz, 1H), 1.75 (s, 1H), 1.59 (dd, J=29.5, 14.2 Hz, 2H), 1.48 (d, J=14.4 Hz, 2H), 1.40 (t, J=7.3 Hz, 4H).

The following EXAMPLES 104-108 were prepared according to the general procedures herein and in an analogous manner to that used to synthesize EXAMPLE 103 using the appropriate intermediates. The intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H\|⁺ |
| 104 | 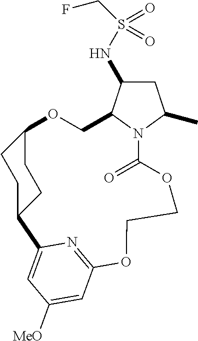 | 1-fluoro-N- ((2¹R,2⁴R,5²R,5³S,5⁵R)- 1⁴-methoxy-5⁵-methyl- 6-oxo-3,7,10-trioxa-1(2, 6)-pyridina-5(2,1)-pyrro- lidina-2(1,4)-cyclohexana- cyclodecaphane-5³- yl)methanesulfonamide | 502.5 |
| 105 | 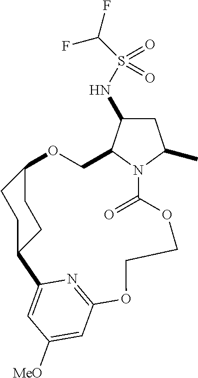 | 1,1-difluoro- N-((2¹R,2⁴R,5²R,5³S,5⁵R)- 14-methoxy-5⁵-methyl- 6-oxo-3,7,10-trioxa- 1(2,6)-pyridina-5(2,1)- pyrrolidina-2(1,4)-cyclohex- anacyclodecaphane-5³- yl)methanesulfonamide | 520.5 |
| 106 | 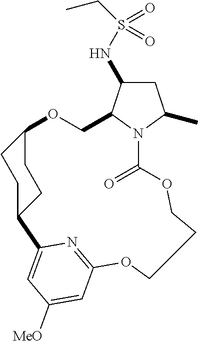 | N-((2¹R,2⁴R,5²R,5³S,5⁵R)- 1⁴-methoxy-5⁵-methyl- 6-oxo-3,7,11-trioxa- 1(2,6)-pyridina-5(2,1)- pyrrolidina-2(1,4)-cyclohex- anacycloundecaphane-5³- yl)ethanesulfonamide | 512.5 |
| 107 | 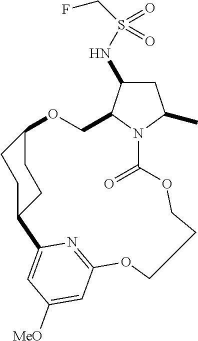 | 1-fluoro-N-((2¹R,2⁴R,5²R, 5³S,5⁵R)-1⁴-methoxy- 5⁵-methyl-6-oxo-3,7,11- trioxa-1(2,6)-pyridina- 5(2,1)-pyrrolidina-2(1,4)- cyclohexanacycloundecaphane- 5³-yl)methanesulfonamide | 516.5 |
| 108 | 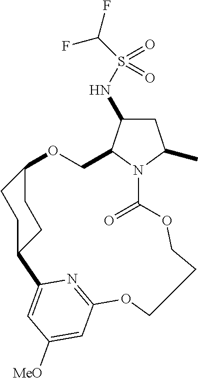 | 1,1-difluoro-N- ((2¹R,2⁴R,5²R,5³S,5⁵R)-14- methoxy-5⁵-methyl-6-oxo- 3,7,11-trioxa-1(2,6)-pyridina- 5(2,1)-pyrrolidina-2(1,4)- cyclohexanacycloundecaphane- 5³-yl)methanesulfonamide | 534.5 |

### Example 109

### N-((2'S,2⁴S,5²R,5³S,5⁵R)-5⁵-methyl-6-oxo-1⁴-(trifluoromethyl)-3,7-dioxa-1(2,6)-pyridina-5(2,1)-pyrrolidina-2(1,4)-cyclohexanacycloundecaphane-5³-yl)methanesulfonamide

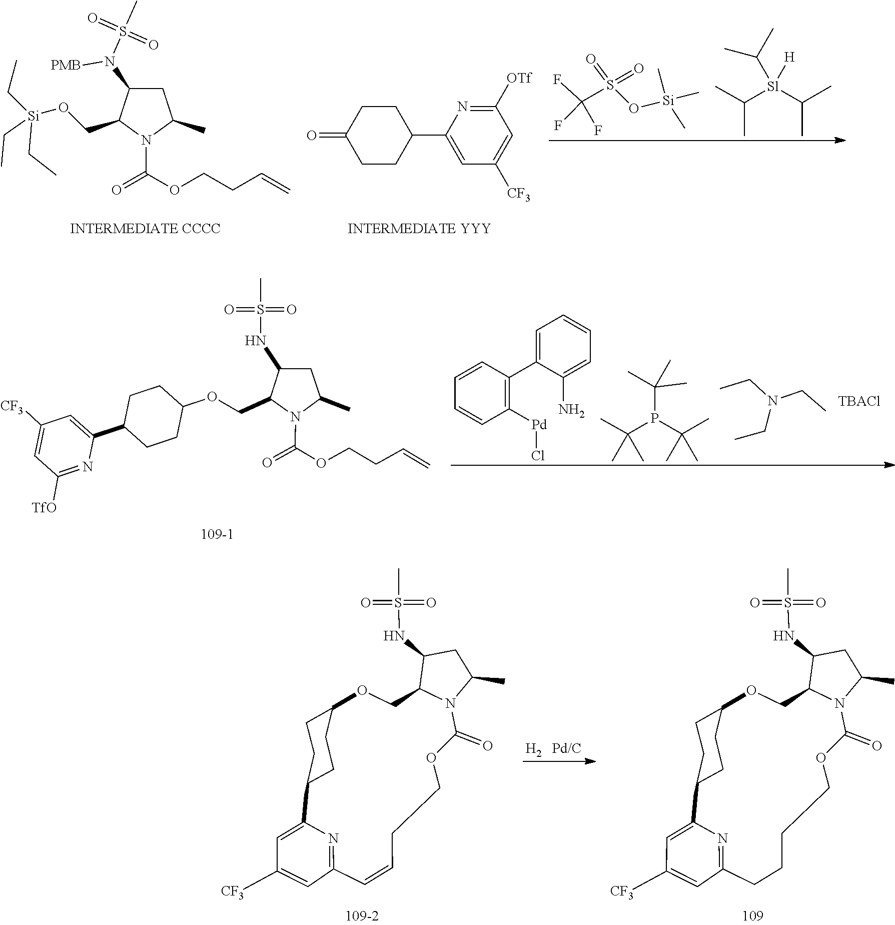

### Step 1: but-3-en-1-yl (2R,3S,5R)-5-methyl-3-(methylsulfonamido)-2-(((4-(4-(trifluoromethyl)-6-(((trifluoromethyl)sulfonyl)oxy)pyridin-2-yl)cyclohexyl)oxy)methyl)pyrrolidine-1-carboxylate (109-1)

To a mixture of INTERMEDIATE CCCC (75 mg, 0.139 mmol) was added acetonitrile (1151 μl). INTERMEDIATE YYY (81 mg, 0.208 mmol) was dissolved in DCM (236 μl) and the solution added. The reaction was cooled to −20° C. and triisopropylsilane (56.8 μl, 0.277 mmol) was added. Trimethylsilyl trifluoromethanesulfonate (25.06 μl, 0.139 mmol) was added dropwise. The reaction was stirred for 30 min. 200 uL of trifluoroacetic acid was added to the mixture and the reaction was stirred for 2 hours at room temperature. The reaction was quenched with saturated NaHCO₃(5 mL), extracted with ethyl acetate (3×5 mL), dried over Na2SO4, and concentrated. The mixture was purified using silica column chromatography (2% to 50% EtOAc/hexanes) to afford the title compound. MS: 682.4 [M+H]⁺.

### Step 2: N-((2¹S,2⁴S,5²R,5³S,5⁵R,Z)-5⁵-methyl-6-oxo-1⁴-(trifluoromethyl)-3,7-dioxa-1(2,6)-pyridina-5(2,1)-pyrrolidina-2(1,4)-cyclohexanacycloundecaphan-10-en-5³-yl)methanesulfonamide (109-2)

To a mixture of 109-1 (57 mg, 0.084 mmol) was added chloro[(tri-tert-butylphosphine)-2-(2-aminobiphenyl)] palladium(II) (21.42 mg, 0.042 mmol), Triethylamine (58.0 μl, 0.418 mmol), tetrabutylammonium chloride (46.5 mg, 0.167 mmol) in DMF (1672 μl). The reaction was sealed and heated at 100 degrees for 24 hours. The solvent was concentrated and the mixture was purified using silica column chromatography (2% to 50% EtOAc/hexanes) to afford the title compound. MS: 532.4 [M+H]⁺.

### Step 3: N-((2¹S,2⁴S,5²R,5³S,5⁵R)-5⁵-methyl-6-oxo-14-(trifluoromethyl)-3,7-dioxa-1(2,6)-pyridina-5(2,1)-pyrrolidina-2(1,4)-cyclohexanacycloundecaphane-5³-yl)methanesulfonamide (109)

To a mixture of 109-2 (8.5 mg, 0.016 mmol) was added MeOH (160 μl) and palladium on carbon (3.40 mg, 3.20 μmol). An H2 balloon was fitted (vacuum purged and backfilled three times) into the vial through the septa. The reaction was stirred at 25 degrees for 18 hours before filtering through celite and washing with MeOH. The reaction was purified using C18 column chromatography (5% to 100% MeCN (0.05% TFA)/H2O (0.05% TFA)) to afford the title compound. MS: 534.4.

The following EXAMPLES 110-111 were prepared according to the general procedures herein and in an analogous manner to that used to synthesize EXAMPLE 109 using the appropriate intermediates. The intermediates were prepared as described in the intermediates section from commercially available or prepared from commercially available reagents using conventional reactions well known in the art.

| | | | Exact Mass |
|---|---|---|---|
| EXAMPLE | Structure | Name | [M + H]⁺ |
| 110 | 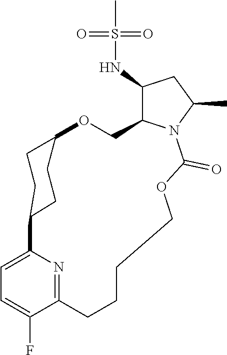 | N-((2¹S,2⁴S,5²R,5³S,5⁵R)-1⁵- fluoro-5⁵-methyl-6-oxo-3,7- dioxa-1(2,6)-pyridina-5(2,1)- pyrrolidina-2(1,4)- cyclohexanacycloundecaphane- 5³-yl)methanesulfonamide | 484.7 |
| 111 | 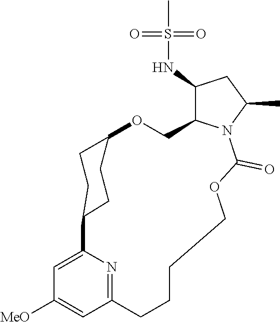 | N-((2¹S,2⁴S,5²R,5³S,5⁵R)-1⁴- methoxy-5⁵-methyl-6-oxo-3,7- dioxa-1(2,6)-pyridina-5(2,1)- pyrrolidina-2(1,4)- cyclohexanacycloundecaphane- 5³-yl)methanesulfonamide | 496.6 |

The following table shows representative data for the compounds of the Examples as orexin receptor agonists as determined by the assays described herein.

| Example | hOX2R_IP IC₅₀ (nM) | Emax (%) |
|---|---|---|
| 1 | 0.09 | 99.8% |
| 2 | 0.11 | 101.7% |
| 3 | 0.14 | 99.5% |
| 4 | 0.37 | 100.8% |
| 5 | 1.81 | 100.1% |
| 6 | 26.1 | 100.5% |
| 7 | 2.2 | 103.6% |
| 8 | 25.2 | 100.3% |
| 9 | 1.3 | 100.4% |
| 10 | 0.30 | 101.4% |
| 11 | 12.8 | 100.6% |
| 12 | 0.34 | 101.0% |
| 13 | 0.20 | 99.3% |
| 14 | 0.18 | 100.0% |
| 15 | 0.10 | 101.1% |
| 16 | 3.8 | 99.1% |
| 17 | 0.18 | 100.5% |
| 18 | 0.14 | 100.0% |
| 19 | 0.30 | 100.6% |
| 20 | 0.10 | 100.9% |
| 21 | 6.7 | 100.5% |
| 22 | 0.23 | 101.9% |
| 23 | 14.3 | 101.5% |
| 24 | 89.0 | 99.2% |
| 25 | 1.9 | 101.0% |
| 26 | 189.3 | 94.1% |
| 27 | >1000 | 4.1% |
| 28 | 50.7 | 102.3% |
| 29 | 6.1 | 101.3% |
| 30 | 1.9 | 100.0% |
| 31 | 968 | 26.2% |
| 32 | 1.3 | 101.4% |
| 33 | >1000 | −3.9% |
| 34 | >1000 | 5.0% |
| 35 | 14.6 | 100.1% |
| 36 | 30.2 | 103.3% |
| 37 | >1000 | 5.8% |
| 38 | 30.7 | 100.8% |
| 39 | >1000 | 4.4% |
| 40 | >1000 | 71.6% |
| 41 | 0.34 | 101.5% |
| 42 | 0.17 | 101.6% |
| 43 | 1.3 | 102.5% |
| 44 | 0.28 | 101.8% |
| 45 | 0.32 | 102.8% |
| 46 | 2.9 | 102.5% |
| 47 | 0.15 | 102.4% |
| 48 | 0.48 | 101.4% |
| 49 | 12.9 | 101.3% |
| 50 | 17.8 | 101.2% |
| 51 | 0.84 | 99.2% |
| 52 | 6.7 | 98.4% |
| 53 | 0.68 | 102.7% |
| 54 | 4.6 | 100.1% |
| 55 | 28.9 | 103.2% |
| 56 | 0.11 | 98.2% |
| 57 | 0.13 | 102.3% |
| 58 | 0.30 | 101.6% |
| 59 | 2.9 | 96.9% |
| 60 | 0.04 | 101.6% |
| 61 | 0.05 | 99.0% |
| 62 | 0.42 | 91.2% |
| 63 | 9.4 | 93.1% |
| 64 | 2.1 | 102.5% |
| 65 | 1.2 | 102.8% |
| 66 | 0.27 | 95.0% |
| 67 | 0.22 | 98.5% |
| 68 | 0.33 | 102.3% |
| 69 | 2.4 | 94.7% |
| 70 | 0.14 | 101.8% |
| 71 | 0.21 | 101.7% |
| 72 | 0.53 | 102.4% |
| 73 | 0.80 | 100.2% |
| 74 | 0.24 | 110.2% |
| 75 | 0.55 | 97.2% |
| 76 | 0.41 | 101.3% |
| 77 | 0.59 | 99.5% |
| 78 | 0.54 | 100.1% |
| 79 | 0.10 | 101.0% |
| 80 | 0.23 | 99.4% |
| 81 | 0.16 | 104.3% |
| 82 | 0.07 | 101.3% |
| 83 | 4.3 | 102.1% |
| 84 | 55.9 | 101.1% |
| 85 | 1.1 | 102.5% |
| 86 | 0.34 | 100.9% |
| 87 | 0.63 | 100.1% |
| 88 | 2.3 | 102.6% |
| 89 | 5.1 | 101% |
| 90 | 0.11 | 101.2% |
| 91 | 0.30 | 101.5% |
| 92 | 0.13 | 98.9% |
| 93 | 0.34 | 103.1% |
| 94 | 437 | 37.5% |
| 95 | 3.9 | 98.4% |
| 96 | 0.32 | 100.1% |
| 97 | 0.68 | 98.4% |
| 98 | 7.4 | 100.1% |
| 99 | 2.3 | 100.0% |
| 100 | 11.7 | 102.2% |
| 101 | 16.7 | 101.6% |
| 102 | 8.8 | 99.8% |
| 103 | 0.21 | 103.0% |
| 104 | 0.14 | 101.8% |
| 105 | 0.14 | 99.7% |
| 106 | 0.38 | 97.3% |
| 107 | 0.40 | 101.0% |
| 108 | 0.41 | 103.5% |
| 109 | 8.6 | 102.4% |
| 110 | 0.99 | 101.8% |
| 111 | 3.4 | 99.6% |

With respect to other compounds such as those disclosed in US 2017/0226137, WO 2017/135306, WO 2018/164191, WO 2018/164192, WO 2019/027003, WO 2019/027058, WO2020/122092, WO2020/122093, WO 2020/158958, U.S. Pat. Nos. 9,527,807, 10,287,305, 10,428,023, or U.S. Pat. No. 10,508,083, it would be desirable that the present compounds exhibit unexpected properties, such as better drug-like properties and better physical and pharmacokinetic properties. For example, in contrast to compounds of US 2017/0226137, WO 2017/135306, WO 2018/164191, WO 2018/164192, WO 2019/027003, WO 2019/027058, WO2020/122092, WO2020/122093, WO 2020/158958, U.S. Pat. Nos. 9,527,807, 10,287,305, 10,428,023, or U.S. Pat. No. 10,508,083, the compounds of the present examples may possess improved potency and/or better metabolic stability and solubility.

As indicated by the data herein, the compounds of the present examples provide unexpected potency as orexin receptor agonists. The distinction in potency as orexin receptor agonists provides greater functional activity and potential for enhanced in vivo efficacy and may provide benefits over other orexin receptor agonists that are known in the art.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula I: 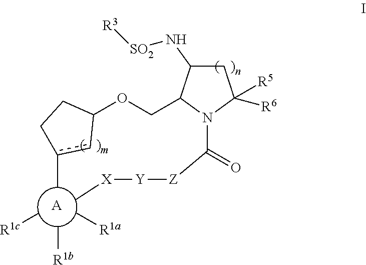
wherein: [symbol]
represents a line that may be absent or present as a double bond;
m is 1 or 2;
n is 1 or 2;
A is a phenyl or pyridyl ring;
X is —O— or —NR—, or X may be a direct bond to Y;
Y is C₁₋₆alkyl or C₂₋₆alkenyl;
Z is —O— or —NR—, or Z may be a direct bond to Y;
R is independently selected from H or C₁₋₆alkyl;
R^{1a}, R^{1b} and R^{1c} as present are independently selected from:
(1) hydrogen,
(2) halogen,
(3) hydroxyl,
(4) C₁₋₆alkyl, which is unsubstituted or substituted with one to three substituents selected from: hydroxy, fluoro and phenyl,
(5) —O—C₁₋₆alkyl, which is unsubstituted or substituted with one to three substituents selected from: fluoro and phenyl,
(6) C₃₋₆cycloalkyl,
(7) C₂₋₆alkynyl,
(8) —NH₂,
(9) —NH(C₁₋₆alkyl),
(10) —N(C₁₋₆alkyl)₂,
(11) —(CO)—O—C₁₋₆alkyl,
(12) keto,
(13) -phenyl,
(14) -pyridyl, and
(15) —CN;
R³ is selected from:
(1) —C₁₋₆alkyl, where the alkyl is unsubstituted or substituted with one to three fluoro,
(2) —C₃₋₆cycloalkyl,
(3) —NH₂,
(4) —NH(C₁₋₆alkyl),
(5) —N(C₁₋₆alkyl)(C₁₋₆alkyl), and
(6) -phenyl;
R⁵ and R⁶ are independently selected from:
(1) hydrogen, and
(2) C₁₋₆alkyl, where the alkyl is unsubstituted or substituted with OR, NR₂, —C(O)NR₂, or one to three fluoro, and
(3) —C₃₋₆cycloalkyl;
or a pharmaceutically acceptable salt thereof.

2. The compound of claim 1 or a pharmaceutically acceptable salt thereof with the structure: 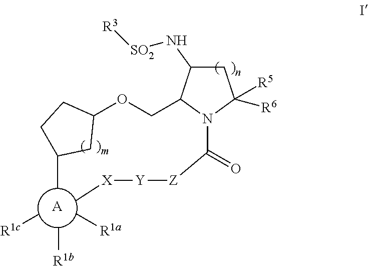
wherein:
m is 1 or 2;
n is 1 or 2;
A is a phenyl or pyridyl ring;
X is —O— or —NH—, or X may be a direct bond to Y;
Y is C₁₋₆alkyl or C₂₋₆alkenyl;
Z is —O— or —NH—, or Z may be a direct bond to Y;
R^{1a}, R^{1b} and R^{1c} as present are independently selected from:
(1) hydrogen,
(2) halogen,
(3) hydroxyl,
(4) C₁₋₆alkyl, which is unsubstituted or substituted with substituents selected from: hydroxy, fluoro and phenyl,
(5) —O—C₁₋₆alkyl, which is unsubstituted or substituted with substituents selected from: fluoro and phenyl,
(6) C₃₋₆cycloalkyl,
(7) C₂₋₆alkynyl,
(8) —NH₂,
(9) —NH(C₁₋₆alkyl),
(10) —N(C₁₋₆alkyl)₂,
(11) —(CO)—O—C₁₋₆alkyl,
(12) keto,
(13) -phenyl,
(14) -pyridyl, and
(15) —CN;
R³ is selected from:
(1) —C₁₋₆alkyl, where the alkyl is unsubstituted or substituted with one to three fluoro,
(2) —C₃₋₆cycloalkyl,
(3) —NH₂,
(4) —NH(C₁₋₆alkyl),
(5) —N(C₁₋₆alkyl)(C₁₋₆alkyl), and
(6) -phenyl;
R⁵ and R⁶ are independently selected from:
(1) hydrogen, and
(2) C₁₋₆alkyl, where the alkyl is unsubstituted or substituted with hydroxyl, —O—C₁₋₆alkyl, or one to three fluoro, and
(3) —C₃₋₆cycloalkyl;
or a pharmaceutically acceptable salt thereof.

3. The compound of claim 1 or a pharmaceutically acceptable salt thereof wherein m is 2.

4. The compound of claim 1 or a pharmaceutically acceptable salt thereof wherein n is 1.

5. The compound of claim 1 or a pharmaceutically acceptable salt thereof wherein A is phenyl or pyridyl.

6. The compound of claim 1 or a pharmaceutically acceptable salt thereof wherein A is 1,2-phenyl, 1,3-phenyl or 2,6-pyridyl.

7. The compound of claim 1 or a pharmaceutically acceptable salt thereof wherein Y is selected from:
(1) —CH₂CH₂—,
(2) —CH₂CH₂CH₂—,
(3) —CH₂CH₂CH₂CH₂—,
(4) —CH₂CH₂CH₂CH₂CH₂—,
(5) —CH═CH—,
(6) —CH═CHCH₂—,
(7) —CH₂CH═CH—,
(8) —CH═CHCH₂CH₂—,
(9) —CH₂CH═CHCH₂—, and
(10) —CH₂CH₂CH═CH—.

8. The compound ofany claim 1 or a pharmaceutically acceptable salt thereof wherein R^{1c} is hydrogen and R^{1a} and R^{1b}, as are present, are independently selected from:
(1) hydrogen,
(2) fluoro,
(3) hydroxyl,
(4) —CH₃,
(5) —CHF₂,
(6) —CF₃,
(7) —CH₂OH,
(8) —CH₂CH₃,
(9) —C(CH₃)OH,
(10) —OCH₃,
(11) —OCHF₂,
(12) —OCH₂CH₂F,
(13) —N(CH₃)₂,
(14) cyclopropyl, and
(15) phenyl.

9. The compound of any of claim 1 or a pharmaceutically acceptable salt thereof wherein R³ is selected from:
(1) methyl,
(2) —CF₃,
(3) —CH₂F,
(4) ethyl,
(5) cyclopropyl,
(6) —CH(CH₃)₂,
(7) —NH(CH₃),
(8) —N(CH₃)₂, and
(9) -phenyl.

10. The compound of any of claim 1 or a pharmaceutically acceptable salt thereof wherein R⁵ is methyl or —CH₂OCH₃, and R⁶ is hydrogen.

11. A compound which is selected from:
N′-((2¹R,2⁴R,5²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)-N,N-dimethyl-sulfamide;
N-((2¹R,2⁴R,5²R,5³S,5⁵R,E)-5⁵-methyl-6-oxo-3,7-dioxa-5(2,1)-pyrrolidina-1(1,2)-benzena-2(1,4)-cyclohexanacyclononaphan-8-en-5³-yl)methanesulfonamide;
N-((2¹R,2⁴R,5²R,5³S,5⁵S,E)-1³-fluoro-5⁵-(methoxymethyl)-6-oxo-3,7-dioxa-5(2,1)-pyrrolidina-1(1,2)-benzena-2(1,4)-cyclohexanacyclononaphan-8-en-5³-yl)methanesulfonamide;
N′-((2¹R,2⁴R,5²R,5³S,5⁵R)-5⁵-methyl-6-oxo-3,7-dioxa-5(2,1)-pyrrolidina-1(1,2)-benzena-2(1,4)-cyclohexanacyclononaphane-5³-yl)-N,N-dimethyl-sulfamide;
N-((2¹R,2⁴R,5²R,5³S,5⁵R)-5⁵-methyl-6-oxo-3,7-dioxa-5(2,1)-pyrrolidina-1(1,2)-benzena-2(1,4)-cyclohexanacyclononaphane-5³-yl)methanesulfonamide;
N-((2¹R,2⁴R,5²R,5³S,55S)-1³-fluoro-5⁵-(methoxymethyl)-6-oxo-3,7-dioxa-5(2,1)-pyrrolidina-1(1,2)-benzena-2(1,4)-cyclohexanacyclononaphane-5³-yl)methanesulfonamide;
N′-((21S,24S,5²R,5³S,5⁵R)-6-oxo-3,11-dioxa-5(2,1)-pyrrolidina-1(1,3)-benzena-2(1,4) cyclohexanacycloundecaphane-5³-yl)-N,N-dimethyl-sulfamide;
N′-((21S,24S,5²R,5³S,5⁵R)-5⁵-methyl-6-oxo-3,12-dioxa-5(2,1)-pyrrolidina-1(1,3)-benzena-2(1,4)-cyclohexanacyclododecaphane-5³-yl)-N,N-dimethyl-sulfamide;
N′-((21S,24S,5²R,5³S, 5⁵R)-5⁵-methyl-6-oxo-3,11-dioxa-5(2,1)-pyrrolidina-1(1,3)-benzena-2(1,4)-cyclohexanacycloundecaphan-7-en-5³-yl)-N,N-dimethyl-sulfamide;
N′-((21S,24S,5²R,5³S, 5⁵R)-5⁵-methyl-6-oxo-3,11-dioxa-5(2,1)-pyrrolidina-1(1,3)-benzena-2(1,4)-cyclohexanacycloundecaphane-5³-yl)-N,N-dimethyl-sulfamide;
N′-((21S,24S,5²R,5³S,5⁵R)-5⁵-methyl-6-oxo-3,7,12-trioxa-5(2,1)-pyrrolidina-1(1,3)-benzena-2(1,4)-cyclohexanacyclododecaphane-5³-yl)-N,N-dimethyl-sulfamide;
N′-((21S,24S,5²R,5³S,5⁵R)-5⁵-methyl-6-oxo-3-oxa-5(2,1)-pyrrolidina-1(1,3)-benzena-2(1,4)cyclohexanacycloundecaphane-5³-yl)-N,N-didimethyl-sulfamide;
N′-((21S,24S,5²R,5³S,5⁵R)-)-5⁵-methyl-6-oxo-3,7,11-trioxa-5(2,1)-pyrrolidina-1(1,3)-benzena-2(1,4)-cyclohexanacycloundecaphane-5³-yl)-N,N-dimethyl-sulfamide;
N′-((21S,24S,5²R,5³S,5⁵R)-5⁵-methyl-6-oxo-3,10-dioxa-5(2,1)-pyrrolidina-1(1,3)-benzena-2(1,4)-cyclohexanacyclodecaphane-5³-yl)-N,N-methyl-sulfamide;
N′-((21S,24S,5²R,5³S,5⁵R)-5⁵-methyl-6-oxo-3,7,10-trioxa-5(2,1)-pyrrolidina-1(1,3)-benzena-2(1,4)-cyclohexanacyclodecaphane-5³-yl)-N,N-dimethyl-sulfamide;
N′-((21S,24S,5²R,5³S)-6-oxo-3,7,11-trioxa-5(2,1)-pyrrolidina-1(1,3)-benzena-2(1,4)-cyclohexanacycloundecaphane-5³-yl)-N,N-dimethyl-sulfamide;
N-((2¹S,2⁴S,5²R,5³S,5⁵R)-5⁵-methyl-6-oxo-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,5³S,5⁵R)-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-3,11-dioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-3,10-dioxa-5(2,1)-pyrrolidina-1(1,3)-benzena-2(1,4)-cyclohexanacyclodecaphane-5³-yl)methanesulfonamide;
N′-((21S,24S,5²R,5³S)-6-oxo-3,7-dioxa-5(2,1)-pyrrolidina-1(1,3)-benzena-2(1,4)-cyclohexanacycloundecaphane-5³-yl)-N,N-dimethyl-sulfamide;
N′-((21S,24S,5²R,5³S,5⁵R)-5⁵-methyl-6-oxo-3,7-dioxa-5(2,1)-pyrrolidina-1(1,3)-benzena-2(1,4)-cyclohexanacycloundecaphane-5³-yl)-N,N-dimethysulfamide;
N′-((21S,24S,5²R,5³S)-6-oxo-3-oxa-11-aza-5(2,1)-pyrrolidina-1(1,3)-benzena-2(1,4)-cyclohexanacycloundecaphane-5³-yl)-N,N-dimethyl-sulfamide;
N-((2¹S,2⁴S,5²R,5³S)-6-oxo-3,1′-dioxa-5(2,1)-piperidina-1(1,2)-benzena-2(1,4)-cyclohexanacycloundecaphane-5³-yl)methanesulfonamide;
N′-((21S,24S,5²R,5³S)-6-oxo-3,7,11-trioxa-5(2,1)-piperidina-1(1,3)-benzena-2(1,4)-cyclohexanacycloundecaphane-5³-yl)-N,N-dimethyl-sulfamide;
N′-((21S,24S,5²R,5³S)-6-oxo-3,7,12-trioxa-5(2,1)-piperidina-1(1,3)-benzena-2(1,4)-cyclohexanacyclododecaphane-5³-yl)-N,N-dimethy-sulfamide;
N′-((2¹R,2⁴R,5²S,5³R)-6-oxo-3,7,12-trioxa-5(2,1)-piperidina-1(1,3)-benzena-2(1,4)-cyclohexanacyclododecaphane-5³-yl)-N,N-dimethyl-sulfamide;
N-((2¹S,2⁴S,5²R,5³S,E)-6-oxo-3,11-dioxa-5(2,1)-piperidina-1(1,3)-benzena-2(1,4)-cyclohexanacycloundecaphan-7-en-5³-yl)methanesulfonamide;
N-((2¹S,2⁴S,5²R,5³S)-6-oxo-3,11-dioxa-5(2,1)-piperidina-1(1,3)-benzena-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹s,2⁴s)-6-oxo-3,11-dioxa-1(2,6)-pyridina-5(2,1)-piperidina-2(1,4)-cyclohexanacycloundecaphane-5³-yl)methanesulfonamide;
N-((2¹S,2³R,5²R,5³S)-6-oxo-3,10-dioxa-5(2,1)-piperidina-1(1,3)-benzena-2(1,3)-cyclopentanacyclodecaphane-5³-yl)methanesulfonamide; and
N′-((21S,24S,5²R,5³S)-6-oxo-3,7-dioxa-5(2,1)-piperidina-1(1,2)-benzena-2(1,4)-cyclohexanacyclononaphane-5³-yl)-N,N-dimethyl-sulfamide;
N-(2⁴S,5²R,5³S,5⁵R)-1⁵fluoro-5⁵-methyl-6-oxo-3,7,11-trioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,53 S,55R)-1⁵-fluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,53 S,5⁵R)-1⁵-fluoro-5⁵-methyl-6-oxo-3,11-dioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,5³S,5⁵R)-1⁵-fluoro-5⁵-methyl-6-oxo-3,11-dioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1⁴-cyano-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-1²-fluoro-5⁵-methyl-6-oxo-3,7,11-trioxa-5(2,1)-pyrrolidina-1(1,3)-benzena-2(1,4)-cyclohexanacycloundecaphan-2¹-en-5³-yl)methanesulfonamide;
N-((2¹S,2⁴S,5²R,5³S,5⁵R)-1²-fluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,53 S,5⁵R)-1²,1⁵-difluoro-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,53 S,5⁵R)-5⁵-methyl-6-oxo-1⁵-(trifluoromethyl)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-1⁶-(trifluoromethoxy)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-14-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁴-(trifluoromethyl)-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,53 S,5⁵R)-5⁵-methyl-6-oxo-16-(trifluoromethoxy)-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,53 S,5⁵R)-5⁵-methyl-6-oxo-1⁴-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁶-fluoro-5⁵-methyl-6-oxo-3,7,11-trioxa-1(3,5)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁵-fluoro-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1³-(trifluoromethoxy)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1³-(trifluoromethoxy)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁶-isopropoxy-5⁵-methyl-6-oxo-3,7,10-trioxa-5(2,1)-pyrrolidina-1(1,3)-benzena-2(1,4)-cyclohexanacyclodecaphane-5³-yl)methanesulfonamide;
N-((2¹S,2⁴S,5²R,5³S,5⁵R)-1⁶-isopropoxy-5⁵-methyl-6-oxo-3,7,11-trioxa-5(2,1)-pyrrolidina-1(1,3)-benzena-2(1,4)-cyclohexanacycloundecaphane-5³-yl)methanesulfonamide;
N-((2¹S,2⁴S,5²R,5³S,5⁵R)-5⁵-methyl-6-oxo-1⁵-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1⁵-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,53S,5⁵R)-1⁴,5⁵-dimethyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,53S,5⁵R)-1⁴,5⁵-dimethyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1³-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁵-(trifluoromethoxy)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-5⁵-methyl-6-oxo-1⁵-(trifluoromethoxy)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-fluoro-1³-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-ethoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-ethoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,53S,5⁵R)-1³,5⁵-dimethyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,53S,5⁵R)-1³,5⁵-dimethyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1³,1⁵-difluoro-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1³,1⁵-difluoro-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵S)-5⁵-(methoxymethyl)-6-oxo-1⁴-(trifluoromethyl)-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵S)-5⁵-(methoxymethyl)-6-oxo-1⁴-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,55S)-14-(difluoromethyl)-5⁵-(methoxymethyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-(difluoromethyl)-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵S)-5⁵-(methoxymethyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁴-methoxy-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,53 S,5⁵R)-5⁵,1¹-dimethyl-6-oxo-1⁴-(trifluoromethyl)-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,53 S,5⁵R)-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5^{5,11}-dimethyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7-dioxa-10-aza-1(2,6)-pyridina-5(2,1)-pyrrolidina-2(1,4)-cyclohexanacyclodecaphane-5³-yl)methanesulfonamide;
N-((2¹S,2⁴S,5²R,5³S,5⁵R)-1⁵-fluoro-5⁵-methyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵,1¹-dimethyl-6-oxo-1³-(trifluoromethyl)-3,7-dioxa-11-aza-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵,1¹-dimethyl-6-oxo-3,7-dioxa-11-aza-1(2,6)-pyridina-5(2,1)-pyrrolidina-2(1,4)-cyclohexanacycloundecaphane-5³-yl)methanesulfonamide;
N-((2¹R,2⁴R,5²R,5³S,5⁵S)-5⁵-(hydroxymethyl)-6-oxo-1³-(trifluoromethyl)-3,7,10-trioxa-1(2,6)-pyridina-5(2,1)-pyrrolidina-2(1,4)-cyclohexanacyclodecaphane-5³-yl)methanesulfonamide;
N-((2¹S,2⁴S,5²R,5³S,55S)-1⁴-(difluoromethyl)-5⁵-(hydroxymethyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹R,2⁴R,5²R,5³S,5⁵S)-5⁵-((dimethylamino)methyl)-6-oxo-13-(trifluoromethyl)-3,7,10-trioxa-1(2,6)-pyridina-5(2,1)-pyrrolidina-2(1,4)-cyclohexanacyclodecaphane-5³-yl) methanesulfonamide;
2-((2¹R,2⁴R,5²R,5³S,5⁵S)-5³-(methylsulfonamido)-6-oxo-1³-(trifluoromethyl)-3,7,10-trioxa-1(2,6)-pyridina-5(2,1)-pyrrolidina-2(1,4)-cyclohexanacyclodecaphane-5⁵-yl)acetamide;
N-((2¹S,2⁴S,5²R,5³S,55S)-1⁴-(difluoromethyl)-5⁵-((dimethylamino)methyl)-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,53 S,5⁵S)-1⁴-(difluoromethyl)-5⁵-((methylamino)methyl)-6-oxo-3,7,11-trioxa-1(2,6)-pyridina-5(2,1)-pyrrolidina-2(1,4)-cyclohexanacycloundecaphane-5³-yl)methanesulfonamide;
2-((21S,2⁴S,5²R,53 S,5⁵S)-1⁴-(difluoromethyl)-5³-(methylsulfonamido)-6-oxo-3,7,11-trioxa-1(2,6)-pyridina-5(2,1)-pyrrolidina-2(1,4)-cyclohexanacycloundecaphane-5⁵-yl)acetamide;
N-((2¹R,2⁴R,5²R, 53S,55S)-5⁵-((dimethylamino)methyl)-6-oxo-13-(trifluoromethyl)-3,7,10-trioxa-1(2,6)-pyridina-5(2,1)-pyrrolidina-2(1,4)-cyclohexanacyclodecaphane-5³-yl)methanesulfonamide;
1-fluoro-N-((2¹R,2⁴R,5²R,5³S,5⁵R)-1⁴-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
1,1-difluoro-N-((2¹R,2⁴R,5²R,5³S,5⁵R)-14-methoxy-5⁵-methyl-6-oxo-3,7,10-trioxa-1(2,6)-pyridina-5(2,1)-pyrrolidina-2(1,4)-cyclohexanacyclodecaphane-5³-yl)methanesulfonamide;
N-((2¹R,2⁴R,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)ethanesulfonamide;
1-fluoro-N-((2¹R,2⁴R,5²R,5³S,5⁵R)-1⁴-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
1,1-difluoro-N-((2¹R,2⁴R,5²R,5³S,5⁵R)-14-methoxy-5⁵-methyl-6-oxo-3,7,11-trioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-5⁵-methyl-6-oxo-1⁴-(trifluoromethyl)-3,7-dioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁵-fluoro-5⁵-methyl-6-oxo-3,7-dioxa-1(2,6)-pyridina-5(2,1)-pyrrolidina-2(1,4)-cyclohexanacycloundecaphane-5³-yl)methanesulfonamide;
N-((2¹S,2⁴S,5²R,5³S,5⁵R)-1⁴-methoxy-5⁵-methyl-6-oxo-3,7-dioxa-1(2,6)-pyridina-5(2,1)-pyrrolidina-2(1,4)-cyclohexanacycloundecaphane-5³-yl)methanesulfonamide;
or a pharmaceutically acceptable salt thereof.

12. A pharmaceutical composition which comprises an inert carrier and a compound of any of claim 1 or a pharmaceutically acceptable salt thereof.

13. A compound of any of claim 1 or a pharmaceutically acceptable salt thereof for use in therapy.

14. (canceled)

15. A method for treating narcolepsy in a mammalian subject which comprises administering to the patient an effective amount of the compound of any of claim 1 or a pharmaceutically acceptable salt thereof.

16. A method for treating hypersomnia in a mammalian subject which comprises administering to the patient an effective amount of the compound of any of claim 1 or a pharmaceutically acceptable salt thereof.
